(19) United States
(12) Patent Application Publication
(10) Pub. No.: US 2006/0293343 A1
(43) Pub. Date: Dec. 28, 2006

(54) Pyrimidine derivatives

(75) Inventors: Kenji Naganuma, Tokyo (JP); Hirotsugu Yokoi, Tokyo (JP)

Correspondence Address: EDWARDS & ANGELL, LLP, P.O. BOX 55874, BOSTON, MA, 02205, US

(73) Assignee: Asahi Kasei Pharma Corporation, Tokyo (JP)

(21) Appl. No.: 11/438,095

(22) Filed: May 18, 2006

Related U.S. Application Data
(60) Provisional application No. 60/682,905, filed on May 19, 2005.
(60) Provisional application No. 60/738,212, filed on Nov. 18, 2005.

(30) Foreign Application Priority Data
May 18, 2005 (JP) 2005-145865

(51) Int. Cl. A61K 31/506; A61K 31/505; C07D 409/02; C07D 403/02

(52) U.S. Cl. 514/256; 544/326; 544/328

(57) ABSTRACT

The object of the invention is to provide a novel compound having an inhibitory action on PDE4 activity with fewer side effects.
The invention provides a compound represented by the following general formula (1), possible stereoisomers thereof or racemates thereof, pharmacologically acceptable salts thereof, hydrates thereof, solvated compounds thereof, or prodrugs thereof,

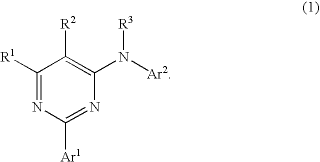

### TECHNICAL FIELD

The invention relates to a novel pyrimidine derivative having anti-inflammatory activity and being useful as an active ingredient of medicines.

### BACKGROUND OF THE INVENTION

Extracellular stimulation by neurotransmitters and hormones is transmitted into cells via a receptor on a cell membrane. This biological signal is known to exhibit its action by increasing the concentrations of cyclic adenosine-3′,5′-monophosphate (cAMP) and cyclic guanosine-3′,5′ monaphosphate (cGMP) as intracllular messengers. Nucleotide phosphodiesterase (PDE) serves for regulating these concentrations by decomposing cAMP and cGMP. PDEs are currently categorized into at least 11 kinds from the difference of gene sequences and pharmacological characteristics (non-patent documents 1 and 2). Type 4 PDE (PDE4) among them selectively decomposes cAMP to reduce the intracellular concentration of cAMP. The presence of PDE4 is recognized in many tissues and cells such as the blood vessel, heart, intestine, brain, pancreas, bronchial smooth muscle, white blood cell and lymphocyte. cAMP having an increased level by inhibiting PDE4 in these cites is known to exhibit an effect for preventing-or improving many diseases.

PDE4 inhibitors suppress activation of inflammatory cells, have many effects such as suppression of release of tumor necrosis factor α (TNF-α), suppression of degranulation reactions, suppression of release of inflammatory cytokines and suppression of chemotaxis (non-patent documents 3 to 5), and are considered to be effective for many inflammatory-related diseases, allergy-related diseases, and immunity-related diseases. For example, the PDE4 inhibitor has been recognized to be effective in respiratory organ-related diseases such as asthma, chronic obstructive pulmonary diseases (COPD), acute bronchitis, chronic bronchitis, inflammatory respiratory tract diseases, pulmonary emphysema and adult respiratory distress syndrome (ARDS), and effectiveness of roflumilast and cilomilast as PDE4 inhibitors has been confirmed particularly for patients with COPD and bronchitis in clinical tests (non-patent document 6). PDE4 has been also reported to be effective in joint-related diseases such as rheumatism, osteoarthritis, acute arthritis and chronic arthritis; skin-related diseases such as atopic dermatitis, psoriasis, seborrheic eczema and allergic contact eczema; gastrointestine-related diseases such as hypersensitive colitis, ulcerative colitis and Crohn's disease; eye and nose-related diseases such as allergic rhinitis, chronic rhinitis and allergic conjunctivitis; and immune system-related diseases such as transplant rejection response, multiple sclerosis and AIDS (non-patent documents 7 to 12). Since PDE4 inhibition at the bronchial smooth muscle has been known to relax the smooth muscle, the PDE4 inhibitor has been considered to be effective for respiratory organ diseases such as COPD.

On the other hand, the PDE4 inhibitor may cause unfavorable side effects such as vomiting. Side effects such as vomiting and nausea have been reported in the clinical test of rolipram and cilomilast. One of the causes of such side effect is selectivity among PDE4 isoforms. Four isoforms (A, B, C and D) have been reported in PDE4, where isoform B is related to pharmaceutical activities such as anti-inflammatory activity, while isoform D is suggested to be related to side effects such as vomiting and nausea (non-patent document 13).

While the PDE4 inhibitor has been reported to be effective for many diseases (patent documents 1 to 3), the currently reported PDE4 inhibitor is not satisfactory with respect to its effect and side effect.

Patent Document 1: WO98/45268

Patent Document 2: WO95/01338

Patent Document 3: WO99/55696

Non-Patent Document 1: Soderling, S. H. and Beavo, J. A., Curr. Opin. Cell Biol., 12, p 174-179, 2000

Non-Patent Document 2: Hetman, J. M. et al., Proc. Natl. Acad. Sci. USA, 97, p 12891-12895, 2000

Non-Patent Document 3: Teixeira, M. M. et al., Trends Pharmacol. Sci., 18, p 164-170, 1997

Non-Patent Document 4: Beavo, J. A. et al., Trends Pharmacol. Sci., 11, p 150-155, 1990

Non-Patent Document 5: Nicholson, C. D et al., Trends Pharmacol. Sci., 12, p 19-27, 1991

Non-Patent Document 6: Lipworth, B. J., Lancet, 365, p 167-175, 2005

Non-Patent Document 7: Dyke, H. J. and Montana, J. G., Expert Opin. Investig. Drugs, 11. p 1-13, 2002

Non-Patent Document 8: Burnouf, C., et al., Curr. Pharm. Des., 8, p 1255-1296, 2002

Non-Patent Document 9: Banner, K. H. and Trevethick, M. A., Trends Pharmacol. Sci., 25, p 430-436, 2004

Non-Patent Document 10: Rickards, K. J., et al., Vet. Immunol, Immunopathol., 98, p 153-165, 2004

Non-Patent Document 11: Sun, Y., et al., J. Immunol., 165, p 1755-1761, 2000

Non-Patent Document 12: Dousa, M. K., et al., Clin. Nephrol., 47, p 187-189, 1997

Non-Patent Document 13: Lipworth, B. J., Lancet, 365, p 167-175, 2005

The problem of the invention is to provide a novel substance having a PDE4 activity inhibitory action. More specifically, the problem of the invention is to provide a PDE4 activity inhibitory action with fewer side effects.

The inventors of the invention have found, through intensive studies for solving the above-mentioned problems, that novel compounds represented by the following general formula (1) have an excellent anti-inflammatory activity. The invention has been completed based on the discovery as described above.

The invention provides:

[A1] a compound represented by the following general formula (1), possible stereoisomers thereof or racemates thereof, pharmacologically acceptable salts thereof, hydrates thereof, solvated compounds thereof, or pro-drugs thereof, 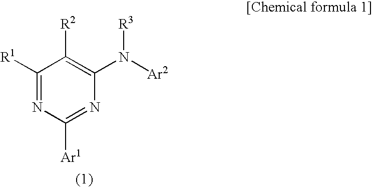
where, in general formula (1), Ar¹ represents a furyl group, thienyl group, triazolyl group, thiazolyl group, oxazolyl group or benzothiazolyl group; Ar¹ may be substituted or non-substituted; Ar² represents -E-Ar²¹-G-Q (Ar²¹ represents a benzene ring or naphthalene ring; E represents a single bond or an alkylene group; G represents a single bond, an alkylene group or an alkenylene group; Q represents a carboxycarboxy group, —CON(R⁴¹) (R⁴²) (R⁴¹ and R⁴² may be the same or different, each independently represents a hydrogen atom, a hydroxy group, an alkyl group that may be substituted or an aryl group that may be substituted, or R⁴¹ and R⁴² are combined to form a 3 to 7 membered ring that represents a cyclic amine as (R⁴¹) (R⁴²), where R⁴² is a group other than a hydroxy group when R⁴¹ is the hydroxy group), or —COOR⁴³ (R⁴³ represents an alkyl group that may be substituted or an aryl group that may be substituted)), E-Ar²¹-G²-G-Q (E, Ar²¹, G and Q are the same as described above, and G² represents —O—, —S—, SO—, SO₂ or —NR^{G21}— (R^{G21} represents a hydrogen atom, an alkyl group that may be substituted, an acyl group that may be substituted, or a sulfonyl group that may be substituted)), or a monocyclic heteroaromatic ring except the pyrazolyl group; Ar² may be substituted; R¹ and R² may be the same or different, and each independently represents a hydrogen atom, an alkyl group that may be substituted, an alkenyl group that may be substituted, an alkynyl group that may be substituted, an alkoxy group that may be substituted, an alkylthio group that may be substituted, an alkylsulfinyl group that may be substituted, or an alkylsulfonyl group that may be substituted, and R³ represents a hydrogen atom or an alkyl group that may be substituted;

[A2] the compound according to [A1] in which Ar¹ is a furyl group or a thienyl group that may be substituted, possible stereoisomers thereof or racemates thereof, or pharmacologically acceptable salts thereof, hydrates, solvated compounds thereof, or prodrugs thereof;

[A3] the compound according to [A1] or [A2], possible stereoisomers thereof or racemates thereof, or pharmacologically acceptable salts thereof, hydrates, solvated compounds thereof, or prodrugs thereof, wherein Ar¹ may be substituted with one or a plurality of groups each independently selected from a group consisting of a halogen atom, an alkylthio group that may be substituted, an alkoxy group that may be substituted, an alkylthio group that may be substituted, an amino group that may be substituted, and an acyl group that may be substituted;

[A4] the compound according to [A1] or [A2], possible stereoisomers thereof or racemates thereof, or pharmacologically acceptable salts thereof, hydrates, solvated compounds thereof, or prodrugs thereof, wherein Ar¹ may be substituted with one or a plurality of groups each independently selected from a group consisting of a halogen atom, an alkyl group that may be substituted, an alkoxy group that may be substituted, and an amino group that may be substituted;

[A5] the compound according to [A1] or [A2], possible stereoisomers thereof or racemates thereof, or pharmacologically acceptable salts thereof, hydrates, solvated compounds thereof, or prodrugs thereof, wherein Ar¹ may be substituted with one or a plurality of groups each independently selected from a group consisting of a halogen atom, a lower alkyl group, a trifluoromethyl group, a hydroxymethyl group, a hydroxyethyl group, a lower alkoxy group, a trifluoromethoxy group, a 2-methoxyethoxy group, a —NH₂ group, a lower alkylamino group, a lower dialkylamino group, an acylamino group and a lower alkylsulfonylamino group;

[A6] the compound according to [A1] or [A2], possible stereoisomers thereof or racemates thereof, or pharmacologically acceptable salts thereof, hydrates, solvated compounds thereof, or prodrugs thereof, wherein Ar¹ may be substituted with one or a plurality of halogen atoms;

[A7] the compound according to [A1] or [A2], possible stereoisomers thereof or racemates thereof, or pharmacologically acceptable salts thereof, hydrates, solvated compounds thereof, or prodrugs thereof, wherein Ar¹ is substituted with one or a plurality of halogen atoms;

[A8] the compound according to [A1] or [A2], possible stereoisomers thereof or racemates thereof, or pharmacologically acceptable salts thereof, hydrates, solvated compounds thereof, or prodrugs thereof, wherein Ar¹ is non-substituted;

[A9] the compound according to any one of [A1] to [A8], possible stereoisomers thereof or racemates thereof, or pharmacologically acceptable salts thereof, hydrates, solvated compounds thereof, or prodrugs thereof, wherein R¹ represents a lower alkyl group;

[A10] the compound according to any one of [A1] to [A9], possible stereoisomers thereof or racemates thereof, or pharmacologically acceptable salts thereof, hydrates, solvated compounds thereof, or prodrugs thereof, wherein R² represents a lower alkyl group or a lower alkenyl group;

[A11] the compound according to any one of [A1] to [A9], possible stereoisomers thereof or racemates thereof, or pharmacologically acceptable salts thereof, hydrates, solvated compounds thereof, or prodrugs thereof, wherein R² represents a lower alkyl group;

[A12] the compound according to any one of [A1] to [A11], possible stereoisomers thereof or racemates thereof, or pharmacologically acceptable salts thereof, hydrates, solvated compounds thereof, or prodrugs thereof, wherein R³ represents a hydrogen atom;

[A13] the compound according to any one of [A1] to [A12], possible stereoisomers thereof or racemates thereof, or pharmacologically acceptable salts thereof, hydrates, solvated compounds thereof, or prodrugs thereof, wherein Ar² represents -E-Ar²¹-G-Q (E, Ar²¹, G and Q are the same as described above). [A14] the compound according to any one of [A1] to [A13], possible stereoisomers thereof or racemates thereof, or pharmacologically acceptable salts thereof, hydrates, solvated compounds thereof, or prodrugs thereof, wherein Ar²¹ represents a benzene ring;

[A15] the compound according to any one of [A1] to [A14], possible stereoisomers thereof or racemates thereof, or pharmacologically acceptable salts thereof, hydrates, solvated compounds thereof, or prodrugs thereof, wherein E represents a single bond, and G represents a single bond or a lower alkylene group;

[A16] the compound according to any one of [A1] to [A14], possible stereoisomers thereof or racemates thereof, or pharmacologically acceptable salts thereof, hydrates, solvated compounds thereof, or prodrugs thereof, wherein E represents a single bond and G represents a lower alkylene group;

[A17] the compound according to any one of [A1] to [A16], possible stereoisomers thereof or racemates thereof, or pharmacologically acceptable salts thereof, hydrates, solvated compounds thereof, or prodrugs thereof, wherein Q represents a carboxy group;

[A18] the compound according to any one of [A1] to [A17], possible stereoisomers thereof or racemates thereof, or pharmacologically acceptable salts thereof, hydrates, solvated compounds thereof, or prodrugs thereof, wherein Ar may be substituted with one or a plurality of groups independently selected from a group consisting of a hydroxy group, a halogen atom, an alkyl group that may be substituted and an alkoxy group that may be substituted;

[A19] the compound according to any one of [A1] to [A17], possible stereoisomers thereof or racemates thereof, or pharmacologically acceptable salts thereof, hydrates, solvated compounds thereof, or prodrugs thereof, wherein Ar² may be substituted with one or a plurality of groups independently selected from a group consisting of a hydroxy group, a halogen atom, a lower alkyl group, a trifluoromethyl group, a lower alkoxy group and a trifluoromethoxy group:

[A20] the compound according to any one of [A1] to [A17], possible stereoisomers thereof or racemates thereof, or pharmacologically acceptable salts thereof, hydrates, solvated compounds thereof, or prodrugs thereof, wherein Ar² may be substituted with one or a plurality of halogen atoms;

[A21] the compound according to any one of [A1] to [A17], possible stereoisomers thereof or racemates thereof, or pharmacologically acceptable salts thereof, hydrates, solvated compounds thereof, or prodrugs thereof, wherein Ar² is substituted with one or a plurality of halogen atoms;

[A22] the compound according to any one of [A1] to [A17], possible stereoisomers thereof or racemates thereof, or pharmacologically acceptable salts thereof, hydrates, solvated compounds thereof, or prodrugs thereof, wherein Ar² is non-substituted;

[A23] the compound according to [A1], possible stereoisomers thereof or racemates thereof, or pharmacologically acceptable salts thereof, hydrates, solvated compounds thereof, or prodrugs thereof, wherein Ar¹ represents a thienyl group, Ar¹ may be substituted with one or a plurality of groups independently selected from a group consisting of a halogen atom, an alkyl group that may be substituted, an alkoxy group that may be substituted, an alkylthio group that may be substituted, an amino group that may be substituted, and an acyl group that may be substituted, Ar² represents -E-Ar²¹-G-Q, Ar²¹ represents a benzene ring or a benzene ring substituted with one or a plurality of halogen atoms, E represents a single bond, G represents a methylene group, Q represents a carboxy group, Ar² has no further substituents, R¹ represents a methyl or an ethyl group, R² represents an aryl group, an ethyl group or a hydroxymethyl group, and R³ represents a hydrogen atom;

[A24] the compound according to [A1], possible stereoisomers thereof or racemates thereof, or pharmacologically acceptable salts thereof, hydrates, solvated compounds thereof, or prodrugs thereof, wherein Ar¹ represents a thienyl group, Ar¹ may be substituted with one or a plurality of groups independently selected from a group consisting of a halogen atom, an alkyl group that may be substituted, an alkoxy group that may be substituted, an alkylthio group that may be substituted, an amino group that may be substituted, and an acyl group that may be substituted, Ar² represents -E-Ar²¹-G-Q, Ar²¹ represents a benzene ring, E represents a single bond, G represents a methylene group, Q represents a carboxy group, Ar² has no further substituents, R¹ represents a methyl or an ethyl group, R² represents an aryl group, an ethyl group or a hydroxymethyl group, and R³ represents a hydrogen atom;

[A25] the compound according to [A1], possible stereoisomers thereof or racemates thereof, or pharmacologically acceptable salts thereof, hydrates, solvated compounds thereof, or prodrugs thereof, wherein Ar¹ represents a thienyl group, Ar¹ may be substituted with one or a plurality of groups independently selected from a group consisting of a halogen atom, an alkyl group that may be substituted, an alkoxy group that may be substituted, an alkylthio group that may be substituted, an amino group that may be substituted, and an acyl group that may be substituted, Ar² represents -E-Ar²¹-G-Q, Ar²¹ represents a benzene ring, E represents a single bond, G represents a methylene group, Q represents a carboxy group, Ar² has no further substituents, R¹ represents a methyl or an ethyl group, R² represents an aryl group, and R³ represents a hydrogen atom;

[A26] the compound according to [A1], possible stereoisomers thereof or racemates thereof, or pharmacologically acceptable salts thereof, hydrates, solvated compounds thereof, or prodrugs thereof, wherein Ar¹ represents a thienyl group, Ar¹ may be substituted with one or a plurality of groups independently selected from a group consisting of a halogen atom, an alkyl group that may be substituted, an alkoxy group that may be substituted, an alkylthio group that may be substituted, an amino group that may be substituted, and an acyl group that may be substituted, Ar² represents -E-Ar²¹-G-Q, Ar²¹ represents a benzene ring, E represents a single bond, G represents a methylene group, Q represents a carboxy group, Ar² has no further substituents, R¹ represents a methyl or an ethyl group, R² represents an ethyl group, and R represents a hydrogen atom;

[A27] the compound according to [A1], possible stereoisomers thereof or racemates thereof, or pharmacologically acceptable salts thereof, hydrates, solvated compounds thereof, or prodrugs thereof, wherein Ar¹ represents a thienyl group, Ar¹ may be substituted with one or a plurality of groups independently selected from a group consisting of a halogen atom, an alkyl group that may be substituted, an alkoxy group that may be substituted, an alkylthio group that may be substituted, an amino group that may be substituted, and an acyl group that may be substituted, Ar² represents -E-Ar²¹-G-Q, Ar²¹ represents a benzene ring or a benzene ring substituted with a plurality of halogen atoms, E represents a single bond, G represents a methylene group, Q represents a carboxy group, Ar² has no further substituents, R¹ represents a methyl or an ethyl group, R² represents a hydroxymethyl group, and R³ represents a hydrogen atom;

[A28] the compound according to [A1], possible stereoisomers thereof or racemates thereof, or pharmacologically acceptable salts thereof, hydrates, solvated compounds thereof, or prodrugs thereof, wherein Ar¹ represents a thienyl group, Ar¹ may be substituted with one or a plurality of groups independently selected from a group consisting of a halogen atom, an alkyl group that may be substituted, an alkoxy group that may be substituted, an alkylthio group that may be substituted, an amino group that may be substituted, and an acyl group that may be substituted, Ar² represents -E-Ar²¹-G-Q, Ar²¹ represents a benzene ring, E represents a single bond, G represents a methylene group, Q represents a carboxy group, Ar² has no further substituents, R¹ represents a methyl or an ethyl group, R² represents a hydroxymethyl group, and R³ represents a hydrogen atom;

[A29] the compound according to [A1], possible stereoisomers thereof or racemates thereof, or pharmacologically acceptable salts thereof, hydrates, solvated compounds thereof, or prodrugs thereof, wherein Ar¹ represents a thienyl group, Ar¹ may be substituted with one or a plurality of groups independently selected from a group consisting of a halogen atom, an alkyl group that may be substituted, an alkoxy group that may be substituted, an alkylthio group that may be substituted, an amino group that may be substituted, and an acyl group that may be substituted, Ar² represents -E-Ar²¹-G-Q, Ar²¹ represents a benzene ring substituted with one or a plurality of halogen atoms, E represents a single bond, G represents a methylene group, Q represents a carboxy group, Ar² has no further substituents, R¹ represents a methyl or an ethyl group, R² represents a hydroxymethyl group, and R³ represents a hydrogen atom;

[A30] the compound according to any one of [A1] to [A29], possible stereoisomers thereof or racemates thereof, or pharmacologically acceptable salts thereof, hydrates, solvated compounds thereof, or prodrugs thereof, wherein Ar¹ may be substituted with one or a plurality of groups each independently selected from a group consisting of a halogen atom, an alkyl group that may be substituted, an alkoxy group that may be substituted, and an amino group that may be substituted;

[A31] the compound according to any one of [A1] to [A29], possible stereoisomers thereof or racemates thereof, or pharmacologically acceptable salts thereof, hydrates, solvated compounds thereof, or prodrugs thereof, wherein Ar¹ may be substituted with one or a plurality of groups each independently selected from a group consisting of a halogen atom, a lower alkyl group, a trifluoromethyl group, a hydroxymethyl group, hydroxyethyl group, a lower alkoxy group, a trifluoromethoxy group, a 2-methoxyethoxy group, a —NH₂ group, a lower alkylamino group, a lower dialkylamino group, an acylamino group and a lower alkylsulfonylamino group;

[A32] the compound according to any one of [A1] to [A29], possible stereoisomers thereof or racemates thereof, or pharmacologically acceptable salts thereof, hydrates, solvated compounds thereof, or prodrugs thereof, wherein Ar¹ may be substituted with one or a plurality of groups each independently selected from a group consisting of a halogen atom, a lower alkyl group, a trifluoromethyl group, a lower alkoxy group, a trifluoromethoxy group, a 2-methoxyethoxy group, a —NH₂ group, a lower alkylamino, a lower dialkylamino group and an acylamino group;

[A33] the compound according to any one of [A1] to [A29], possible stereoisomers thereof or racemates thereof, or pharmacologically acceptable salts thereof, hydrates, solvated compounds thereof, or prodrugs thereof, wherein Ar¹ may be substituted with one or a plurality of halogen atoms;

[A34] the compound according to any one of [A1] to [A7] or [A9] to [A29], possible stereoisomers thereof or racemates thereof, or pharmacologically acceptable salts thereof, hydrates, solvated compounds thereof, or prodrugs thereof, wherein Ar¹ is substituted with one or a plurality of halogen atoms;

[A35] the compound according to any one of [A1] to [A29], possible stereoisomers thereof or racemates thereof, or pharmacologically acceptable salts thereof, hydrates, solvated compounds thereof, or prodrugs thereof, wherein Ar¹ is non-substituted;

[A36] a medicine comprising the compound according to any one of [A1] to [A35], possible stereoisomers thereof or racemates thereof, or pharmacologically acceptable salts thereof, hydrates, solvated compounds thereof, or prodrugs thereof as an active ingredient;

[A37] the medicine according to [A36] as a preventive and/or therapeutic agent of inflammatory diseases;

[A38] the medicine according to [A36] as a preventive and/or therapeutic agent of chronic obstructive lung diseases;

[A39] the medicine according to [A36] to [A38] as an inhibitor of PDE4 activity;

[A40] an inhibitor of PDE4 activity comprising the compound according to any one of [A1] to [A35], possible stereoisomers thereof or racemates thereof, or pharmacologically acceptable salts thereof, hydrates, solvated compounds thereof, or prodrugs thereof as an active ingredient;

[A41] the compound according to any one of [A1] to [A35], possible stereoisomers thereof or racemates thereof, or pharmacologically acceptable salts thereof, hydrates, solvated compounds thereof, or prodrugs thereof for producing the medicine according to any one of [A36] to [A39];

[A42] a method for preventing and/or treating inflammatory diseases-comprising the step of administering the compound according to any one of [A1] to [A35], possible stereoisomers thereof or racemates thereof, or pharmacologically acceptable salts thereof, hydrates, solvated compounds thereof, or prodrugs thereof to mammals including a human in an amount effective for prevention and/or treatment;

[A43] a method for inhibiting the PDE4 activity in the body of mammals including a human, comprising the step of administering an effective amount of the compound according to any one of [A1] to [A35], possible stereoisomers thereof or racemates thereof, or pharmacologically acceptable salts thereof, hydrates, solvated compounds thereof, or prodrugs thereof to the mammals including the human;

[B1] a compound represented by the following general formula (1), possible stereoisomers thereof or racemates thereof, pharmacologically acceptable salts thereof, hydrates thereof, solvated compounds thereof, or pro-drugs thereof,

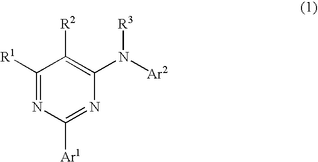

where, in general formula (1), Ar¹ represents a furyl group, a thienyl group, a triazolyl group, a thiazolyl group, an oxazolyl group or a benzothiazolyl group; Ar¹ may be substituted, Ar² represents -E-Ar²¹-G-Q (Ar²¹ represents a benzene ring or a naphthalene ring; E represents a single bond or an alkylene group; G represents a single bond, an alkylene group or an alkenylene group; Q represents a carboxy group, —CON(R⁴¹)(R⁴²) (R⁴¹ and R⁴²may be the same or different, each independently represents a hydrogen atom, a hydroxy group, an alkyl group that may be substituted or an aryl group that may be substituted, or R⁴¹ and R⁴² are combined to form a 3 to 7 membered ring representing a cyclic amine as N(R⁴¹) (R⁴²), wherein R⁴² is a group other than a hydroxy group when R⁴¹ represents the hydroxy group)) or —COOR⁴³ (R⁴³ represents an alkyl group that may be substituted or an aryl group that may be substituted)) or a monocyclic heteroaromatic ring other than the pyrazolyl group; Ar² may be substituted; R¹ and R² may be the same or different, and each independently represents a hydrogen atom, an alkyl group that may be substituted, an alkenyl group that may be substituted, or an alkynyl group that may be substituted; and R³ represents a hydrogen atom or an alkyl group that may be substituted;

[B2] the compound according to [B1], possible stereoisomers thereof or racemates thereof, pharmacologically acceptable salts thereof, hydrates thereof, solvated compounds thereof, or pro-drugs thereof, wherein Ar¹ represents a furyl group or a thienyl group that may be substituted;

[B3] the compound according to [B1] or [B2], possible stereoisomers thereof or racemates thereof, pharmacologically acceptable salts thereof, hydrates thereof, solvated compounds thereof, or pro-drugs thereof, wherein Ar¹ may be substituted with one or a plurality of groups each independently selected from a group consisting of a halogen atom, an alkyl group that may be substituted, an alkoxy group that may be substituted, an alkylthio group that may be substituted and an acyl group that may be substituted;

[B4] the compound according to any one of [B1] to [B3], possible stereoisomers thereof or racemates thereof, pharmacologically acceptable salts thereof, hydrates thereof, solvated compounds thereof, or pro-drugs thereof, wherein R¹ represents a lower alkyl group;

[B5] the compound according to any one of [B1] to [B4], possible stereoisomers thereof or racemates thereof, pharmacologically acceptable salts thereof, hydrates thereof, solvated compounds thereof, or pro-drugs thereof, wherein R² represents a lower alkyl group or a lower alkenyl group;

[B6] the compound according to any one of [B1] to [B5], possible stereoisomers thereof or racemates thereof, pharmacologically acceptable salts thereof, hydrates thereof, solvated compounds thereof, or pro-drugs thereof, wherein R² represents a lower alkyl group;

[B7] the compound according to any one of [B1] to [B5], possible stereoisomers thereof or racemates thereof, pharmacologically acceptable salts thereof, hydrates thereof, solvated compounds thereof, or pro-drugs thereof, wherein R² represents a lower alkenyl group;

[B8] the compound according to any one of [B1] to [B7], possible stereoisomers thereof or racemates thereof, pharmacologically acceptable salts thereof, hydrates thereof, solvated compounds thereof, or pro-drugs thereof, wherein R³ represents a hydrogen atom;

[B9] the compound according to any one of [B1] to [B8], possible stereoisomers thereof or racemates thereof, pharmacologically acceptable salts thereof, hydrates thereof, solvated compounds thereof, or pro-drugs thereof, wherein Ar² represents -E-Ar²¹-G-Q;

[B10] the compound according to any one of [B1] to [B9], possible stereoisomers thereof or racemates thereof, pharmacologically acceptable salts thereof, hydrates thereof, solvated compounds thereof, or pro-drugs thereof, wherein Ar²¹ represents a benzene ring;

[B11] the compound according to any one of [B1] to [B10], possible stereoisomers thereof or racemates thereof, pharmacologically acceptable salts thereof, hydrates thereof, solvated compounds thereof, or pro-drugs thereof, wherein E represents a single bond, and G represents a single bond or a lower alkylene group;

[B12] the compound according to any one of [B1] to [B11], possible stereoisomers thereof or racemates thereof, pharmacologically acceptable salts thereof, hydrates thereof, solvated compounds thereof, or pro-drugs thereof, wherein E represents a single bond, and G represents a lower alkylene group;

[B13] the compound according to any one of [B1] to [B12], possible stereoisomers thereof or racemates thereof, pharmacologically acceptable salts thereof, hydrates thereof, solvated compounds thereof, or pro-drugs thereof, wherein Q represents a carboxy group;

[B14] the compound according to any one of [B1] to [B13], possible stereoisomers thereof or racemates thereof, pharmacologically acceptable salts thereof, hydrates thereof, solvated compounds thereof, or pro-drugs thereof, wherein Ar² may be substituted with one or a plurality of groups independently selected from a group consisting of a hydroxy group, a halogen atom, an alkyl group that may be substituted and an alkoxy group that may be substituted;

[B15] the compound according to [B1], possible stereoisomers thereof or racemates thereof, pharmacologically acceptable salts thereof, hydrates thereof, solvated compounds thereof, or pro-drugs thereof, wherein Ar¹ represents a thienyl group, Ar¹ may be substituted with one or a plurality of groups each independently selected from a group consisting of a halogen atom, an alkyl group that may be substituted, an alkoxy group that may be substituted, an alkylthio group that may be substituted and an acyl group that may be substituted, Ar² represents -E-Ar²¹-G-Q, Ar²¹ represents a benzene ring, E represents a single bond, G represents a methylene group, Q represents a carboxy group, Ar² has no further groups, R¹ represents a methyl or ethyl group, R² represents an allyl group or an ethyl radial, and R³ represents a hydrogen atom;

[B16] a medicine comprising the compound according to any one of [B1] to [B15], possible stereoisomers thereof or racemates thereof, pharmacologically acceptable salts thereof, hydrates thereof, solvated compounds thereof, or pro-drugs thereof as an active ingredient;

[B17] the medicine according to [B16] as a preventive and/or a therapeutic agent of inflammatory diseases;

[B18] the medicine according to [B16] or [B17] as an inhibitor of PDE4 activity;

[B19] an inhibitor of PDE4 activity comprising the compound according to any one of [B1] to [B15], possible stereoisomers thereof or racemates thereof, pharmacologically acceptable salts thereof, hydrates thereof, solvated compounds thereof, or pro-drugs thereof as an active ingredient;

[B20] use of the compound according to any one of [B1] to [B15], possible stereoisomers thereof or racemates thereof, pharmacologically acceptable salts thereof, hydrates thereof, solvated compounds thereof, or pro-drugs thereof for producing the medicine according to any one of [B16] to [B18];

[B21] a method for preventing or treating inflammatory diseases comprising the step of administering the compound according to any one of [B1] to [B15], possible stereoisomers thereof or racemates thereof, pharmacologically acceptable salts thereof, hydrates thereof, solvated compounds thereof, or pro-drugs thereof to mammals including a human in an amount effective for prevention and/or treatment;

[B22] a method for inhibiting the PDE4 activity in the body of mammals including a human comprising the step of administering an effective amount of the compound according to any one of [B1] to [B15], possible stereoisomers thereof or racemates thereof, pharmacologically acceptable salts thereof, hydrates thereof, solvated compounds thereof, or pro-drugs thereof to mammals including a human;

[C1] a compound represented by general formula (1), possible stereoisomers thereof or racemates thereof, pharmacologically acceptable salts thereof, hydrates thereof, solvated compounds thereof, or pro-drugs thereof:

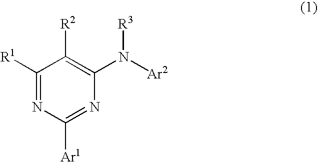

where, in the formula (1), Ar¹ represents a furyl group, a thienyl group, a triazolyl group, a thiazolyl group, an oxazolyl group or a bemzothiazolyl group; Ar¹ may be substituted; Ar² represents a carboxyphenyl group, a carboxyalkylphenyl group, a carboxynaphthyl group, a carboxyalkylnaphthyl group or a monocyclic heteroaromatic ring other than a pyrazolyl group; Ar² may be substituted; R¹ and R² may be the same or different, and each independently represents a hydrogen atom, an alkyl group that may be substituted, an alkenyl group that may be substituted and an alkynyl group that may be substituted; and R³ represents a hydrogen atom or an alkyl group;

[C2] the compound according to [C1], possible stereoisomers thereof or racemates thereof, pharmacologically acceptable salts thereof, hydrates thereof, solvated compounds thereof, or pro-drugs thereof, wherein Ar2 represents a carboxyalkylnaphthyl group, carboxyphenyl group, carboxyalkylphenyl group or a monocyclic heteroaromatic ring other than a pyrazolyl group;

[C3] the compound according to [C1], possible stereoisomers thereof or racemates thereof, pharmacologically acceptable salts thereof, solvated compounds thereof, or pro-drugs thereof, wherein Ar² represents a carboxyphenyl group, a carboxyalkylphenyl group or a monocyclic heteroaromatic ring other than a pyrazolyl group;

[C4] the compound according to [C1] to [C3], possible stereoisomers thereof or racemates thereof, pharmacologically acceptable salts thereof, hydrates thereof, solvated compounds thereof, or pro-drugs thereof, wherein Ar¹ may be substituted with an alkoxy group that may be substituted;

[C5] the compound according to any one of [C1] to [C4], possible stereoisomers thereof or racemates thereof, pharmacologically acceptable salts thereof, hydrates thereof, solvated compounds thereof, or pro-drugs thereof, wherein R¹ represents a lower alkyl group;

[C6] the compound according to any one of [C1] to [C4], possible stereoisomers thereof or racemates thereof, pharmacologically acceptable salts thereof, hydrates thereof, solvated compounds thereof, or pro-drugs thereof, wherein R¹ represents an ethyl group;

[C7] the compound according to any one of [C1] to [C6], possible stereoisomers thereof or racemates thereof, pharmacologically acceptable salts thereof, hydrates thereof, solvated compounds thereof, or pro-drugs thereof, wherein R² represents a lower alkyl group;

[C8] the compound according to any one of [C1] to [C6], possible stereoisomers thereof or racemates thereof, pharmacologically acceptable salts thereof, hydrates thereof, solvated compounds thereof, or pro-drugs thereof, wherein R² represents a lower alkenyl group;

[C9] the compound according to any one of [C1] to [C8], possible stereoisomers thereof or racemates thereof, pharmacologically acceptable salts thereof, hydrates thereof, solvated compounds thereof, or pro-drugs thereof, wherein R³ represents a hydrogen atom;

[C10] a medicine-comprising the compound according to any one of [C1] to [C9], possible stereoisomers thereof or racemates thereof, pharmacologically acceptable salts thereof, hydrates thereof, solvated compounds thereof, or pro-drugs thereof as an active ingredient;

[C11] a medicine according to [C10] as a preventive and/or therapeutic agent for inflammatory diseases;

[C12] a medicine according to [C10] or [C11] as an inhibitor of PDE4 activity;

[C13] an inhibitor of PDE4 activity comprising the compound according to any one of [C1] to [C9], possible stereoisomers thereof or racemates thereof, pharmacologically acceptable salts thereof, hydrates thereof, solvated compounds thereof, or pro-drugs thereof as an active ingredient;

[C14] use of the compound according to any one of [C1] to [C9], possible stereoisomers thereof or racemates thereof, pharmacologically acceptable salts thereof, hydrates thereof, solvated compounds thereof, or pro-drugs thereof for producing the medicine according to any one of [C10] to [C12];

[C15] a method for preventing and/or treating inflammatory diseases comprising the step of administering the compound according to any one of [C1] to [C9], possible stereoisomers thereof or racemates thereof, pharmacologically acceptable salts thereof, hydrates thereof, solvated compounds thereof, or pro-drugs thereof to mammals including a human in an amount sufficient for prevention and/or treatment; and

[C16] a method for inhibiting PDE4 activity in the body of mammals including a human comprising the step of administering the compound according to any one of [C1] to [C9], possible stereoisomers thereof or racemates thereof, pharmacologically acceptable salts thereof, hydrates thereof, solvated compounds thereof, or pro-drugs thereof to the mammals including the human in an amount sufficient.

### EFFECT OF INVENTION

Since the compound of the invention or a salt thereof exhibits a potent PDE4 inhibitory action in the body of mammals including a human, it is useful, for example, for preventing and/or treating various diseases caused by inflammation. The compound of the invention or a salt thereof has so low toxicity that it can be safely used as an active ingredient of a medicine.

### BEST MODE FOR CARRYING OUT THE INVENTION

The invention will be described in detail hereinafter.

Examples of halogen atoms in the specification of the invention denote fluorine, chlorine, bromine and iodine atoms unless otherwise indicated.

The term “lower” of the substituents refers to substituents having a carbon number of at most 10, for example from 1 to 6, preferably from 1 to 3.

Examples of the alkyl group include linear, branched or cyclic saturated hydrocarbon groups as a combination thereof, and lower alkyl groups are preferable. The lower alkyl groups refer to those having a carbon number in the range from 1 to 10, preferably 1 to 6, and more preferably 1 to 3. The same description is valid for alkyl moieties of other substituents (for example an alkoxy group) having the alkyl moiety.

Examples of the suitable alkyl group with a carbon number in the range from 1 to 3 include methyl, ethyl, n-propyl, isopropyl or cyclopropyl group, and examples of the suitable alkyl group with a carbon number in the range from 4 to 6 include n-butyl, isobutyl, s-butyl, t-butyl, cyclobutyl, cyclopropylmethyl, n-pentyl, cyclopentyl, cyclopropylethyl, cyclobutylmethyl, n-hexyl, cyclohexyl, cyclopropylpropyl, cyclobutylethyl or cyclopentylmetyl group. The methyl, ethyl, n-propyl or isopropyl group is particularly preferable as the alkyl group. An example of the most preferable alkyl group is the methyl group, and the ethyl group is the most preferable example of the alkyl group in another embodiment.

An example of the alkenyl group is a lower alkenyl group containing one or a plurality of double bonds, and a lower alkenyl group having one double bond is preferable. The lower alkenyl group refers to an alkenyl group having a carbon number in the range from 2 to 10. An alkenyl group with a carbon number in the range from 2 to 5 is preferable, and an alkenyl group with a carbon number of 2 to 4 is particularly preferable. Preferred examples of the alkenyl group with a carbon number in the range from 2 to 4 include vinyl, allyl, propenyl, butylidene, but-1-enyl, but-2-enyl or but-3-enyl group, and preferred examples of the alkenyl group with a carbon number of 5 include pentylidene, pent-1-enyl, pent-2-enyl, pent-3-enyl or pent-4-enyl group. The vinyl, allyl or propenyl group is preferable, the vinyl or allyl group is more preferable, and the allyl group is the most preferable, as the alkenyl group.

An example of the alkynyl group is a lower alkynyl group including one or a plurality of triple bonds, and a lower alkenyl group containing one triple bond is preferable. An alkynyl group having a carbon number in the range from 2 to 5 is preferable as the lower alkynyl group. Specifically, preferred examples include ethynyl, prop-1-ynyl, prop-2-ynyl, but-1-ynyl, but-2-ynyl, but-3-ynyl, pent-1-ynyl, pent-2-ynyl, pent-3-ynyl or pent-4-ynyl group. The ethynyl, prop-2-ynyl or but-3-ynyl group is more preferable, the ethynyl or prop-2-ynyl group is further preferable, and prop-2-ynyl group is particularly preferable.

An example of the alkylene group is a divalent residue obtained by removing an arbitrary hydrogen atom from the above mentioned alkyl group such as a divalent group of a linear, branched or cyclic saturated hydrocarbon or a combination thereof. A lower alkylene group is preferable. The lower alkylene group refers to an alkylene group with a carbon number in the range from 1 to 10, and the alkylene group with a carbon number in the range from 1 to 6 is preferable, an alkylene group with a carbon number in the range from 1 to 3 is more preferable, and an alkylene group with a carbon number of 1 is more preferable. An example of the alkylene group with a carbon number 1 is the methylene group, preferred examples of the alkylene group with a carbon number of 2 and 3 include ethylene, n-propylene, isopropylene or cyclopropylene group, and a preferred example of the alkylene group with a carbon number in the range from 4 to 6 includes a divalent residue obtained by removing an arbitrary hydrogen atom from the group exemplified as the preferred alkyl group with a carbon number in the range from 4 to 6. The methylene, ethylene, n-propylene or isopropylene group is particularly preferable as the alkylene group. The methylene group is the most preferable alkylene group. The ethylene group is the most preferable alkylene group in another embodiment.

An example of the alkenylene group is a divalent residue obtained by removing an arbitrary hydrogen atom from the above-described alkenyl group. An example is a lower alkenylene radial having one or a plurality of double bonds,)and the lower alkenylene group having one double bond is preferable. The lower alkenylene group refers to an alkenylene group having a carbon number in the range from 2 to 10. The alkenylene group having a carbon number in the range from 2 to 5 is referable, and the alkenylene group having a carbon number in the range from 2 to 4 is particularly preferable. Preferred examples of the alkenylene group having a carbon number in the range from 2 to 4 are vinylene, propenylene, but-1-enylene, but-2-enylene or but-3-enylene group, while an example of the alkenylene group having a carbon number of 5 is a divalent residue obtained by removing an arbitrary hydrogen atom from the alkenyl group having a carbon number of 5 in the above-mentioned preferred example. The vinylene or propenylene group is more preferable, and the vinylene group is particularly preferable as the alkenylene group.

An example of the alkoxy group is a linear, branched or cyclic saturated alkyl ether or a combination thereof, and a lower alkoxy group is preferable. While an example of the lower alkoxy group includes an alkoxy group with a carbon number in the range from 1 to 6, the alkoxy group with a carbon number in the range from 1 to 4 is preferable. Preferred examples of the alkoxy group with a carbon number in the range from 1 to 4 include methoxy, ethoxy, n-propoxy, isopropoxy, cyclopropoxy, n-butoxy, isobutoxy, s-butoxy, t-butoxy, cyclobutoxy or cyclopropylmethoxy group, and preferred examples of the alkoxy group with a carbon number of 5 or 6 include n-pentyloxy, cyclopentyloxy, cyclopropylethyloxy, cyclobutylmethyloxy, n-hexyloxy, cyclohexyloxy, cyclopropylpropyloxy, cyclobutylethyloxy or cyclopentylmethyloxy group.

An example of the alkylthio group is a linear, branched or cyclic saturated alkylthioether group or a combination thereof, and a lower alkylthio group is preferable. An alkylthio group with a carbon number in the range from 1 to 4 is preferable as the lower alkylthio group. Specifically, preferred examples include methylthio, ethylthio, n-propylthio, isopropylthio, cyclopropylthio, n-butylthio, isobutylthio, s-butylthio, t-butylthio, cyclobutylthio or cyclopropylthio groups.

An example of the alkoxycarbonyl group includes a group having a carbonyl group attached at the end of the above-mentioned alkoxy group, and a lower alkoxycarbnyl group is preferable. While an example of the lower alkoxycarbonyl group includes an alkoxycarbonyl group obtained by attaching a carbonyl group at the end of the alkoxy group with a carbon number in the range from 1 to 6, a group having the carbonyl group attached at the end of an alkoxy group having a carbon number in the range from 1 to 4 is preferable. Specifically, preferred examples include methoxycarbonyl, ethoxycarbonyl, n-propoxycarbonyl, isopropoxycarbonyl, cyclopropoxycarbonyl, n-butoxycarbonyl, isobutoxycarbonyl, s-butoxycarbonyl, t-butoxycarbonyl, cyclobutoxycarbonyl or cyclopropylmethoxy-carbonyl groups.

An example of the alkenyloxy group is an alkenylether group having an oxygen atom attached at the end of the alkenyl group. A lower alkenyloxy group having one or a plurality of double bonds is preferable, and the lower alkenyloxy group having one double bond is more preferable. The lower alkenyloxy group having a carbon number in the range from 2 to 4 is preferable as the lower alkenyloxy group. Specifically, vinyloxy, allyloxy or propenyloxy groups are preferable, and the allyloxy group is particularly preferable.

An example of the alkenylthio group is an alkenylthioether group having a sulfur atom attached at the end of the alkenyl group. A lower alkenylthio group having one or a plurality of double bonds is preferable, and a lower alkenylthio group having one double bond is more preferable. An alkenylthio group with a carbon number in the range from 2 to 4 is preferable as the lower alkenylthio group. Specifically, vinylthio, allylthio or propenylthio groups are preferable, and the allylthio group is particularly preferable.

A preferable example an acyl group is, for example, an alkanoyl group or an arylcarbonyl group, where the alkanoyl group is preferable, and the lower alkanoyl group is more preferable. While an example of the alkanoyl group includes a linear, branched or cyclic saturated alkylcarbonyl group or a combination thereof, the alkyl moiety may include one or a plurality of unsaturated bonds. An acyl group with a carbon number in the range from 2 to 5 is preferable as the lower alkanoyl group. Specifically, preferable examples include acetyl, propanoyl, butanoyl, 2-methylpropanoyl, cyclopropylcarbonyl, pentanoyl, 3-methylbutanoyl, 2,2-dimethylpropanoyl or cyclobutylcarbonyl group.

While a preferable example of the acyloxy group is an alkanoyloxy group (alkylcarbonyloxy group) or arylcarbonyloxy group, the alkanoyloxy group is preferable, and the lower alkanoyloxy group is more preferable. The alkyl moiety of the alkanoyloxy group may contain one or a plurality of unsaturated bonds. The acyloxy group with a carbon number in the range from 2 to 5 is preferable as the lower alkanoyloxy group. Specifically, preferred examples include acetoxy, propanoyloxy, butanoyloxy, 2-methylpropanoyloxy, cyclopropylcarbonyloxy, pentanoyloxy, 3-methylbutanoyloxy, 2,2-dimethylpropanoyloxy or cyclobutylcarbonyloxy groups.

A lower alkylsulfinyl group is preferable as the alkylsulfinyl group. The lower alkylsulfinyl group preferably has a carbon number in the range from 1 to 4. Specifically, preferred examples include methylsulfinyl, ethylsulfinyl, n-propylsulfinyl, isopropylsulfinyl, cyclopropylsulfinyl, n-butylsulfinyl, isobutylsulfinyl, s-butylsulfinyl, t-butylsulfinyl, cyclobutylsulfinyl or cyclopropylmethyl sulfinyl groups.

A preferable example of the alkylsulfonyl group is a lower alkylsulfonyl group, which preferably has a carbon number in the range from 1 to 4. Specifically, preferred examples include methylsulfonyl, ethylsulfonyl, n-propylsulfonyl, isopropylsulfonyl, cyclopropylsulfonyl, n-butylsulfonyl, isobutylsulfonyl, s-butylsulfonyl, t-butylsulfonyl, cyclobutylsulfonyl or cyclopropylsulfonyl groups.

A preferable example of the alkylcarbamoyl group is a lower alkylcarbamoyl group, which preferably has a carbon number in the range from 1 to 4. Specifically, preferred examples include methylcarbamoyl, ethylcarbamoyl, n-propylcarbamoyl, isopropylcarbamoyl, cyclopropylcarbamoyl, n-butylcarbamoyl, isobutylcarbamoyl, s-butylcarbamoyl, t-butylcarbamoyl, cyclobutylcarbamoyl or cyclopropylmethylcarbamoyl groups.

An example of the amino group is —NH₂ group.

An example of the aryl ring is a monocyclic aromatic ring or a fused polycyclic aromatic ring. While the aryl ring may be a hydrocarbon ring, it may contain one or a plurality of heteroatoms, for example 1 to 3 heteroatoms, selected from the group consisting of nitrogen, sulfur and oxygen atoms as ring-constituting atoms other than the carbon atom.

An example of the monocyclic aromatic ring is a monocyclic aromatic hydrocarbon or a monocyclic heteroaromatic ring containing one or a plurality of heteroatoms. The example of the monocyclic aromatic hydrocarbon is a benzene ring. An example of the monocyclic heteroaromatic ring is a five or six membered heteroaromatic ring containing one or a plurality of heteroatoms. Specifically, preferred examples of the five or six membered heteroaromatic ring include thiophene, pyridine, furan, thiazole, oxazole, pyrazole, pyrazine, pyrimidine, pyrrole, imidazole, pyridazine, isothiazole, isooxazole, 1,2,4-oxadiazole, 1,3,4-oxadiazole, 1,2,4-thiadiazole, 1,3,4-thiadiazole or furazan.

An example of the condensed polycyclic aromatic ring includes a condensed polycyclic aromatic hydrocarbon or a condensed polycyclic heteroaromatic ring having one or a plurality of heteroatoms. An example of the condensed aromatic hydrocarbon includes a condensed polycyclic ring having a carbon number in the range from 9 to 14, or a condensed polycyclic ring having a carbon number of 2 or 3. Preferred specific-examples are naphthalene, indene, fluorene or anthracene. An example of the condensed heteroaromatic ring includes a 9 to 14 membered, preferably 9 or 10 membered condensed heteroaromatic ring having at least one heteroatom. Preferred specific examples thereof are benzofuran, benzothiophene, benzimidazole, benzoxazole, benzothiazole, benzisothiazole, naphtho[2,3-b]thiophene, quinoline, isoquinoline, indole, quinoxaline, phenanthridine, phenothiazine, phenoxazine, phthalazine, naphthylizine, quinazoline, cinnoline, carbazole, β-carboline, acridine, phenazine, phthalimide or thioxanthene.

An example of the aryl group is a monocyclic aromatic group or a condensed polycyclic aromatic group, and includes a monovalent residue formed by removing one arbitrary hydrogen atom from the above-mentioned aryl ring.

An example of the monocyclic aromatic group is a monovalent residue formed by removing one arbitrary hydrogen atom from the monocyclic aromatic ring. Specific preferred examples of the monocyclic aromatic group include phenyl, thienyl (2- or 3-thienyl), pyridyl (2-, 3- or 4-pyridyl), furyl (2- or 3-furyl), thiazolyl (2-, 4- or 5-thiazolyl), oxazolyl (2-, 4- or 5-oxazolyl), pyrazolyl (1-, 3- or 4-pyrazolyl), 2-pyrazinyl, pyrimidinyl (2-, 4- or 5-pyrimidinyl), pyrrolyl (1-, 2- or 3-pyrrolyl), imidazolyl (1-, 2- or 4-imidazolyl), pyridazinyl (3- or 4-pyridazinyl), 3-isothiazolyl, 3-isooxazolyl, 1,2,4-oxadiazole-5-yl or 1,2,4-oxadiazole-3-yl groups.

An example of the condensed polycyclic aromatic group include a monovalent residue formed by removing one arbitrary hydrogen atom from a condensed polycyclic aromatic ring comprising 2 to 4 rings, preferably 2 or 3 rings.

Preferred specific examples of the condensed polycyclic aromatic group include 1-naphthyl, 2-naphthyl, 2-indenyl, 2-anthryl, quinolyl (2-, 3-, 4-, 5-, 6-, 7- or 8-quinoryl), isoquinolyl (2-, 3-, 4-, 5-, 6-, 7- or 8-isoquinoryl), indolyl (1-, 2-, 3-, 4-, 5-, 6- or 7-indolyl), isoindolyl (1-, 2-, 4- or 5-isoindolyl), phthalazinyl (1-, 5- or 6-phthalazinyl), quinoxalinyl (2-, 3- or 5-quinoxalinyl), benzofuranyl (2-, 3-, 4-, 5- or 6-benzofuranyl), benzothiazolyl (2-, 4-, 5- or 6-benzothiazolyl), benzimidazolyl (1-, 2-, 4-, 5- or 6-benzimidazolyl), fluorenyl (1-, 2-, 3- or 4-fluorenyl) or thioxanthenyl groups.

Preferred examples of the substituent in an alkyl group that may be substituted include a hydroxy group, cyano group, halogen atom, alkoxy group, alkylthio group, an alkoxy group arbitrarily substituted with at least one halogen atom, alkenyloxy group, alkenylthio group, acyl group, acyloxy group, alkylsulfinyl group, alkylsulfonyl group, alkylcarbamoyl group, alkylamino group, dialkylamino group, alkylamino group arbitrarily substituted with at least one halogen atom, acylamino group, acyl(alkyl)amino group, alkylsulfonylamino group, alkylsulfonyl(alkyl)amino group, alkylsulfonylamino group arbitrarily substituted with at least one halogen atom, alkylsulfonyl(alkyl)amino group arbitrarily substituted with at least one halogen atom or aryl group that may be substituted. Preferable examples include a hydroxy, halogen or alkoxy group; more preferable examples include the alkoxy group or fluorine atom; and particularly preferable examples include the alkoxy group. Fluorine atom is particularly preferable in another embodiment.

While the number of the substituents in the group that may be further substituted (alkyl, alkenyl, alkynyl or alkoxy group) is not particularly restricted in the specification of the invention, it is usually in the range from 1 to several substituents, and the number is preferably one. In another embodiment, the number of the substituents is preferably in the range from 1 to 3 when the substituent in the group that may be substituted is a halogen atom.

The alkyl groups that may be substituted are those described above as preferable alkyl groups. In another embodiment, the alkyl groups that may be substituted are those substituted with above-mentioned preferable substituents. Preferred examples of the alkyl group that may be substituted include methyl, ethyl, n-propyl, isopropyl, cyclopropyl, n-butyl, isobutyl, s-butyl, t-butyl, cyclobutyl, cyclopropylmethyl, n-pentyl, cyclopentyl, cyclopropylethyl, cyclobutylmethyl, n-hexyl, cyclohexyl, cyclopropylpropyl, cyclobutylethyl, cyclopentylmethyl, trifluoromethyl, hydroxymethyl, 2-hydroxymethyl, methdxymethyl or 2-methoxyethyl groups. More preferred examples include methyl, ethyl, n-propyl, isopropyl, trifluoromethyl, hydroxymethyl, 2-hydroxyethyl, methoxymethyl or 2-methoxyethyl group; and particularly preferable examples include methyl, ethyl, hydroxymethyl, 2-hydroxyethyl or methoxymethyl groups.

The substituent in the alkenyl group that may be substituted, and the substituent in the alkenyl group that may be substituted are the same as the substituents in the alkyl group that may be substituted.

The alkenyl group that may be substituted is preferably included in the preferable example of the above-mentioned alkenyl group, and the alkynyl group that may be substituted is preferably included in the preferable example of the above-mentioned alkynyl group.

The substituent in the alkoxy group that may be substituted is the same as the substituent of the above-mentioned alkyl group that may be substituted. The alkoxy-group that may be substituted is preferably included in the preferred example of the alkoxy group. In another embodiment, the alkoxy group that may be substituted is the alkoxy group that is substituted with the substituent in the above-mentioned alkoxy group that may be substituted. Specific examples include methoxy, ethoxy, n-propoxy, isopropoxy, n-butoxy, isobutoxy, s-butoxy, t-butoxy, trifluoromethoxy or 2-methoxyethoxy groups; more preferable examples include methoxy, ethoxy, trifluoromethoxy or 2-methoxyethoxy group; a particularly preferable example includes the methoxy or ethoxy group; and the most preferable example is the methoxy group.

The substituent in the alkylthio group that may be substituted is the same as the substituent in-the above-mentioned substituent that may be substituted. The alkylthio group that may be substituted is included in the preferred example of the above-mentioned alkylthio group. Preferred specific examples include methylthio, ethylthio, n-propylthio, isopropylthio, cyclopropylthio, n-butylthio, isobutylthio, s-butylthio, t-butylthio, cyclobutylthio, cyclopropylmethylthio or 2-methoxyethylthio groups; more preferable examples include methylthio, ethylthio or 2-methoxyethylthio groups; and a particularly preferable example is the methylthio or ethylthio group.

A preferred example of the alkoxycarbonyl group that may be substituted is the alkoxycarbonyl group having a carboxy group attached at the end of the alkoxy group. Preferred specific examples include methoxycarbonyl, ethoxycarbonyl, n-propoxycarbonyl, isopropoxycarbonyl, cyclopropoxycarbonyl, n-butoxycarbonyl, isobutoxycarbonyl, s-butoxycarbonyl, t-butoxycarbonyl, cyclobutoxycarbonyl or cyclopropylmethoxycarbonyl groups; more preferable examples include methoxycarbonyl, ethoxycarbonyl or t-butoxycarbonyl groups; and a particularly preferable example is the methoxycarbonyl or ethoxycarbonyl group.

Examples of the amino group that may be substituted inclue —NH₂, alkylamino group, dialkylamino group, acylamino group, acyl(alkyl)amino group, alkyl-sulfonylamino group, alkylsulfonyl(alkyl)amino group, alkylamino group that may be arbitrarily substituted with at least one halogen atom, alkylsulfonylamino group that may be arbitrarily substituted with at least one halogen atom, or alkylsulfonyl(alkyl)amino group that may be arbitrarily substituted with at least one halogen atom. A further example of the amino group that may be substituted includes amino group substituted with one or a plurality of alkyl groups that may be substituted, amino group substituted with one or a plurality of aryl groups that may be substituted, amino group substituted with one or a plurality of alkyl groups that may be substituted, amino group substituted with one or a plurality of acyl groups that may be substituted, alkylamino group substituted with one or a plurality of acyl groups that may be substituted, amino group substituted with one or a plurality of alkylsulfonyl groups that may be substituted, alkylamino group substituted with one or a plurality of alkylsulfonyl groups that may be substituted, alkylcarbamoylamino group that may be substituted, alkylthiocarbamoylamino group that may be substituted, arylcarbamoylamino group that may be substituted, arylthiocarbamoylamino group that may be substituted, arkyloxycarbonylamino group that may be substituted or aryloxycarbonylamino group that may be substituted.

A preferable example of the alkylamino group includes a lower alkylamino group. The alkylamino group having a carbon number in the range from 1 to 4 is preferable as the lower alkylamino group. Preferred specific examples include methylamino, ethylamino, n-propylamino, isopropylamino, cyclopropylamino, n-butylamino, isobutylamino, s-butylamino, t-butylamino, cyclobutylamino or cyclopropylmethylamino groups. More preferable examples include methylamino, ethylamino or isopropylamino groups, and the methylamino or ethylamino group is a particularly preferable example.

An example of the dialkylamino group is an amino group substituted with the same or different alkyl groups, and a lower dialkylamino group is usually preferable. The lower dialkylamino group is preferably substituted with alkyl groups with a carbon number in the range from 1 to 4. Preferred specific examples include dimethylamino, ethyl(methyl)amino, diethylamino, methyl(n-propyl)amino, isopropyl(methyl)amino, cyclopropyl(methyl)-amino, n-butyl(methyl)amino, isobutyl(methyl)amino, s-butyl(methyl)amino, t-butyl(methyl)amino, cyclobutyl-(methyl)amino or cyclopropylmethyl(methyl)amino groups. More preferable examples include dimethylamino, diethylamino or ethyl(methyl)amino groups, and particularly preferable examples include dimethylamino or diethylamino groups.

The example of the acylamino group is the amino group substituted with the above-mentioned acyl group, and the preferable example of the acyl group is the same as described-above. Preferred specific examples are acetylamino, propanoylamino, butanoylamino, 2-methylpropanoylamino, cyclopropylcarbonylamino, pentanoylamino, 3-methylbutanoylamino, 2,2-dimethylpropanoylamino or cyclobutylcarbonylamino groups. More preferable examples include acetylamino, propanoylamino or 2,2-dimethylpropanoylamino groups, and the acetylamino or propanoylamino group is a particularly preferable example.

An example of the acyl(alkyl)amino group is an amino group simultaneously substituted with one acyl group and one alkyl group, and preferable examples of the acyl group and alkyl group are the same as those described above. Preferred specific examples include acetyl(methyl)amino, methyl(propanoyl)amino, butanoyl-(methyl)amino, methyl(2-methylpropanoyl)amino, cyclopropylcarbonyl(methyl)amino, methyl (pentanoyl)amino, methyl (3-methylbutanoyl) amino, 2,2-dimethylpropanoyl-(methyl)amino or cyclobutylcarbonyl(methyl)amino groups. More preferable examples include acetyl(methyl)amino or methyl(propanoyl)amino groups, and the acetyl(methyl)amino group is particularly preferable.

An example of the alkylsulfonylamino group is the amino group substituted with alkylsulfonyl group as described above, and the preferable example of the alkylsulfonyl group is as described above. Preferred specific examples include methylsulfonylamino, ethyl-sulfonylamino, n-propylsulfonylamino, isopropylsulfonylamino, cyclopropylsulfonylamino, n-butylsulfonylamino, isobutylsulfonylamino, s-butylsulfonylamino, t-butylsulfonylamino, cyclobutylsulfonylamino or cyclopropylsulfonylamino groups. More preferable examples include methylsulfonylamino, ethylsulfonylamino or n-propylsulfonylamino groups, and the methylsulfonylamino or ethylsulfonylamino group is particularly preferable.

An example of the alkylsulfonyl(alkyl)amino group is an amino group simultaneously substituted with one alkylsulfonyl group and one alkyl group, and preferable examples of the alkylsulfonyl group and alkyl group are the same as described above. Preferred specific examples include methyl(methylsulfonyl)amino, ethylsulfonyl-(methyl)amino, methyl(n-propylsulfonyl)amino, isopropyl-sulfonyl(methyl)amino, cyclopropylsulfonyl(methyl)amino, n-butylsulfonyl(methyl)amino, isobutylsulfonyl(methyl)amino, s-butylsulfonyl(methyl)amino, t-butylsulfonyl(methyl)amino, cyclobutylsulfonyl(methyl)amino or cyclopropylmethylsulfonyl(methyl)amino groups. More preferable examples are methyl-(methylsulfonyl)amino or ethylsulfonyl(methyl)amino groups, and the methyl(methylsulfonyl)amino group is particularly preferable.

An example of the alkylamino group arbitrarily substituted with the halogen atom is the alkylamino group in which hydrogen atoms of at least one alkylamino group are substituted with arbitrary kinds of halogen atoms, and the alkylamino group arbitrarily substituted with at least one halogen atom and having a carbon number in the range from 1 to 4 is preferable. The halogen atoms may be the same or different to one another when substituted with a plurality of halogen atoms. Preferred specific examples include chloromethylamino, dichloromethylamino, trichloromethylamino, fluoromethylamino, difluoromethylamino, trifluoromethylamino, fluoroethylamino or 2,2,2-trifluoroethylamino group. More preferable examples are trifluoromethylamino or 2,2,2-trifluoroethylamino groups, and the trifluoromethylamino group is particularly preferable.

An example of the alkylsulfonylamino group arbitrarily substituted with at least one halogen atom is the alkylsulfonylamino group in which at least one hydrogen atom of the above-mentioned alkylsulfonylamino group is substituted with arbitrary kinds of halogen atoms. The alkylsulfonylamino group arbitrarily substituted with at least one halogen atom and having a carbon number in the range from 1 to 4 is usually preferable. The halogen atoms may be the same or different-to one another when the substituted with a plurality of halogen atoms. A specific example is trifluoromethyl.

An example of the alkylsulfonyl(alkyl)amino group arbitrarily substituted with at least one halogen atom is an alkylsulfonyl(alkyl)amino group in which at least one hydrogen atom of the alkylsulfonyl(alkyl)amino group is substituted wit-h arbitrary kinds of halogen atoms, and an alkylsulfonyl(alkyl)amino group arbitrarily substituted with at least one halogen atom and having a carbon number in the range from 1 to 4 is usually preferable. The halogen atoms may be the same or different when substituted with a plurality of halogen atoms. A specific example is methyl(trifluoromethylsulfonyl)amino group.

The alkyl group that may be substituted in the amino group substituted with one or two-alkyl groups that may be substituted is preferably the preferable example of the above-mentioned alkyl group that may be substituted. The amino group substituted with one or two alkyl group that may be substituted is included in the preferable example as the alkylamino group and dialkylamino group. In another embodiment, the amino group substituted with one or two alkyl group that may be substituted is preferably included in the amino group substituted with one or two alkyl groups substituted with preferable substituents in the alkyl group that may be substituted. In a different embodiment, the amino group substituted with one or two alkyl group that may be substituted is preferably included in the amino group substituted with one alkyl group substituted with preferable substituents of the alkyl group that may be substituted. In a further different embodiment, the amino group substituted with one or two alkyl groups that may be substituted is preferably included in the alkyl group substituted with one alkyl group and one lower alkyl group substituted with preferable substituents in the alkyl groups that may be substituted. Preferred specific examples include dimethylamino, diethylamino, ethylmethylamino, methoxyethylamino, methyl(methoxyethyl)-amino, hydroxyethylamino or hydroxyethyl(methyl)amino groups. A particularly preferable example is a dimethylamino, methoxyethylamino group or a hydroxyethylamino group.

A preferable example of the aryl group that may be substituted in the amino group substituted with one or two aryl groups that may be substituted is preferably included in the aryl group that may be substituted to be described hereinafter. The amino group substituted one or two aryl groups that may be substituted is preferably the amino group substituted with one aryl group in the above-mentioned preferable example. The phenylamino group is a particularly preferable example.

A preferred example of the aryl group that may be substituted, or the aryl group that may be substituted in the amino group substituted with the alkyl group is the same as described above, and the substituent in the alkyl group that may be substituted is included in the preferable example of the above-mentioned alkyl group that may be substituted. The amino group substituted with the aryl group that may be substituted and with the alkyl group that may be substituted is preferably the amino group substituted with the aryl group that may be substituted. A particularly preferable example is a methyl(phenyl)amino group.

The substituent of the amino group substituted with the acyl group that may be substituted, and the substituent of the acyl group in the alkylamino group substituted with the acyl group that may be substituted are preferably included in the preferable example of the acyl group that may be substituted as will be described hereinafter. Particularly preferable examples of the amino group substituted with the acyl group that may be substituted include acetylamino, propanoylamino, butanoylamino or phenylacetylamino groups.

The substituent of the alkylsulfonyl group in the amino group substituted with the alkylsulfonyl group that may be substituted, and the substituent of the alkylsulfonyl group in the in the alkylamino group substituted with the alkylsulfonyl group that may be substituted are preferably included in the preferable example of the substituent in the alkylsulfonyl group to be described hereinafter. Particularly preferable examples of the amino group substituted with the alkylsulfonyl group that may be substituted include methanesulfonylamino, trifluoromethanesulfonylamino, methyl(methanesulfonyl)amino or methyl(trifluoromethanesulfonyl)amino groups.

The substituent in the alkylcarbamoylamino group that may be substituted is included in the preferable example of the substituent in the above-mentioned alkyl group that may be substituted when the substitution site of the substituent is on the alkyl group. Otherwise, the substituent includes the preferable example of the above-mentioned alkyl group that may be substituted or a hydrogen atom when the substitution site of the substituent is on the nitrogen atom of the alkylcarbamoylamino group. The number of the substituent of the alkylcarbamoylamino group that may be substituted may be one or more, and the substituents may be the same or different to one another. When the alkylcarbamoylamino group is substituted with one substituent, the substituent is preferably on the alkyl group. A particularly preferable example is a ethylcarbamoylamino group or a methylcarbamoylamino group.

Preferable examples of the alkylthiocarbamoylamino group that may be substituted are the same as the examples corresponding to respective examples of the above-mentioned alkylcarbamoylamino group. A particularly preferable example includes an ethylthiocarbamoylamino group or a methylthiocarbamoylamino group.

Preferable examples of the alkyloxycarbonylamino group that may be substituted are the same as the examples corresponding to respective examples of the above-mentioned alkylcarbamoylamino group. A particularly preferable example includes the ethoxycarbonylamino group or methoxycarbonylamino group.

The substituent of the arylcarbamoylamino group that may be substituted is included in the preferable example of the aryl group that may be substituted to be described hereinafter when the substitution site of the substituent is on the aryl group, while the substituent may be included in the preferred example of the above-mentioned alkyl group that may be substituted or may be a hydrogen atom when the substitution site of the substituent is on the nitrogen of the arylcarbamoylamino group. The number of the substituent in the arylcarbamoylamino group that may be substituted may be one or more, and the substituents may be the same or different to one another. The arylcarbamoyl-amino group that may be substituted may have one substituent on the arylcarbamoylamino group that may be substituted, and the substitution site of the substituent is preferably on the aryl group. A particularly preferable example is phenylcarbamoylamino group that may be substituted.

The preferable example of the arylthiocarbamoylamino group that may be substituted is the same as respective examples corresponding to the above-mentioned arylcarbamoyl-amino group.

The preferable example of the aryloxycarbonylamino group that may be substituted is the same as respective examples corresponding to the arylcarbamoylamino group. A particularly preferable example is phenylthiocarbamoylamino group that may be substituted.

While the substituent of the acyl group that may be substituted is the same as the substituent of the above-mentioned alkyl group that may be substituted, at least one halogen atom is preferable as the substrate.

The acyl group that may be substituted is preferably an acyl group arbitrarily substituted with at least one halogen atom, or an acyl group arbitrarily substituted with at least one halogen atom and having a carbon number in the range from 2 to 5. The halogen atoms may be the same or different to one another when substituted with a plurality of halogen atoms. A preferable example is a trifluoroacetyl group.

Another preferable examples of the acyl group that may be substituted include acetyl, propanoyl, butanoyl, 2-methylpropanoyl, cyclopropylcarbonyl, pentanoyl, 3-methylbutanoyl, 2,2-dimethylpropanoyl or cyclobutylcarbonyl groups. The acetyl, propanyol or 2,2-dimethylpropanoyl group is more preferable, and acetyl or propanoyl group is particularly preferable.

A preferable acyloxy group that may be substituted is that having an oxygen atom at the end of the acyl group. A preferable example is a trifluoroacetoxy group.

Another preferable example of the acyloxy group that may be substituted includes acetoxy, propanoyloxy, butanoyloxy, 2-methylpropanoyloxy, cyclopropylcarbonyloxy, pentanoyloxy, 3-methylbutanoyloxy, 2,2-dimethylpropanoyloxy or cyclobutylcarbonyloxy group. A-more preferable example is the acetoxy, propanoyloxy or 2,2-dimethylpropanoyloxy group, and the acetoxy or propanoyloxy group is particularly preferable.

While the substituent of the alkylsulfinyl group that may be substituted is the same as the substituent in the abobe-mentioned alkyl group that may be substituted, at least one halogen atom is preferable.

The alkylsulfinyl group that may be substituted is preferably an alkylsulfinyl group arbitrarily substituted with at least one halogen atom, or an alkylsulfinyl group arbitrarily substituted with at least one halogen atom and having a carbon number in the range from 1 to 4. The halogen atoms may be the same or different to one another when substituted with a plurality of halogen atoms. A preferable example is a trifluoromethanesulfinyl group.

Other preferable examples of the alkylsulfinyl group that may be substituted include methylsulfinyl, ethylsulfinyl, n-propylsulfinyl, isopropylsulfinyl, cyclopropylsulfinyl, n-butylsulfinyl, isobutylsulfinyl, s-butylsulfinyl, t-butylsulfinyl, cyclobutylsulfinyl or cyclopropylmethylsulfinyl groups. A more preferable example is the methylsulfinyl or ethylsulfinyl group, and the methylsulfinyl group is particularly preferable.

While the substituent in the alkylsulfonyl group that may be substituted is the same as the substituent in the above-mentioned alkyl group that may be substituted, at least one halogen atom is preferable.

The substituted alkylsulfonyl group is preferably an alkylsulfonyl group arbitrarily substituted with at least one halogen atom, or an alkylsulfonyl group arbitrarily substituted with at least one halogen atom and having a carbon number in the range from 1 to 4. The halogen atoms may be the same or different to one another when substituted with a plurality of halogen atoms. A preferred example is a trifluoromethanesulfonyl group.

Other preferable examples of the alkylsulfonyl group that may be substituted include methylsulfonyl, ethylsulfonyl, n-propylsulfonyl, isopropylsulfonyl, cyclopropylsulfonyl, n-butylsulfonyl, isobutylsulfonyl, s-butylsulfonyl, t-butylsulfonyl, cyclobutylsulfonyl or cyclopropylmethylsulfonyl group. A more preferable example includes a methylsulfonyl group or a ethylsulfonyl group, and the methylsulfonyl group is particularly preferable.

A preferred example of the substituent of the aryl ring that may be substituted, or the substituent of the aryl group that may be substituted includes a hydroxy group, an alkyl group arbitrarily substituted with at least one hydroxy group, a halogen atom, an alkyl group arbitrarily substituted with at least one halogen atom, an alkoxy group, an alkylthio group, an alkoxy group substituted with at least one halogen atom, an alkenyloxy group, an alkenylthio group, an acyl group, an acyloxy group, an alkylsulfinyl group, an alkylsulfonyl group, an alkylcarbamoyl group, an alkylamino group, a dialkylamino group, an alkylamino group arbitrarily substituted with at least one halogen atom, an acylamino group, an acyl(alkyl)amino group, an alkylsulfonylamino group, an alkylsulfonyl(alkyl)amino group, an alkylsulfonylamino group arbitrarily substituted with at least one halogen atom, or an alkylsulfonyl(alkyl)amino group arbitrarily substituted with at least one halogen atom.

The aryl ring or the aryl group having the above-mentioned substituent is a preferable example in the preferable example of the aryl ring or aryl group as the aryl ring that may be substituted or the aryl group that may be substituted.

Ar¹ in general formula (1) represents a furyl group, thienyl group, triazolyl group, thiazolyl group, oxyazolyl group or banzothiazolyl group, and Ar¹ may be substituted. While Ar¹ may be substituted or not substituted, it is preferably substituted. However, Ar¹ is preferably not substituted in another embodiment. Ar¹ is more preferably the furyl group, thienyl group or thiazolyl group, further preferably the furyl group or thienyl group, and particularly the thienyl group. The furyl group is particularly preferable in another embodiment.

Preferable examples of the position of Ar¹ linked to the pyrimidine ring, and the number and position of the substituent when Ar¹ has the substituent are shown below. In the structure below, V denotes the position of Ar¹ bonded to position 2 of the pyrimidine ring, W¹ and W² denote the positions of substituents, respectively.

When Ar¹ represents the furyl group, Ar1-1 to Ar1-14 are preferable; 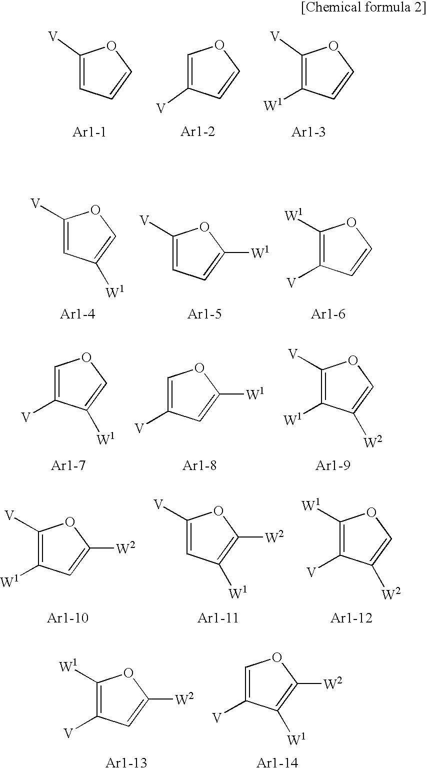
Ar1-4 to Ar1-8 are more preferable; 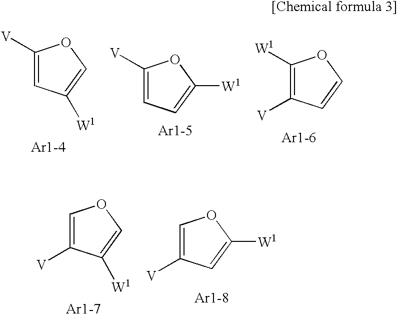
and
Ar1-4, Ar1-5 and Ar1-8.are particularly preferable:

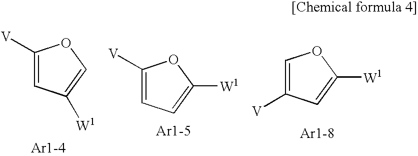

When Ar¹ represents the thienyl group, Ar1-15 to AR1-28 are preferable; 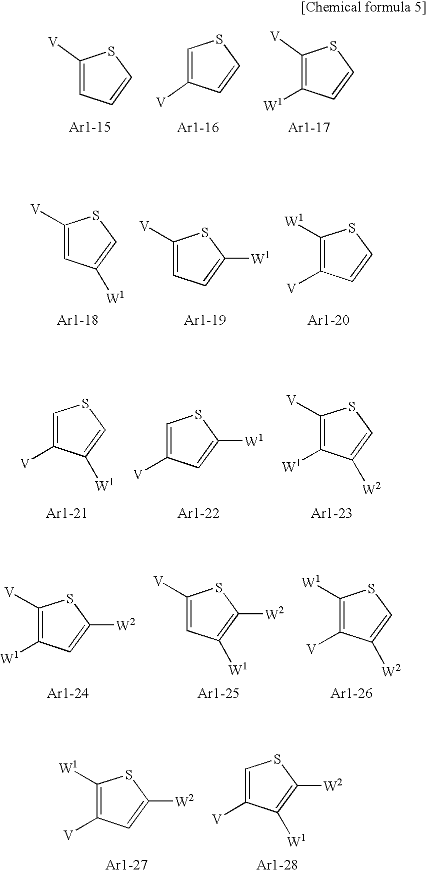
Ar1-17 to Ar1-22 are more preferable; 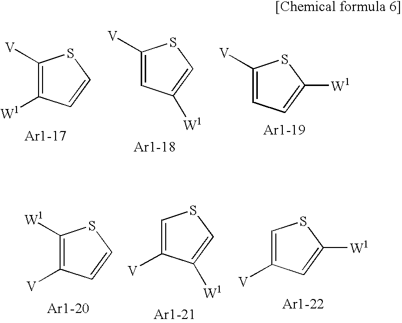
Ar1-18, Ar1-19 and Ar1-22 are further preferable; 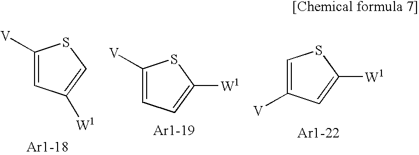
and
Ar1-19 is particularly preferable:

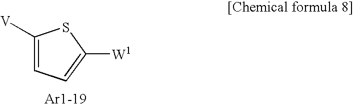

When Ar¹ represents the triazolyl group, Ar1-29 to Ar1-43 are preferable;

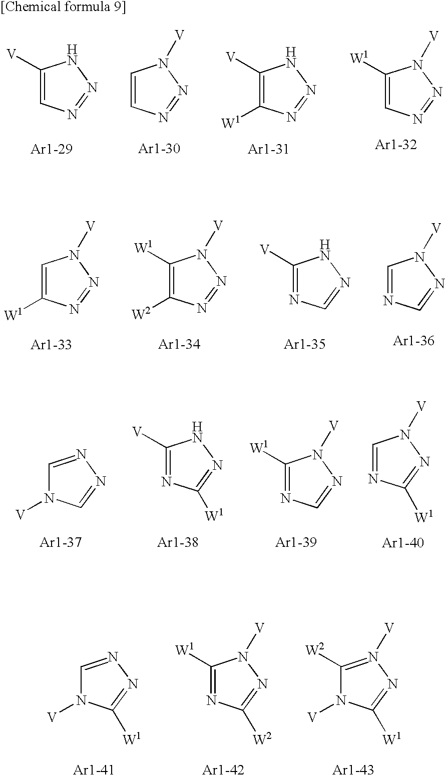

Ar1-31 to Ar1-33 and Ar1-38 to Ar1-41 are more preferable; 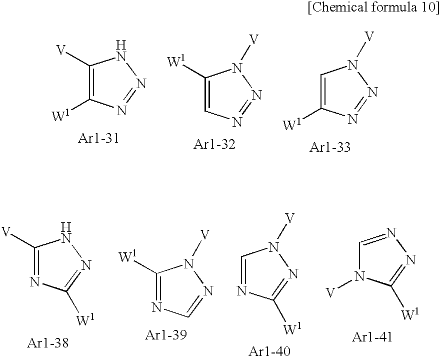
and
Ar1-33, Ar1-38 and Ar1-40 are particularly preferable:

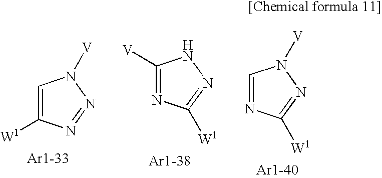

When Ar¹ represents the thiazolyl group, Ar1-44 to Ar1-55 are preferable; 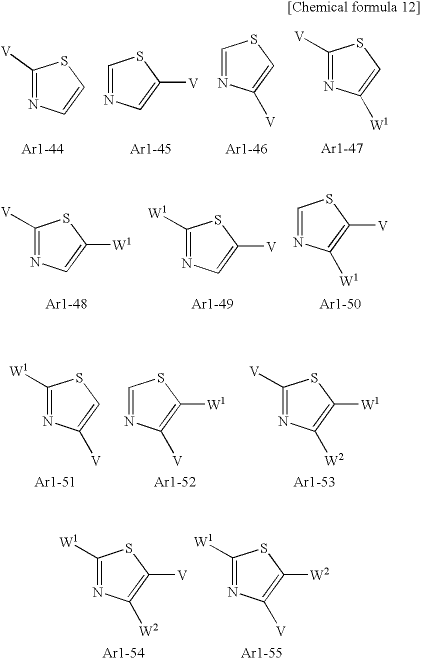
Ar1-47 to Ar1-52 are more preferable; 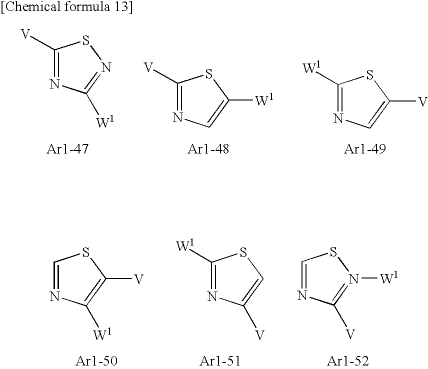
and
Ar1-47 to Ar1-49 and Ar1-51 are particularly preferable:

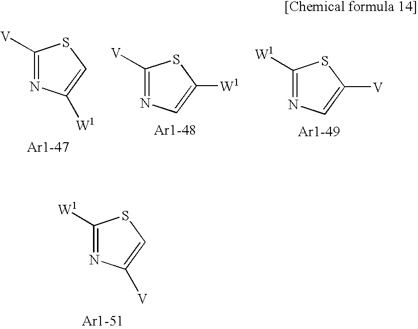

When Ar¹ represents the oxazolyl group, Ar1-56 to Ar1-67 are preferable; 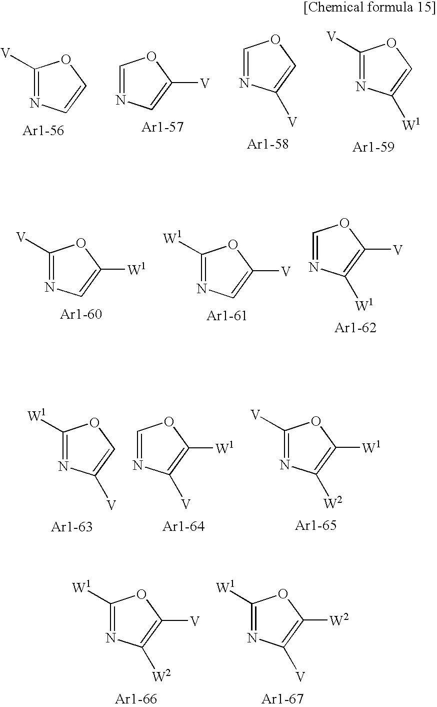
Ar1-59 to Ar1-64 are more preferable; 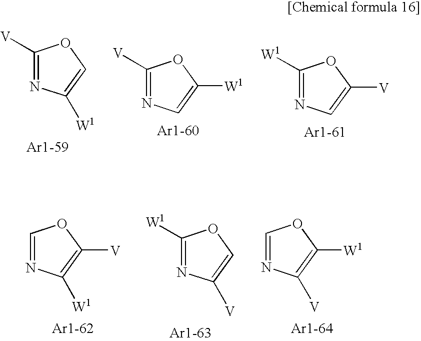
and
Ar1-59 to Ar1-61 and Ar1-63 are particularly preferable:

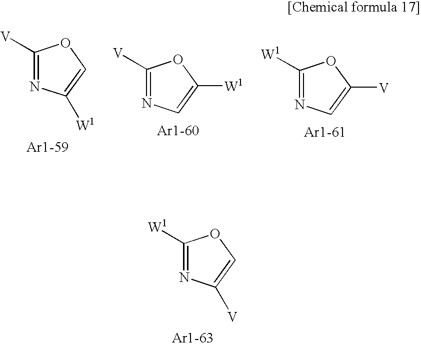

When Ar¹ represents the benzothiazolyl group, Ar1-68 to Ar1-92 are preferable;

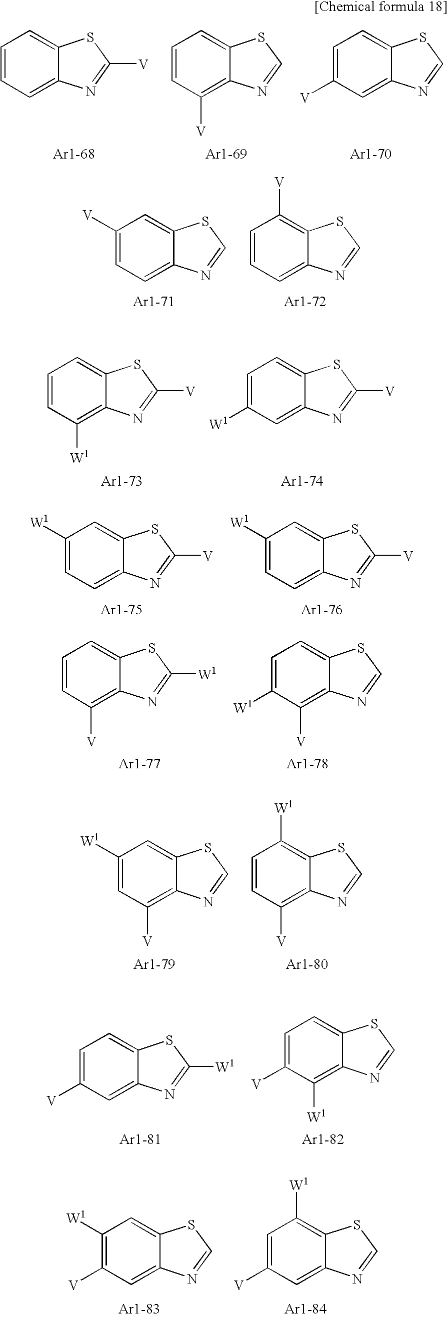

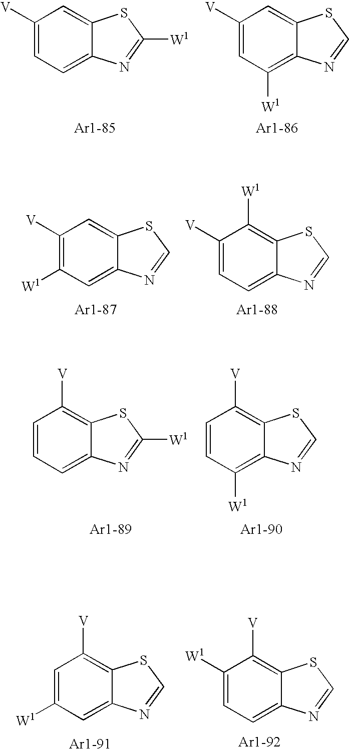

Ar1-68 to Ar1-72 are more preferable; 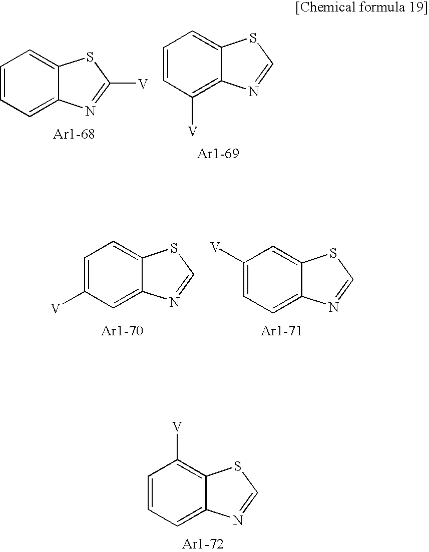
and
Ar1-68, Ar1-70 and Ar1-71 are particularly preferable:

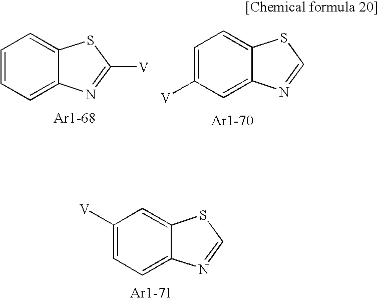

When Ar¹ is substituted, the substituent is at least one group, preferably one or two groups, and more preferably one group preferably selected from the group consisting of halogen atom, hydroxy group, cyano group, carboxy group, alkyl group that may be substituted, alkenyl group that may be substituted, alkynyl group that may be substituted, alkoxy group that may be substituted, alkylthio group that may be substituted, amino group that may be substituted, alkoxycarbonyl group that may be substituted, acyl group that may be substituted, alkylsulfinyl group that may be substituted, alkylsulfonyl group that may be substituted, aryl group that may be substituted, —CON(R¹¹) (R¹²) (R¹¹ and R¹² may be the same or different, each independently represents hydrogen atom, alkyl group that may be substituted or aryl group that may be substituted, or R¹¹ and R¹² are combined to form 3 to 7 membered ring representing a cyclic amine as N(R¹¹)(R¹²)), and —SO₂N(R¹³) (R⁴) (R¹³ and R¹⁴ may be the same or different, each independently represents hydrogen atom, alkyl group that may be substituted or aryl group that may be substituted, or R¹³ and R¹⁴ are combined to form 3 to 7 membered ring representing a cyclic amine N(R¹³) (R¹4). Ar¹ is preferably substituted with two groups in another embodiment. When Ar¹ is substituted, the substituent is more preferably an arbitrary group selected from the group consisting of halogen atom, hydroxy group, cyano group, alkyl group that may be substituted, alkoxy group that may be substituted, alkylthiogroup that may be substituted, amino group that may be substituted, alkoxycarbonyl group that may be substituted, acyl group that may be substituted, alkylsulfonyl group that may be substituted, aryl group that may be substituted, —CON(R¹¹) (R¹¹) (R¹¹ and R¹² are the same as described above), and —SO₂N(R¹³)(R¹⁴) (R¹³ and R¹⁴ are the same as described above); and further preferably an arbitrary group selected from the group consisting of halogen atom, alkyl group that may be substituted, alkoxy group that may be substituted, amino group that may be substituted, alkylsulfonyl group that may be substituted, aryl group that may be substituted, and —CON(R¹¹) (R¹²) (R¹¹ and R¹² are the same as described above). In another embodiment, the substituent is preferably selected from the group consisting of halogen atom, alkyl group that may be substituted, alkoxy group that may be substituted, alkylthio group that may be substituted, amino group that may be substituted, and acyl group that may be substituted when Ar¹ is substituted. In a different embodiment, the substituent is preferably selected from the group consisting of halogen atom, alkyl group that may be substituted, alkoxy group that may be substituted and amino group that may be substituted when Ar¹ is substituted. In a further different embodiment, the substituent is preferably alkoxy group that may be substituted when Ar¹ is substituted. In a further different embodiment, amino group that may be substituted is a particularly preferable substituent when Ar¹ is substituted. While the substituent is not particularly restricted when the substituent in Ar¹ is amino group that may be substituted, —NH₂, alkylamino group, dialkylamino group, acylamino group, acyl(alkyl)amino group, alkylsulfonylamino group, alkylsulfonyl(alkyl)amino group, alkylamino group arbitrarily substituted with at least one halogen atom, alkylsulfonylamino group arbitrarily substituted with at least one halogen atom, or alkylsulfonyl(alkyl)amino group arbitrarily substituted with at least one halogen atom is preferable; and —CON(R¹¹) (R¹²) )(R¹¹ and R¹² are the same as described above) is particularly preferable as the substituent in another embodiment when Ar¹ is substituted. In a different embodiment, halogen atom is particularly preferable as the substituent when Ar¹ is substituted. Fluorine, chlorine and bromine are preferable as the halogen atom, and bromine is particularly preferable. In another embodiment, chlorine and bromine are preferable as the substituent. Chlorine is preferable as the substituent in a different embodiment, and fluorine is preferable as the substituent in a further different embodiment. When Ar¹ is substituted with a plurality of substituents, they may be the same or different to one another.

In an example of a particularly preferable combination, Ar¹ is thienyl group substituted with one halogen atom, and has the bonding mode of above-mentioned Ar1-19.

Ar² represents -E-Ar²¹-G-Q (Ar²¹ represents a benzene ring or naphthalene ring; E represents a single bond or alkylene group; G represents a single bond, alkylene group or alkenylene group; Q represents carboxy group, —CON(R⁴¹) (R⁴²) (R⁴¹ and R⁴² may be the same or different, and each independently represents hydrogen atom, hydroxy group, alkyl group that may be substituted or aryl group that may be substituted, or R⁴¹ and R⁴² are combined to form a 3 to 7 membered ring representing a cyclic amine as N(R⁴¹) (R⁴²), where R⁴² is a group other than hydroxy group when R⁴¹ represents hydroxy group); or —COOR⁴³ (R⁴³ represents alkyl group that may be substituted or aryl group that may be substituted)), -E-Ar²¹-G²-G-Q (E, Ar²¹, G and Q are the same as described above, and G² represents —O—, —S—, —SO—, —SO²— or —NR^{G21} (R^{G21} represents hydrogen atom, alkyl group that may be substituted, acyl group that may be substituted, or sulfonyl group that may be substituted)), or monocyclic heteroaromatic ring other than pyrazolyl group. Ar² may be substituted.

An example of Ar² is -E-Ar²¹-G-Q, -E-Ar²¹-G²-G-Q or a monocyclic heteroaromatic ring other than pyrazolyl group. Ar² is preferably -E-Ar²¹-G-Q or -E-Ar²¹-G²-G-Q, more preferably -E-Ar²¹-G-Q. -E-Ar²¹-G²-G-Q is more preferable in another embodiment.

While Ar² may be substituted or non-substituted, Ar²is preferably non-substituted. Ar² is preferably substituted in another embodiment.

When Ar² is substituted, the substituent is at least one group arbitrarily selected from the group consisting of halogen atom, hydroxy group, cyano group, nitro group, carboxy group, alkyl group that may be substituted, alkenyl group that may be substituted, alkynyl group that may be substituted, alkoxy group that may be substituted, alkylthio group that may be substituted, amino group that may be substituted, alkoxycarbonyl group group that may be substituted, acyl group that may be substituted, alkylsulfinyl group that may be substituted, alkylsulfonyl group that may be substituted, aryl group that may be substituted, —CON(R¹¹) (R¹²) (R¹¹ and R¹² may be the same or different, each independently represents hydrogen atom, alkyl group that may be substituted or aryl group that may be substituted, or R¹¹ and R¹² are combined to form 3 to 7 membered ring representing a cyclic amine as N(R¹¹)(R¹²)), and —SO₂N(R¹³ )(R¹⁴) (R¹³ and R¹⁴ may be the same or different, each independently represents hydrogen atom, alkyl group that may be substituted or aryl group that may be substituted, or R¹³ and R¹⁴ are combined to form 3 to 7 membered ring representing a cyclic amine N(R¹³)(R¹⁴)) More preferable, the substituent is at least one group arbitrarily selected from the group consisting of halogen atom, hydroxy group, cyano group, alkyl group that may be substituted, alkoxy group that may be substituted, alkylthio group that may be substituted, amino group that may be substituted, alkoxycarbonyl group that may be substituted, acyl group that may be substituted, alkylsulfonyl group that may be substituted, aryl group that may be substituted, —CON(R¹¹)(R¹²) (R¹¹ and R¹² are the same as described above), and —SO₂N(R¹³)(R¹⁴) (R¹³ and R¹⁴ are the same as described above). Further preferably, the substituent is at least one group arbitrarily selected from the group consisting of halogen atom, alkyl group that may be substituted, alkoxy group that may be substituted, and amino group that may be substituted. When Ar² is substituted, the particularly preferable substituent is halogen atom, alkyl group that may be substituted, alkoxy group that may be substituted or alkylthio group that may be substituted. In a particularly preferable embodiment, Ar² is substituted with one or a plurality of groups selected from the group consisting of hydroxy group, halogen atom, alkyl group that may be substituted, and alkoxy group that may be substituted. When Ar² is substituted with halogen atom, the halogen atom is preferably fluorine, chlorine or bromine, and bromine is particularly preferable. In another embodiment, the halogen atom is preferably chlorine or fluorine. In a different embodiment, the halogen atom is preferably chlorine, and fluorine is preferable in a further different embodiment. When Ar² is substituted with a plurality of substituents, the substituents may be the same or different to one another. The substituent is preferably substituted with one or two groups, and is more preferably substituted with one group. In another embodiment, the substituent is more preferably substituted with two groups. The substituent that may be substituted on Ar² may be bonded together to another substituent that may be substituted on Ar² or to a part of Ar², or may be bonded to oxygen atom, sulfur atom or nitrogen atom on the same substituent to form a ring. An example when the substituent on Ar² is bonded together to a part of Ar² or bonded together to oxygen atom, sulfur atom or nitrogen atom on the same substituent is the case in which Ar² is phthalimide-5-yl group or 1,4-dioxo-1,2,3,4-tetrahydrophthaladine-6-yl group. When Ar2 is substituted with two alkyl groups, it is also preferable that the alkyl groups form a ring together.

When Ar² is substituted, the substitution site of the substituent is a site capable of substituting on Ar². When Ar² is -E-Ar²¹-G-Q and is substituted, the substitution site of the substituent is preferably on E, Ar²¹ or G, more preferably on Ar²¹ or G, and further preferably on Ar²¹. When Ar² is -E-Ar²¹-G-Q and is substituted, and when the substitution site of the substituent on G and G is alkylene group, a preferable example of the substituent is the same as the example shown in the above-mentioned substituent of Ar². However, another preferable example is that two bonds formed by removing two hydrogen atoms on the same carbon of the alkylene group is bonded to the same oxygen atom together to form an oxo-substituent.

A specific example of Ar²¹ is benzene ring or naphthalene ring, and benzene ring is preferable.

While a specific example of E includes a single bond or lower alkylene group, the single bond, methylene group or ethylene group is preferable, the single bond or ethylene group is more preferable, and the single bond is further preferable.

While a specific example of G includes a single bond, lower alkylene group or lower alkenylene group, the single bond, methylene group, ethylene group or vinylene group is preferable, the single bond, methylene group or vinylene group is more preferable, the single bond or methylene group is further preferable, and methylene group is particularly preferable. In another embodiment, G is preferably a single bond or lower alkylene group, and lower alkylene group is more preferable. In the combination of E and G, it is preferable that E is a single bond and G is a single bond or lower alkylene group; and it is more preferable that E is a single bond and G is lower alkylene group.

Specific examples of the substitution sites of E and G in Ar²¹ as E-Ar²¹-G are Ar21-1 to Ar21-17;

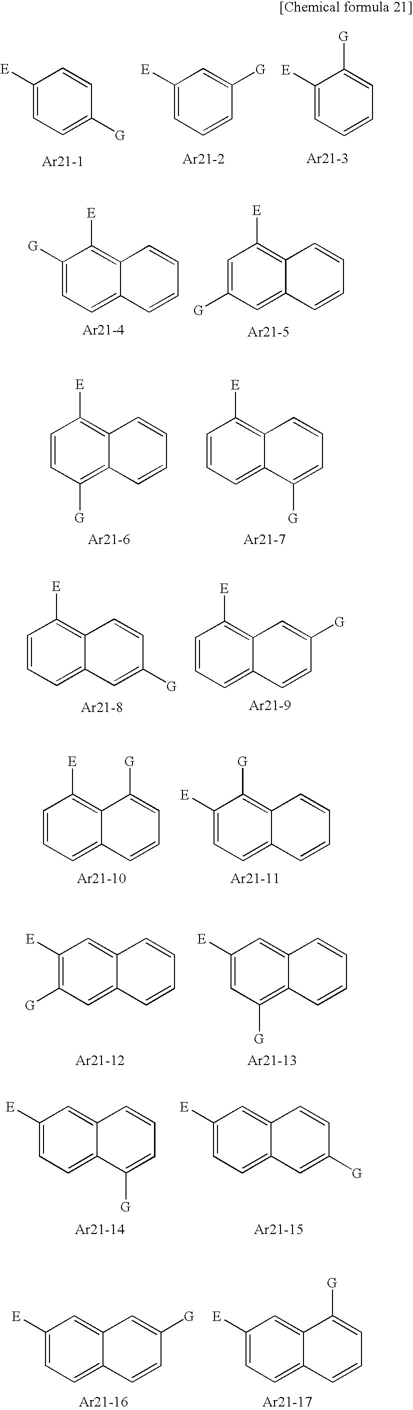

preferably Ar21-1, Ar21-2, Ar21-5 to At21-9 and Ar21-13 to Ar21-17;

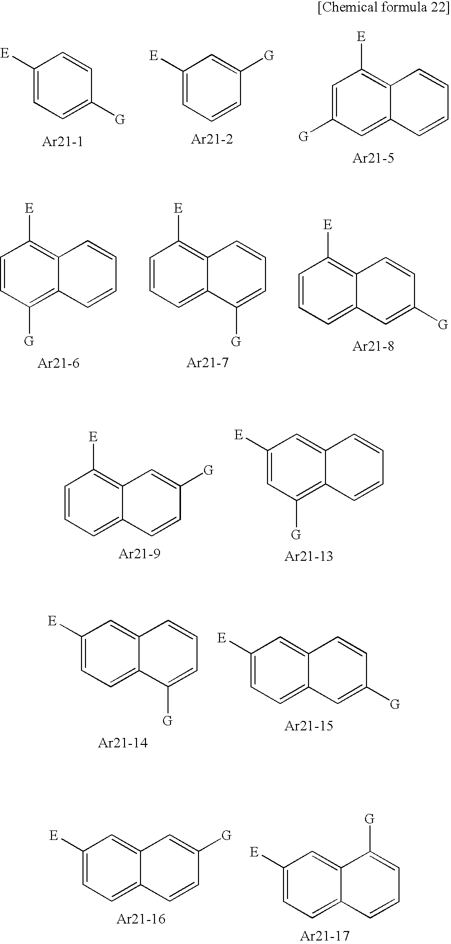

more preferably Ar21-1, Ar21-2, Ar21-7, Ar21-8 and Ar21-14 to Ar21-16;

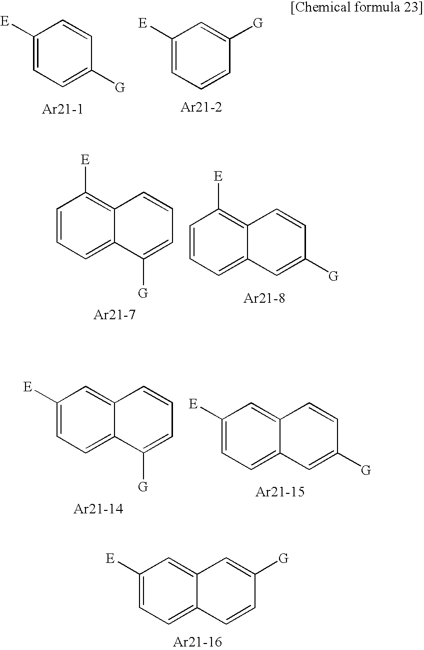

further preferably Ar21-1 and Ar21-15; 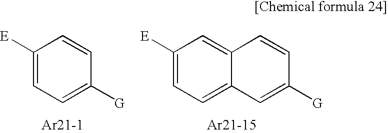
and
particularly Ar21-1:

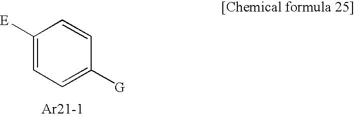

A specific example of Q is a carboxycarboxy group, —CON(R⁴¹) (R⁴²) or —COOR⁴³. A carboxycarboxy group or —CON(R⁴¹) (R⁴²) is preferable, and the carboxycarboxy group is more preferable.

R⁴¹ and R⁴² may be the same or different to one another, and each independently represents hydrogen atom, hydroxy group, alkyl group that may be substituted or aryl group that may be substituted, or R⁴¹ and R⁴² are combined together to form a 3 to 7 membered ring representing a cyclic amine as N(R⁴¹) (R⁴²). R⁴² is a group other than hydroxy group when R⁴¹ is hydroxy group. R⁴¹ is preferably hydrogen atom or alkyl group that may be substituted, more preferably hydrogen atom or methyl group, and most preferably hydrogen atom. R⁴² is preferably hydrogen atom, hydroxy group, alkyl group that may be substituted with aryl group that may be substituted, or aryl group that may be substituted with alkoxy group; more preferably hydrogen atom, hydroxy group or alkyl group; and further preferably hydrogen atom, hydroxy group or methyl group.

A specific example of R⁴³ is alkyl group that may be substituted or aryl group that may be substituted; preferably alkyl group that may be substituted; more preferably lower alkyl group; and particularly methyl or ethyl group.

While G² is not particularly restricted so long as it is —O—, —S—, —SO—, —SO₂— or —NR^{G21}— (R^{G21} represents hydrogen atom, alkyl group that may be substituted, acyl group that may be substituted, or sulfonyl group that may be substituted) when Ar² represents -E-Ar²¹-G²-G-Q, —O—, —S— or —NH— is preferable, and —S— is particularly preferable. R^{G21} in —NR^{G21} is more preferably hydrogen atom. Preferable examples of E, Ar²¹, G and Q are as described above when Ar² is -E-Ar²¹-G²-G-Q.

The example described below is also preferable as Ar². Ar² represents carboxyphenyl group, carboxyalkylphenyl -group, carboxynaphthyl group, carboxyalkylnaphthyl group or a monocyclic heteroaromatic ring other than pyrazolyl group, and Ar² may be substituted. Ar² is preferably carboxyphenyl group, carboxyalkylphenyl group or carboxynaphthyl group. In another embodiment, Ar² is preferably carboxyphenyl group, carboxyalkylphenyl group or monocyclic heteroaromatic ring other than pyrazolyl group. Carboxyphenyl group is particularly preferable. Carboxyalkylphenyl group is particularly preferable as Ar² in a different embodiment. Carboxynaphthyl group is particularly preferable as Ar² in a further different embodiment.

When Ar² is carboxyalkylphenyl group or carboxyalkylnaphthyl group, “alkyl” in these groups preferably has a carbon number in the range from 1 to 6, more preferably a carbon number in the range from 1 to 3, further preferably a carbon number of 1 or 2, and particularly a carbon number of 1.

Preferable examples showing the site and number of substituents are shown below when Ar² is in the site bonding to an amino group and has substituents on it in general formula (1). In the structured below, X represents the site for binding to amino group, Y¹ and Y² represent the sites to which substituents are bonded, and Z represents alkylene group in general formula (1).

When Ar² is carboxyphenyl group, Ar2-1 to Ar2-13 are preferable; 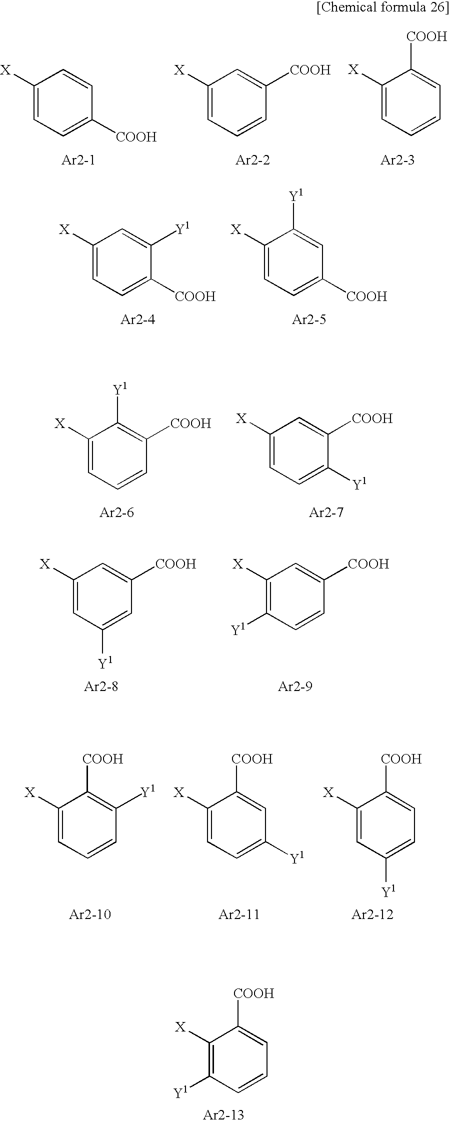
Ar2-1, Ar2-2 and Ar2-4 to Ar2-9 are more preferable; 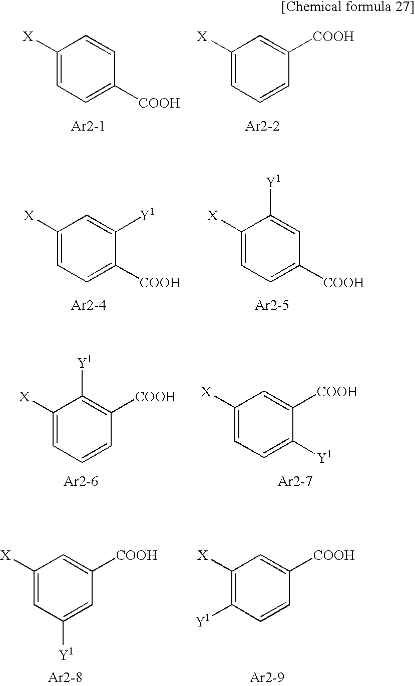
Ar2-1, Ar2-2, Ar2-4 and Ar2-5 are further preferable; 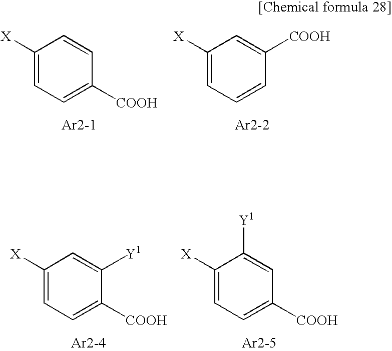
Ar2-1, Ar2-4 and AR2-5 are particularly preferable; 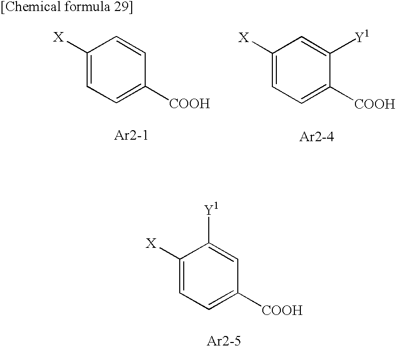
and
Ar2-1 is most preferable:

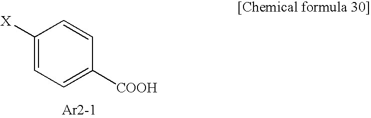

When Ar² is carboxyalkylphenyl group, Ar2-14 to Ar2-26 are preferable; 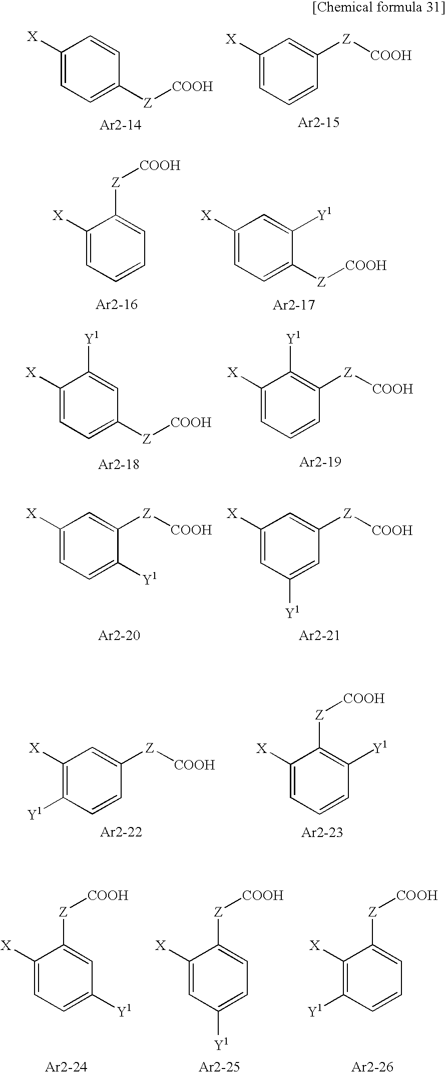
Ar2-14 to Ar2-22 are more preferable;

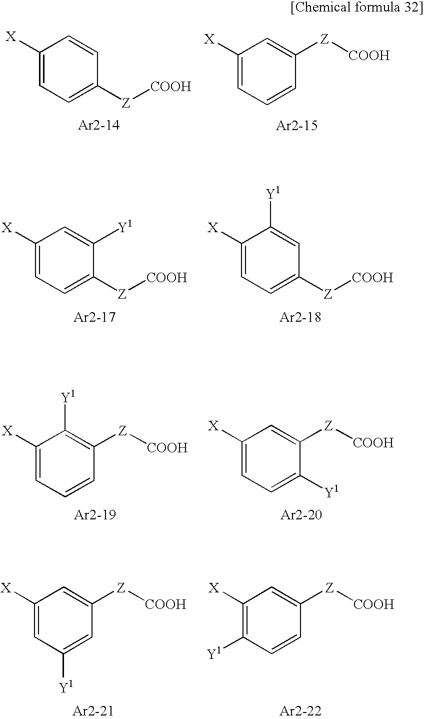

Ar2-14, Ar2-17 and Ar2-18 are further preferable; 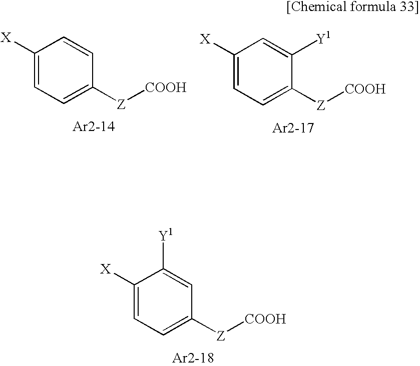
and
Ar2-14 is particularly preferable:

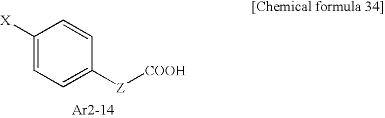

When Ar² is carboxynaphthyl group, Ar2-27 to Ar2-29 are preferable;

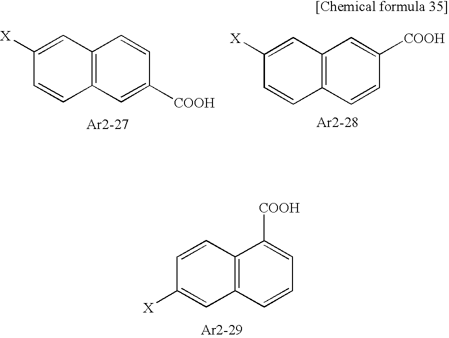

Ar2-27 and Ar2-28 are more preferable; 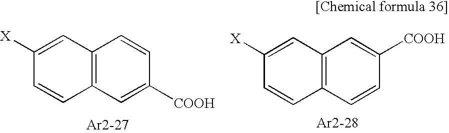
and
Ar2-27 is particularly preferable:

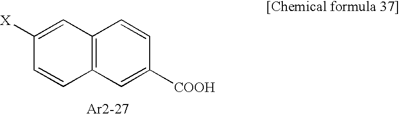

When Ar² is carboxyalkylnaphthyl group, Ar2-30 to Ar2-32 are preferable;

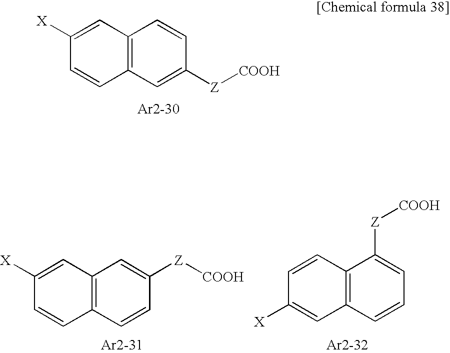

Ar2-30 and Ar2-31 are more preferable; 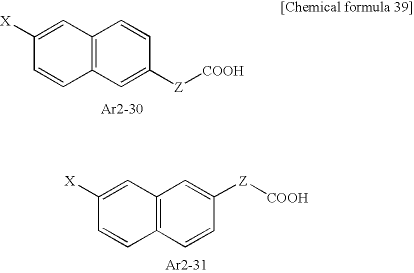
and
Ar2-30 is particularly preferable:

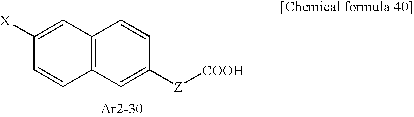

When Ar² is substituted, the substituent is preferably at least one group arbitrarily selected form the group consisting of halogen atom, hydroxy group, cyano group, carboxy group, alkyl group that may be substituted, alkenyl group that may be substituted, alkynyl group that may be substituted, alkoxy group that may be substituted, alkylthio group that may be substituted, amino group that may be substituted, alkoxycarbonyl group that may be substituted, acyl group that may be substituted, alkylsulfinyl group that may be substituted, alkylsulfonyl group that may be substituted, aryl group that may be substituted, —CON(R¹¹) (R¹²) (R¹¹ and R¹² may be the same or different, each independently represents hydrogen atom, alkyl group that may be substituted or aryl group that may be substituted, or R¹¹ and R¹² are combined to form 3 to 7 membered ring representing a cyclic amine as N(R¹¹)(R¹²)), and —SO₂N(R¹³)(R¹⁴) (R¹³ and R¹⁴ may be the same or different, each independently represents hydrogen atom, alkyl group that may be substituted or aryl group that may be substituted, or R¹³ and R¹⁴ are combined to form 3 to 7 membered ring representing a cyclic amine N(R¹³)(R¹⁴)); more preferably an arbitrary group selected from the group consisting of halogen atom, hydroxy group, cyano group, alkyl group that may be substituted, alkoxy group that may be substituted, alkylthio group that may be substituted, amino group that may be substituted, alkoxycarbonyl group that may be substituted, acyl group that may be substituted, alkylsulfonyl group that may be substituted, aryl group that may be substituted, —CON(R¹¹) (R¹²) (R¹¹ and R¹² have the same meanings as described above), and —SO₂N(R¹³) (R¹⁴) (R¹³ and R¹⁴ are the same described above); and further preferably an arbitrary group selected from the group consisting of halogen atom, alkyl group that may be substituted, alkoxy group that may be substituted, and amino group that may be substituted. When Ar² is substituted, the particularly preferable substituent is halogen atom, alky group that may be substituted, or alkoxy group that may be substituted. The substituents may be the same or different to one another when Ar² is substituted with a plurality of substituents.

A particularly preferable combination of the above-mentioned substituents comprises an example in which carboxyalkylphenyl group, the alkyl group is a methylene group, the phenyl group is substituted with one halogen atom, and the combination is represented by the chemical structure of above-mentioned Ar2-18; or an example in which Ar² is carboxyalkylphenyl group, the alkyl group is a methylene group, and the combination is represented by the chemical structure of above-mentioned Ar2-14.

R¹ and R² in general formula (1) may be the same or different, and each independently represents hydrogen atom, alkyl group that may be substituted, alkenyl group that may be substituted, alkoxy group that may be substituted, alkylthio group that may be substituted, alkylsulfinyl group that may be substituted, or alkylsulfonyl group that may be substituted. Alkyl group that may be substituted, or alkenyl group that may be substituted are preferable as R¹ and R². R¹ is preferably alkyl group that may be substituted; more preferably lower alkyl group with a carbon number in the range from 1 to 3; particularly methyl group or ethyl group; and most preferably ethyl group. Methyl group is most preferable as R¹ in another embodiment. R² is more preferably alkyl group that may be substituted or alkenyl group that may be substituted; further preferably lower alkyl group with a carbon number in the range from 1 to 4, or lower alkenyl group with a carbon number in the range from 2 to 4; particularly methyl group, ethyl group, propyl group or allyl group; and most preferably ethyl group or allyl group. Allyl group is most preferable in another embodiment. Ethyl group is most preferable in a different embodiment, or methyl group substituted with hydroxy group is most preferable in a further different embodiment.

R³ is hydrogen atom or alkyl group that may be substituted. R³ is preferably hydrogen atom or lower alkyl group, more preferably hydrogen atom or methyl group, and particularly hydrogen atom.

While the combination of the substituents of the compound represented by general formula (1) is not particularly restricted, the following compounds are preferable:
<1> the compound in which Ar¹ is furyl group;
<2> the compound in which Ar¹ is thienyl group;
<3> the compound in which Ar¹ is triazolyl group;
<4> the compound in which Ar¹ is thiazolyl group;
<5> the compound in which Ar¹ is oxazolyl group;
<6> the compound in which Ar¹ is benzothiazolyl group;
<7> the compound in which Ar¹ is substituted with alkoxy group that may be substituted;
<8> the compound in which Ar¹ is substituted with amino group that may be substituted;
<9> the compound in which Ar¹ is substituted with —CON(R¹¹) (R¹²) (R¹¹ and R¹² are the same as described above);
<10> the compound in which Ar² is carboxyphenyl group;
<11> the compound in which Ar² is carboxyalkylphenyl group;
<12> the compound in which Ar² is carboxynaphthyl group;
<13> the compound in which Ar² is carboxyalkylnaphthyl group;
<14> monocyclic heteroaromatic ring other than pyrazolyl group;
<15> the compound in which Ar² is not substituted;
<16> the compound in which Ar² is substituted with halogen;
<17> the compound in which Ar² is substituted with alkyl group that may be substituted;
<18> the compound in which Ar² is substituted with alkoxy group that may be substituted;
<19> the compound in which R¹ is lower alkyl group with a carbon number in the range from 1 to 3;
<20> the compound in which R² is lower alkyl group with a carbon number in the range from 1 to 4;
<21> the compound in which R² is lower alkenyl group with a carbon number in the range from 2 to 4;
<22> the compound in which R³ is hydrogen atom;
<23> the compounds of <1> and <7>;
<24> the compounds of <1> and <8>;
<25> the compounds of <1> and <9>;
<26> the compounds of <1> and <10>;
<27> the compounds of <1> and <11>;
<28> the compounds of <1> and <12>;
<29> the compounds of <1> and <13>;
<30> the compounds of <1> and <14>;
<31> the compounds of <1> and <15>;
<32> the compounds of <2> and <7>;
<33> the compounds of <2> and <8>;
<34> the compounds of <2> and <9>;
<35> the compounds of <2> and <10>;
<36> the compounds of <2> and <11>;
<37> the compounds of <2> and <12>;
<38> the compounds of <2> and <13>;
<39> the compounds of <2> and <14>;
<40> the compounds of <2> and <15>;
<41> the compounds of <4> and <7>;
<42> the compounds of <4> and <8>;
<43> the compounds of <4> and <9>;
<44> the compounds of <4> and <10>;
<45> the compounds of <4> and <11>;
<46> the compounds of <4> and <12>;
<47> the compounds of <4> and <13>;
<48> the compounds of <4> and <14>;
<49> the compounds of <4> and <15>;
<50> the compound of <19> according to any one of <23> to <49>;
<51> the compound of <20> according to any one of <23> to <50>;
<52> the compound of <21> according to any one of <23> to <50>;
<53> the compound of <22> according to any one of <23> to <52>;
<54> the compound in which Ar¹ is substituted with halogen atom;
<55> the compound in which Ar¹ is substituted with alkyl group that may be substituted;
<56> the compound in which Ar¹ is substituted with alkylthio group that may be substituted;
<57> the compound in which Ar¹ is substituted with acyl group that may be substituted;
<58> the compound in which Ar¹ is substituted with alkyl group;
<59> the compound in which Ar¹ is substituted with alkylthio group;
<60> the compound in which Ar¹ is substituted with acyl group;
<61> the compound in which Ar¹ is substituted with alkoxy group;
<62> the compound in which Ar² is -E-Ar²¹-G-Q;
<63> the compound in which above-mentioned <62> and Ar²¹ are benzene rings;
<64> the compound in which above-mentioned <62> and Ar²¹ are naphthalene rings;
<65> the compound in which above-mentioned <62> and E are single bonds;
<66> the compound in which above-mentioned <62> and G are single bonds or lower alkylene groups;
<67> the compound in which above-mentioned <62> and G are lower alkylene groups;
<68> the compound in which above-mentioned <62> and G are lower alkylene groups with a carbon number in the range from 1 to 3;
<69> the compound in which above-mentioned <62> and G are methylene groups;
<70> the compound in which above-mentioned <62> and Q are carboxy groups;
<71> the compound in which Ar² is substituted with hydroxy group;
<72> the compound in which Ar² is substituted with alkyl group;
<73> the compound in which Ar² is substituted with alkoxy group;
<74> the compound in which R¹ is lower alkyl group;
<75> the compound in which R² is lower alkyl group;
<76> the compound in which R² is lower alkenyl group;
<77> the compound of <7> according to <8> and <9>, and any one of <54> to <61>;
<78> the compound of <8>, according to <9> and any one of <54> to <61>;
<79> the compound of <9> according to any one of <54> to <61>;
<80> the compound of <54> according to any one of <55> to <61>;
<81> the compound of <55> according to any one of <56> to <61>;
<82> the compound of <56> according to any one of.<57> to <61>;
<82> the compound of <57> according to any one of <58> to <61>;
<83> the compound of <58> according to any one of <59> to <61>;
<84> the compound of <59> according to any one of <60> and <61>;
<85> the compound of <60> and <61>;
<86> the compound of <16> according to <17> and <18>, and any one of <71> to <73>;
<87> the compound of <17>, according to <18> and any one of <71> to <73>;
<88> the compound of <18> according to any one of <71> to <73>;
<89> the compound of <71> according to any one of <72> and <73>;
<90> the compound of <72> and <73>;
<91> the compound of <65> according to any one of <63> and <64>;
<92> the compound of <91> according to any one of <66> to <69>;
<93> the compound of <70> and <92>;
<94> the compound of any on of <7> to <9>, <54> to <61> and <77> to <85> according to any one of <1> and <2>;
<95> the compound of <94> according to any one of <15> to <18>, <71> to <73> and <86> to <90>;
<96> the compound of <95> according to any one of <10> to <14>, <62> to <70> and <91> to <93>;
<97> the compound of <96> according to any one of <19> and <74>;
<98> the compound of <97> according to any one of <20>, <21>, <75> and <76>;
<99> the compound of <22> and <98>;
<100> the compound in which R² is methyl group substituted with lower alkoxy group;
<101> the compound in which R² is methoxymethyl group or ethoxymethyl group;
<102> the compound in which R² is hydroxymethyl group;
<103> the compound in which R² is methyl group substituted with lower alkylthio group;
<104> the compound in which R² is methyl group substituted with lower alkylsulfinyl group;
<105> the compound in which R² is methyl group substituted with lower alkylsulfonyl group;
<106> the compound in which R² is ethyl group;
<107> the compound of <96> according to any one of <100> to <106>;
<108> the compound of <97> according to any one of <100> to <106>; and
<109> the compound of <22> according to any one of <107>and <108>.

The following compounds, salts and prodrugs are also preferable:

the compound represented by general formula (1), or salts of the compound <1> to <109> in which the combination of substituents in the compound represented by general formula (1) is restricted;

the compound represented by general formula (1), or pharmacologically acceptable salts of the compound <1> to <109> in which the combination of substituents in the compound represented by general formula (1) is restricted;

the compound represented by general formula (1), or prodrugs of the compound <1> to <109> in which the combination of substituents in the compound represented by general formula (1) is restricted;

the compound represented by general formula (1), or prodrugs of the salts of the compound <1> to <109> in which the combination of substituents in the compound represented by general formula (1) is restricted; and

the compound represented by general formula (1), or prodrugs of the pharmacologically acceptable salts of the compound <1> to <109> in which the combination of substituents in the compound represented by general formula (1) is restricted.

While specific and preferable examples of the compound of the invention represented by general formula (1) are the following compounds, the scope of the invention is not restricted to these preferable examples:

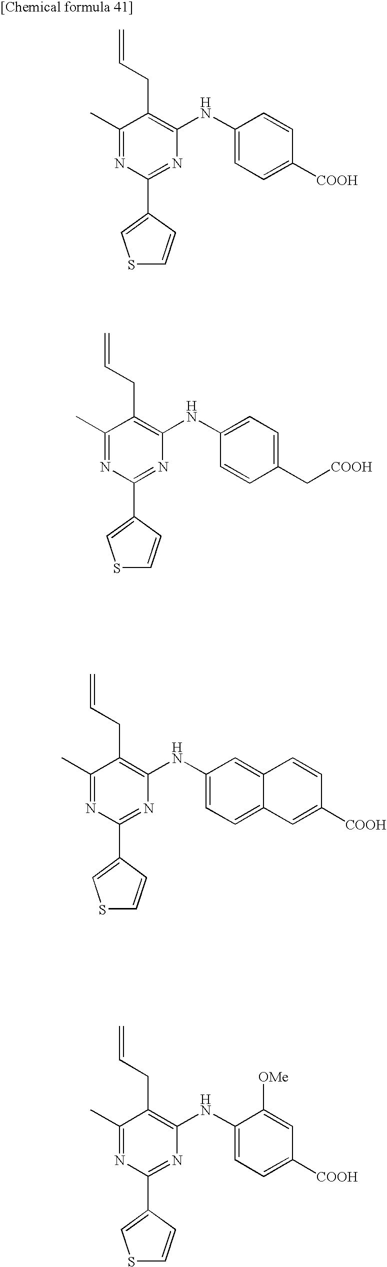

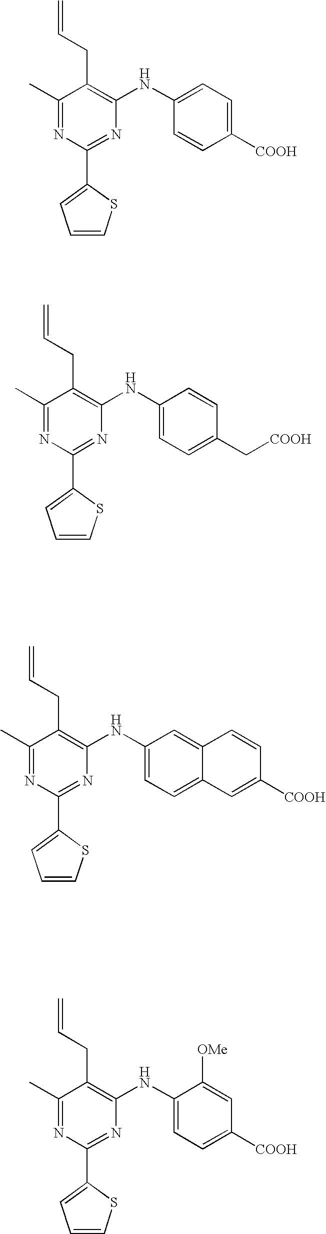

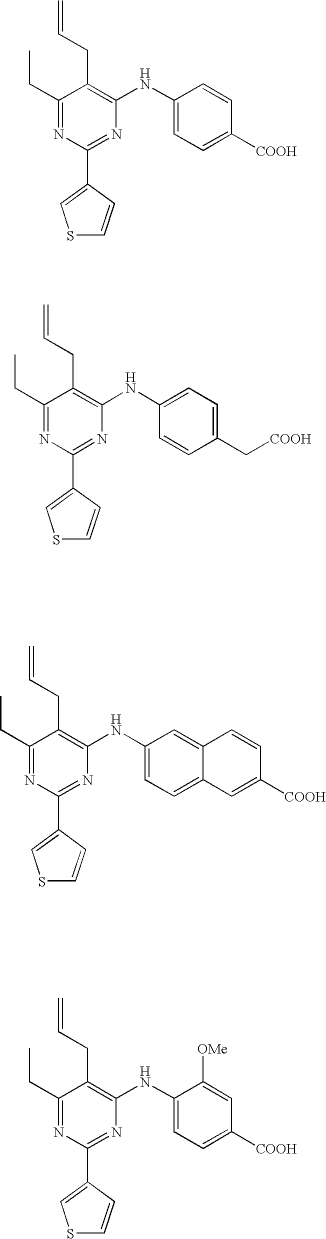

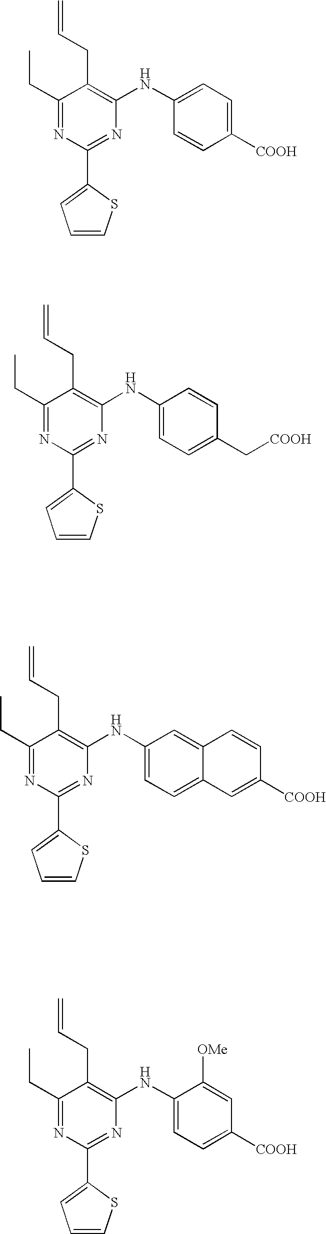

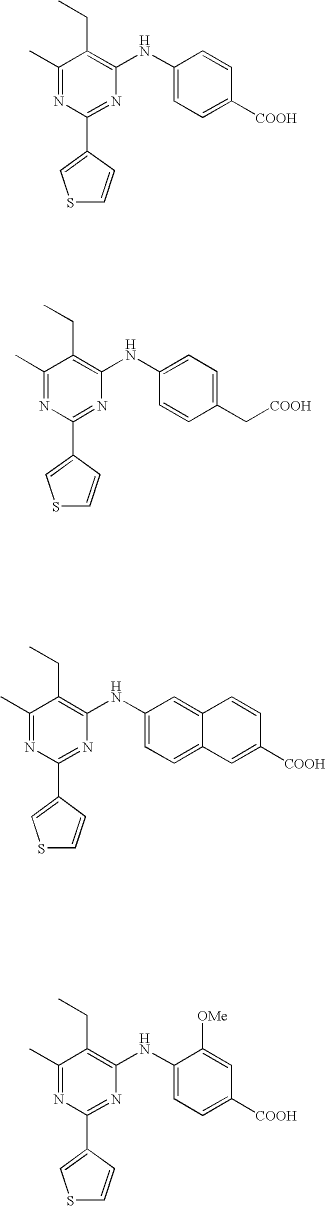

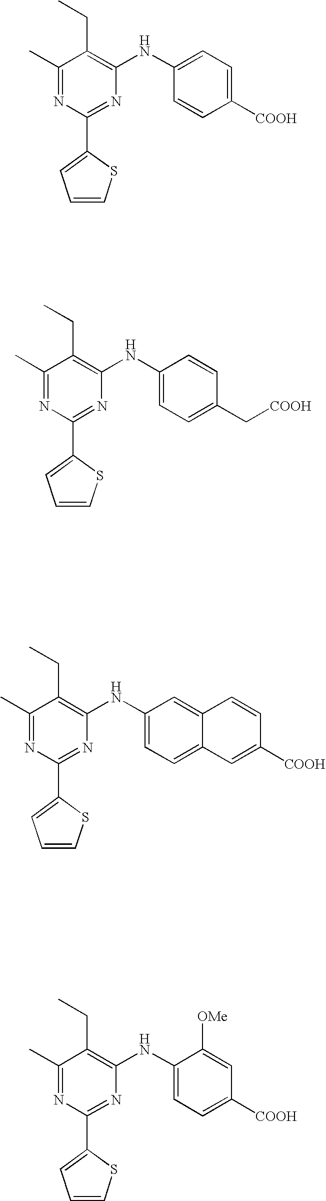

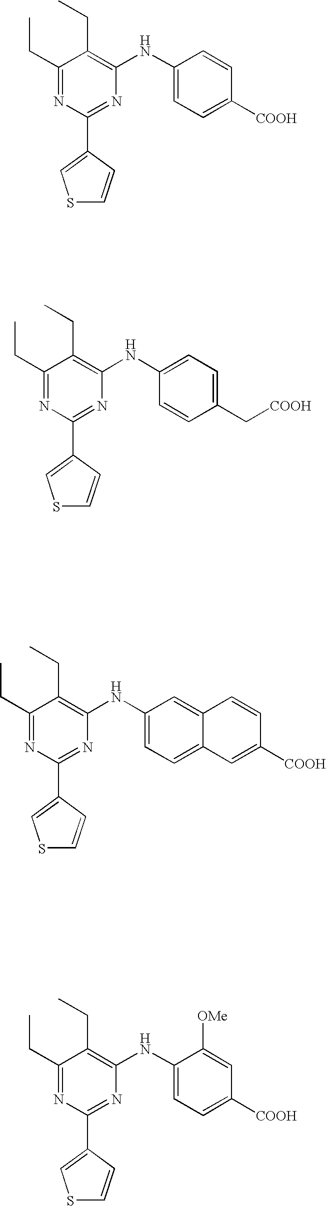

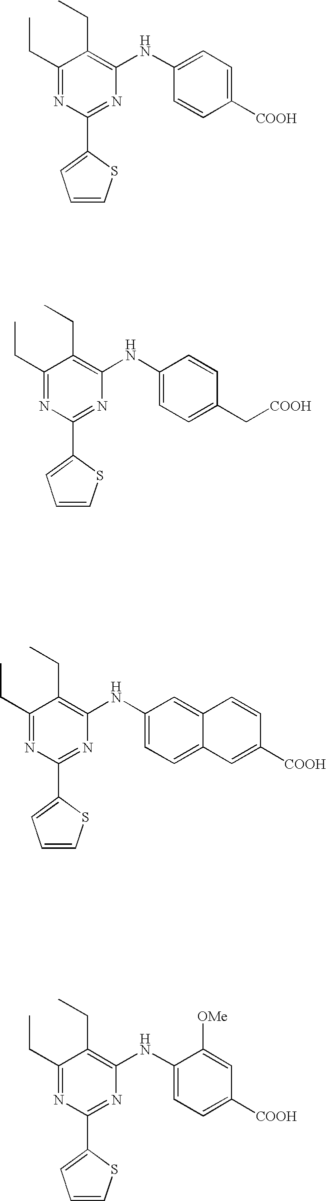

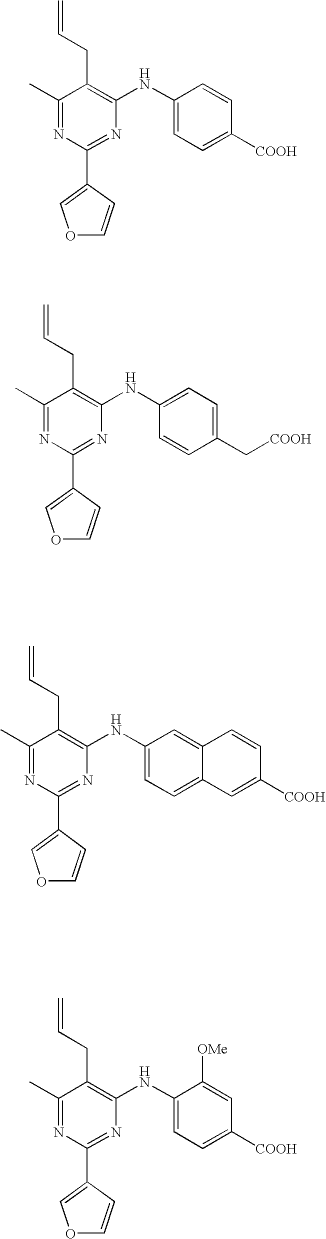

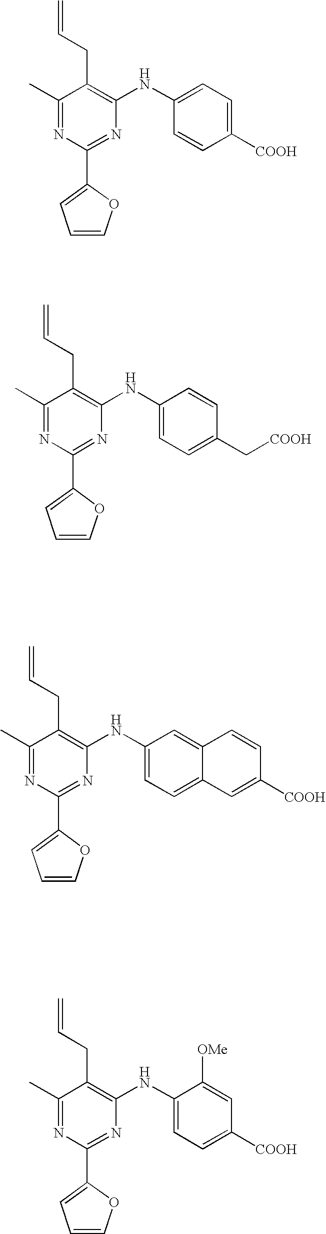

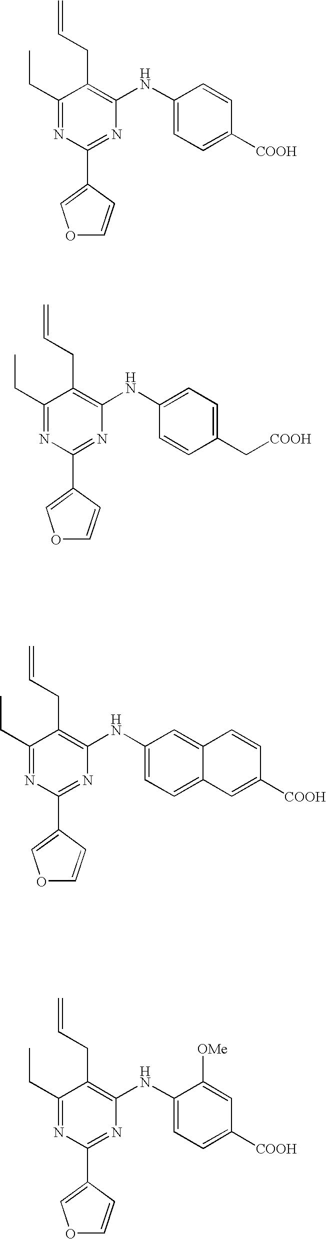

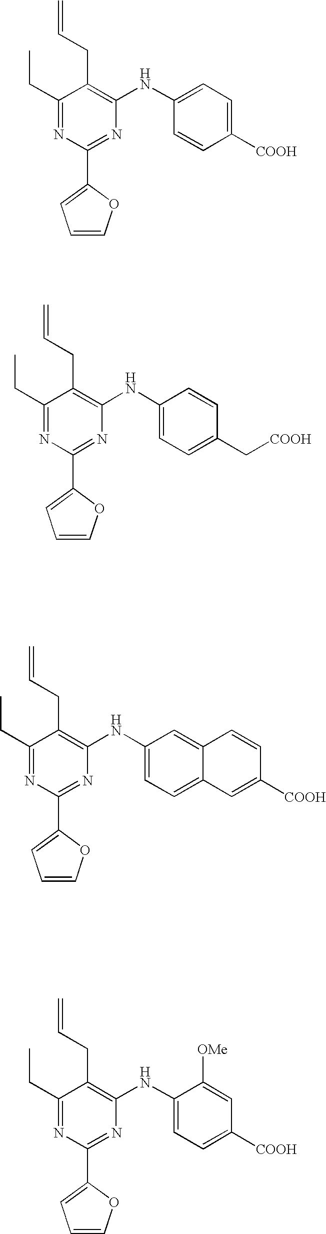

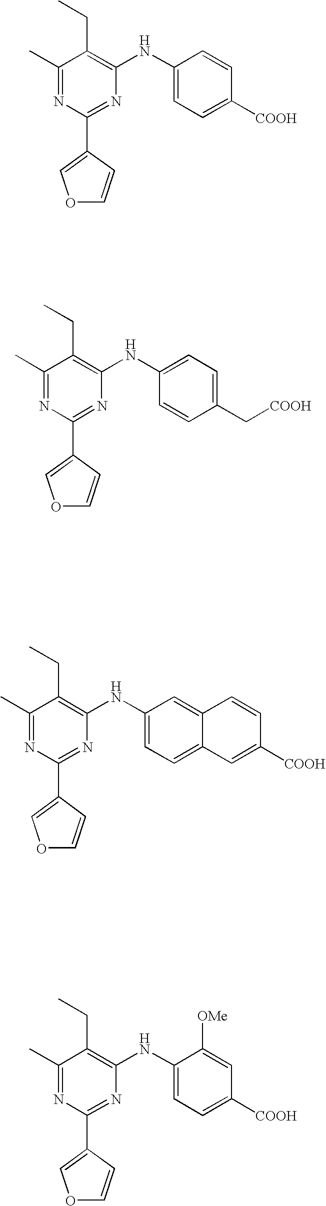

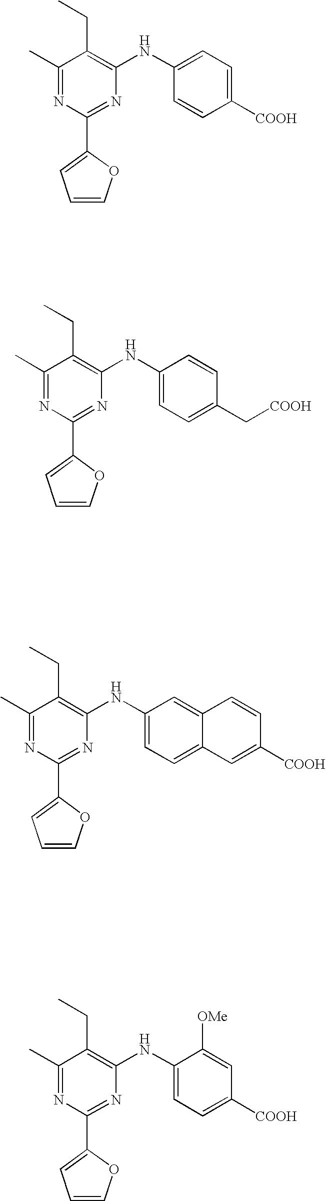

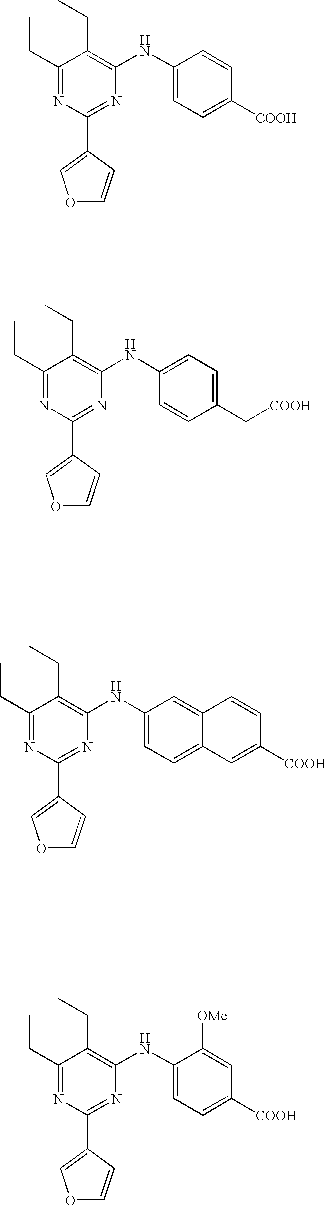

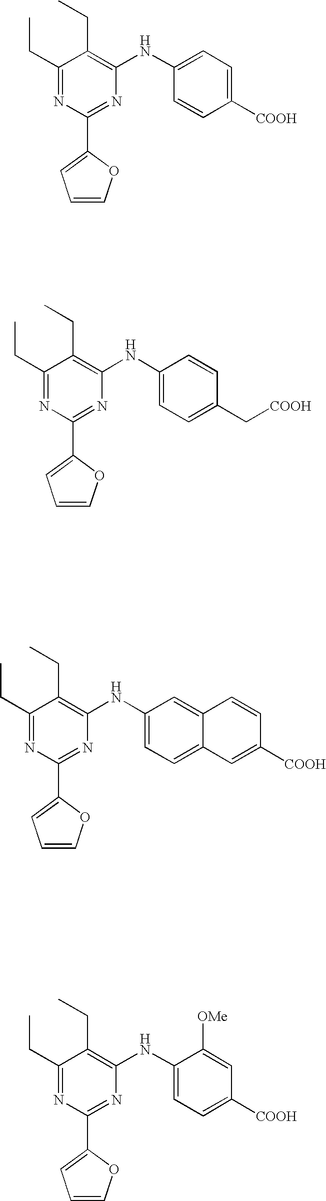

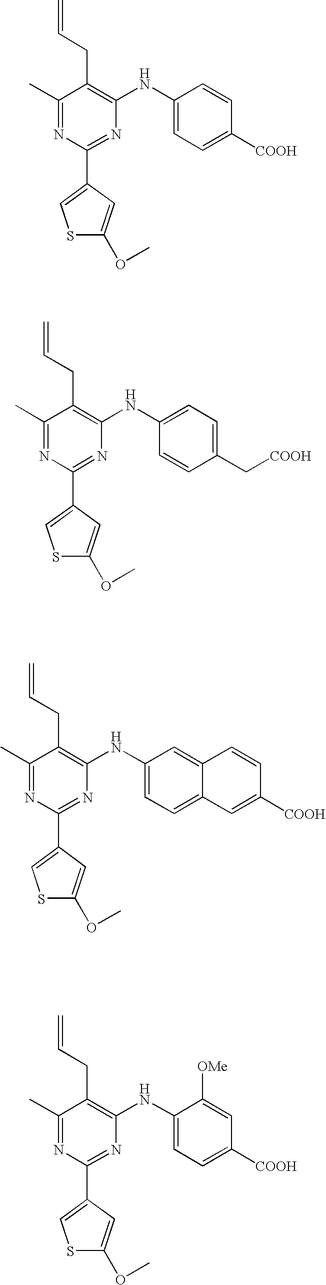

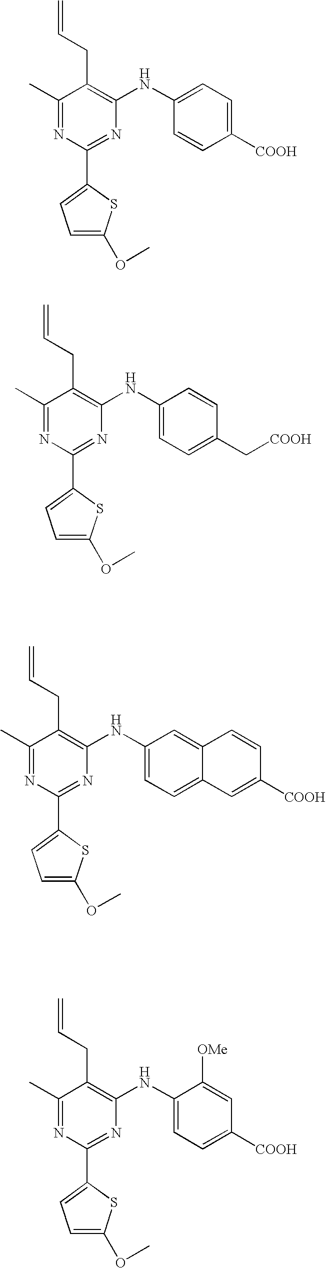

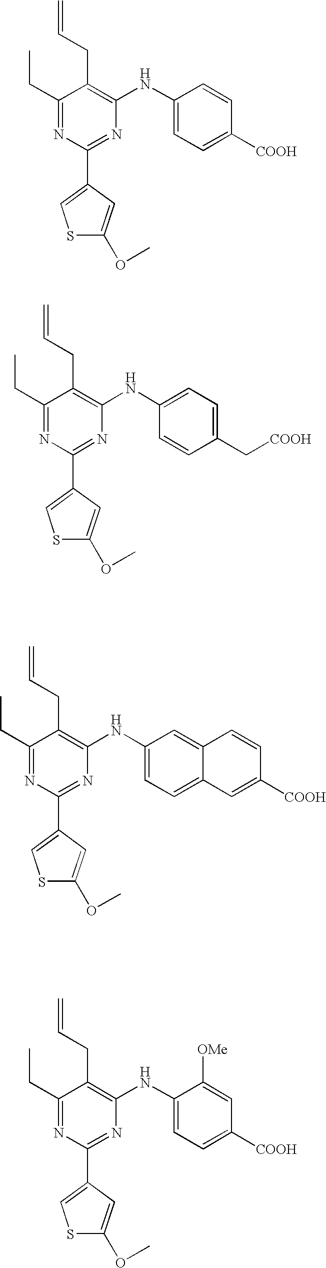

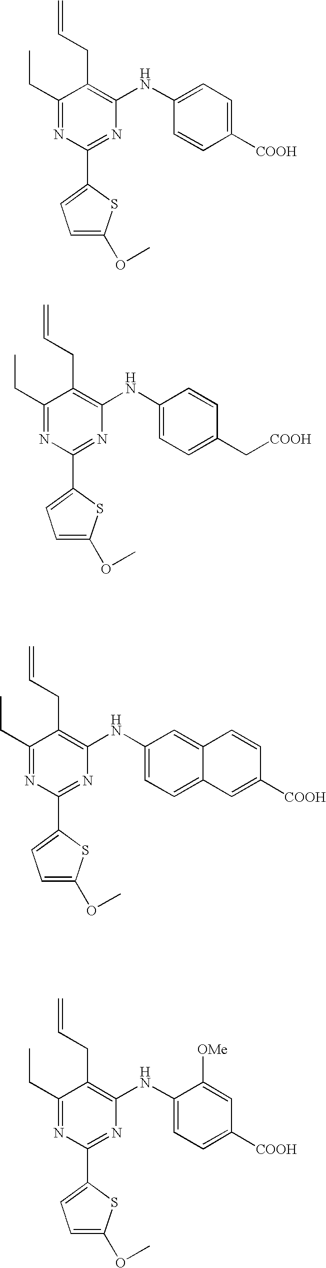

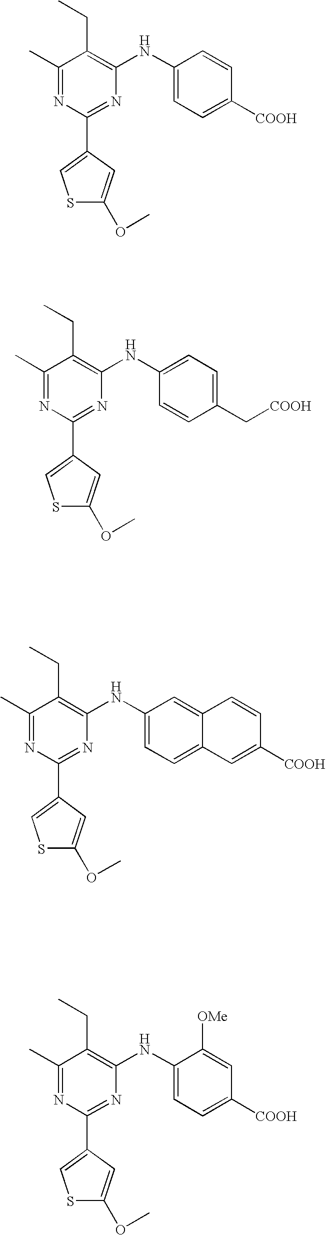

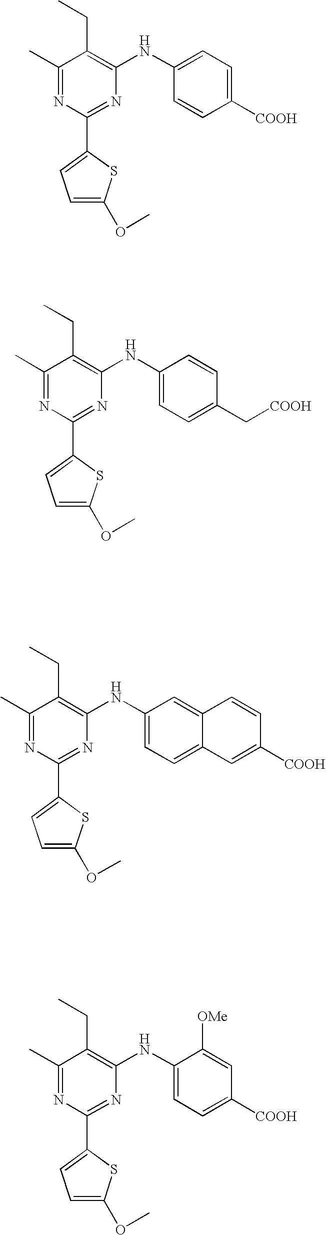

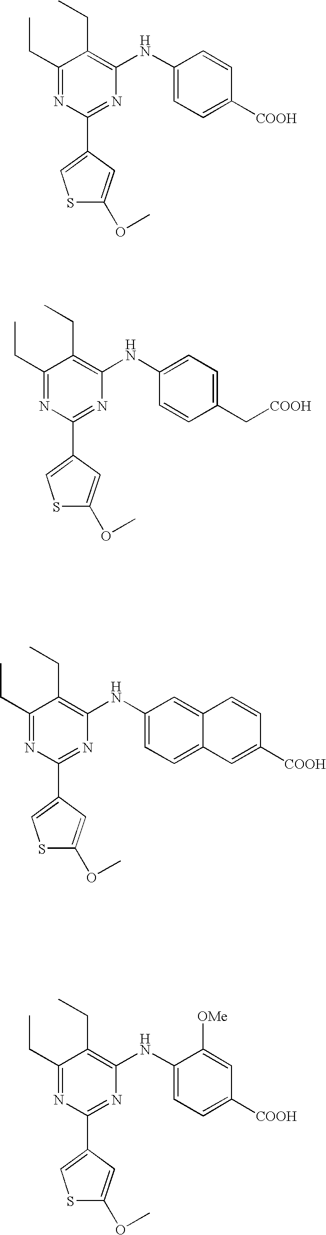

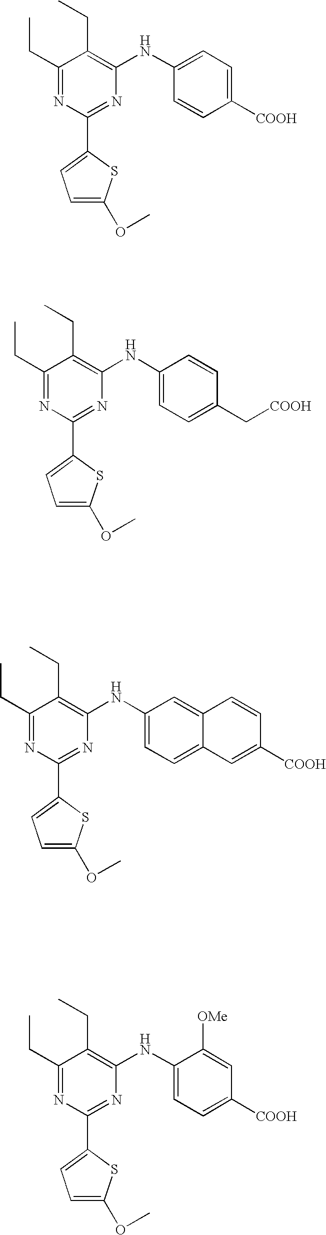

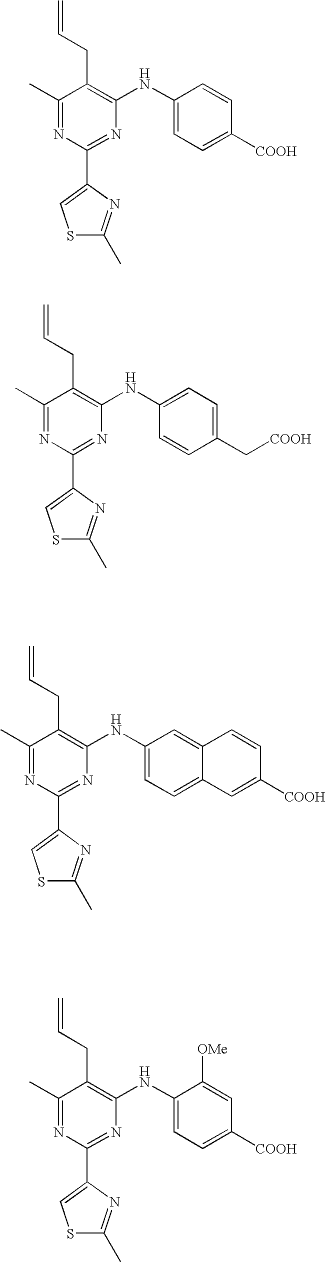

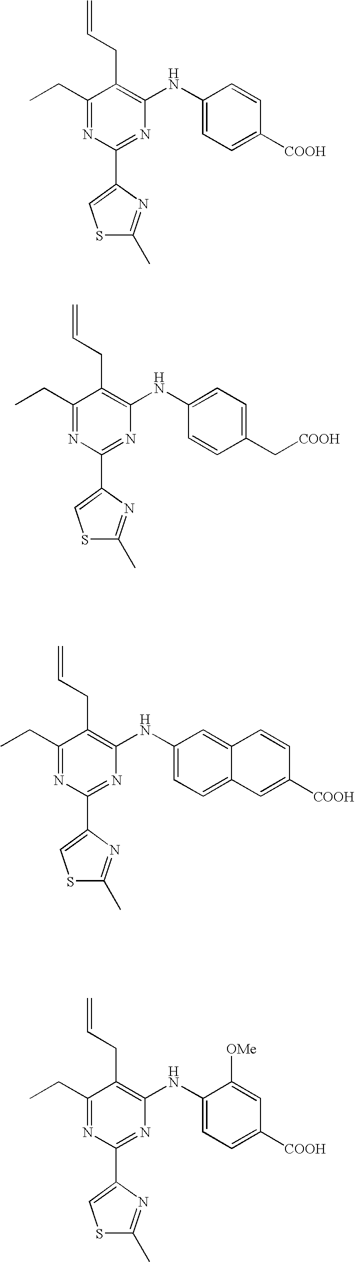

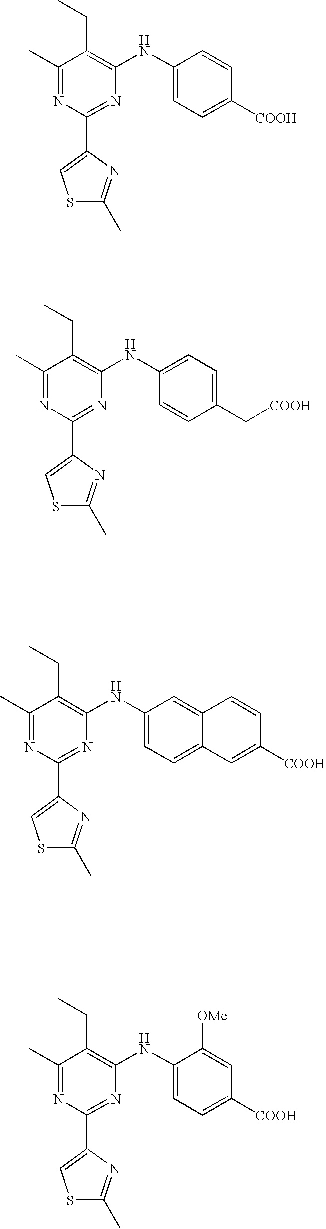

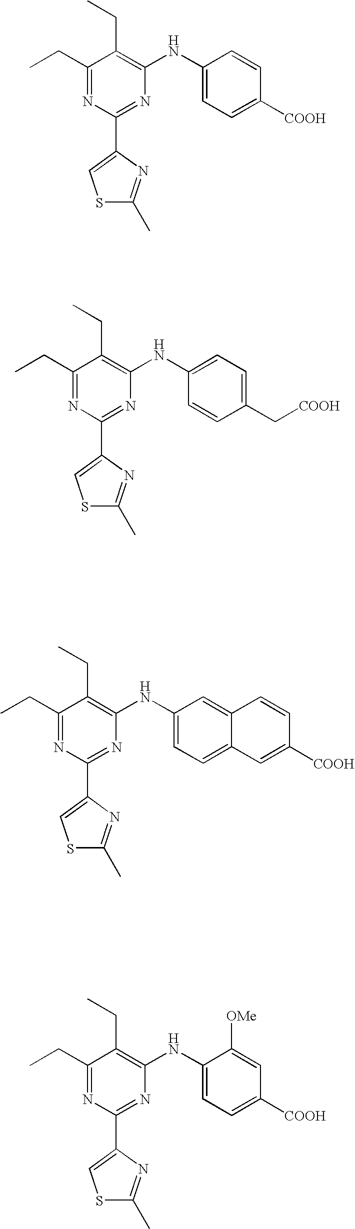

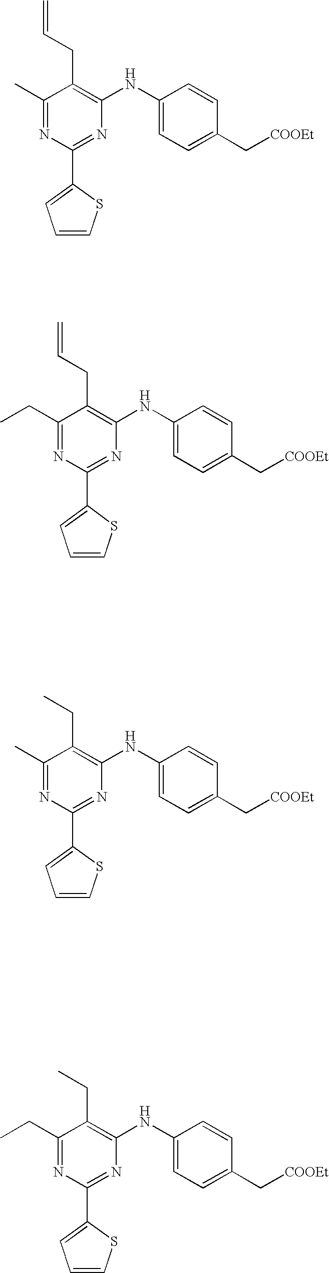

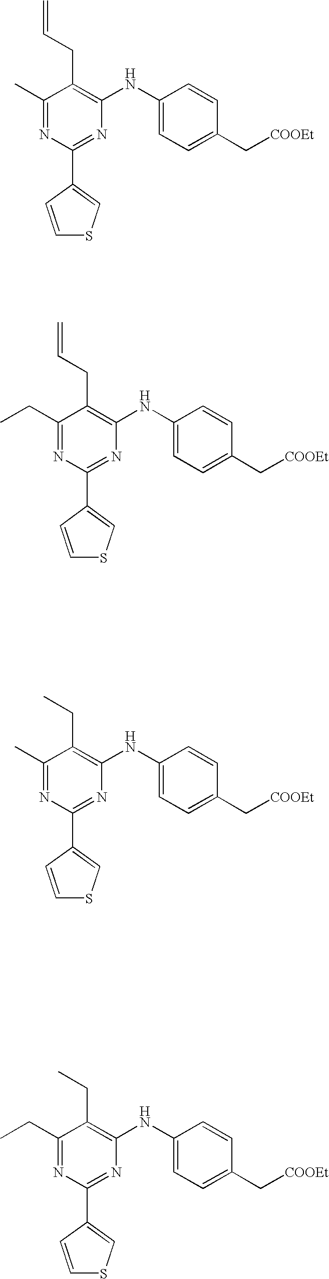

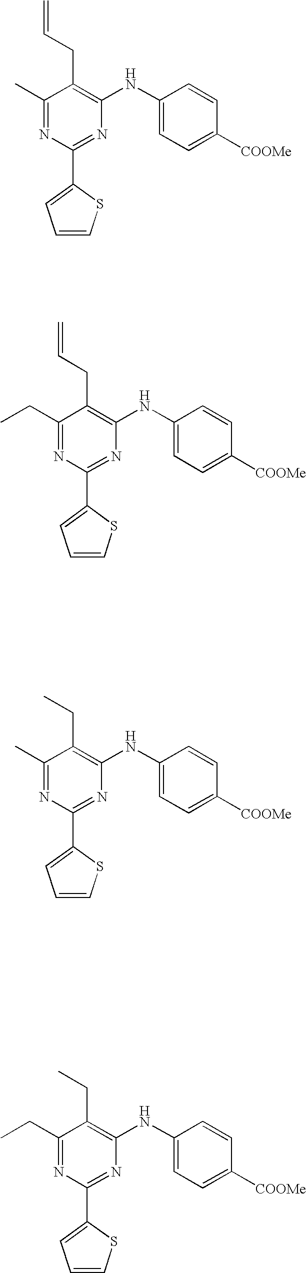

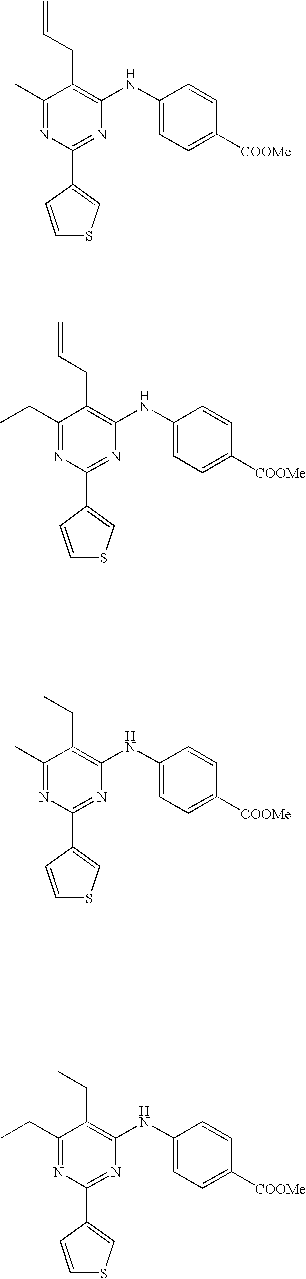

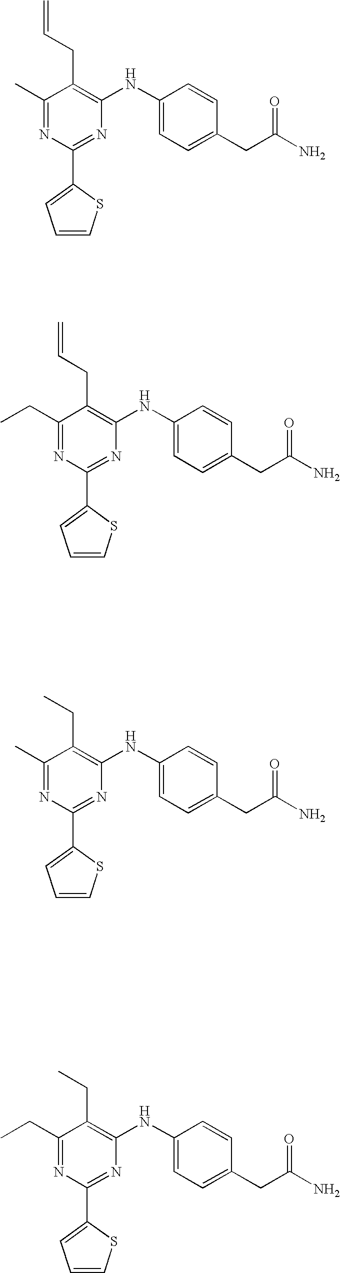

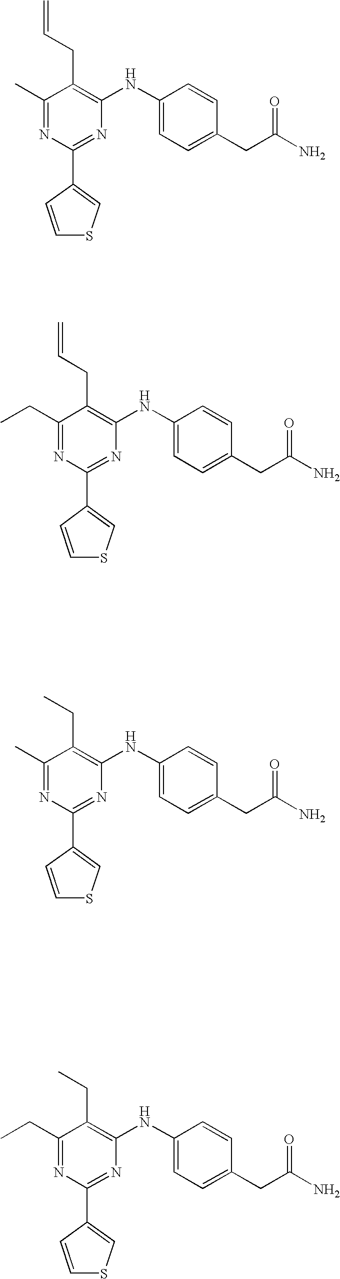

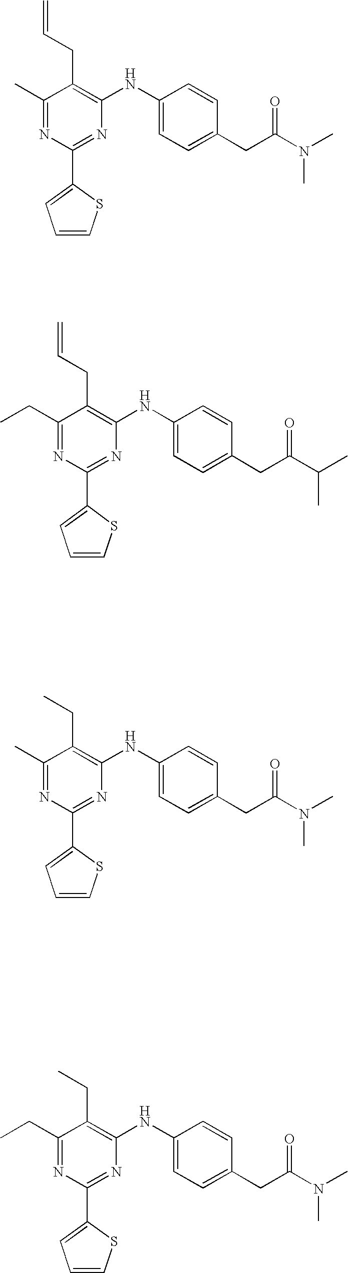

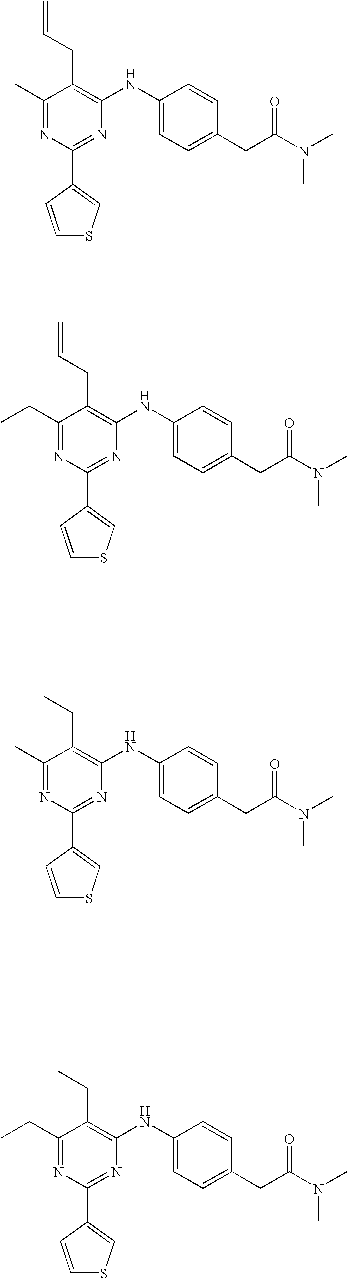

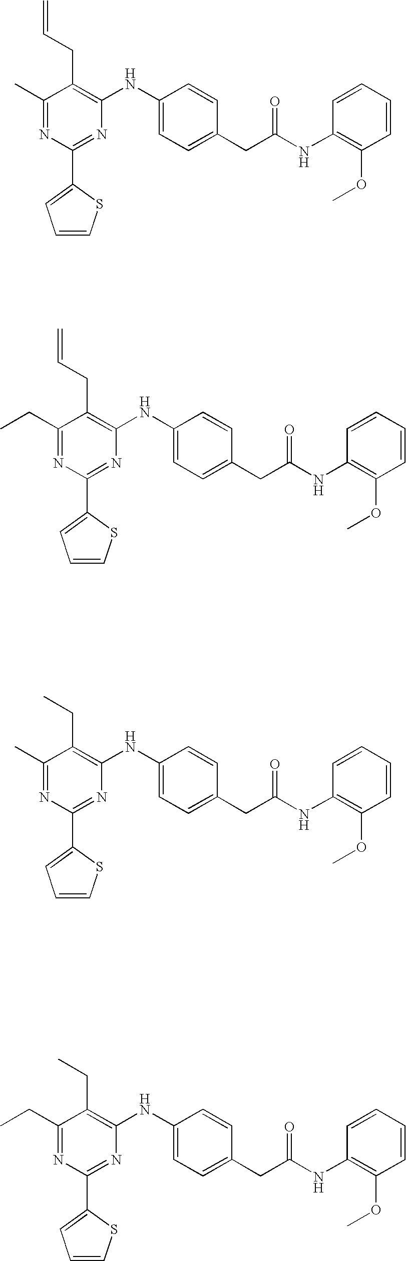

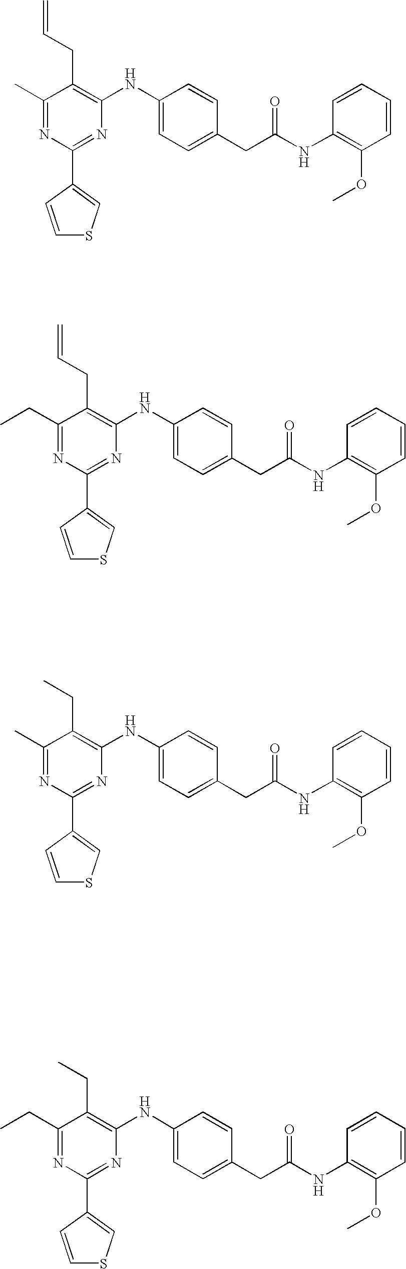

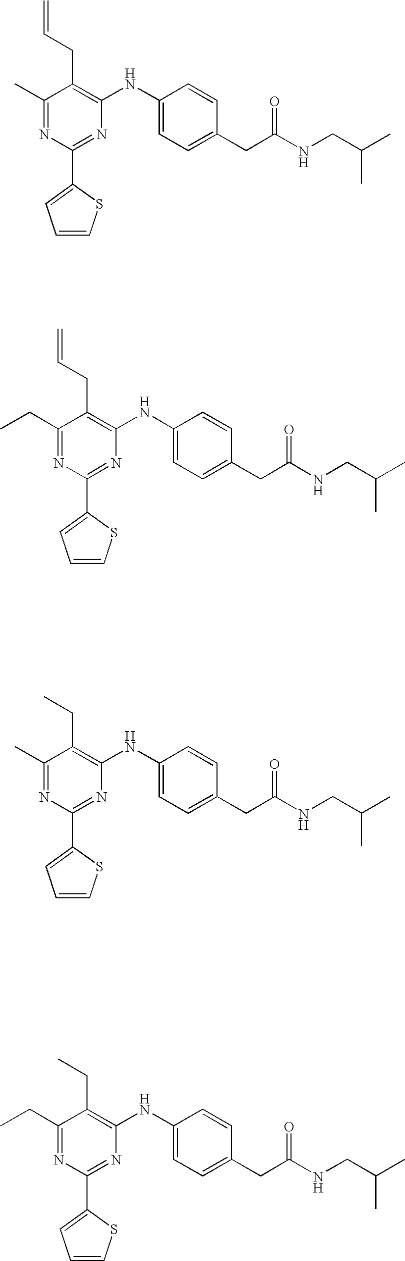

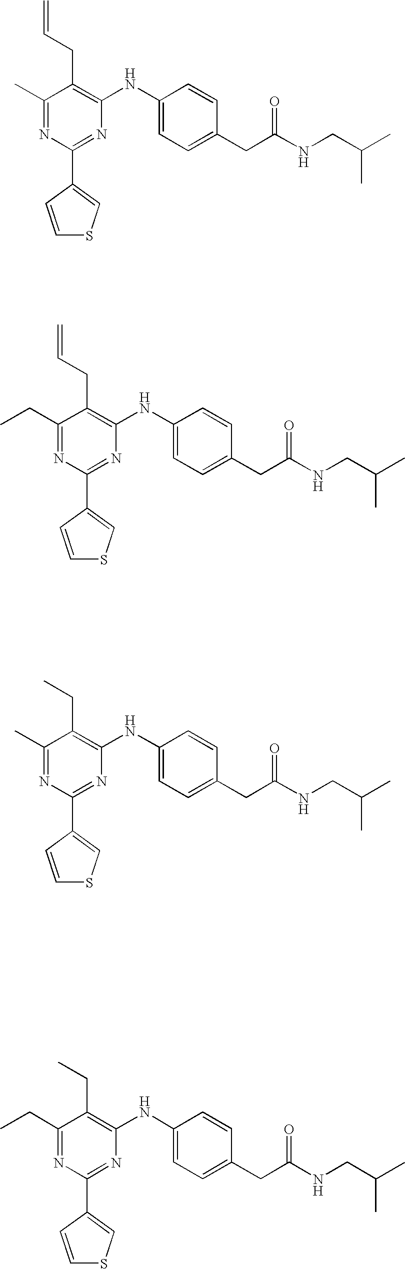

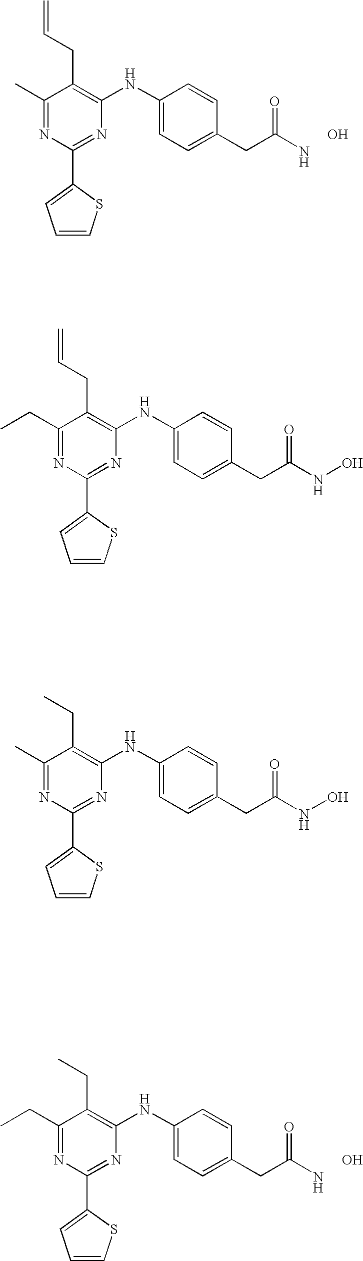

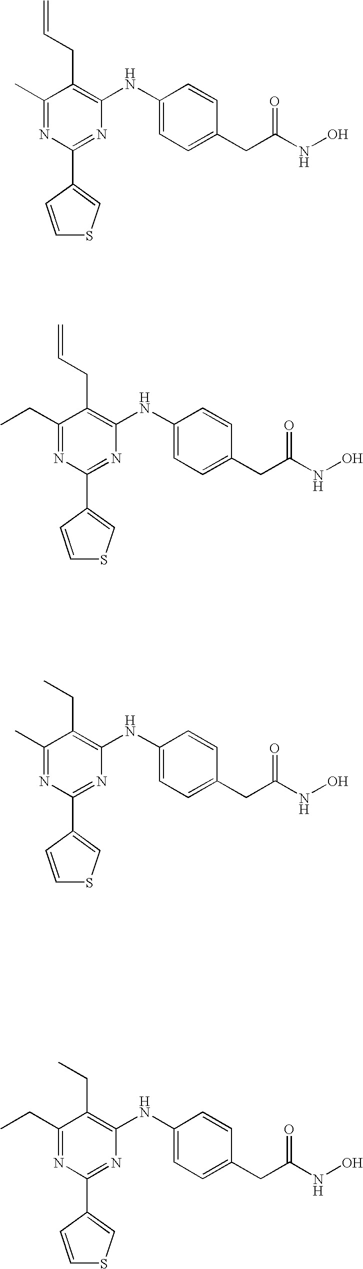

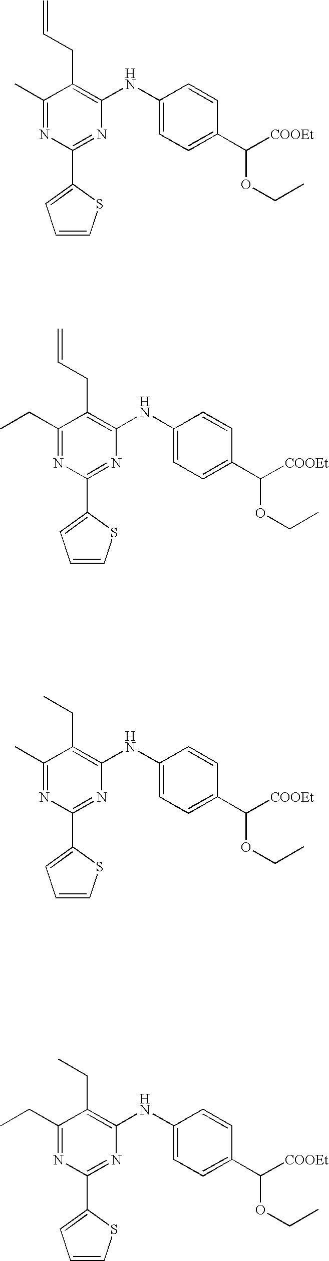

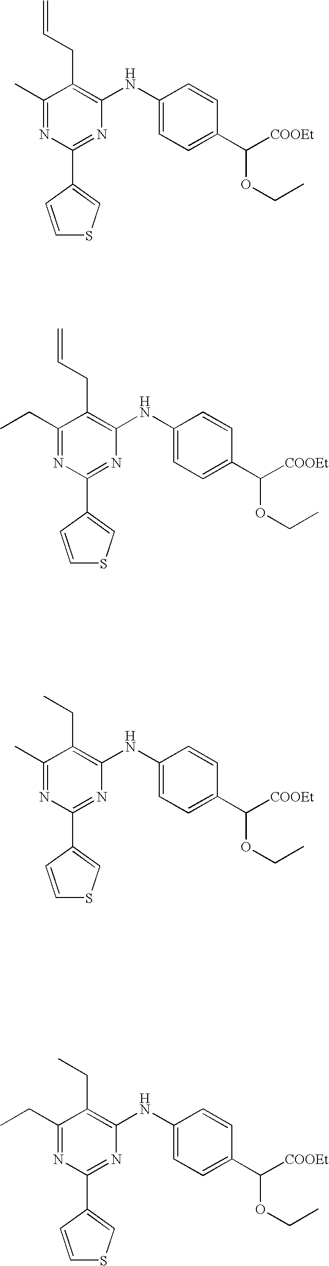

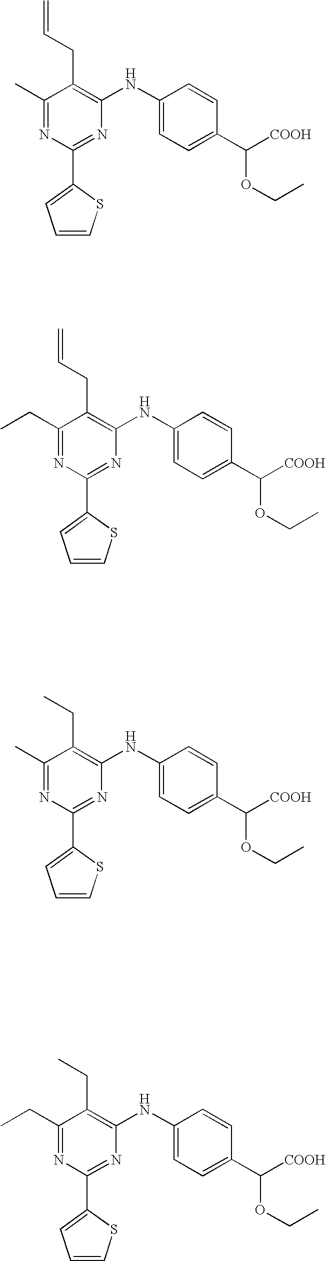

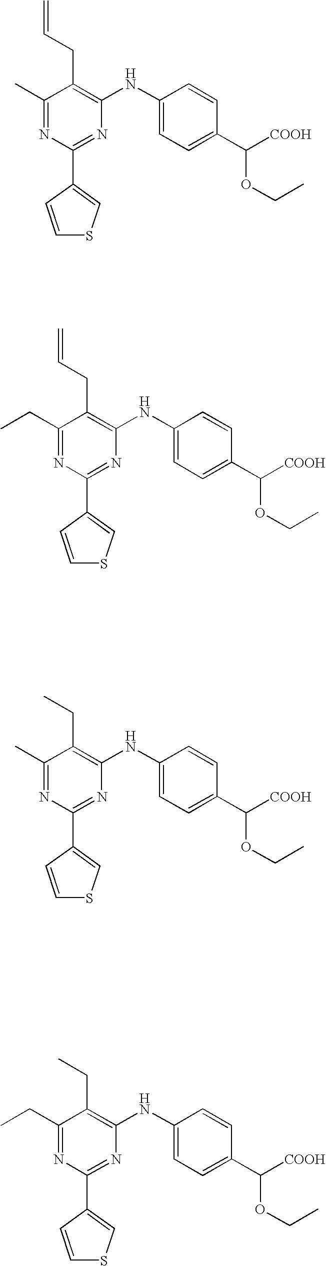

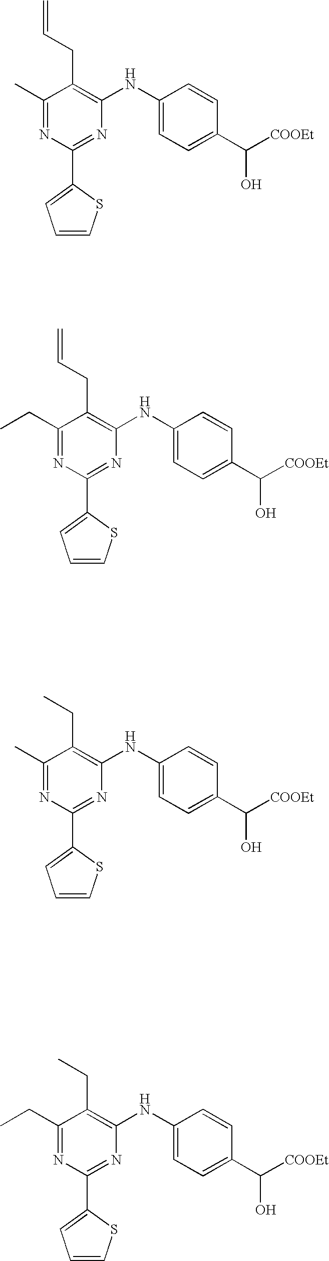

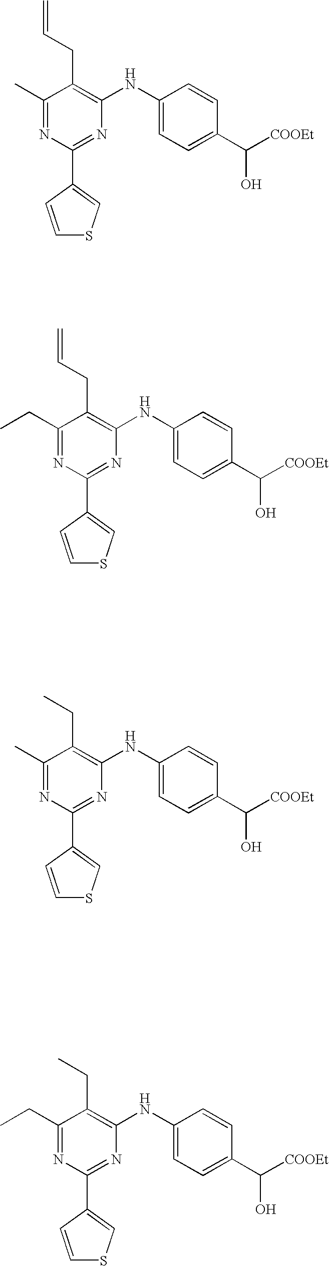

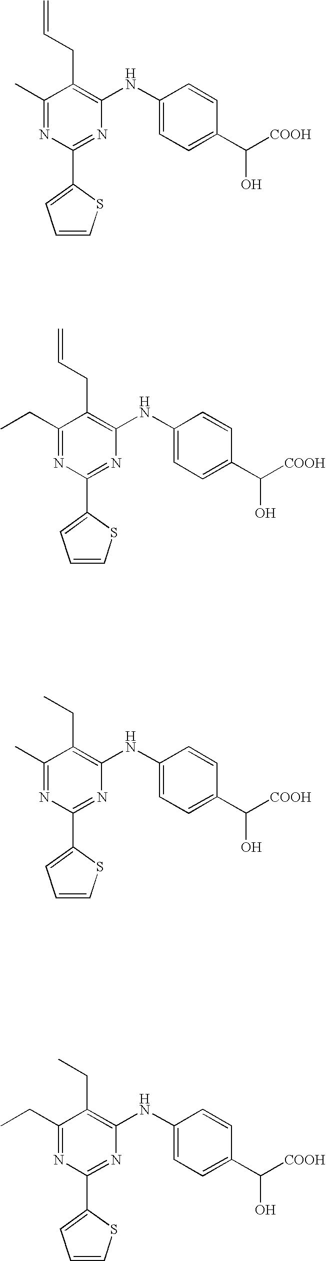

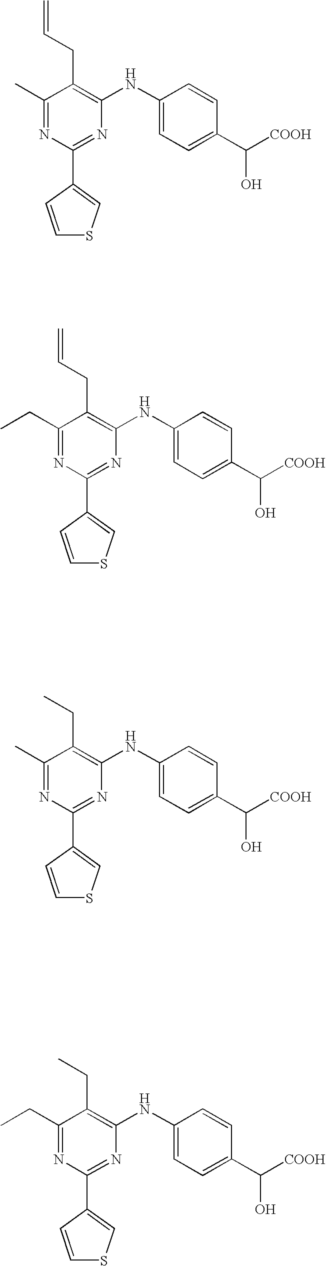

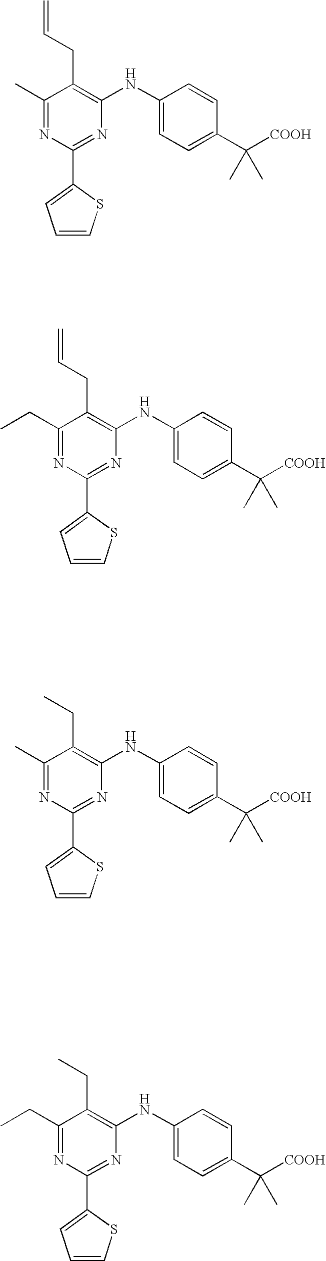

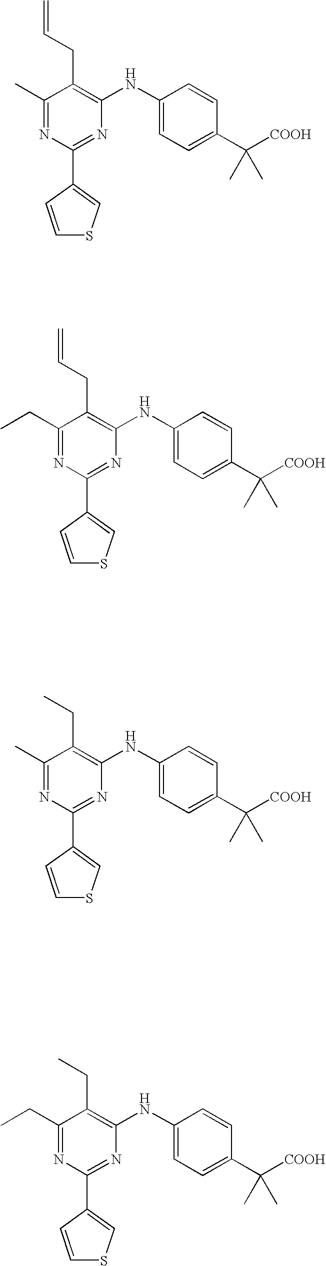

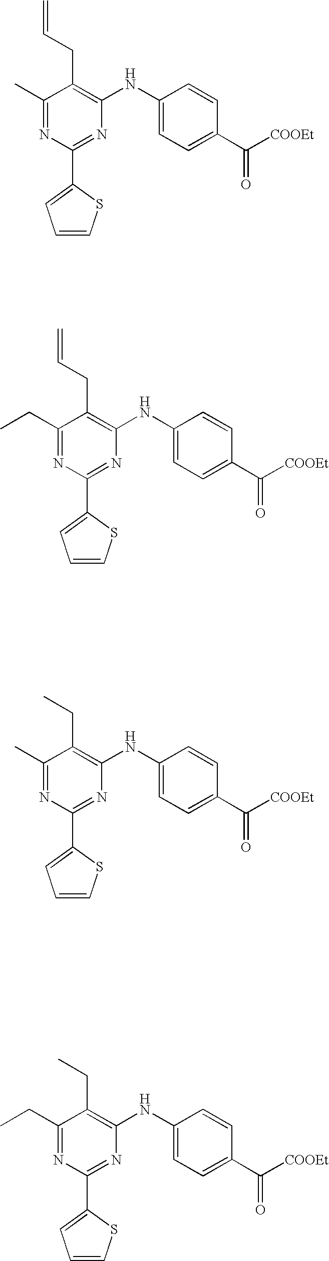

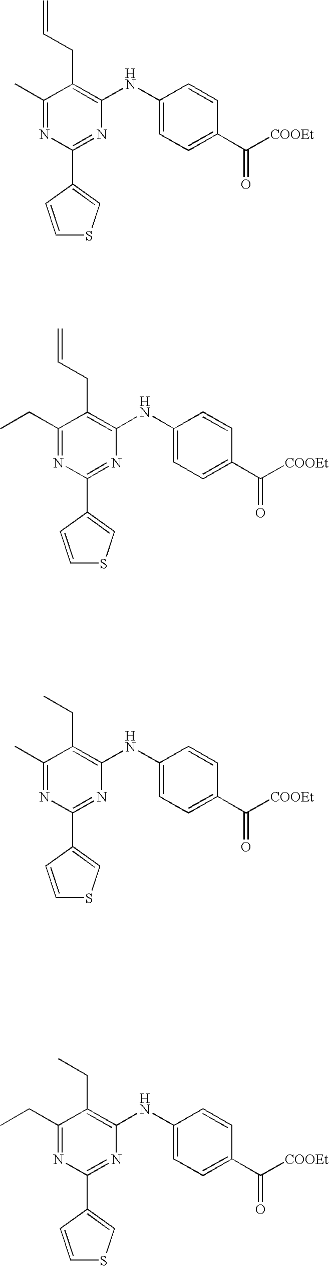

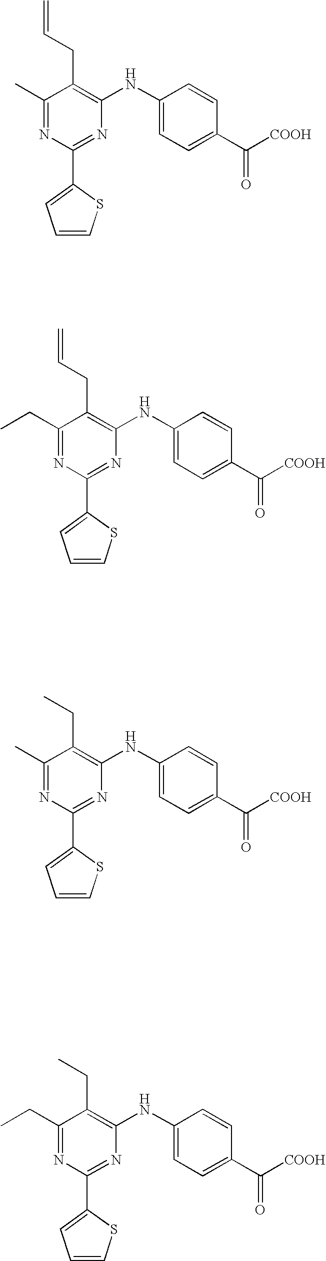

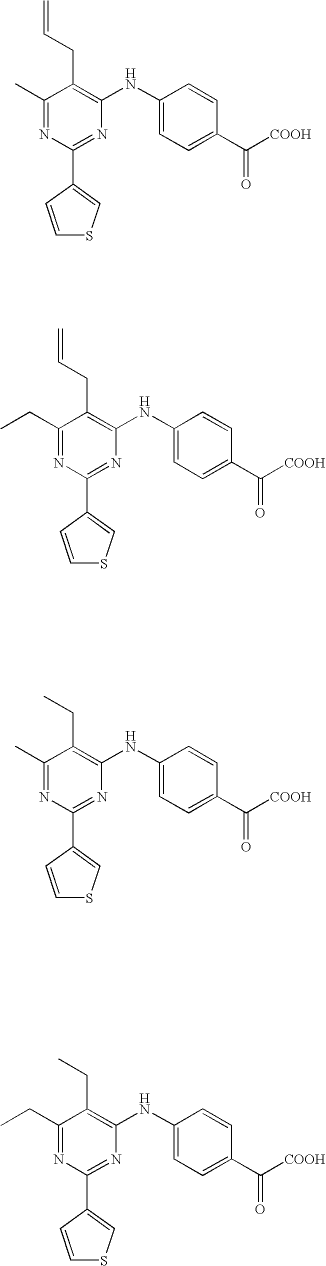

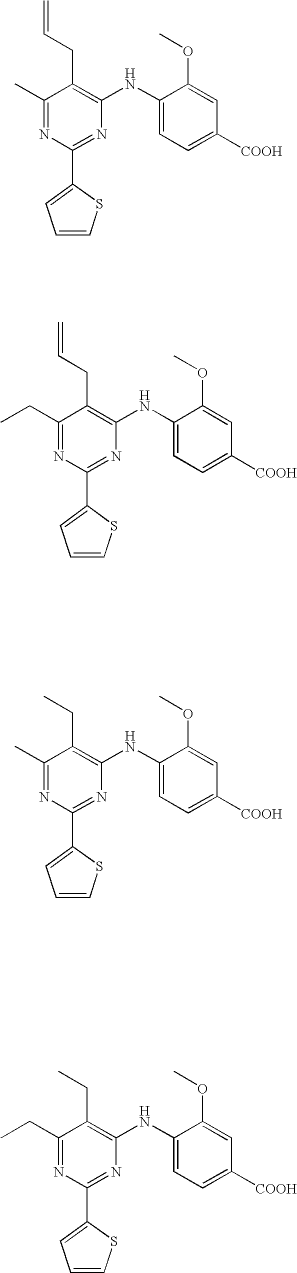

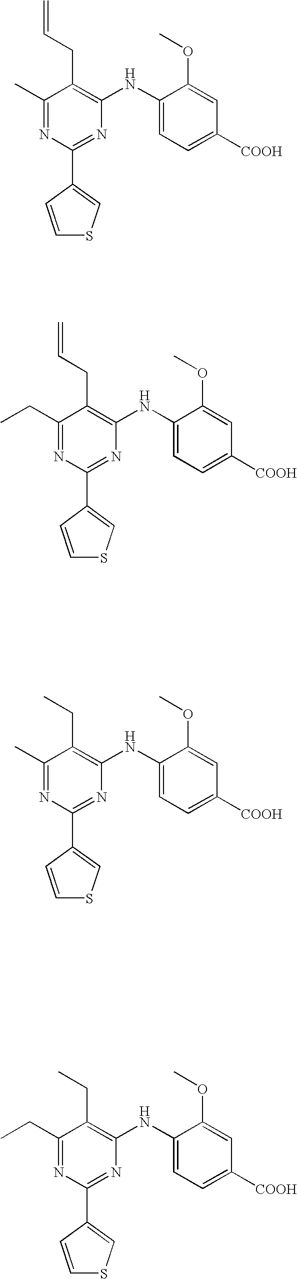

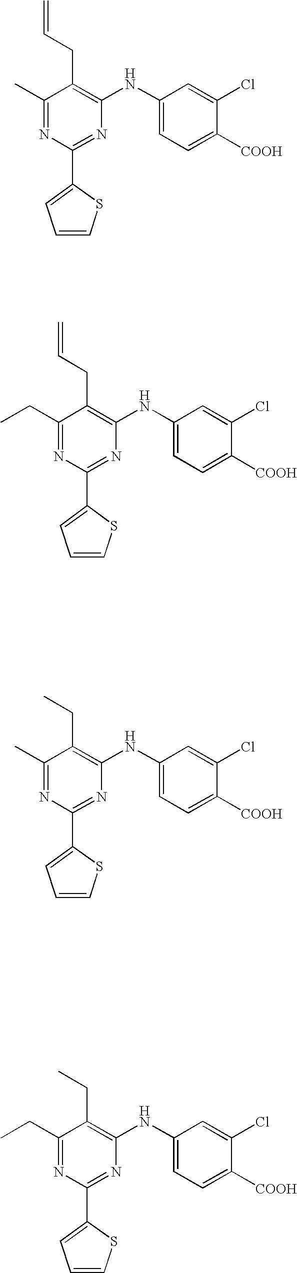

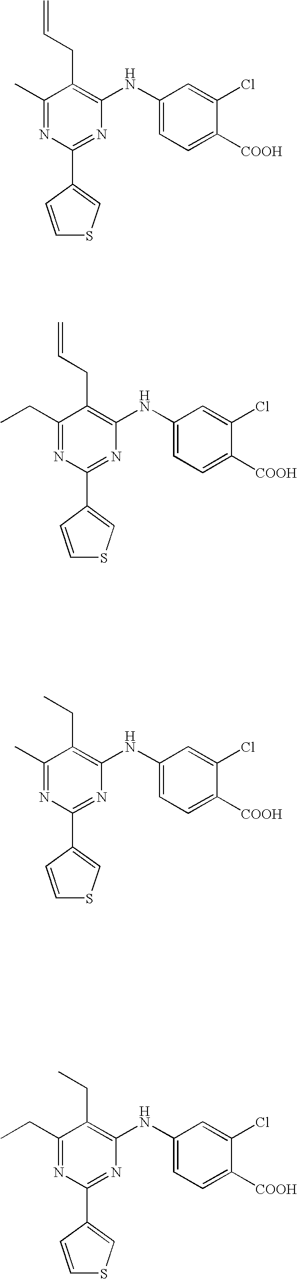

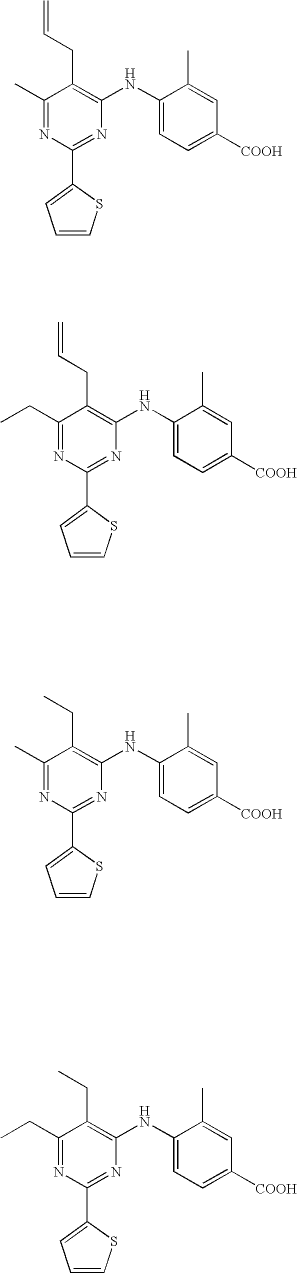

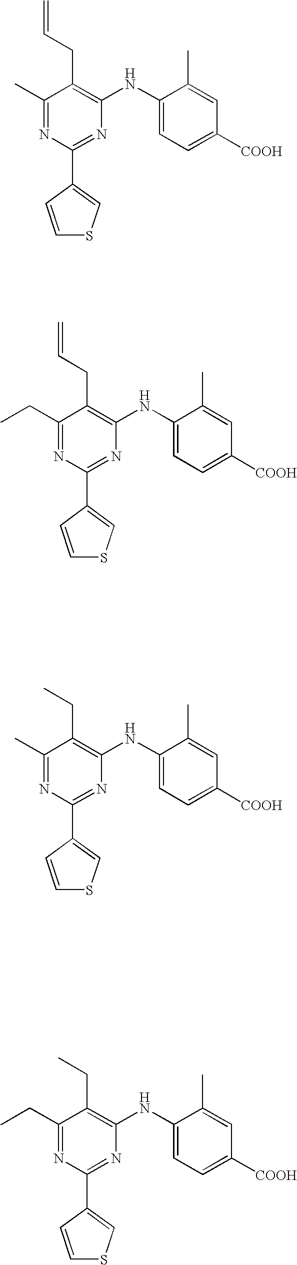

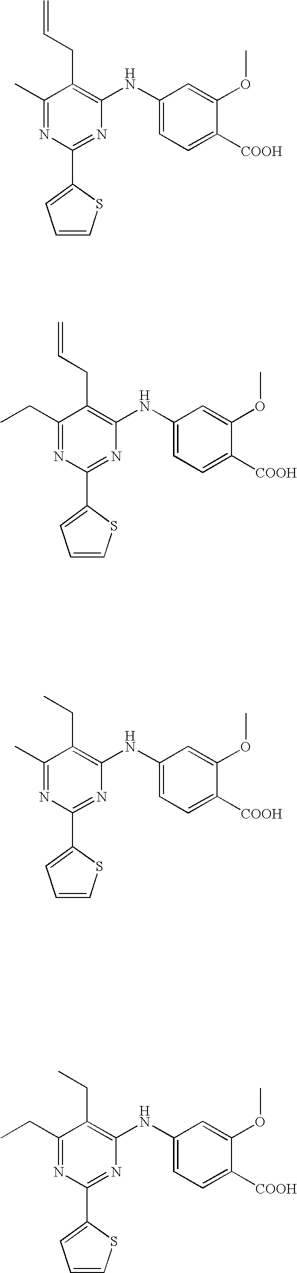

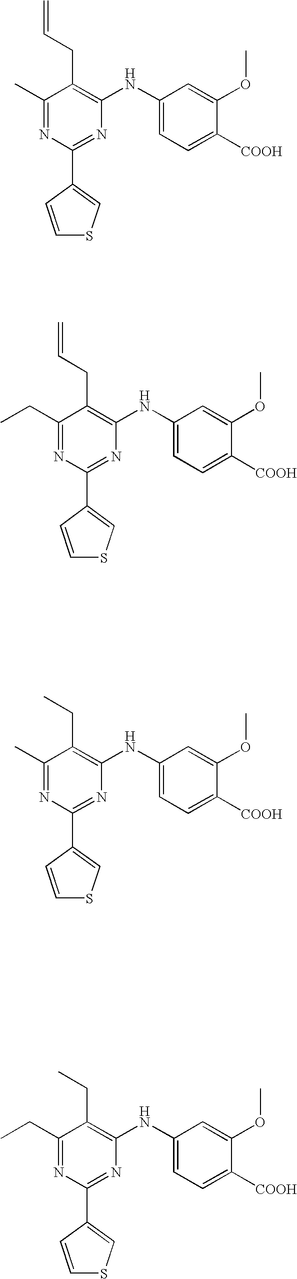

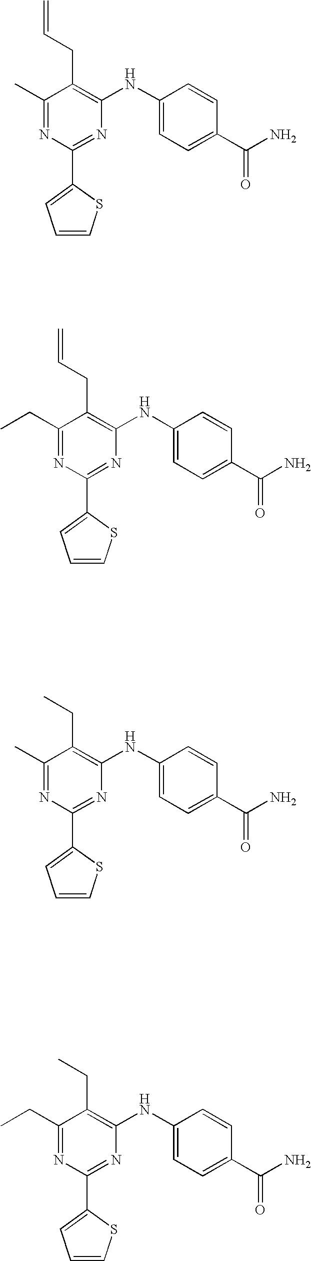

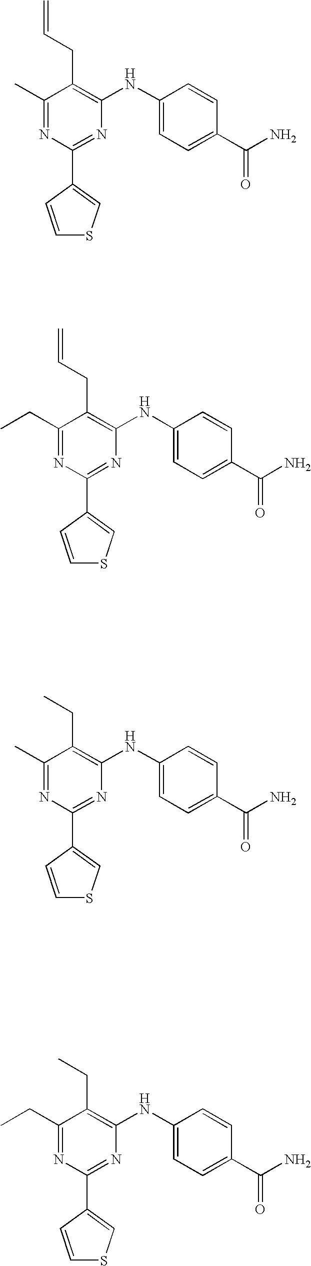

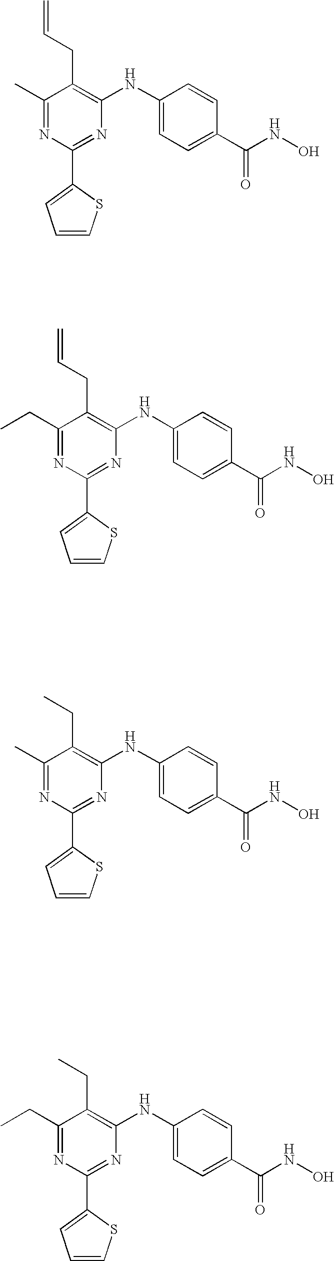

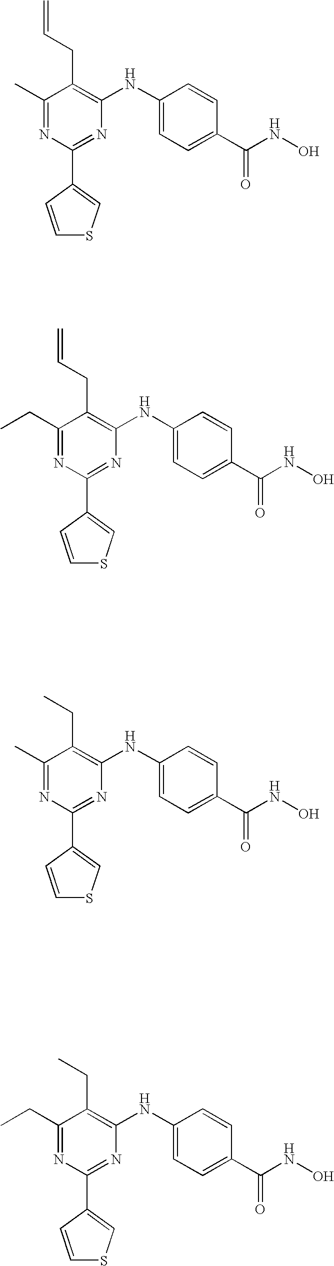

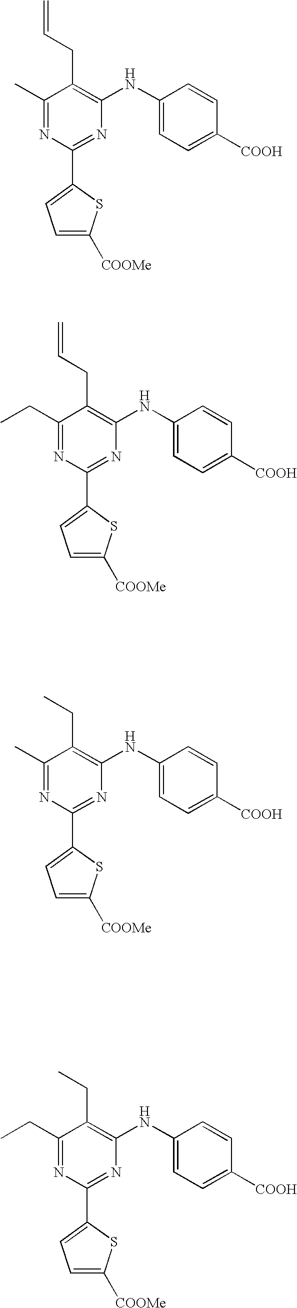

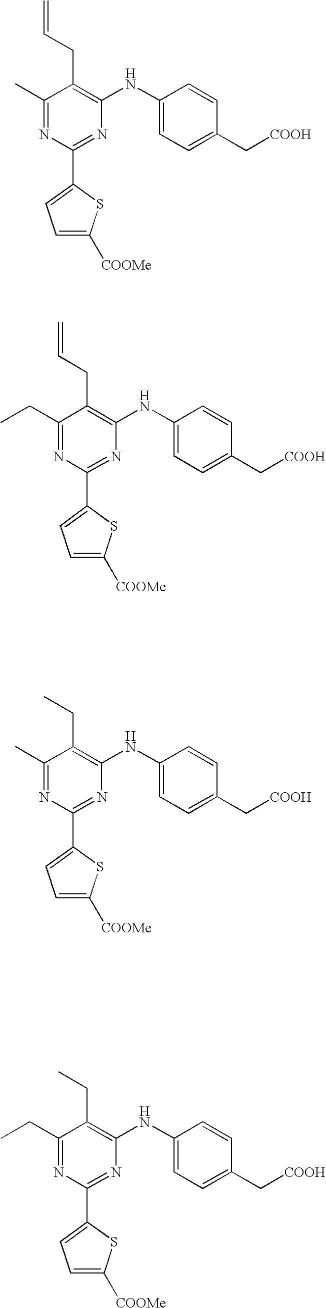

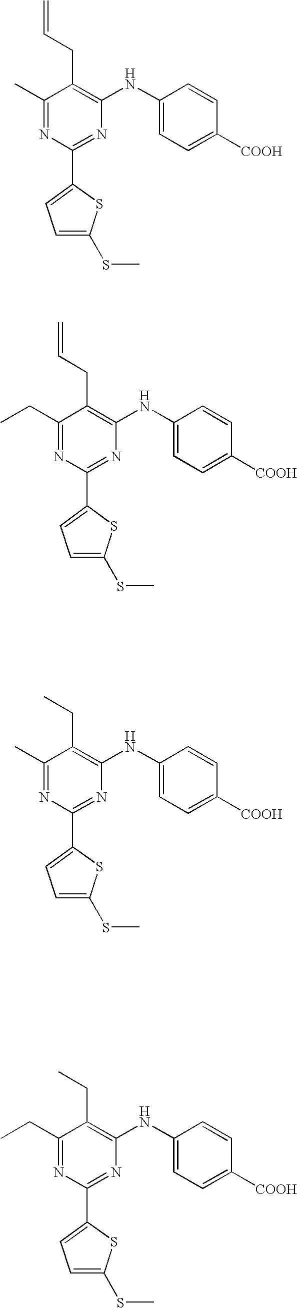

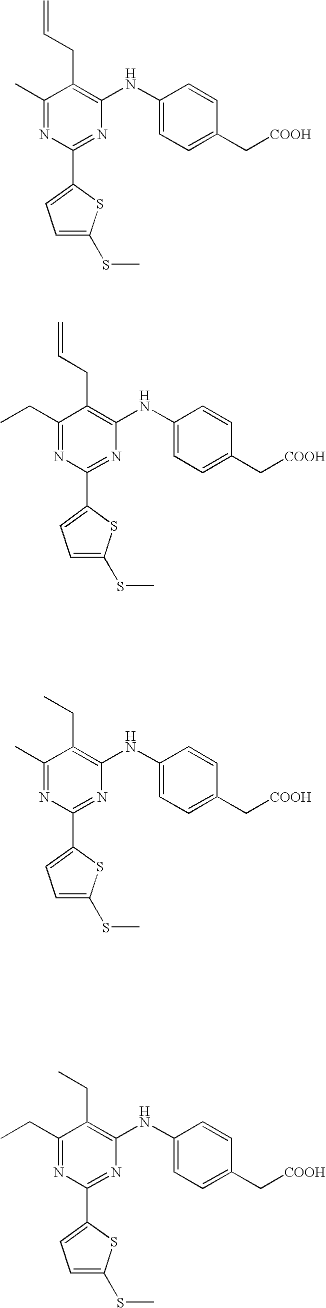

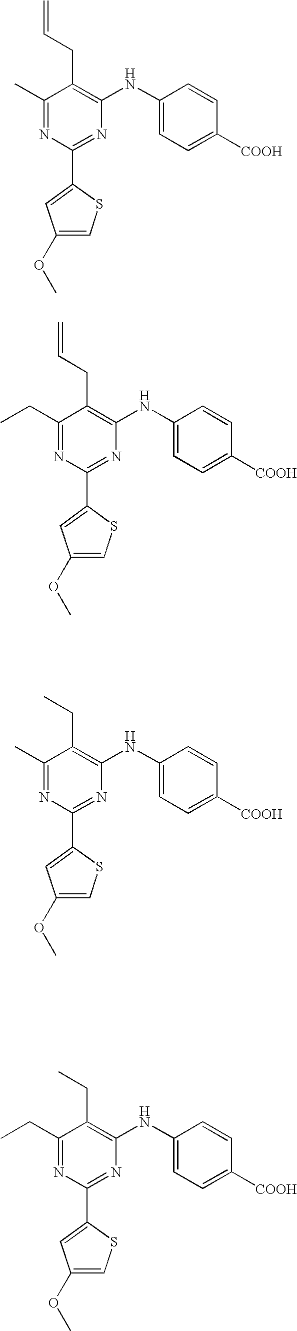

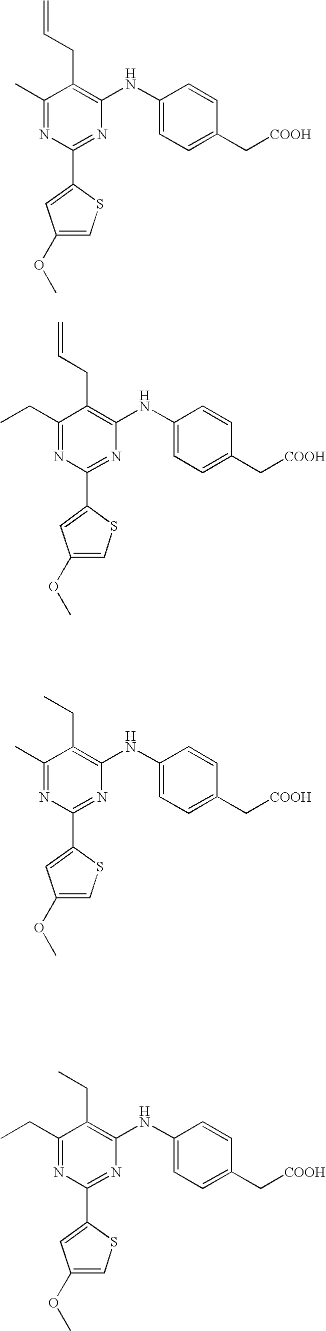

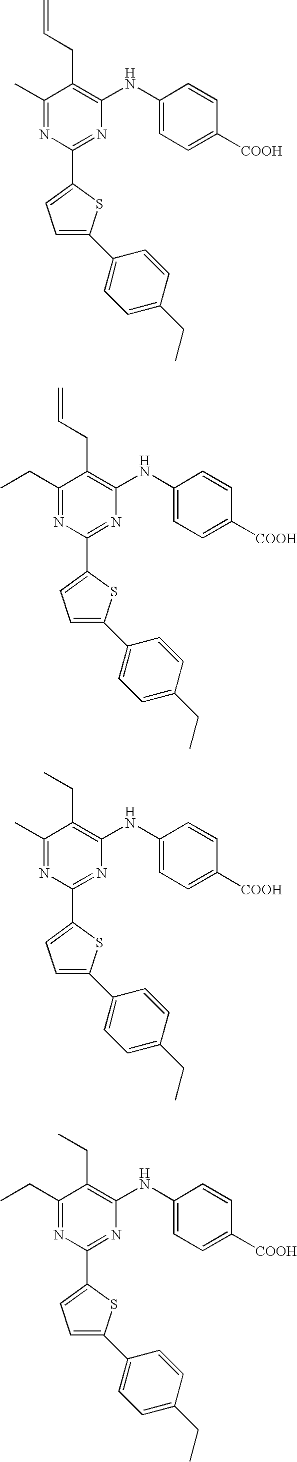

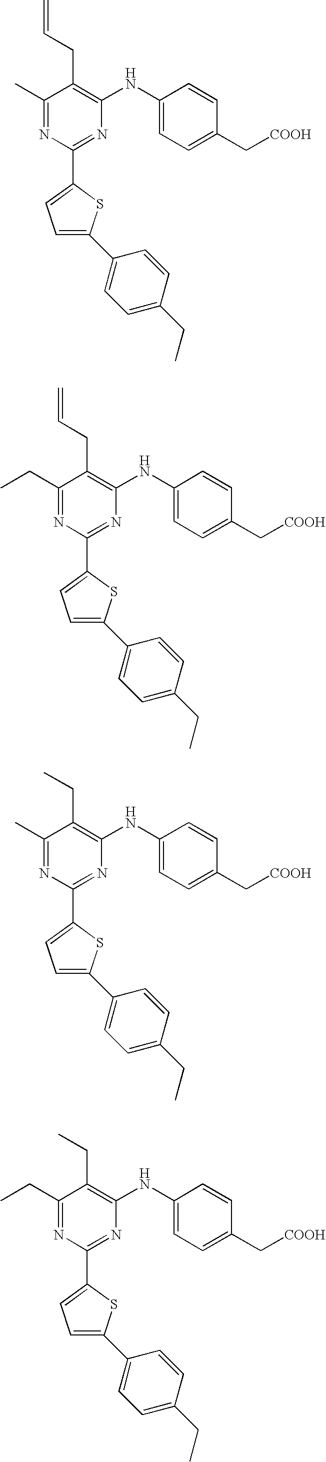

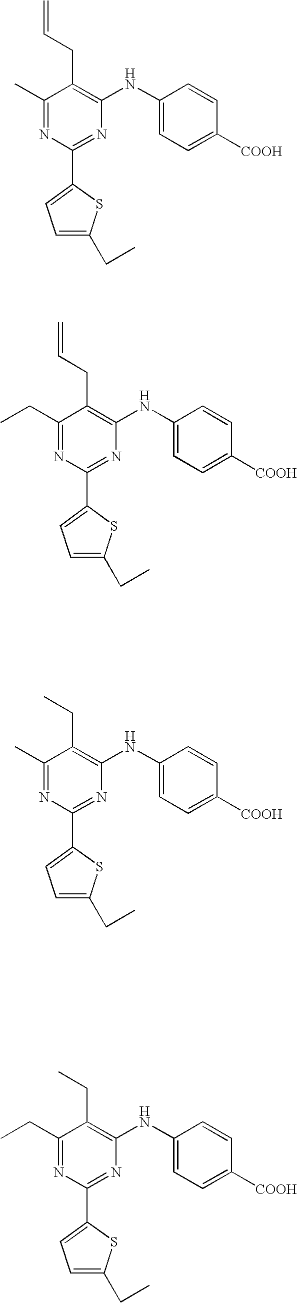

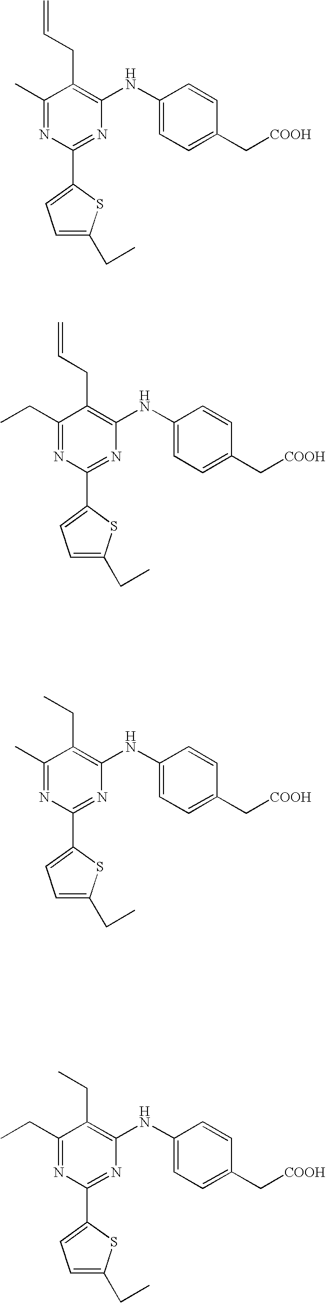

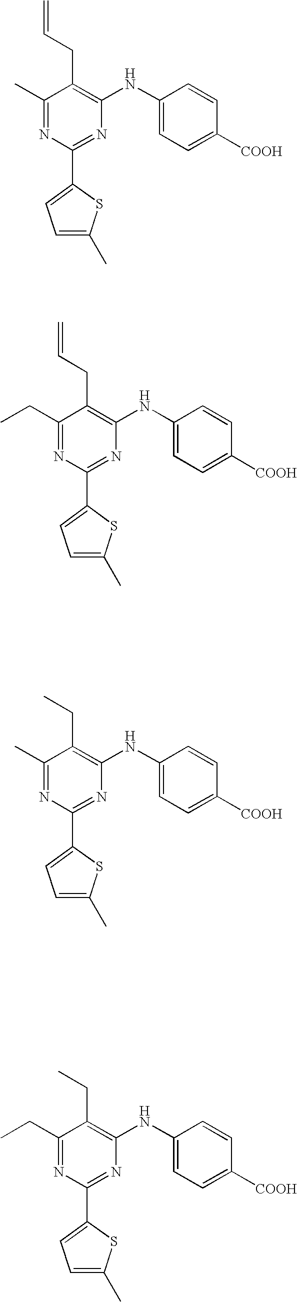

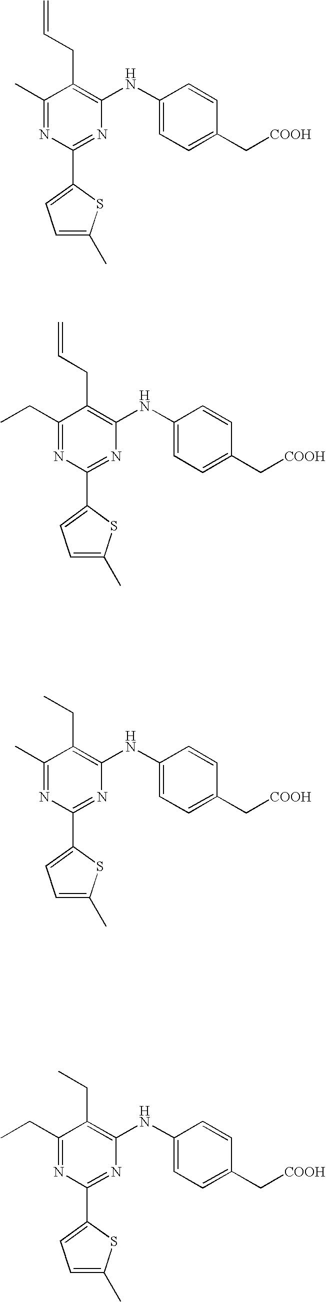

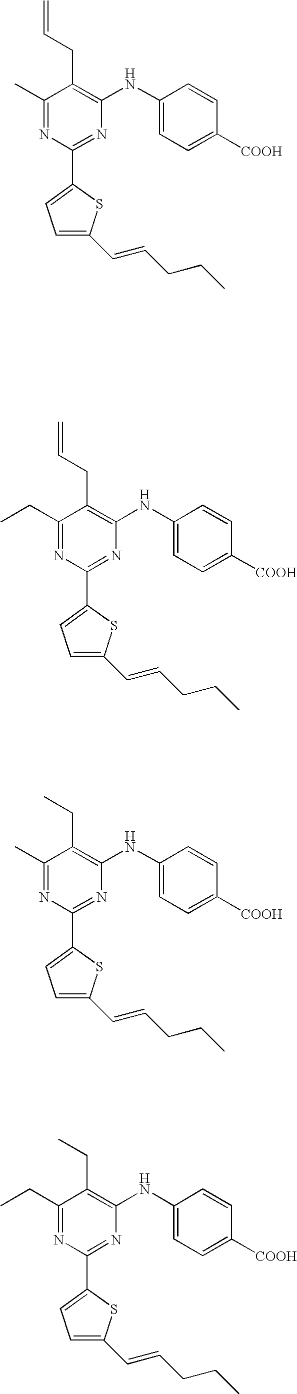

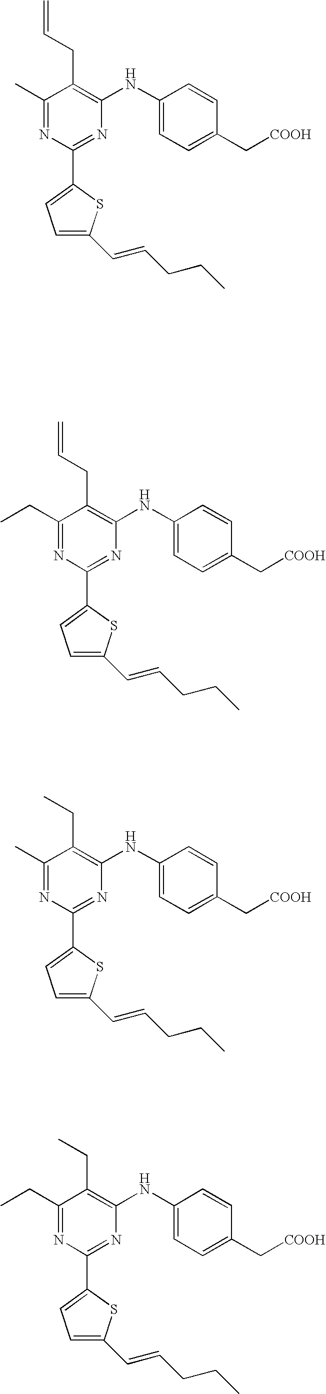

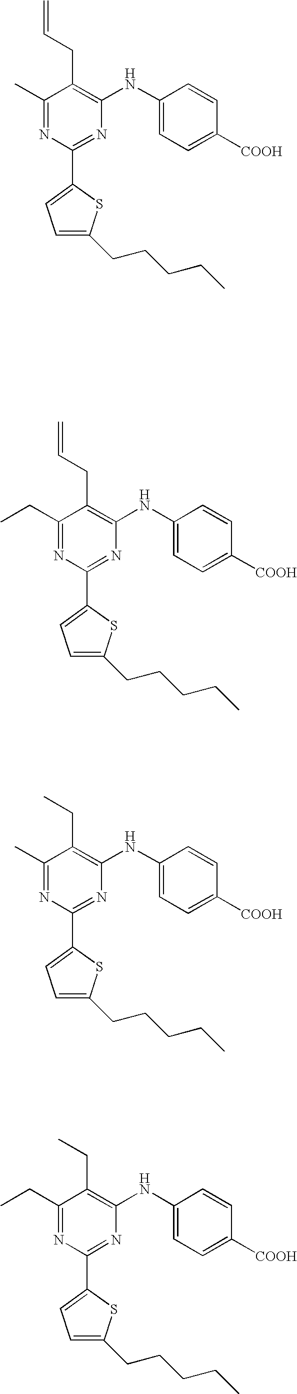

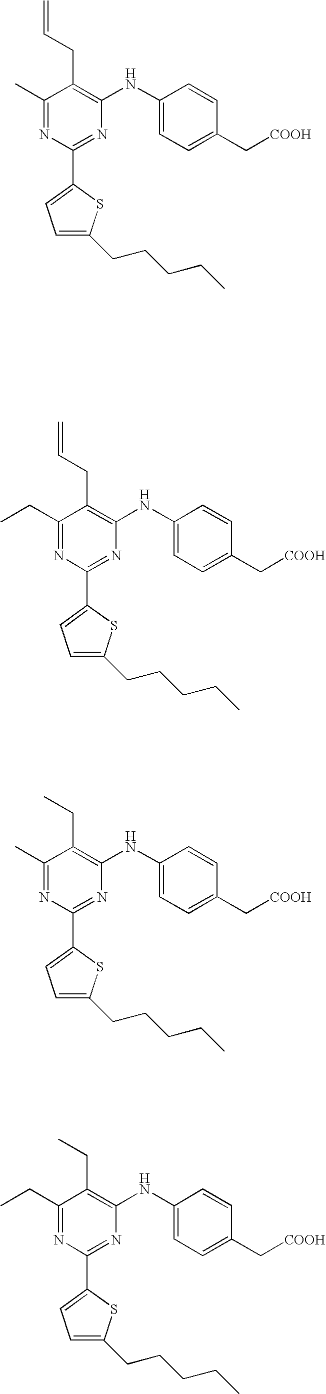

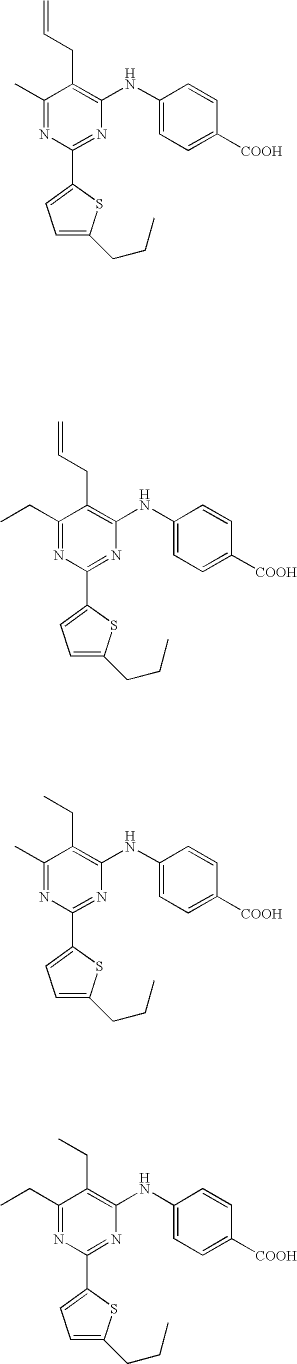

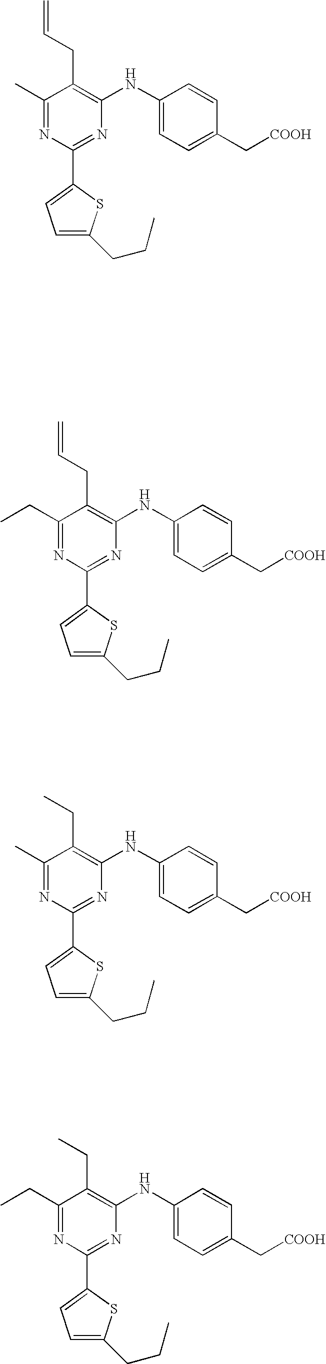

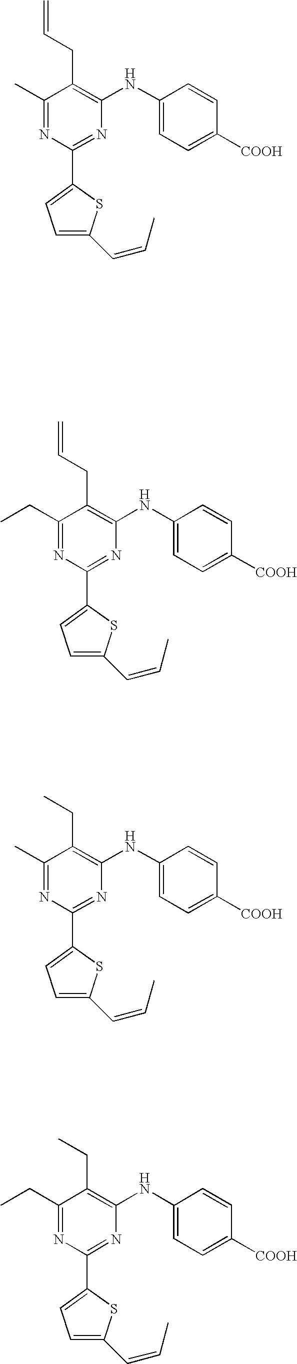

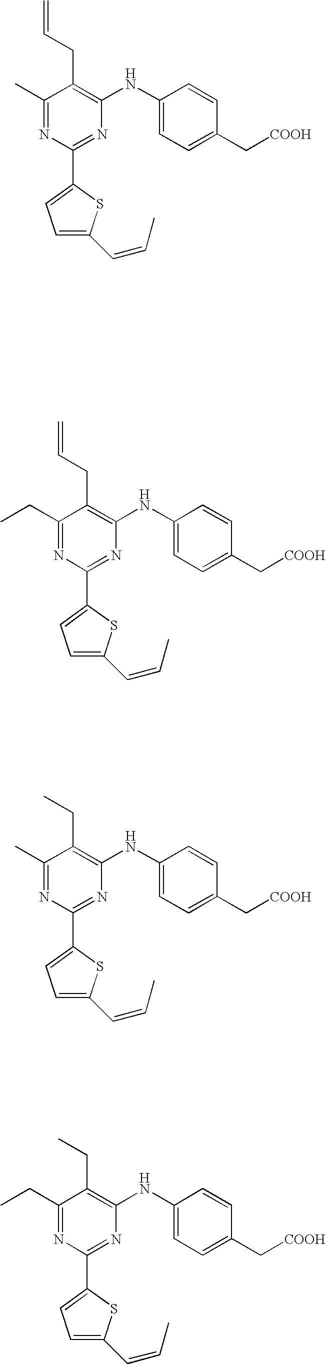

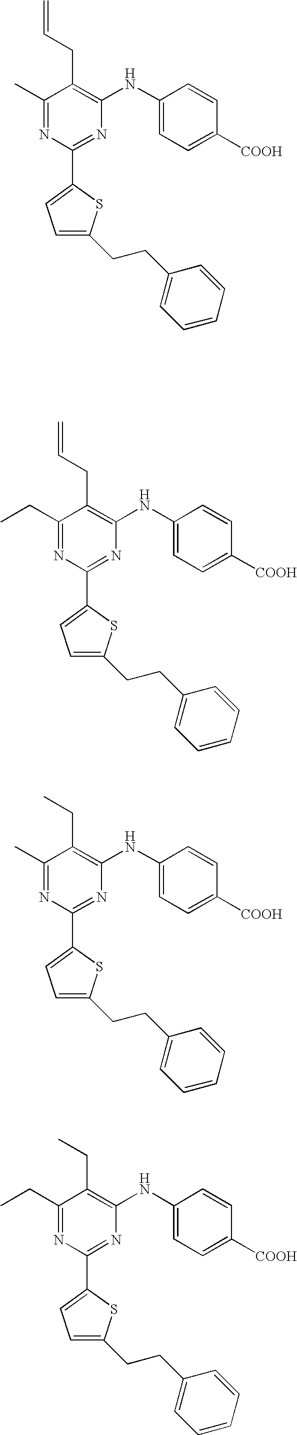

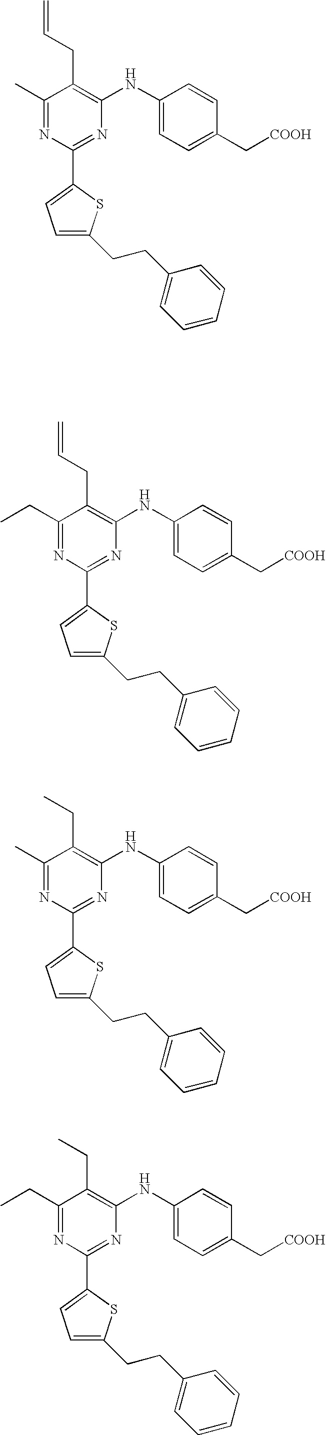

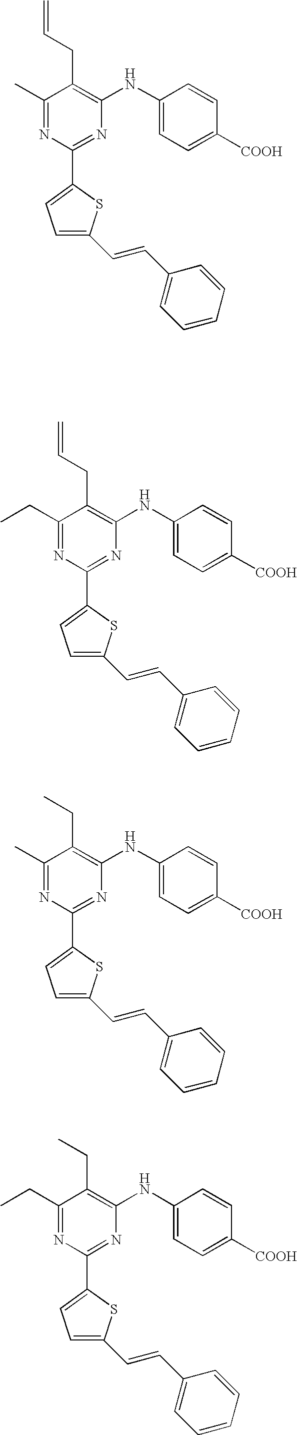

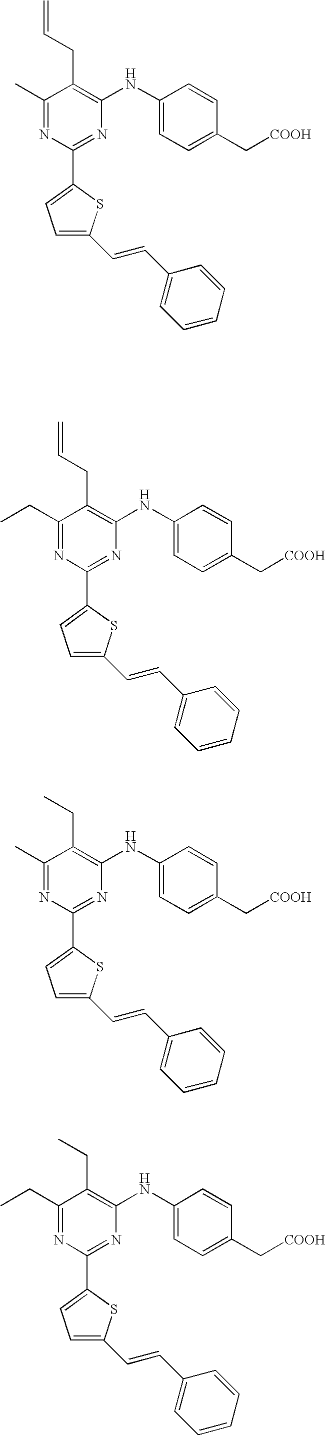

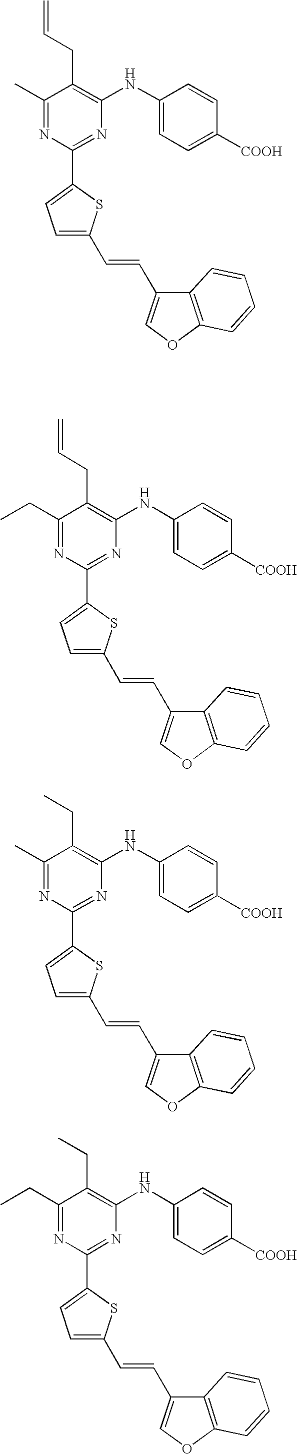

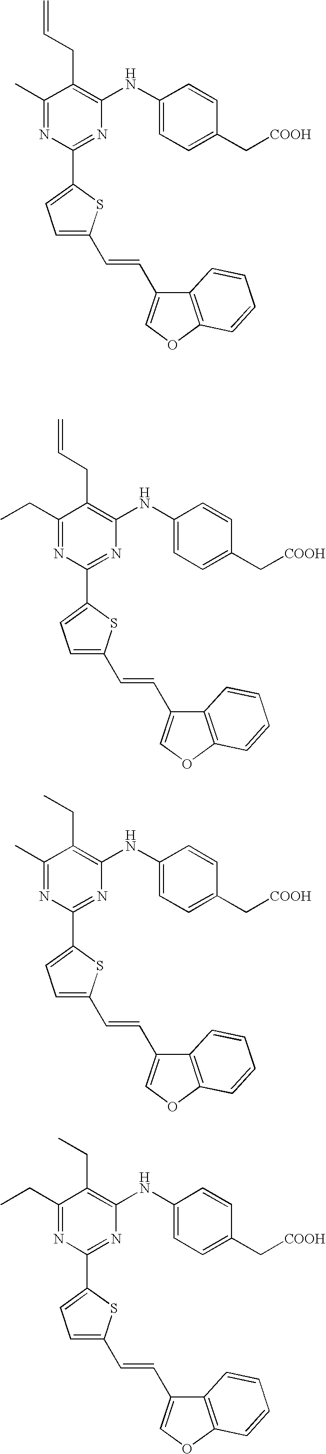

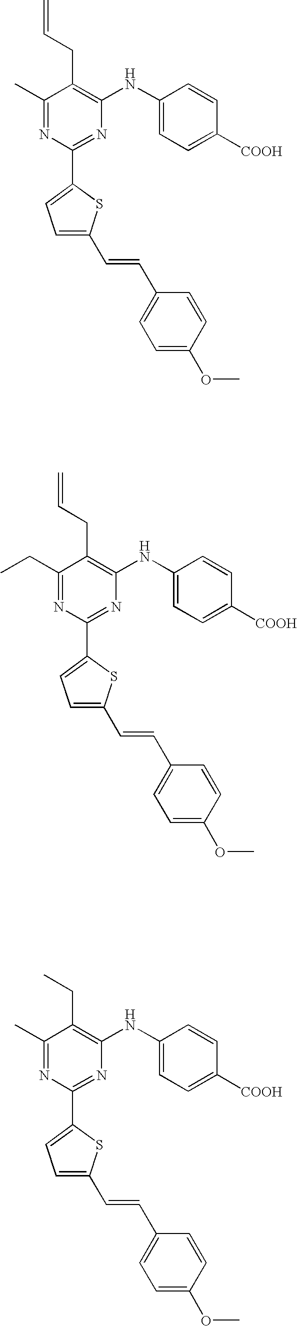

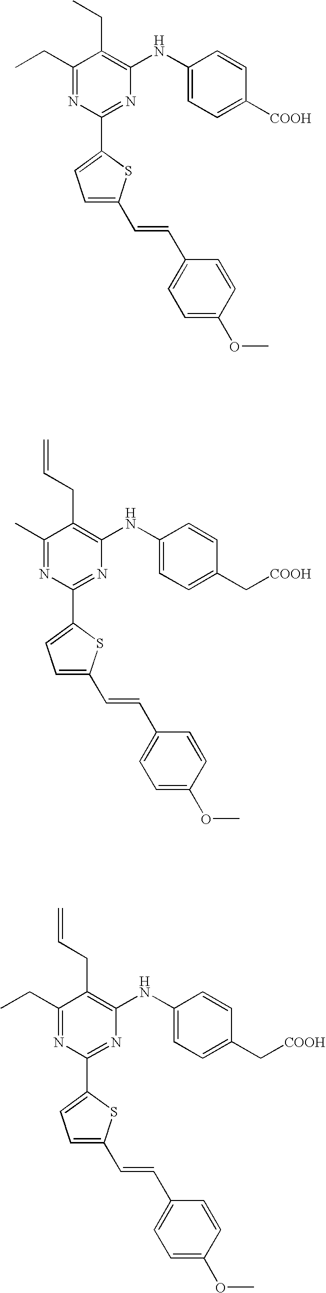

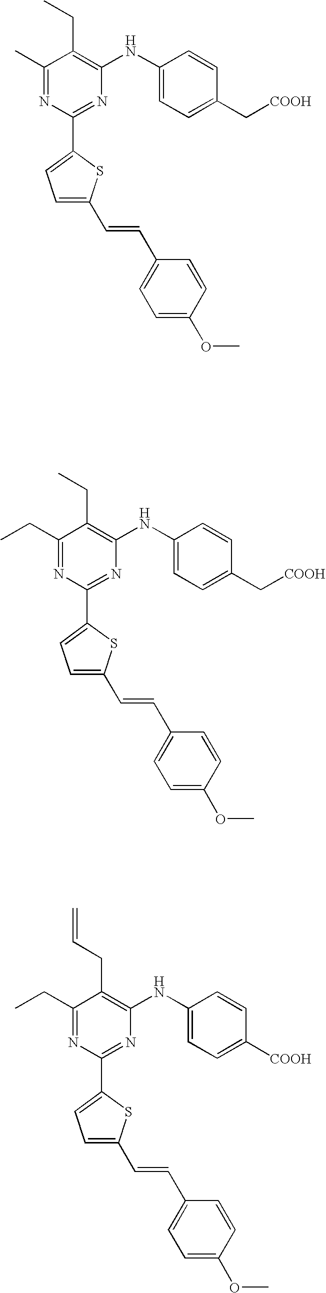

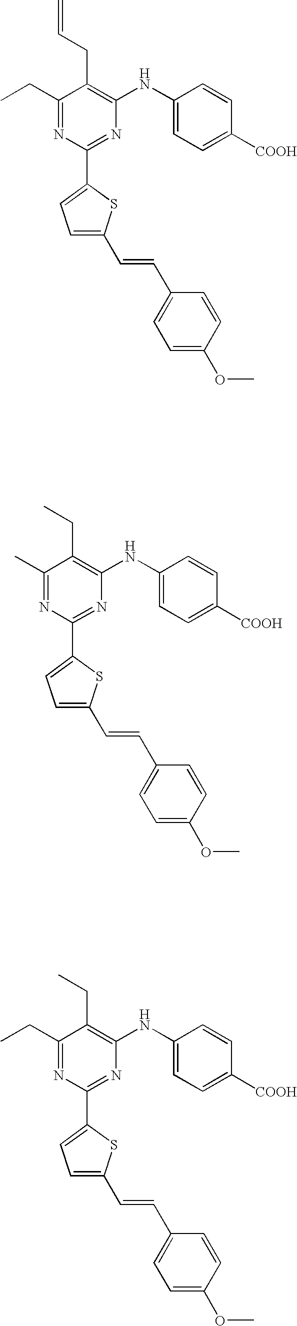

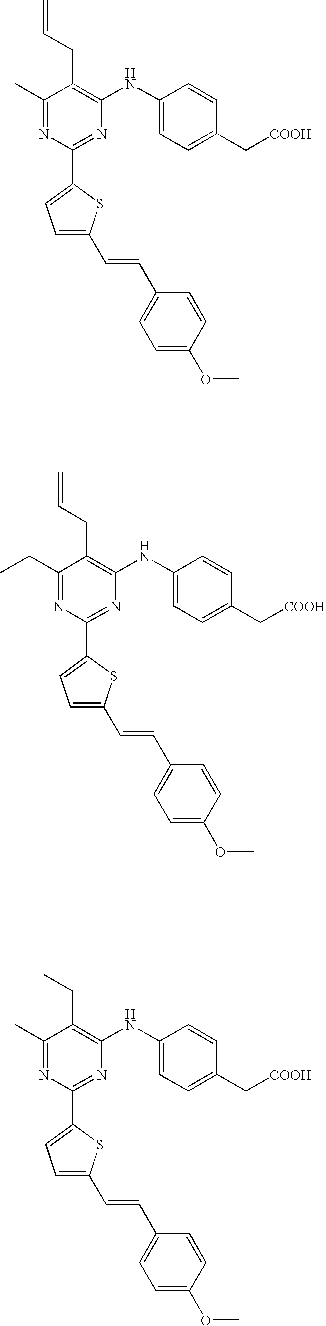

Possible stereoisomers or racemates, or pharmacologically acceptable salts thereof, hydrates and solvated compounds thereof, or prodrugs thereof also fall within the scope of the invention.

The compound of the invention is a novel compound that has not been reported in literatures. While the compound of the invention represented by general formula (1) can be produced, for example, by the following methods, the production method of the invention is not restricted thereto.

While the reaction time is not particularly restricted in each reaction, the reaction may be completed when the yield of a desired product is maximum since progress of the reaction can be readily traced by analytical means known in the art.

The compound of the invention represented by general formula (1) can be produced according to an retro-synthetic route of the reaction process described below:

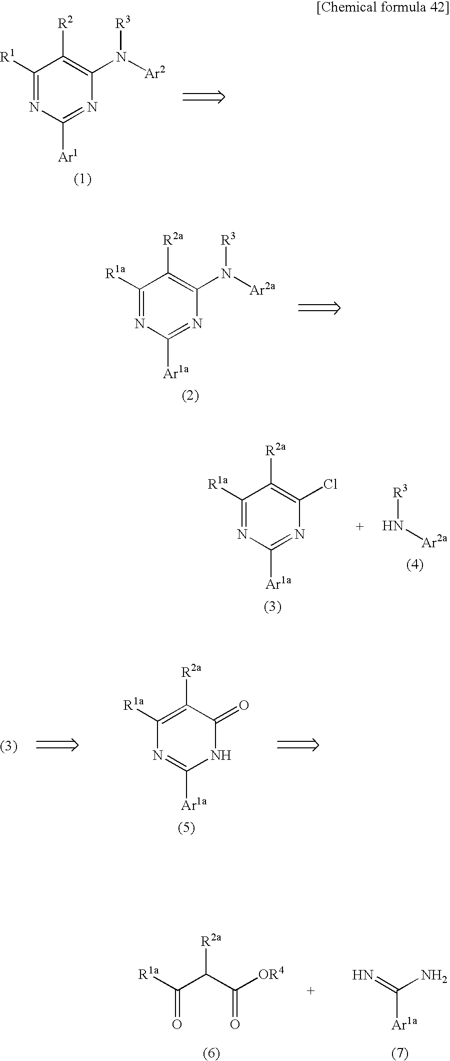

For example, the compound represented by general formula (1) may be produced by simultaneously or successively deprotecting all the protective groups in the compound represented by formula (2) (in formula (2), R³ is the same as described previously; and Ar^{1a}, Ar^{2a}, R^{1a} and R^{2a} are either the same as above-mentioned Ar¹, Ar², R¹ and R², respectively, or at least one of these groups may be protected). The deprotection reaction may be effected according to known methods, for example the method described in Protective Groups in Organic Synthesis, John Wiley and Sons, 1999.

The compound represented by general formula (2) can be produced by allowing the compound represented by general formula (3) (in general formula (3), Ar^{1a}, R^{1a} and R^{2a} are the same as described above) to react with the compound represented by general formula (4) (in general formula (4), Ar^{2a} and R³ are the same as described above) in the presence of an acid. While the proportion of use of the compound represented by general formula (4) may be in the range from 1/10 to 10 equivalent, preferably from 1/5 to 5 equivalent, and more preferably from 1 to 3 equivalent in the reaction between the compound represented by general formula (3) and general formula (4), the reaction condition may be appropriately designed considering the purity of the compound represented by general formula (2), the yield and purification efficiency. The compound represented by general formula (4) may be preferably used by converting it into a salt by addition of an acid, for example, hydrochloric. Examples of the acid include inorganic acids such as hydrochloric acid, nitric acid and sulfuric acid, and organic acids such as acetic acid, trifluoroacetic acid, methanesulfonic acid and p-toluenesulfonic acid, and preferable examples include acetic acid and trifluoroacetic acid. The amount of use of the acid is preferably in molar equivalent or in excess, for example 1 to 10 equivalent, more preferably 1 to 20 equivalent. The amount may be in excess, for example in an amount used as a solvent. Examples of the solvent used in the reaction include dichloromethane, chloroform, carbon tetrachloride, benzene, toluene, xylene, diethylether, tetrahydrofuran, dioxane, dimethoxyethane, ethyl acetate, butyl acetate and acetonitrile, where chloroform, toluene and tetrahydrofuran are preferable. These solvent may be used as a mixture of a plurality of them. The acid used in the above-mentioned reaction can be preferably used as a solvent. In a preferably reaction condition, acetic acid is used in an amount as a solvent. The reaction temperature may be in the range from 0 to 200° C., preferably in the range from 50 to 150° C. While the reaction time is not particularly restricted, it is usually 1 to 96 hours, preferably 3 to 60 hours.

While the compound of the compounds represented by general formula (2) having carboxylate ester portions in the structure may be produced by allowing it to react in the presence of an acid, a preferable example of the acid used is hydrochloric acid and a preferable solvent used in the invention is an alcohol. An example of the alcohol is methanol, ethanol, 1-propanol or 2-propanol, methanol or ethanol is preferable.

When E in Ar² in general formula (1) corresponding to the Ar^{2a} portion in general formula (2) of the compounds represented by general formula (2) represents an alkylene group, the compound represented by general formula (2) can be produced by allowing the compound represented by general formula (3) to react with the compound represented by general formula (4) in the presence of hydrochloric acid. While the proportion of use of the compound represented by general formula (4) in the reaction between the compound represented by general formula (3) and the compound represented by general formula (4) is in the range from 1/10 to 10 equivalent, preferably in the range from 1/5 to 5 equivalent, and more preferably 1 to 3 equivalent relative to the compound represented by general formula (3), the reaction condition may be appropriately designed depending on the purity of the compound represented by general formula (2), the yield and purification efficiency. While an example of the base is an organic base such as triethylamine, diisopropylethylamine or 1,8-azabicyclo[5.4.0]undec-7-ene, 1,8-diazabicyclo[5.4.0]undec-7-ene is preferable. The amount of use of the base is preferably in equivalent or in excess relative to the compound represented by general formula (3), and the preferable amount is in the range form 1 to 10 equivalent, more preferably in the range from 1 to 20 equivalent. While examples of the solvent used in the reaction include dichloromethane, chloroform, carbon tetrachloride, benzene, toluene, xylene, diethylether, tetrahydrofuran, dioxane, dimethoxyethane, methanol, ethanol, propanol, butanol, ethyl acetate, butyl acetate and acetonitrile, butanol is preferable. These solvents may be used as a combination of a plurality of them. While the reaction temperature is in the range from 0 to 200° C., the preferable temperature is in the range from 50 to 150° C. While the reaction time is not particularly restricted, an example of the reaction time is in the range from 0.1 to 96 hours, and a reaction time in the range from 1 to 60 hours is preferable.

When Q (Q is the same as described above) in Ar² in general formula (1) corresponding to the Ar^{2a} part in general formula (2) of the compounds represented by general formula (2) represents —CON(R⁴¹)(R⁴²) (R⁴¹ and R⁴² are the same as described above), the compound can be produced by allowing a compound in which Q (Q is the same as described above) in Ar² in general formula (1) corresponding to the Ar^{2a} portion in general formula (2) represents a carboxy group to react with ammonia, primary amine, secondary amine or hydroxyamine in the presence of a condensing agent and, if necessary, in the presence of a base. When Q (Q is the same as described above) in Ar² in general formula (1) corresponding to the Ar^{2a} part in general formula (2) of the compounds represented by general formula (2) represents —CON(R⁴¹)(R⁴²) (R⁴¹ and R⁴² are the same as described above), the proportion of use of ammonia, primary amine, secondary amine or hydroxyamine may be in the range from 1/10 to 100 equivalent, preferably in the range from 1 to 10 equivalent, relative to the compound in which Q (Q is the same as described above) in Ar² in general formula (1) corresponding to Ar^{2a} in general formula (2) represents a carboxy group. Examples of the condensing agent include dicyclohexyl carbodiimide (DCC), O-(7-azabenzotriazole-1-yl)N,N,N′,N′-tetramethyluronium hexafluorophosphate (HATU) and 1-ethyl-3-[3-dimethylaminopropyl]carbodiimide (EDC) hydrochloride. When Q (Q is the same as described above) in Ar² in general formula (1) corresponding to Ar^{2a} portion in general formula (2) represents —CON(R⁴¹)(R⁴²) (R⁴¹ and R⁴² are the same as described above), the proportion of use of the condensing agent may be in an equivalent amount or in excess, for example in the range from 1 to 10 equivalent, more preferably from 1 to 5 equivalent, relative to the compound in which Q (Q is the same as described above) in Ar² in general formula (1) corresponding to Ar² represents a carboxy group. An auxiliary agent may be added to the condensation reaction, and an example of the auxiliary agent is hydroxybenzotriazole (HOBT). Examples of the base include trimethylamine, triethylamine, diisopropylethylamine, N-methylmorpholine and pyridine. Triethylamine, diisopropylethylamine, N-methyl morpholine and pyridine are preferable. Examples of the solvent used in the reaction include dichloromethane, chloroform, carbon tetrachloride, benzene, toluene, xylene, diethylether, tetrahydrofuran, dioxane, dimethoxyethane, pyridine and dimethylformamide. A mixture of a plurality of these solvents may be preferably used. Dimethylformamide and tetrahydrofuran are preferable. The reaction temperature is usually in the range from −80 to 100° C. While the reaction time is not particularly restricted, it is usually in the range from 1 to 96 hours, preferably from 2 to 48 hours. Ammonia, primary amine, secondary amine and. protected hydroxyamine may be used in the reaction, and an example of protected hydroxyamine is O-(2-methoxypropane-2-yl)hydroxyamine. The protective group can be de-protected according to known methods (for example, Mori, K., Tetrahedron, 44, 6013(1988)).

Of the compounds represented by general formula (2), the compound—in which Ar¹ in general formula (1) corresponding to Ar^{1a} portion in general formula (2) is substituted with an aryl group that may be substituted or with an alkenyl group that may be substituted—can be produced from a compound in which Ar¹ in general formula (1) corresponding to Ar^{1a} portion in general formula (2) is substituted with bromine or iodine. The reaction may be effected under the conditions according to Suzuki reaction or Heck reaction, which are described in published reports (R. F. Heck, Organic Reactions, 27, 345 (1982); R. F. Heck, Palladium Reagents in Organic Synthesis, Academic Press, 1985; N. Miyaura et al., J. Am. Chem. Soc., 107, 972 (1985); N. Miyaura, A, Suzuki, Chem. Rev. 95, 2457 (1995)).

Of the compounds represented by general formula (2), the compound—in which Ar¹ in general formula (1) corresponding to Ar^{1a} portion in general formula (2) is substituted with an alkyl group that may be substituted—can be produced from a compound in which Ar¹ in general formula (1) corresponding to Ar^{1a} portion in general formula (2) is substituted with an alkenyl group that may be substituted or with an alkynyl group that may be substituted. An example of the production method of this compound is a catalytic reduction method, which may be effected using a catalyst under a hydrogen atmosphere in a solvent. Examples of the catalyst include palladium-carbon, platinum oxide, platinum-carbon and palladium hydroxide. Examples of the solvent used in the reaction include dichloromethane, chloroform, carbon tetrachloride, benzene, toluene, xylene, diethylether, tetrahydrofuran, dioxane, dimethoxyethane, methanol and ethanol, preferably tetrahydrofuran and methanol. A mixture of a plurality of these solvents may be preferably used. The reaction time is usually in the range form −80 to 100° C., preferably from 0 to 50° C. While the reaction time is not particularly restricted, it is usually 1 to 96 hours, preferably 3 to 48 hours.

Of the compounds represented by general formula (2), the compound—in which Ar¹ in general formula (1) corresponding, to Ar^{1a} portion in general formula (2) is substituted with an amino group that may be substituted—can be produced from a compound in which Ar¹ in general formula (1) corresponding to Ar^{1a} portion in general formula (2) is substituted with bromine or iodine. The compound can be produced via Buchwald-Hartwig reaction known in the art, and reaction conditions thereof are in accordance with published reports (A. R. Muci, S. L. Buchwald,. Top. Curr. Chem., 219, 131 (2002); J. F. Hartwig, Angew. Chem., Int. Ed., 37, 2046 (1998); D. Baranano, G. Mann, Hartwig, J. F., Curr. Org. Chem.1, 287 (1997); C. G. Frost, P. Mendonca, J. Chem. Soc. Perkin Trans. 1, 1998, 2615). While the reaction product may be obtained while its amino group remains protected depending on the reaction conditions, a derivative having amino groups may be produced by applying an appropriate deprotection reaction.

Of the compounds represented by general formula (2), the compound—in which Ar¹ in general formula (1) corresponding to Ar^{1a} portion in general formula (2) is substituted with an amino group substituted with an acyl group that may be substituted, an alkylsulfonyl group that may be substituted, an alkylcarbamoyl group that may be substituted, an alkylthiocarbamoyl group that may be substituted, an arylcarbamoyl group that may be substituted, an arylthiocarbamoyl group that may be substituted, an alkyloxycarbonyl group that may be substituted or an aryloxycarbonyl group that may be substituted—can be producing by allowing a compound, in which Ar¹ in general formula (1) corresponding to Ar^{1a} in general formula (2) is substituted with an amino group, to react with an electrophilic reagent. Examples of the electrophilic reagent include acyl chlorides, acyl bromides, acid anhydrides, alkylsulfonyl chlorides, alkylsulfonic acid anhydrides, arylsulfonyl chlorides, arysulfonic acid anhydrides, isocyanate, isothiocyanate, carbamoyl chloride and chloroformate. The proportion of use of the electrophilic reagent is in a molar equivalent or in excess, for example 1 to 10 equivalent, preferably 1 to 3 equivalent, relative to the compound in which Ar¹ in general formula (1) corresponding to Ar^{1a} portion in general formula (2) is substituted with an amino group. A base may be used in the reaction, if necessary. The base may be either organic or inorganic bases, and examples of the base include sodium hydroxide, potassium hydroxide, potassium carbonate, sodium carbonate, trimethylamine, triethylamine, diisopropylethyl amine, N-methylmorphorine and pyridine. The proportion of use of the base may be in a molar equivalent or in excess, more preferably 1 to 100 equivalent, particularly 1 to 10 equivalent, relative to the compound in which Ar¹ in general formula (1) corresponding to Ar^{1a} portion in general formula (2) is substituted with an amino group. An inert solvent can be used for the reaction. Examples of the inert solvent include dichloromethane, chloroform, carbon tetrachloride benzene, toluene, xylene, diethylether, tetrahydrofuran, dioxane, dimethoxyethane, ethyl acetate and butyl acetate. Dichloromethane, diethylether and tetrahydrofuran are preferable. A mixture of a plurality of these solvents may be preferably used. The reaction temperature is usually in the range from −20 to 100° C., preferably from −10 to 50° C. While the reaction time is not particularly restricted, it is usually in the range from 0.2 to 24 hours, preferably from 1 to 5 hours.

Of the compounds represented by general formula (2), the compound—in which Ar² in general formula (1) corresponding to Ar^{2a} portion in general formula (2) is represented by -E-Ar²¹-G-Q-, wherein G represents an alkylene group, two bonds formed by removing two hydrogen atoms on the same carbon atom on the alkylene group are linked to an oxygen atom together so as to be substituted as an oxo-form—can be produced by oxidizing a compound of the compounds represented by general formula (2), in which Ar² in general formula (1) corresponding to Ar^{2a} portion in general formula (2) represents -E-Ar²¹-G-Q , wherein G represents an alkylene group which is substituted with a hydroxy group. Examples of the oxidation reaction include a method using a Dess-Martin reagent, a Swern oxidation method and an oxidation method using chromic acid. These oxidation methods are readily available to those skilled in the art.

Of the compounds represented by formula (2), the compound—in which Ar¹ in general formula (1) corresponding to Ar^{1a} portion in general formula (2) is substituted with an alkyl group substituted with a hydroxy group—can be produced by reducing a compound of the compounds represented by general formula (2) in which Ar¹ in general formula (1) corresponding to Ar^{1a} portion in general formula (2) is substituted with an acyl group. An example of the reduction condition is hydride reduction. An example of the hydride reduction condition is to allow a metal hydride to react in the presence of an acid, if necessary. Examples of the metal hydride include hydrosilanes such as triethylsilane, sodium borohydride and sodium tellurium hydride. The proportion of use of the metal hydride may be in an equimolar amount or in excess, for example 1 to 20 equivalent, preferably 1 to 10 equivalent, relative to the compound of the compounds represented by general formula (2) in which Ar¹ in general formula (1) corresponding to Ar^{1a} in general formula (2) is substituted with an acyl group. The reaction may be performed in the presence of an acid, if necessary. The acid may be an organic or inorganic acid, and examples of the acid include hydrochloric acid, sulfuric acid, hydrobromic acid, formic acid, acetic acid, trifluoroacetic acid and boron trifluoride/diethylether complex. Trifluoroacetic acid is preferable. The proportion of use of the acid may be in an equimolar amount or in excess, preferably 1 to 20 equivalent, more preferably 1 to 10 equivalent, relative to the compound of the compounds represented by general formula (2) in which Ar¹ in general formula (1) corresponding to Ar^{1a} portion in general formula (2) is substituted with an acyl group. The acid may be preferably used as a solvent. Examples of the solvent used in the reaction include dichloroethane, chloroform, carbon tetrachloride, benzene, toluene, xylene, diethylether, tetrahydrofuran, dioxane, dimethoxyethane, methanol and ethanol. Tetrahydrofuran and dichloromethane are preferable. A mixture of a plurality of these solvents may be preferably used. While the reaction temperature is in the range from −80 to 100° C., a temperature in the range from 0 to 50° C. is preferable. While the reaction time is not particularly restricted, it is usually in the range from 1 to 48 hours, preferably from 3 to 24 hours.

Of the compounds represented by general formula (2), the compound—in which Ar¹ in general formula (1) corresponding to Ar^{1a} in general formula (2) is substituted with an alkyl group substituted with a hydroxy group—can be produced by a hydroxylation reaction by hydroboration of a compound of the compounds represented by general formula (2), in which Ar¹ in general formula (1) corresponding to Ar^{1a} in general formula (2) is substituted with an alkenyl group. Hydroxylation by the hydroboration reaction may be readily performed by those skilled in the art.

Of the compounds represented by general formula (2), the compound—in which Ar¹ in general formula (1) corresponding to Ar^{1a} in general formula (2) is substituted with an amino group that may be substituted with an alkyl group—can be produced by allowing a compound—in which Ar¹ in general formula (1) corresponding to Ar^{1a} in general formula (2) is substituted with an amino group—to react with an alkylating reagent in the presence of a base, if necessary. A corresponding alkyl halide may be used as the alkylating reagent, and examples of the alkyl halide include alkyl iodide, alkyl bromide, alkyl chloride and 2-bromoethylmethyl ether. The proportion of use of the alkylating reagent is preferably in an equimolar amount or in excess, for example in the range from 1 to 10 equivalent, preferably from 1 to 3 equivalent, relative to the compound, of the compounds represented by general formula (2) in which Ar¹ of general formula 1 corresponding to Ar^{1a} of the general formula (2) is substituted with an amino group. A base may be used in the reaction, if necessary. The base may be either an organic base or an inorganic base, and examples of the base include sodium hydroxide, potassium hydroxide, potassium carbonate, sodium carbonate, trimethylamine, triethylamine, diisopropylethylamine, N-methylmorpholine, pyridine and 2,6-lutidine. The proportion of use of the base is preferably in an equivalent amount or in excess, more preferably in the range from 1 to 100 equivalent, further preferably in the range from 1 to 10 equivalent, relative to the compound in which Ar¹ in general formula (1) corresponding to Ar^{1a} portion in general formula (2) is substituted with an amino group. An inert solvent may be used for the reaction. While examples of the inert solvent include dichloromethane, chloroform, carbon tetrachloride, benzene, toluene, xylene, diethylether, tetrahydrofuran, dioxane, dimethoxyethane, ethyl acetate and butyl acetate, dichloromethane, diethylether and tetrahydrofuran are preferable. A mixture of a plurality of these solvents may be preferably used. The reaction temperature is usually in the range from −20 to 100° C., preferably from −10 to 50° C. While the reaction time is not particularly restricted, it is usually from 0.2 to 24 hours, preferably from 1 to 5 hours. The compound—in which Ar¹ of general formula 1 corresponding to Ar^{1a} of the general formula (2) is substituted with an amino group substituted with an alkyl group that may be substituted—can be also produced under a reductive amination reaction condition in which a reducing agent is allowed to react with a compound—in which Ar¹ of general formula 1 corresponding to Ar^{1a} of the general formula (2) is substituted with an amino group—in a solvent in the presence of an aldehyde or a ketone and, if necessary, an acid. While examples of the solvent used for the reaction include diethylether, tetrahydrofuran, dioxane, dimethoxyethane, methanol, ethanol and acetonitrile, methanol and acetonitrile are preferable. A mixture of a plurality of these solvents may be preferably used. Examples of the aldehyde and ketone include formaldehyde, acetaldehyde and acetone. The aldehyde is preferably used in an equivalent amount or in excess, more preferably in the range from 1 to 10 equivalent, relative to the compound in which Ar¹ of general formula 1 corresponding to Ar^{1a} of the general formula (2) is substituted with an amino group. A preferable example of the acid used when necessary is acetic acid or an acidic buffer solution. Sodium hydrogen cyanoborate is preferable as the reducing agent, which is preferably used in an equivalent amount or in excess, more preferably in the range from 1 to 10 equivalent, relative to the compound in which Ar¹ of general formula 1 corresponding to Ar^{1a} of the general formula (2) is substituted with an amino group. The reaction temperature is in the range from −20 to 100° C., preferably from −10 to 50° C. While the reaction time is not particularly restricted, it is usually in the range from 0.2 to 36 hours, preferably from 1 to 24 hours.

Of the compounds represented by general formula (2), a compound—in which Ar¹ of general formula 1 corresponding to Ar^{1a} of the general formula (2) is substituted with iodine—can be produced by allowing a compound in which Ar¹ of general formula 1 corresponding to Ar^{1a} of the general formula (2) is substituted with bromine—to react with an iodinating agent in the presence of a copper salt and an amine, if necessary. Examples of the iodinating reagent include sodium iodide and potassium iodide. The amount of use is preferably in an equivalent amount or in excess. An example of the copper salt is copper (I) iodide, which is preferably used in an catalytic quantity. An example of the amine is N,N′-dimethyl-1,2-diaminocyclohexane, which is also used in a catalytic quantity. Inert solvent such as dioxane and tetrahydrofuran may be used as a solvent. The reaction temperature is preferably in the range from room temperature to 150° C. An example of the reaction time is in the range from 1 to 50 hours, preferably from 3 to 30 hours.

Of the compounds represented by general formula (2), a compound—in which Ar¹ of general formula 1 corresponding to Ar^{1a} of the general formula (2) is substituted with chlorine—can be produced by allowing a compound in which Ar¹ of general formula 1 corresponding to Ar^{1a} of the general formula (2) is substituted with bromine to react with copper chloride. A preferable example of the copper chloride is copper (I) chloride, which is preferably used in an equivalent amount or in excess. Examples of the solvent available are inert solvents such as N,N-dimethylformamide, dioxane and tetrahydrofuran. The reaction time is preferably from room temperature to 150° C. An example of the reaction time is in the range from 1 to 50 hours, preferably from 3 to 30 hours.

Of the compounds represented by general formula (2), a compound—in which Ar¹ of general formula 1 corresponding to Ar^{1a} of the general formula (2) is substituted with an alkynyl group that may be substituted—can be produced from a compound in which Ar¹ of general formula 1 corresponding to Ar^{1a} of the general formula (2) is substituted with bromine or iodine. The reaction can be effected by Sonogashira reaction, and the reaction condition thereof is as described in a published report (K. Sonogashira et al., Tetrahedron Lett. 50, 4467 (1975) and J. Organomet. Chem., 653, 46 (2002)).

Of the compounds represented by general formula (2), a compound—in which Ar¹ of general formula 1 corresponding to Ar^{1a} of the general formula (2) is substituted with an alkylsulfonyl group that may be substituted—can be produced by allowing an oxidizing agent to react, in a solvent, with the compound in which Ar¹ of general formula 1 corresponding to Ar^{1a} of the general formula (2) is substituted with an alkylthio group that may be substituted. Examples of the solvent used in the reaction are dichloromethane, chloroform and carbon tetrachloride. Particularly, dichloromethane is preferable. A mixture of a plurality of these solvents may be preferably used. The reaction temperature is usually in the range from −80 to 100° C., preferably from 0 to 50,C. While the reaction time is not particularly restricted, an example of the reaction time is from 0.1 to 48 hours, preferably from 0.3 to 24 hours.

The compound represented by general formula (3) can be produced by allowing a compound represented by general formula (5) (in general formula (5), Ar^{1a}, R^{1a} and R^{2a} are the same as described above) to react in the presence of a chlorinating agent. While examples of the chlorinating agent include acid chlorides such as phosphorus oxychloride, thionyl chloride and oxalyl chloride, phosphorous oxychloride is preferable as the acid chloride. The proportion of use of the chlorinating reagent is in the range from 1/10 to 10 equivalent, preferably from 1/5 to 5 equivalent, and more preferably from 1 to 3 equivalent relative to the compound represented by general formula (5). It is also preferable to use an excess amount of the chlorinating reagent relative to the compound represented by general formula (5) in another embodiment, or the reagent may be more preferably used as a solvent. The reaction is preferably effected in the presence of an amine such as triethylamine or N,N-dimethylaniline, and the proportion of the amine is preferably in the range from 0.5 to 10 equivalent, more preferably from 1 to 3 equivalent. Examples of the solvent used in the reaction include dichloromethane, chloroform, carbon tetrachloride, benzene, toluene, xylene, diethylether, tetrahydrofuran, dioxane, dimethoxymethane, ethyl acetate, butyl acetate and acetonitrile. The chlorinating reagent may be preferably used as a solvent in another embodiment. The reaction time is usually in the range from 0 to 200° C., preferably from 50 to 150° C. While the reaction time is not particularly restricted, an example of it is in the range from 0.1 to 100 hours, preferably from 0.5 to 10 hours.

Of the compounds represented by general formula (3), a compound—in which Ar¹ corresponding to Ar^{1a} portion in general formula (3) is substituted with an alkoxy group that may be substituted—can be produced from a compound in which Ar¹ corresponding to Ar^{1a} portion in general formula (3) is substituted with a hydroxy group. The compound can be produced by Mitsunobu reaction known in the art, and the reaction condition is as described in a published report (Mitsunobu, O., Synthesis, 1981, 1; Hughes, D. L., Org. React. 42, 335 (1992)).

Of the compounds represented by general formula (3), a compound—in which Ar¹ corresponding to Ar^{1a} portion in general formula (3) is substituted with a hydroxy group—can be produced from a compound in which Ar¹ corresponding to Ar^{1a} portion in general formula (3) is substituted with a methoxy group. The production method is based on a demethylation reaction, which can be effected according to the method described in Protective Groups in Organic Synthesis, John Wiley and Sons (1999).

Of the compounds represented by general formula (3), a compound—in which Ar¹ corresponding to Ar^{1a} portion in general formula (3) is substituted with an aminomethyl group that may be substituted with an alkyl group—can be produced by allowing a primary or secondary amine to react, in the presence of a base in the solvent if necessary, with a compound in which Ar¹ corresponding to Ar^{1a} portion in general formula (3) is substituted with a chloromethyl group. An example of the base that is used if necessary is triethylamine. The proportion of use of the primary or secondary amine may be in an equivalent amount or in excess, and preferably in the range from 1 to 10 equivalent relative to the compound in which Ar¹ corresponding to Ar^{1a} portion in general formula (3) is substituted with a chloromethyl group. The primary amine used for the reaction is, for example, methylamine. The secondary amine used for the reaction is, for example, dimethylamine. Examples of the solvent used in the reaction include dichloromethane, chloroform, carbon tetrachloride, benzene, toluene, xylene, diethylether, tetrahydrofuran, dioxane, dimethoxyethane, ethyl acetate, butyl acetate and acetonitrile. The reaction temperature is usually in the range from 0 to 200° C., preferably from 20 to 100° C. While the reaction time is not particularly restricted, an example of it is in the range from 0.1 to 100 hours, preferably from 1 to 30 hours.

Of the compounds represented by general formula (3), a compound—in which Ar¹ corresponding to Ar^{1a} portion in general formula (3) is substituted with a chloromethyl group—can be produced by allowing a compound in which Ar¹ corresponding to Ar^{1a} portion in general formula (3) is substituted with a hydroxymethyl group to react in the presence of a chlorinating reagent. While examples of the chlorinating reagent include acid chloride such as phosphorous oxychloride, thionyl chloride and oxalyl chloride, phosphorous oxychloride is preferable as the acid chloride. The proportion of use of the chlorinating agent may be in the range from 1/10 to 10 equivalent, preferably in the range from 1/5 to 5 equivalent, and more preferably from 1 to 3 equivalent relative to the compound in which Ar¹ corresponding to Ar^{1a} portion in general formula (3) is substituted with the hydroxymethyl group. It is also preferably to use the chlorinating reagent in excess relative to the compound in which Ar¹ corresponding to Ar^{1a} portion in general formula (3) is substituted with the hydroxymethyl group. The reaction may be preferably effected in the presence of an amine such as triethylamine and N,N-dimethylaniline, and the amount of use of the amine is preferably in the range from 0.5 to 10 equivalent, more preferably from 1 to 3 equivalent. Examples of the solvent used in the reaction include dichloromethane, chloroform, carbon tetrachloride, benzene, toluene, xylene, diethylether, tetrahydrofuran, dioxane, dimethoxyethane, ethyl acetate, butyl acetate and acetonitrile. The chlorinating agent may be preferably used as the solvent in another embodiment. The reaction temperature is usually in the range from 0 to 200° C., preferably from 50 to 150° C. While the reaction time is not particularly restricted, an example of it is in the range from 0.1 to 100 hours, preferably from 0.5 to 10 hours.

A protective group may be introduced into the compound represented by general formula (3), if necessary, or the compound may be used after deprotection. The reaction for introducing the protective group and deprotection reaction may be effected according to the method described in Protective Groups in Organic Synthesis, John Wiley and Sons (1999).

Commercially available 4-aminobenzoic acid (manufactured by Aldrich Co.), 6-amino-2-naphthalene carboxylic acid (manufactured by Oakwood Co.) 4-aminophenyl acetic acid (manufactured by Aldrich Co.) and 4-amino-3-methoxybenzoic acid (manufactured by Aldrich Co.) may be used as the compound represented by general formula (4).

The compound represented by general formula (4) can be produced by a reduction reaction of a corresponding nitro compound. Examples of the reduction reaction include a catalytic hydrogenation method and a method using a metal hydride complex. The catalytic hydrogenation method may be performed using a catalyst in a solvent under a hydrogen atmosphere. Examples of the catalyst include palladium-carbon, platinum oxide, platinum-carbon and palladium hydroxide. While examples of the solvent used in the reaction include dichloromethane, chloroform, carbon tetrachloride, benzene, toluene, xylene, diethylether, tetrahydrofuran, dioxane, dimethoxyethane, methanol and ethanol, tetrahydrofuran and methanol are preferable. A mixture of a plurality of these solvents may be suitably used. While the reaction temperature is usually in the range from −80 to 100° C., preferably from 0 to 50° C. While the reaction time is not particularly restricted, it is usually in the range from 1 to 96 hours, preferably from 3 to 48 hours. The reduction reaction using the metal hydride complex may be effected by adding an additive, if necessary. A preferable example of the metal hydride complex is sodium borohydride. The proportion of use of the metal hydride complex may be in an equivalent amount or in excess relative to the nitro compound, preferably in the range from 1 to 10 equivalent. While examples of the solvent used in the reaction include diethylether, tetrahydrofuran, dioxane, dimethoxyethane, methanol and ethanol, tetrahydrofuran and methanol are preferable. A mixture of a plurality of these solvents may be suitably used. A Lewis acid or metal salt may be added as the additive, and a preferable example is nickel chloride. The proportion of the additive may be in a catalytic amount or in excess, relative to the nitro compound, preferably in the range from 0.01 to 1 equivalent. The reaction temperature is usually in the range from −80 to 100° C., preferably from −20 to 50° C. While the reaction time is not particularly restricted, it is usually in the range from 0.1 to 96 hours, preferably from 0.5 to 24 hours. Examples of the nitro compound used in the reaction are commercially available compounds such as nitrophenyl glyoxylate (manufactured by Lancaster Co.). Corresponding nitro compounds may be produced by a nitration reaction of aromatic compounds. The nitration reaction can be effected by allowing nitric acid or a nitrate to react in sulfuric acid by a method known in the art. An intermediate having a carboxymethyl group of the nitro compounds can be produced as follows. A cyanomethyl derivative is produced by using trimethylsilyl acetonitrile, a metal complex such as Pd₂(dba)₃, a complex ligand such as Xantphos and a metal salt such as zinc fluoride to a corresponding bromo-derivative. The derivative can be produced by a known method (Wu, L. et al., J. Am. Chem. Soc. 127, 15824 (2005)). Subsequently, a carboxymethyl derivative can be produced by hydrolyzing the cyanomethyl derivative. The hydrolysis reaction can be effected by allowing water and an acid to react in a solvent. Water is preferably used in excess or as a solvent, and using water as a solvent is more preferable. Examples of the preferably used solvent include water, THF, 1,4-dioxane, methanol and ethanol. Hydrochloric acid or sulfuric acid is preferably used as the acid, and sulfuric acid is more preferably used. The proportion of use of the acid is preferably in excess relative to the cyanomethyl derivative. An example is to use an aqueous solution prepared by diluting conc. sulfuric acid twice with water as a solvent. The reaction temperature is usually in the range from 0 to 200° C., preferably from 20 to 150° C. While the reaction time is not particularly restricted, it is usually in the range from 0.1 to 96 hours, preferably from 1 to 10 hours.

The compound represented by general formula (4) can be produced from a commercially available halogen compound through Buchwald-Hartwig reaction known in the art, and the reaction condition is as described in published reports (A. R. Muci, S. L. Buchwald, Top. Curr, Chem. 219, 131 (2002); J. F. Hartwig, Angew. Chem., Int. Ed. 37, 2046 (1998); D. Baranano, G. Mann, Hartwig, J. F., Curr. Org. Chem. 1, 287 (1997); C. G. Frost, P. Mendonca, J. Chem. Soc. Perkin Trans. 1, 1988, 2615). The reaction product may be obtained while the amino group remains protected, and a derivative having the amino group may be obtained by an appropriate deprotection reaction. An example of the commercially available halogen compounds is methyl 2-(4-bromophenyl)-2,2-dimethylacetate (manufactured by Tronto Co.).

The compound represented by general formula (4) may be used by introducing a protective group or by eliminating the protective group when necessary. Introduction and elimination of the protective group may be effected according to a method known in the art, for example the method described in Protective Groups in Organic Synthesis, John Wiley and Sons, 1999.

The compound represented by general formula (5) can be produced by allowing a compound represented by general formula (6) (in general formula (6), R^{1a} and R^{2a} are the same as described above, and R⁴ denotes a lower alkyl group) to react with a compound represented by general formula (7) (in general formula (7), Ar^{1a} is the same as described above) in the presence of a base. While the proportion of use of the compound represented by general formula (7) in the reaction between the compound represented by general formula (6) and the compound represented by general formula (7) is in the range from 1/1 to 10 equivalent, preferably from 1/5 to 5 equivalent, and more preferably from 0.5 to 2 equivalent relative to the compound represented by general formula (6), the reaction may be appropriately designed considering the purity of the compound represented by general formula (5), the yield and the purification efficiency. A compound converted into an acid addition salt with hydrochloric acid may be suitably used as the compound represented by general formula (7). While examples of the base used in the reaction include metal alkoxide such as sodium methoxide and sodium ethoxide, carbonates such as sodium carbonate, potassium carbonate and cesium carbonate, metal hydride such as sodium hydride, and sodium hydroxide and potassium hydroxide, sodium ethoxide, sodium methoxide and sodium hydride are preferable. The proportion of the base is in the range from 1/5 to 10 equivalent, preferably from 1/2 to 3 equivalent, relative to the compound represented by general formula (7). When the compound represented by general formula (7) is used as an acid addition salt, a base in one equivalent excess is preferably used for neutralizing the acid addition salt. Examples of the solvent used include ethanol, methanol, propanol, isopropanol, ethylene glycol, propyleneglycol, diethyleneglycol monomethyl ether, dichloromethane, chloroform, carbon tetrachloride, benzene, toluene, xylene, diethylether, tetrahydrofuran, dioxane, dimethoxyethane, diethyleneglycol dimethyl ether, ethyl acetate, butyl acetate and acetonitrile. Ethanol, methanol, propanol, isopropanol, ethylene glycol, propyleneglycol and diethyleneglycol monomethyl ether are preferable examples, and ethanol and ethylene glycol are more preferable. A mixture of a plurality of these solvents may be used. The reaction temperature is usually in the range from 0 to 200° C., preferably from 50 to 150° C. While the reaction time is not particularly restricted, it is usually in the range from 1 to 96 hours, preferably 3 to 36 hours.

Of the compounds represented by general formula (5), a compound—in which R² corresponding to R^{2a} portion in general formula (5) is a methyl group substituted with either an alkoxy group, acyloxy group or amine that may be substituted with an alkyl group—can be produced by allowing an alcohol, a carboxylic acid or an amine to react, as a nucleophilic reagent, with a compound in which R^{2a} in general formula (5) is substituted with a bromomethyl group. Examples of the alcohol are methanol and ethanol, an example of the carboxylic acid is acetic acid, and an example of the amine is dimethylamine. The alcohol or carboxylic acid is preferably used as a solvent used for the production when the nucleophilic reagent is the alcohol or carboxylic acid, and an alcoholic solvent such as methanol and ethanol is preferably used when an amine is used as the nucleophilic reagent. An acid such as hydrochloric acid is desirably added when necessary when the nucleophilic reagent is an alcohol or a carboxylic acid. The proportion of use of the nucleophilic reagent is in an equivalent amount or in excess, preferably in the range from 1 to 100 equivalent. The reaction temperature is usually in the range from 0 to 200° C., preferably from 0 to 50° C. While the reaction time is not particularly restricted, it is usually in the range from 0.1 to 99 hours, preferably from 0.5 to 36 hours. The compound in which R^{2a} in general formula (5) is substituted with a bromomethyl radial can be produced by allowing N-bromosuucinimide (NBS) to react, in the presence of a radical initiator in a solvent, with a compound in which R^{2a} in general formula (5) is a methyl group. Examples of the preferable solvent include dichloromethane, chloroform, carbon tetrachloride, benzene, toluene and xylene, and carbon tetrachloride is preferable. A preferable radical initiator is 2,2′-azobis(isobutyronitrile), which is preferably used in a catalytic quantity relative to the compound in which R^{2a} in general formula (5) is a methyl group. The proportion of use of NBS is in the range from 1 equivalent to in excess, preferably from 1 to 10 equivalent. The reaction temperature is usually in the range from 0 to 200° C., preferably from 50 to 150° C. While the reaction time is not particularly restricted, it is usually in the range from 0.1 to 96 hours, preferably from 0.5 to 36 hours.

The compound represented by general formula (5) may be used by introducing a protective group or by eliminating the protective group when necessary. Introduction of the protective group and deprotection may be effected according to a known method, for example the method described in Protective Groups in Organic Synthesis, John Wiley and Sons, 1999.

Commercially available chemicals such as ethyl acetoacetate (manufactured by Aldrich Co.), ethyl benzoylacatate (manufactured by Aldrich Co.), ethyl 2-benzoylacetoacetate (manufactured by Aldrich Co.), ethyl 4,4,4-trifluoroacetoacetate (manufactured by Aldrich Co.), ethyl propyonylacetate (manufactured by Aldrich Co.), ethyl 2-cyanoacetoacetate (manufactured by Alfa Aesar Co.) and ethyl 2-ethylacetate (manufactured by Aldrich Co.) may be used as the compound represented by general formula (6), or ethyl 2-allylacetoacetate obtained by a known method (J. Org. Chem. 60, 856-862, 1995), ethyl 2-ethyl-3-oxopentanate obtained by a known method (J. Org. Chem. 42, 459, 1977) or methyl 2-methoxyacetoacetate obtained by a known method (Tetrahedron, 44, 1603, 1988) may be used.

Commercially available chemicals such as 2-thiophene carboximidamide hydrochloride (manufactured by Maybridge Co.), 3-thiophene carboximidamide hydrochloride (manufactured by Maybridge Co.) or 3-furan carboximidamide hydrochloride (manufactured by Maybridge Co.) may be used as the compound represented by general formula (7), or a commercially available derivative of an aromatic compound may be amidinated by a known method (Jikken Kagaku Koza (Handbook of Experiment in Chemistry) Vol. 20 and Vol. 21, 4-th edition, Maruzen Co.). For example, 5-methoxythiophene-2-carboximidamide hydrochloride can be produced from 2-methoxythiophene (manufactured by Aldrich Co.) by this method.

Specifically, a commercially available derivative of an aromatic compound is formylated to produce an intermediate of an aromatic aldehyde. The formylation reaction may be effected by known Vismeier reaction. Then, the intermediate of the aromatic aldehyde is converted into an oxime compound, followed by a dehydration reaction to produce an intermediate of aromatic nitrile. The oxime-forming reaction and dehydration reaction can be produced by allowing hydroxyamine or a salt thereof to react with the intermediate of the aromatic aldehyde. A base such as sodium acetate or triethylamine may be used as a reaction accelerating agent. It is preferable to use a dehydrating agent in accordance with completion of the oxime-forming reaction. Examples of the dehydrating agent are phthalic anhydride and tosyl chloride. Subsequently, the compound—represented by general formula (7) can be produced by subjecting the intermediate of the aromatic nitrile to an amidinating reaction. Examples of the amidinaing reaction include a method for allowing lithium bis(trimethylsilyl)amide to react, a method for allowing an aluminum amide compound to react, or a method for allowing a metal alkoxide to react followed by allowing ammonium chloride to react. Of the intermediates of aromatic aldehyde in the above-mentioned production methods, the compound in which corresponding Ar¹ is substituted with bromine can be produced by a bromination reaction of the aromatic aldehyde compound. The bromination reaction may be effected using a brominating reagent in a solvent. Bromine is preferable as the brominating reagent. An acidic solvent is preferably used as the solvent, and acetic acid is specifically a preferable example. The reaction temperature is usually in the range from −20 to 100° C., preferably from 0 to 30° C. While the reaction time is not particularly restricted, it is usually in the range from 0.1 to 96 hours, preferably from 0.5 to 36 hours. Of the intermediates of aromatic nitrile used in the production method, 2-alkylthiothiazole can be produced by allowing an alkyl thiolate metal salt to react with a corresponding 2-bromothiazole derivative. An example of the alkyl thiolate metal salt is sodium methanethiolate. The proportion of use of the alkyl thiolate metal salt is in the range from 1 to 10 equivalent, preferably 1 to 3 equivalent, relative to the 2-bromothiazole derivative. Examples of the solvent used in the reaction include ethanol, methanol, propanol, isopropanol, ethylene glycol, propyleneglycol, diethyleneglycol monomethyl ether, dichloromethane, chloroform, carbon tetrachloride, benzene, toluene, xylene, diethylether, tetrahydrofuran, dioxane, dimethoxyethane, diethyleneglycol dimethyl ether, ethyl acetate, butyl acetate, acetonitrile, N,N-dimethylformamide and dimethylsulfoxide, and ethanol, methanol and N,N-dimethylformamide are preferable. A mixture of a plurality of these solvents may be used. The reaction temperature is usually in the range from −50 to 200° C., preferably from 0 to 100° C. While the reaction time is not particularly restricted, it is usually in the range from 0.1 to 96 hours, preferably from 1 to 36 hours.

The compound represented by general formula (6) and the compound represented by general formula (7) can be used by introducing protective groups or by eliminating the protective groups. The method for introducing the protective group and the deprotection method may be conducted according to known methods, for example the method described in Protective Groups in Organic Synthesis, John Wiley and Sons, 1999.

The compounds of the invention, respective starting materials and intermediates can be isolated and purified by conventional methods such as extraction, distillation and chromatography.

Salts can be produced from the compound represented by general formula (1). While the method for producing the salt is not particularly restricted, an example of the method for producing an acid addition salt comprises the steps of dissolving the compound represented by general formula (1) in an alcohol such as methanol and ethanol, and adding an acid component to the solution in an equivalent amount or an amount of several times in excess. The acid component used may be an acid component corresponding to the acid addition salt described hereinafter, and preferable examples include pharmacologically acceptable inorganic or organic acids such as hydrochloric acid, hydrobromic acid, sulfuric acid, hydrogen sulfuric acid, dihydrogen phosphoric acid, citric acid, maleic acid, tartaric acid, fumaric acid, glucuronic acid and methanesulfonic acid. The base addition salt may be also produced by using a base component in place of the acid component as in the method for producing the acid addition salt. The base component used may be a base component corresponding to the base addition salt described below, and preferable examples thereof include pharmacologically acceptable bases such as sodium hydroxide, potassium hydroxide, N-methyl-D-glucamine, N,N′-dibenzylethylene diamine, 2-aminoethanol, tris(hydroxymethyl)aminomethane, arginine and lysine.

The kind of the salt of the compound represented by general formula (1) in the invention is not particularly restricted, and may be either the acid addition salt or the base addition salt, or may be in a form of a intramolecular ion pair. Examples of the acid addition salt include hydrochloride, hydrobromide, sulfate, hydrogen sulfate, dihydrogen phosphate, citrate, maleate, tartarate, fumarate, gluconate and methanesulfonate, or acid addition salts with optically active acids such as camphorsulfonic acid, mandelic acid or substituted mandelic acid. Examples of the base addition salt include metal salts such a sodium salt and potassium salt, and base addition salts of organic bases such as N-methyl-D-glucamine, N,N′-dibenzyl ethylenediamine, 2-aminoethanol, tris(hydroxymethyl)aminomethane, arginine and lysine. However, it is needless to say that the kind of the base is not restricted to those described above, and they may be appropriately selected by those skilled in the art. Of the salts represented above, pharmacologically acceptable salts are preferable. While the compound of the invention may be a hydrate or solvated compound, these substances are also falls within the scope of the invention.

When a prodrug is produced from the compound represented by general formula (1), a group constituting the prodrug is appropriately introduced into at least one arbitrary group selected from the hydroxy group and amino group in the compound represented by formula (1) using a prodrug-forming reagent such as a corresponding halogenated compound according to a conventional method, and the reaction product is isolated and purified by a conventional method. A group constituting the prodrug can be appropriately introduced into the carboxy group in the compound represented by general formula (1) by a conventional method using a prodrug-forming reagent such as corresponding alcohols or amines. The protective group in the compound represented by general formula (2) may be utilized in the production process for obtaining the prodrug.

While the prodrug of the compound in general formula (1) of the invention is not particularly restricted, an example is a compound in which the group constituting the prodrug is introduced into at least one group selected from the group consisting of hydroxy group, amino group and carboxy group of the compound represented by general formula (1). An example of the group constituting the prodrug with respect to the hydroxy group and amino group is an acyl group and an alkoxycarbonyl group. A preferable example includes an acetyl group, a propionyl group, a methoxycarbonyl group or an ethoxycarbonyl group, and the ethoxycarbonyl group is particularly preferable. The acetyl group is preferable in another embodiment, the propionyl group is preferable in a different embodiment, and the methoxycarbonyl group is preferable in a further different embodiment. Examples of the group constituting the prodrug with respect to the carboxy group include methyl, ethyl, n-propyl, isopropyl, n-butyl, isobutyl, s-butyl, t-butyl, amino, methylamino, ethylamino, dimethylamino and diethylamino groups. The ethyl group, n-propyl group or isopropyl group are preferable, while the ethyl group is particularly preferable. In another embodiment, the n-propyl group is particularly preferable, while the isopropyl group is particularly preferable in a different embodiment.

The compound represented by general formula (1) may contain asymmetric carbon atoms. The stereochemical configuration of these asymmetric carbon atoms is not particularly restricted, and may be in an S-configuration or R-configuration, or a mixture of both of them. Stereoisomers such as pure optically active forms or diastereomers based on the asymmetric carbon atoms, arbitrary mixtures of the stereoisomers, and racemates are also fall within the scope of the invention.

Since the compound represented by general formula (1) of the invention, salts thereof or prodrugs thereof have a potent inhibiting action of PDE4 activity and is highly stable against metabolism, the compound is useful as an effective ingredient of medicines. It has been known that inhibition of the PDE4 activity increases the intracellular concentration of cAMP to enable various therapeutic and/or preventive effects to be elicited. For example, the compound suppresses activation of inflammatory cells (such as eosinophilic lymphocytes, neutrophilic lymphocytes, monocytes, macrophages, mast cells, CD4+T-lymphocytes and CD8+T-lymphocytes). The compound also evokes suppression of production and release of inflammatory cytokines (such as IL-1β, IL-6, IL-8) represented by tumor necrosis factors α (TNF-α). These facts suggest that the compound of the invention is effective for prevention and relax of diseases related to various inflammatory, allergic or immune systems. While TNF-α has been known to be a causative substance of many inflammatory diseases, the anti-inflammatory action of the compound of the invention may be indirectly proved by observing the suppression effect of production of TNF-α in the total blood or cells of mammals stimulated with a lipopolysaccharide (LPS). The compound of the invention is expected to exhibit a potent anti-inflammatory action since it exhibit a potent action for suppressing TNF-α production in both in vitro and in vivo tests as will be shown in Test Examples 2 and 4 to be described hereinafter.

Examples of special uses of the compound represented by general formula (1), salts thereof and prodrugs thereof include prevention and/or therapy of respiratory organ-related diseases such as asthma, chronic obstructive pulmonary diseases (COPD), pneumoconiosis, bronchial asthma, acute bronchitic asthma, chronic bronchitic asthma, inflammatory air tract diseases, pulmonary emphysema, eosinophilic granuloma, adult respiratory distress syndrome (ADRS) and pulmonary fibrosis. Examples of joint-related diseases include rheumatism, osteoarthritis, acute arthritis, chronic arthritis, gouty arthritis, infective arthritis, Lyme arthritis, proliferative arthritis and spondylarthritis. Examples of skin-related diseases include atopic dermatitis, psoriasis, seborrheic eczema, allergic contact eczema and all types of urticaria. Examples of gastrointestine-related diseases include irritable bowel syndrome, ulcerative colitis, Crohn's disease, collagenous colitis and polypous colitis. Examples of eye and nose-related diseases include allergic rhinitis, chronic rhinitis, allergic conjunctivitis, actinic conjunctivitis, catarrhal conjunctivitis, pink-eye and allergic pharyngitis. Examples of immune-related diseases include transplant rejection, multiple sclerosis and AIDS. The compound may be considered to be effective for relief of pain related to inflammation, since inflammation of tissues causes pain. Symptoms of above-mentioned diseases include pain, fever and gout.

The PDE4 inhibitor has been shown to be effective in an animal model of depression (such as forced swimming test) and animal model of memory (such as maze test). See Saccomano, N. A. et al., J. Ned. Chem. 34, p 291-298, 1991; O'Donnell, J. M. and Zhang, H. T., Trends Pharmacol. Sci., 25, p 158-163 (2004; Zhang, H. T. and O'Donnell, J. M, Psychopharmacology, 150, p 311-316, 2000. Since these. improvements are supposed to be caused by activation of the central nerve system as a result of increase of the intracellular cAMP level, the compound of the invention, salts thereof and prodrugs thereof are expected to be effective in diseases that are improved by activation of the central nerve system. Examples of such diseases include degradation of learning and memory ability, Alzheimer's disease, arteriosclerotic dementia, depression, Parkinson's disease, Huntington's disease and late motor disorder.

The compound of the invention, salts thereof or prodrugs thereof are expected to be effective against infectious diseases. Examples of the infectious disease include those in which symptoms become worse by an increase or decrease of TNF-α production in the body of the host, for example infection with HIV, cytomegalovirus (CMV), influenza virus and herpes virus (for example herpes zoster virus and herpes simplex virus).

Since inhibition of PDE4 activity is able to inhibit travel or infiltration of proliferative cells, the compound of the invention, salts thereof or prodrugs thereof can be used for preventing tumors growth and invasion into normal tissues.

It has been also shown in the field of circulatory organs from animal experiments and clinical researches that production of inflammatory cytokines during ischemia in the heart and brain, and in arteriosclerosis layers are also related to infiltration of macrophages and neutrophils. Accordingly, the compounds of the invention, salts thereof or prodrugs thereof are applicable to diseases of circulatory organs. Examples of the disease include ischemia of heart and brain, heart failure, arteriosclerosis and restenosis in the stent.

Since the PDE4 inhibitor has been shown to be effective in a osteoporosis model in ovariectomy rats, the compound of the invention, salts thereof or prodrugs thereof are expected to be used for bone diseases represented by osteoporosis (Waki, Y. et al., Jpn J. Pharmacol. 79, p 477-483, 1999; Miyamoto, K. et al., Biochem. Pharmacol. 54, p 613-617, 1997).

Since the PDE4 inhibitor has been shown to be effective in a mouse diabetes (NOD mouse), the compound of the invention, salts thereof or prodrugs thereof are expected to be used for diabetes (Pyne, N. J. and Furman, B. L., Diabetologia, 46, p 1179-1189, 2003).

It is possible to confirm that the compound of the invention represented by general formula (1), salts thereof or prodrugs thereof are effective as effective ingredients of medicines for prevention and/or therapy of asthma, for example bronchial asthma, using suppression of contraction of extracted bronchus, bronchial asthma model animals and inhibition of migration of white blood cells in the human peripheral blood (Kunihiko Iizuka, Allergy, 47, p 943, 1998; Kunihiko Iizuka and Akihiro Yoshii, Japanese Journal of pneumology, 37, p 196, 1999). Usefulness of the compound of the invention, salts thereof or prodrugs thereof as therapeutic agents of bronchial asthma can be confirmed by measurement of the increases in bronchial resistance due to acetylcholine inhalation after oral administration, intravenous administration or intraperitoneal administration of the compound to model animals at a dosage in the range from 0.1 to 1000 mg/kg, preferably from 0.1 to 100 mg/kg,and further by histological analysis.

It is possible to confirm that the compound of the invention represented by general formula (1), salts thereof or prodrugs thereof are effective as effective ingredients of medicines for prevention and/or therapy of chronic obstructive pulmonary diseases (COPD) using, for example, suppression of contraction of extracted bronchus, bronchial asthma model animals and guinea pig model of tobacco smoke exposure (Junichi Huchigami et al., Proceedings of 73th Meeting of Japanese Society of Pharmacology, 2000), and inhibition of migration of human peripheral white blood cells. Usefulness of the compound of the invention, salts thereof or prodrugs thereof as a therapeutic agent of COPD can be confirmed by counting the number of migrating white blood cells in bronchoalveolar lavage fluid after orally, intravenously or intraperitoneally administering the compound to guinea pig exposed to tobacco smoke at a dosage in the range from 0.1 to 1000 mg/kg, preferably from 0.1 to 100 mg/kg, or by histological analysis.

Usefulness of the compound of the invention, salts thereof or prodrugs thereof as an effective ingredient of the medicine for prevention and/or therapy of pulmonary fibrosis can be confirmed by using a bleomycin-induced pulmonary fibrosis animal model, for example according to the method described in Am. J. Respir. Crit. Care Med., 163, 210-217, 2001. Usefulness of the compound of the invention, salts thereof or prodrugs thereof as a therapeutic agent of pulmonary fibrosis can be confirmed by measuring respiratory function and the amount of hydroxyproline in the lung tissue after orally, intravenously or intraperitoneally administering the compound to the pulmonary fibrosis mouse model at a dosage in the range from 0.1 to 1000 mg/kg, preferably from 0.1 to 100 mg/kg.

Usefulness of the compound of the invention represented by general formula (1), salts thereof or prodrugs thereof as an effective ingredient of the medicine for prevention and/or therapy of chronic articular rheumatism can be confirmed using a collagen-invoked arthritis mouse model (Griffith, M. M. et al., Arthritis Rheumatism, 24, p 781, 1981; Wooley, P. H. et al., J. Exp. Med. 154, p 688, 1981). Usefulness of the compound of the invention, salts thereof or prodrugs thereof as a therapeutic agent of chronic articular rheumatism can be confirmed by measuring the volume of the heel or by measuring the progression of bone destruction after orally, intravenously or intraperitoneally administering the compound to the model mouse or model rat at a dosage in the range from 0.1 to 1000 mg/kg, preferably from 0.1 to 100 mg/kg.

Usefulness of the compound of the invention represented by general formula (1), salts thereof or prodrugs thereof as an effective ingredient of the medicine for prevention and/or therapy of irritable bowel syndrome can be confirmed by administering the compound to a stress-burdened model animal. Examples of the stress-burdened model animal are a restraint stress burdened rat (Miyata, K. et al., J. Pharmacol. Exp. Ther., 259, p 815-819, 1991) and CRH administered rat model (Miyata, K. et al., Am. J. Physiol. 274 (1998), G827-832). The compound of the invention, salts thereof or prodrugs thereof are orally, intravenously or intraperitoneally administered to the stress burdened model animal in a dosage in the range from 0.1 to 1000 mg/kg, preferably from 0.1 to 100 mg/kg, and the number of defecation is measured. Effectiveness of the compound as a therapeutic agent of irritable bowel syndrome can be confirmed by a defecation decreasing effect.

Usefulness of the compound of the invention represented by general formula (1), salts thereof or prodrugs thereof as an effective ingredient of the medicine for prevention and/or therapy of allergy can be confirmed using an atopic dermatitis mouse model, for example according to the method described in Allergy, 50(12), 1152-1162 (2001). Eczema of the skin is induced in Nc/Nga mouse pretreated with a surfactant or an organic solvent using dermatophagoides antigen. Usefulness of the compound of the invention, salts thereof or prodrugs thereof as a therapeutic agent of allergy can be confirmed by measuring the level of the plasma IgE level and the number of eosinophils after orally, intravenously or intraperitoneally administering the compound at a dosage from 0.1 to 1000 mg/kg, preferably from 0.1 to 100 mg/kg.

Usefulness of the compound of the invention represented by general formula (1), salts thereof or prodrugs thereof as an effective ingredient of the medicine for prevention and/or therapy of bone diseases can be confirmed using an osteoporosis mouse model (OVX mouse) prepared by extracting the ovary (Golub, L. M. et al., Ann. N. Y. Acad. Sci., 878, p 290-310, 1999). The compound of the invention, salts thereof or prodrugs thereof are orally, intravenously or intraperitoneally administered at a dosage from 0.1 to 1000 mg/kg, preferably from 0.1 to 100 mg/kg, and deciduous root of tooth and the weight of bone frame are measured. Effectiveness of the compound as the therapeutic agent of abnormal dentary and osteoporosis can be confirmed from the suppressing action of deciduous root of tooth and decrease of the weight of the bone frame.

Fewer incidence of vomiting as an undesirable side effect of the compound of the invention represented by general formula (1), salts thereof or prodrugs thereof can be confirmed by an experiment using, for example, a ferret (Robichaud, A. et al., Neuropharmacology, 38, p 289-297, 1999; Endo, T. et al., Biogenic Amines, 9, p 163-175, 1992). Occurrence of the undesirable side effect, if any, can be confirmed by observing the times of vomiting and vomiting behavior after orally, intravenously or intraperitoneally administering the compound to the ferret at a dosage from 0.1 to 1000 mg/kg, preferably from 0.1 to 100 mg/kg.

Fewer incidence of vomiting action may be also confirmed by a gastric emptying test using a mouse (Haga, K. et al., Arch. Int. Pharmacodyn. 328, p 344-355, 1994; Sato, Y. et al., Biol. Pharm. Bull. 20, p 752-755, 1997). While the transfer rate of the ingredient in the stomach to the small intestine is quantitatively measured in this test, a drug having the vomiting action decreases this transfer rate. The compound is orally, intravenously or intraperitoneally administered to the mouse followed by orally administering a test meal containing a pigment such as phenol red, and the quantity of the test meal remaining in the stomach is measured after a predetermined time period to confirm the undesirable side effect, if any.

Fewer incidence of digestive tract disorder as an undesirable side effect of the compound of the invention represented by general formula (1), salts thereof or prodrugs thereof can be confirmed by measuring gastric acid hyper secretion using, for example, rabbit gastric gland cells (BergLindh et. al., Acta Physiol. Scand., 97:401-414, 1976; Sack et al., Am. J. Physiol. 243: G313-G319, 1982). In particular, the dissociation between the drug anti-inflammatory effects and the digestive tract disorder can be proved by comparing a TNF-α production inhibitory activity, which is an index of the drug effect for therapy of the inflammatory disease, using mouse mononuclear cells with the gastric acid hyper secretion activity using the rabbit gastric gland cells.

The dissociation between the drug anti-inflammatory effects and vomiting and nausea as undesirable side effects can be proved by investigating the selectivity between PDE4 isoform B and D on the compound of the invention represented by general formula (1), salts thereof or prodrugs thereof. Selectivity against each PDE4 isoform may be shown by the inhibitory activity in usual enzyme assay. For example, it may be possible to measure in vitro inhibitory activities of PDE 4B and 4D as a means for investigating selectivity between PDE 4B and 4D. In this method, a test compound is added to a reaction mixture (100 μl) containing 40 mM of Tris-HCl (pH 7.4), 5 mM of MgCl₂, 4 mM of 2-mercaptoethanol, 3 μM of cAMP, 0.83 μCi[³H]-cAMP and a catalytic site of human PDE 4B, and the mixture is allowed to react at room temperature for 10 minutes. After stopping the reaction by adding 25 μl of trichloroacetic acid to the reaction mixture, the solution is mixed with neutral alumina equilibrated with 0.1 M of 2-[[tris(hydroxymethyl)methyl]-amino]-1-ethane sulfonic acid (TES) buffer solution (pH 8.0). The supernatant is removed and, after washing neutral alumina with a sufficient volume of 0.1 M TES buffer solution, alumina is eluted with 2N NaOH. A 500 μl fraction of the eluant containing [³H]-5′-AMP is filled into a scintillation vial containing 3 mL of a scintillation cocktail to measure the radioactivity, and the in vitro inhibitory activity of the test compound against PDE 4B is measured (Catherine Bardelle et al., Analytical; Biochemistry, 275, 148-155, 1999). Inhibitory activity against PDE 4D may be measured likewise.

It may be possible to investigate selectivity between PDE 4B and 4D by using crude enzymes PDE 4B and 4D obtained from a specified cancer cell line and tissue under a given condition (International Immunopharmacology, 2, 1647-1656, 2002). A HL60 cell line is cultivated in RPMI 1640 medium containing 10% of inactivated fetal calf serum and 1.3% of DMSO at 37° C. for 10 days in an environment comprising 95% of air and 5% of CO₂. After 10 days' cultivation, a lysate is prepared after washing the cells. The lysate is fractionated with anion exchange chromatography, cAMP is selectively hydrolyzed, and a specified fraction showing strong inhibition by rolipram that is a selective inhibitor of PDE4 is used as a partially purified PDE 4B enzyme for measuring the in vitro inhibitory activity against PDE 4B. The in vitro inhibitory activity against PDE 4D can be also measured using a partially purified standard sample of the PDE 4D enzyme obtained from U937 cell line cultivated by a conventional method (C. Shepherd et al., British Journal of Pharmacology, 142, 339-351, 2004).

Another usefulness of the compound of the invention represented by general formula (1), salts thereof and prodrugs thereof may be also confirmed by the level of the PDE 4B inhibitory activity as an index of the drug effect for the therapy of inflammatory diseases.

While the medicine of the invention can be formulated as a medicine comprising the compound represented by general formula (1) or a salt thereof as an effective ingredient, a compound administered as a prodrug or a salt thereof may also fall within the scope of the medicine of the invention when the compound is metabolized in vivo to form the compound represented by general formula (1) or a pharmacologically acceptable salt thereof.

While one of the compounds represented by general formula (1) or pharmacologically acceptable salts thereof, or a mixture of a plurality of them may be directly used as the medicine of the invention, it is preferable to administer a pharmaceutical composition formulated by adding one or a plurality of pharmacologically acceptable carriers to the compound represented by general formula (1) or a pharmacologically acceptable salt thereof, or to a mixture of a plurality of the compounds represented by the general (1) or pharmacologically acceptable salts thereof. While the kind of the pharmacologically acceptable carrier is not particularly restricted, examples of the carrier include an excipient, a binder, a disintegrant, a lubricant and an additive. An example of the diluents is D-mannitol. An example of the binder is carboxymethyl cellulose. An example of the disintegrant is corn starch. An example of the lubricant is glycerin. Examples of the additive are paraoxybenzoic acid ester as well as surfactants such as polyoxyethylene sorbitan monooleate (Tween 80) and HC60.

The medicine of the invention may be orally administered to a human as a tablet, powder, granules, capsule, sugar coating tablet, liquid or syrup, or may be administered as an injection drag, drip infusion, suppository or a percutaneous absorption agent. Inhalation as a spray agent such as an aerosol and a dry powder is also preferable medication.

While the period of administration of the medicine of the invention is not particularly restricted, a period that may be judged to express clinical symptoms of the disease can be selected as the administration period when the medicine is administered for therapeutic purposes. While administration is general continued in the period from several weeks to one year, it is possible to further continue administration depending on the disease condition, or to continuously administer after the recovery of clinical symptoms. The medicine may be administered for preventive purposes by the judgment of a doctor in charge even when no clinical symptoms are expressed. While the dose of the medicine of the invention is not particularly restricted, 0.01 to 2000 mg of the effective ingredient may be administered to an adult per day in one or several dosage. While the frequency of administration may be in the range from once a month to administration every day, dosage in the range from once a week to 3 times a week, or 5 times a week, or administration every day is preferable. Dosage a day, the period of administration and frequency of administration may be appropriately increased or decreased depending on the age, weight, physical healthiness, the kind of the disease to be treated, and severeness of the disease.

The medicine of the invention and other drugs that does not adversely affect the action of the medicine of the invention may be concomitantly used for the purpose of enhancing the therapeutic effect. Examples of the coadministration agent in combinations with the medicine of the invention are as described in (A) to (TT) below:
(A) ipratropium bromide cholinergic blocking agent: thiotropium bromide, oxytropium bromide;
(B) β-1 or β-2 adrenalin receptor agonist: metaprotelenol, isoprotelenol, isoprenaline, albuterol, salbutamol, formoterol, salmeterol, terbutaline, orciprenaline, bitolterol, pirbuterol;
(C) theophylline and aminophylline;
(D) leukotriene biosynthesis inhibitor (5-ripoxygenase inhibitor);
(E) leukotriene (LTB4, LTC4, LTD4 and LTE4) receptor antagonist: pranlukast, zafirlukast, montelukast;
(F) thromboxane A2 synthetase inhibitor: ozagrel hydrochloride;
(G) chemical mediator release suppressing agent: sodium cromoglicate, tranilast, amlexanox, repirinast, ibudilast, tazanolast, pemirolast;
(H) histamine H1 receptor antagonist: ketotifen fumarate, azelastine hydrochloride, oxatomide, mequitazine, terfenadine, emedastine fumarate, epinastine fumarate, astemizole, ebastine, fexofenadine hydrochloride, olopatadine hydrochloride, bepotastine besilate, cetirizine hydrochloride;
(I) Th2 cytokine inhibitor: suplatast tosylate;
(J) histamine H2 receptor antagonist;
(K) muscarinic receptor (M1, M2, M3) antagonist;
(L) matrix metalloprotease (MMP) inhibitor;
(M) glucocorticoid: flunisolide, triamcinolone acetonide, beclomethasone dipropionate, budesonide, fluticasone propionate, mometasone furoate;
(N) COX-1 inhibitor (NSAID);
(O) COX-2 inhibitor;
(P) antibody against tumor necrosis factor (TNF-α): remicade;
(Q) antibody against interleukin 6 receptor;
(R) antibody against intrinsic inflammatory substance;
(S) DMARD: leflunomide;
(T) immunosuppressive agent: ciclosporin, mizoribine, methotrexate;
(U) anti-gout agent: colchicines;
(V) xanthine oxidase inhibitor: allopurinol;
(W) uric acid excretion accelerator: probenecid, sulfinpyrazone, benzbromarone;
(X) transforming growth factor (TGF-β) receptor agonist;
(Y) transforming growth factor (TGF-β) receptor kinase inhobitor;
(Z) anti-depressive agent;
(AA) anti-Alzheimer disease agent;
(BB) capsaicin;
(CC) tryptase inhibitor;
(DD) platelet-aggregating factor (PAF) antagonist;
(EE) interleukin conversion enzyme (ICE) inhibitor;
(FF) adhesive molecule inhibitor: VLA-4 antagonist;
(GG) IMPDH inhibitor;
(HH) serotonin 3 (5-HT3) receptor and serotonin 4 (5-HT4) receptor antagonist;
(II) bradykinin-B1-receptor antagonist, bradykinin-B2-receptor antagonist;
(JJ) cathepsin;
(KK) MAP kinase inhibitor;
(LL) glucose-6-phosphate dehydrogenase inhibitor;
(MM) anti-tumor agent;
(NN) insulin-like growth factor type I (IGF-1) mimetic;
(OO) platelet-derived growth factor (PDGF);
(PP) fibroblast growth factor: bFGF;
(QQ) granulocyte macrophage colony stimulating factor (GM-CSF);
(RR) elastase inhibitor;
(SS) adenosine A2a receptor antagonist; and
(TT) various diabetic agent.

The administration period of the above-mentioned coadministration agents are not particularly restricted. The medicine of the invention and the coadministration agent may be simultaneously administered, or may be administered with a time interval to the subject. The dosage of the coadministration agent may be in accordance with the clinically used dosage, and may be appropriately selected depending on the subject being administered, administration route, diseases and combinations of the medicine of the invention with the coadministration agent.

Medication of the coadministration agent is not particularly restricted, and the medicine of the invention may be combined with the coadministration agent before administration. Examples of such medication includes (1) administration of a single preparation obtained by simultaneously preparing the compound of the invention, salts thereof or prodrugs thereof as effective ingredients of the medicine of the invention and the coadministration agent together; (2) concomitant administration of two preparations obtained by independently preparing the medicine of the invention and coadministration agent; (3) administration of two kinds of preparations obtained by independently preparing the medicine of the invention and coadministration agent through the same administration route with a time difference between the medicine and coadministration agent; (4) simultaneous administration of two kinds of preparations obtained by independently preparing the medicine of the invention and coadministration agent through different administration routes to one another; and (5) administration of two kinds of preparations obtained by independently preparing the medicine of the invention and coadministration agent through different administration routes with a time difference between the medicine and coadministration agent (for example administration in the order of the medicine of the invention and coadministration agent, or in the reversed order).

The blending ratio between the medicine of the invention and coadministration agent may be appropriately selected depending on the subject of administration, administration route and diseases.

Usefulness of the medicine of the invention can be confirmed by the effect and/or changes of the concentration of the medicine in the blood (for example maximum concentration in the blood, duration of the effective concentration in the blood, half-life in the blood and AUC) with respect to the compound of the invention, salts thereof or prodrugs thereof using a mammal including a human (for example mouse, rat, hamster, ferret, dog and monkey) as well as a mammal cell including a human (for example immunocytes, inflammatory cells, tumor cells, primary cells, living cells). A higher level of usefulness of the compound of the invention, salts thereof or prodrugs thereof may be also confirmed by investigating low toxicity of them. Furthermore, usefulness may be confirmed from the changes of the concentration in the blood, enzyme induction, enzyme inhibition and stability against microsomes.

### EXAMPLES

While the invention is described in more detail with reference to examples and comparative examples, the scope of the invention is by no means restricted to the examples as set for the below.

Various analyses were carried out as follows in the examples below, unless otherwise stated. Precoated silica gel 60 F254 (manufactured by Merck Co.) was used for thin layer chromatography (TLC), and spots were detected by UV irradiation (254 nm). Wako gel C-300 (manufactured by Wako Pure Chemical Industries, Inc.) was used for the silica gel column. The term “concentration” in the experimental operations refers to removal of solvents or excess reagents by evaporation in vacuum using an evaporator (manufactured by Tokyo Rikakikai Co.)

Columns (Develosil C30-UG-5, 4.6×50 mm) manufactured by Nomura Chemical Co. (Japan) were used for LC-MS-HPLC, and desired components were eluted by gradient elution.

Details of the Elution Condition were as Follows:

Flow rate: 2 mL/min

Solvent: liquid A=water containing 0.1% (v/v) acetic acid, liquid B=acetonitrile containing 0.1% (v/v) acetic acid,

0 min to 5 min: linear gradient from [liquid A 95%+liquid B 5% (v/v)] to [liquid A 2%+liquid B 98% (v/v)],

5 min to 6 min: maintain at [liquid A 2%+liquid B 98% (v/v)],

6 min to 7.5 min: maintain at [liquid A 95%+liquid B 5% (v/v)]

Examples and reference examples in which LC conditions are specifically described mean that measurements were performed under the following solvent conditions:

(LC Condition 1)

Solvent: liquid A=water containing 0.1% (v/v) acetic acid, solvent B=acetonitrile containing 0.1% (v/v) acetic acid,

0 min to 5 min: linear gradient from [liquid A 95%+liquid B 5% (v/v)] to [liquid A 2%+liquid B 98% (v/v)],

5 min to 6 min: maintain at [liquid A 2%+liquid B 98% (v/v)],

6 min to 7.5 min: maintain at [liquid A 95%+liquid B 5% (v/v)]

(LC Condition 2)

Solvent: liquid A=water containing 0.1% (v/v) acetic acid, liquid B=acetonitrile containing 0.1% (v/v) acetic acid

0 to 5 min: linear gradient from [liquid A 95%+liquid B 5% (v/v)] to [liquid A 0%+liquid B 100% (v/v)],

5 to 9 min: maintain at [liquid A 0%+liquid B 100% (v/v)]

9 to 10 min: maintain at liquid A 95%+liquid B 5% (v/v)]

(LC Condition 3)

Solvent: liquid A=water containing 0.1% (v/v) acetic acid, liquid B=acetonitrile containing 0.1% (v/v) acetic acid

0 to 5 min: linear gradient from [liquid A 70%+liquid B 30% (v/v)] to [liquid A 2%+liquid B 98% (v/v)],

5 to 6 min: maintain at [liquid A 2%+liquid B 98% (v/v)],

6 to 7.5 min: maintain at liquid A 70%+liquid B 30% (v/v)

(LC Condition 4)

Solvent: liquid A=water containing 0.1% (v/v) acetic acid, liquid B=acetonitrile containing 0.1% (v/v) acetic acid

0 to 5 min: linear gradient from [liquid A 50%+liquid B 50% (v/v) ] to [liquid A 2%+liquid B 98% (v/v) ],

5 to 6 min: maintain at [liquid A 2%+liquid B 98% (v/v) ],

6 to 7.5 min: maintain at liquid A 50%+liquid B 50% (v/v)

### Reference Example 1

### 5-allyl-6-methyl-2-(thiophene-2-yl)pyrimidin-4(3H)-one

Ethyl 2-allylactoacatate (255 mg) obtained according to the method described in a published report (J. Org. Chem. 1995, 60, 856-862) and thiophene-2′-carboximidamide hydrochloride (244 mg, manufactured by Maybridge Co.) were dissolved in ethylene glycol (7.5 mL), and the solution was stirred at 120° C. for 15 hours in nitrogen atmosphere by adding sodium ethoxide (206 mg, manufactured by Wako Pure Chemical Industry Co.). After allowing the reaction mixture to cool to room temperature, 2 M hydrochloric acid (1.0 ml) and Water (50 mL) was added, the reaction product was extracted with ethyl acetate, washed with saturated brine, and dried over anhydrous magnesium sulfate followed by concentration. The residue obtained was applied on a silica gel column (hexane/ethyl acetate=3/1) to obtain the titled compound (135 mg).

### Reference Example 2

### 5-ethyl-6-methyl-2-(thiophene-2-yl)pyrimidin-4(3H)-one

Ethyl 2-ethylacetoacetate (1.90 g, manufactured by Wako Pure Chemical Industries, Inc.) and thiophene-2-carboximidamide hydrochloride (1.63 g, manufactured by Maybridge Co.) were dissolved in methanol (80 mL), and the solution was stirred at 55° C. for 24 hours after adding sodium hydride (40% in mineral oil, 3.21 g, manufactured by Wako Pure Chemical Industries, Inc.). After allowing the reaction mixture to cool to room temperature, the solution was neutralized using 2 M hydrochloric acid. Water (200 mL) was added to the solution and the mixed solution was extracted with ethyl acetate. The extract solution was washed with saturated brine, and was concentrated after drying over anhydrous magnesium sulfate. The residue obtained was washed with diethylether to obtain the titled compound (979 mg).

### Reference Example 3

### 5-allyl-4-chloro-6-methyl-2-(thiophene-2-yl)pyrimidine

The compound (130 mg) obtained in Reference Example 1 was dissolved in phosphorous oxychloride (8 mL), and the solution was stirred at 100° C. for 2 hours in nitrogen atmosphere. The reaction mixture was concentrated, and a saturated aqueous sodium hydrogen carbonate solution (7 mL) and water (30 mL) were added to the residue obtained. The mixed solution was extracted with ethyl acetate, and the extract solution was washed with saturated brine. The ethyl acetate solution was dried over anhydrous magnesium sulfate, and was concentrated to obtain the titles compound (132 mg).

### Reference Example 4

### 5-methoxythiophne-2-carbaldehyde

2-methoxythiophene (4.41 mL, manufactured by Aldrich Co.) was dissolved in N,N-dimethylformamide (45 mL), and phosphorous oxychloride (6.12 mL) was added dropwise to the solution with cooling in an ice bath while leaving the solution to be stirred for 1 hour. Sodium hydroxide (2 M, 35 mL) was added to the solution thereafter, and the mixed solution was stirred at 90° C. for 10 minutes. Water (150 mL) was added to the reaction mixture, and the solution was extracted with ethyl acetate. The ethyl acetate was washed with saturated brine, and was concentrated after drying over anhydrous magnesium sulfate. The residue obtained was purified by column chromatography (hexane/ethyl acetate=5/1) to obtain the titled compound (5.86 g).

### Reference Example 5

### 5-methoxythiophene-2-carbonitrile

The compound (3.20 g) obtained in Reference Example 4, hydroxyamine hydrochloride (6.27 g, manufactured by Wako Pure Chemical Industries, Inc.) and sodium acetate (7.37 g, manufactured by Wako Pure Chemical Industries, Inc.) were dissolved in acetic acid (150 mL), and the solution was stirred at 100° C. for 11 hours. The reaction mixture was filtered, the filtrate was concentrated, and water (250 mL) was added to the residue obtained. The mixed solution was extracted with ethyl acetate, and the organic layer was washed with saturated aqueous sodium hydrogen carbonate solution. The ethyl acetate solution was dried over anhydrous magnesium sulfate and was concentrated. The residue obtained was purified by column chromatography (hexane/ethyl acetate=3/1) to obtain the titled compound (2.20 g).

### Reference Example 6

### 5-methoxythiophene-2-carboxamidine

The compound (281 mg) obtained in Reference Example 5 was dissolved in diethylether (12 mL), and a 2M hexane solution (4 mL) of lithium bis(trimethylsilyl)amide was added dropwise to the diethylether solution at 0° C. in nitrogen atmosphere followed by stirring at room temperature for 1 hour. 2M hydrochloric acid (6 mL) and water (10 mL) were added to the reaction solution at 0° C., and 2M aqueous sodium hydroxide solution (9 mL) was added to the separated aqueous layer. The aqueous solution was extracted with chloroform, and the organic layer was dried over anhydrous magnesium sulfate followed by concentration to obtain the titled compound (267 mg).

### Example 1

### 4-(5-allyl-6-methyl-2-thiophen-2-yl)pyrimidin-4-ylamino)benzoic acid

The compound (37 mg) obtained in Reference Example 3 and 4-aminobenzoic acid (40.9 mg, manufactured by Nakarai Tesk Co.) were dissolved in acetic acid (2 mL), and the solution was stirred at 100° C. for 43 hours. An aqueous saturated sodium hydrogen carbonate solution (1 mL) and water (25 mL) were added to the residue obtained by concentrating the reaction mixture. The aqueous solution was extracted with ethyl acetate, and the ethyl acetate solution was washed with saturated brine and concentrated after drying over anhydrous magnesium sulfate. The residue obtained was purified by preparative thin layer chromatography (chloroform/methanol=15/1) to obtain the titled compound (27 mg). LC-MS:HPLC, retention time 3.98 min, m/z=352 (M+H).

### Examples 2 to 27

Compounds in Examples 2 to 27 were synthesized according to the method in Example 1. Details of Examples 2 to 27 are shown in Table 1. Notations in Table 1 mean as follows. Exp.; No. of example, Str.: compound of example, RT: retention time of liquid chromatography in LCMS, MS: mass spectrometer data in LCMS, Ref: production method of corresponding intermediate compounds. Notations in the column of Ref show the production method of the intermediate as described below. A: production method shown in Reference Example 1, B: production method shown in Reference Example 2, C: production method shown in Reference Example 3, D: production method shown in Reference Example 4, E: production method shown in Reference Example 5, F: production method shown in Reference Example 6.

TABLE 1

| Exp. | Str | R.T. | MS | Ref. |
|---|---|---|---|---|
| 2 | 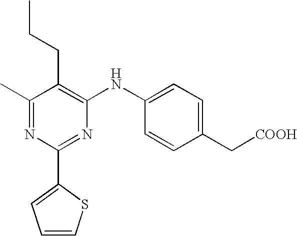 | 3.35 | 366 (M + H) | A, C |
| 3 | 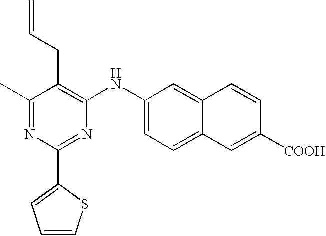 | 4.77 | 402 (M + H) | A, C |
| 4 | 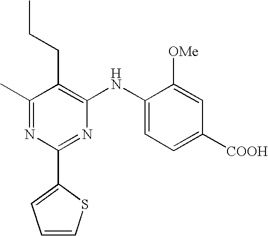 | 4.89 | 382 (M + H) | A, C |
| 5 | 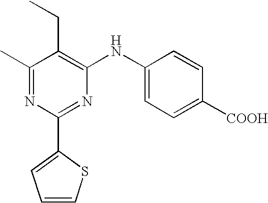 | 3.73 | 340 (M + H) | B, C |
| 6 | 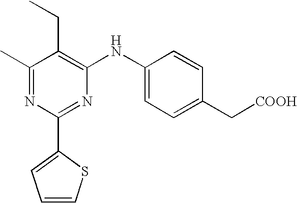 | 2.98 | 354 (M + H) | B, C |
| 7 | 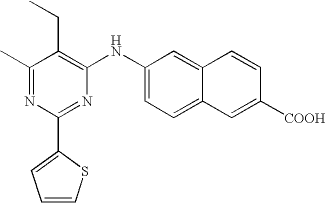 | 4.21 | 390 (M + H) | B, C |
| 8 | 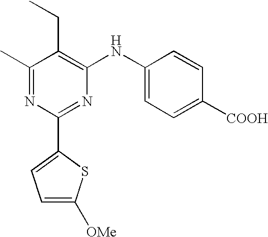 | 3.75 | 370 (M + H) | D, E, F, B, C |
| 9 | 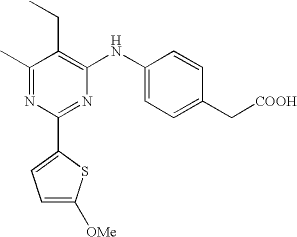 | 3.31 | 384 (M + H) | D, E, F, B, C |
| 10 | 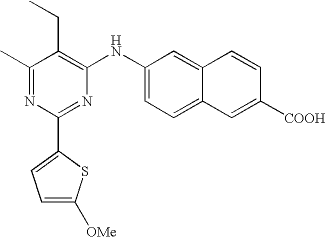 | 4.23 | 420 (M + H) | D, E, F, B, C |
| 11 | 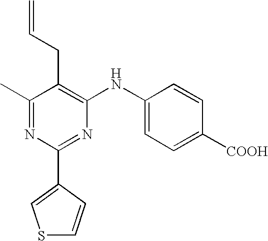 | 3.37 | 352 (M + H) | B, C |
| 12 | 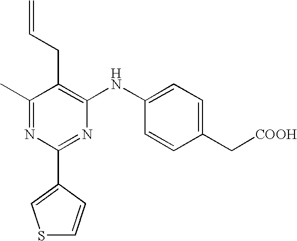 | 2.81 | 368 (M + H) | B, C |
| 13 | 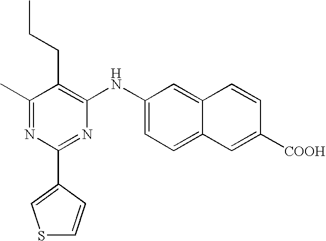 | 3.84 | 402 (M + H) | B, C |
| 14 | 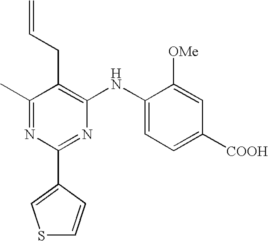 | 4.19 | 382 (M + H) | B, C |
| 15 | 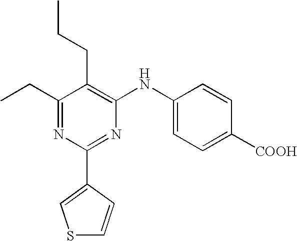 | 4.27 | 368 (M + H) | B, C |
| 16 | 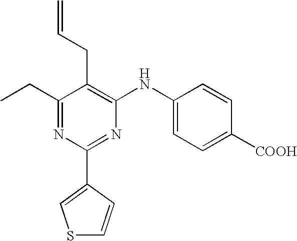 | 3.57 | 380 (M + H) | B, C |
| 17 | 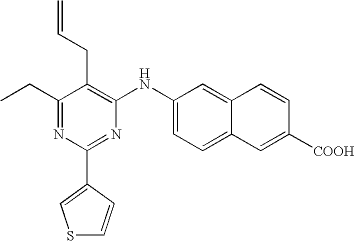 | 4.97 | 416 (M + H) | B, C |
| 18 | 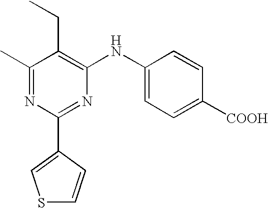 | 2.99 | 340 (M + H) | B, C |
| 19 | 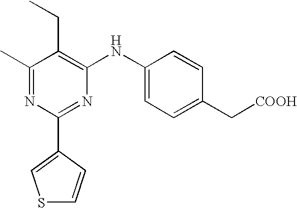 | 2.59 | 354 (M + H) | B, C |
| 20 | 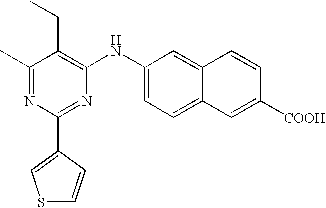 | 3.43 | 390 (M + H) | B, C |
| 21 | 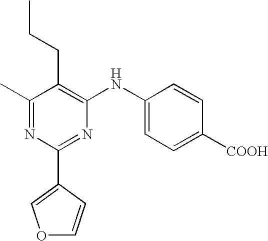 | 3.1 | 336 (M + H) | B, C |
| 22 | 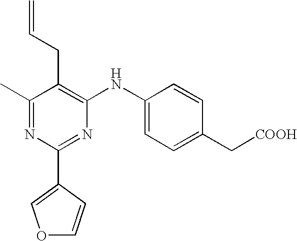 | 2.65 | 350 (M + H) | B, C |
| 23 | 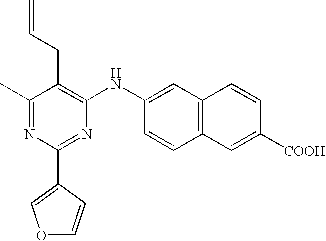 | 3.63 | 386 (M + H) | B, C |
| 24 | 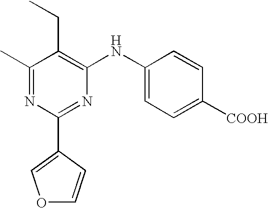 | 2.95 | 324 (M + H) | B, C |
| 25 | 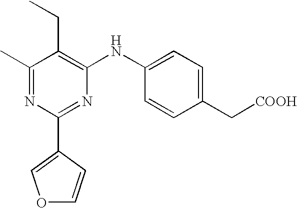 | 2.46 | 338 (M + H) | B, C |
| 26 | 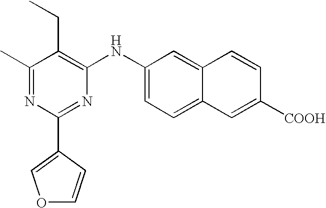 | 3.16 | 374 (M + H) | B, C |
| 27 | 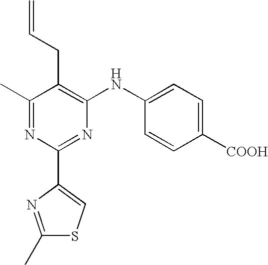 | 2.91 | 367 (M + H) | A, C |

### Reference Example 7

### methyl 5-formylthiophene-2-carboxylate

5-formylthiophene-2-carboxylic acid (3.13 g, manufactured by Tokyo Chemical Industry Co.) was dissolved in N,N-dimethylformamide (100 mL), and sodium carbonate (8.64 g, manufactured by Wako Pure Chemical Industries, Inc.) and iodomethane (2.49 mL, manufactured by Tokyo Chemical Industry Co.) were sequentially added at room temperature followed by stirring for 11 hours. Insoluble substances were removed from the reaction solution by filtration. Water (200 mL) was added to the filtrate, and the mixed solution was extracted with ethyl acetate. The extract solution was washed with saturated brine and was concentrated after drying over anhydrous magnesium sulfate to obtain the titled compound (3.26 g).

### Reference Example 8

### methyl 5-cyanothiophene-2-carboxylate

Hydroxyamine hydrochloride (1.44 g, manufactured by Wako Pure Chemical Co.) was suspended in acetonitrile (80 mL), and triethylamine (2.88 mL, manufactured by Wako Pure Chemical Co.), an acetonitrile solution (100 mL) of the compound (2.93 g) obtained in Reference Example 7 and phthalic anhydride (2.82 g, manufactured by Aldrich Co.) were sequentially added at room temperature, and the solution was stirred at 90° C. for 48 hours in nitrogen atmosphere. After concentrating the reaction solution, ethyl acetate was added to the residue obtained, and the ethyl acetate solution was washed with saturated aqueous sodium hydrogen carbonate solution followed by concentration after drying over anhydrous magnesium sulfate. The residue obtained was purified by column chromatography (hexane/ethyl acetate=6/1) to obtain the titled compound (2.48 g)

### Reference Example 9

### ethyl 2-isobutyrylpent-4-enate

Ethyl isobutyrylacetate (3.16 g, manufactured by Wako Pure Chemical Industries, Inc.) and allyl trimethylsilane (4.15 mL, manufactured by Aldrich Co.) were dissolved in methanol (400 mL), and diammonium sulfate (23.06 g, manufactured by Kanto Chemical Co.) was added to the solution at room temperature followed by stirring the solution for 1 hour. After concentrating the reaction solution, water (250 mL) was added to the residue obtained and then extracted with diethyl ether. The extract solution was dried over anhydrous magnesium sulfate, and was concentrated. The residue obtained was purified by column chromatography (hexane/ethyl acetate=10/1) to obtain the titled compound (2.03 g).

### Reference Example 10

### 4-methoxythiophene-3-carboxamidine

Ammonium chloride (157 mg, manufactured by Aldrich Co.) was added to anhydrous toluene (8 mL. manufactured by Kanto Chemical Co.) in nitrogen atmosphere, and a toluene solution of trimethyl aluminum (2.0 M, 1.47 mL, manufactured by Kanto Chemical Co.) was added dropwise into the solution while cooling the solution in an ice bath followed by stirring at room temperature for 1 hour. A toluene solution (2 mL, manufactured by Kanto Chemical Co.) of 4-methoxythiophene-3-carbonitrile (272 mg) was added dropwise to the solution at room temperature thereafter, and the solution was stirred at 80° C. for 18 hours. After allowing the reaction mixture to room temperature, it was transferred into a mixture of silica gel 60 (14 g, manufactured by Merck Co.) and chloroform (20 mL, manufactured by Wako Pure Chemical Industries, Inc.). After stirring the mixture for 5 minutes, it was filtered with a glass filter followed by concentration of the filtrate to obtain the titled compound (458 mg) after drying.

### Reference Example 11

### 5-bromothiophene-2-carboxamidine

5-bromothiophene-2-carbonitrile (333 μl, manufactured by Aldrich Co.) was dissolved in anhydrous methanol (7 mL, manufactured by Wako Pure Chemical Industries, Inc.), and sodium methoxide (28% methanol solution, 288 μl) was added dropwise to the solution at room temperature followed by stirring for 1 hour. Sodium chloride (321 mg, manufactured by Aldrich Co.) and anhydrous ethanol (7 mL, manufactured by Wako Pure Chemical Industries, Inc.) were added to the reaction solution, and the solution was stirred at 90° C. for 14 hours. The solution was allowed to cool to room temperature followed by filtering with filter paper. The residue obtained by concentrating the filtrate was washed with diethylether several times, and the titled compound (660 mg) was obtained after filtration.

### Reference Example 12

### ethyl 2-ethyl-3-oxohexanoate

Ethyl burylylacetate (8.20 mL, manufactured by Tokyo Chemical Industry Co.) was dissolved in acetone (50 mL). Bromoethane (4.85 mL, manufactured by Aldrich Co.) and potassium carbonate (13.82 g, manufactured by Wako Pure Chemical Industries, Inc.) were sequentially added to the solution, and the mixed solution was refluxed for 20 hours with heating. After removing insoluble substances from the reaction mixture by filtration and concentrating the filtrate, the residue obtained was purified by column chromatography (hexane/ethyl acetate=10/1) to obtain the titled compound (5.75 g).

### Reference Example 13

### 4-bromo-2-(diethoxymethyl)thiophene

4-bromothiophen -2-carbaldehyde (2.3 g, manufactured by Aldrich Co.) was dissolved in ethanol (30 mL), and ammonium chloride (706 mg, manufactured by Aldrich Co.) and triethoxymethane (2.2 ml, manufactured by Wako Pure Chemical Industries, Inc.) were added to the solution followed by stirring at 60° C. for 1 hour. The reaction mixture was allowed to cool to room temperature, and was filtered with filter paper. The filtrate was concentrated, and the titled compound (2.6 g) was obtained after drying.

### Reference Example 14

### 2-(diethoxymethyl)-4-methoxythiophene

The compound (0.65 g) obtained in Reference Example 13, sodium methoxide (1.76 mL, 28% methanol solution, manufactured by Wako Pure Chemical Industries, Inc.), copper oxide (236 mg, manufactured by Wako Pure Chemical Industries, Inc.) and potassium iodide (498 mg, manufactured by Wako Pure Chemical Industries, Inc.) were dissolved in methanol (2 mL), and the solution was stirred at 120° C. for 20 hours in nitrogen atmosphere. The reaction mixture was allowed to cool to room temperature and filtered with celite. After washing with methanol several times, the solution was concentrated to obtain the titled compound (670 mg).

### Reference Example 15

### 4-methoxythiophene-2-carbaldehyde

The compound (2.63 g) obtained in Reference Example 14 was dissolved in methanol (30 mL), and an aqueous solution of 5 N hydrochloric acid (5 mL, manufactured by Wako Pure Chemical Industries, Inc.) was slowly added dropwise followed by stirring at room temperature for 15 minutes. The reaction mixture was concentrated after neutralizing by adding 5 N aqueous solution of sodium hydroxide (2 mL, manufactured by Wako Pure Chemical Industries, Inc.). The residue obtained was extracted with ethyl acetate after adding saturated aqueous sodium hydrogen carbonate solution. The organic phase was washed with saturated brine and dried over anhydrous magnesium sulfate followed by concentration. The residue obtained was purified by column chromatography (hexane/ethyl acetate=20/1) to obtain the titled compound (842 mg).

### Reference Example 16

### 5-bromothiophene-3-carbaldehyde

Thiophene-3-carbaldehyde (3.56 ml, manufactured by Aldrich Co.) was dissolved in dichloromethane (100 mL), and bromine (1.13 ml, manufactured by Wako Pure Chemical Industries, Inc.) and anhydrous aluminum chloride (8.78 g, manufactured by Aldrich Co.) were added to the solution followed by stirring at 45° C. for 2 hours. The reaction mixture was stirred by cooling in an ice bath, and saturated aqueous ammonium chloride solution was slowly added to the solution. The solution was extracted with dichloromethane, and the extract was washed with saturated brine. The organic layer was concentrated after drying over anhydrous magnesium sulfate, and the titled compound was obtained as a mixture (1:1, 5.94 g) with thiophene-3-carbaldehyde.

### Reference Example 17

### ethyl 2-(4-aminophenyl)-2-hydroxyacetate

Ethyl 2-(4-nitrophenyl)-2-oxoacetate (0.99 g) was dissolved in methanol (40 mL) in nitrogen atmosphere, and 10% Pd/C (0.48 g) was added to the solution. The nitrogen atmosphere was replaced with hydrogen atmosphere under a reduced pressure, and the solution was stirred at room temperature for 3 hours. The mixture obtained by filtering the reaction solution was concentrated, and the residue was applied on a silica gel column (hexane/ethyl acetate=1/1) to obtain the titled compound (837.2 mg).

### Reference Example 18

### 5-(4-methoxyphenyloxy)thiophene-2-carbonitrile

4-methoxyphenol (149 mg, manufactured by Wako Pure Chemical Industries, Inc.) was dissolved in DMF (3 mL), and sodium hydride (48 mg, manufactured by Kanto Chemical Co.) was added to the solution followed by stirring for 45 minutes. 5-bromothiophene-2-carbonitrile (111 μl. manufactured by Aldrich Co.) was added to the reaction mixture, and the solution was stirred at 120° C. for additional 22 hours. The reaction solution was allowed to cool to room temperature, the solvent was removed by distillation, and was extracted with ethyl acetate after adding water. The organic layer was concentrated after drying over anhydrous magnesium sulfate. The residue obtained was purified by column chromatography (hexane/ethyl acetate=20/1) to obtain the titled compound (68.5 g).

### Reference Example 19

### methyl 2-(4-(benzyloxycarbonylamino)phenyl)-2-mehtylpropanoate

Methyl 2-(4-bromophenyl)-2-methylpropanoic acid (203.5 mg, manufactured by Tronto Co.) was dissolved in 1,4-dioxane (5 mL), and cesium chloride (311.3 mg), tris(benzylidene acetone) dipalladium (0) (69.9 mg), xanthophos (180.3 mg, manufactured by Strem Co.) and benzyl carbamate (145.9 mg) were added to the solution. The mixed solution was stirred at 100° C. for 15.5 hours in nitrogen atmosphere. A large excess of methylene chloride was added to the solution and the solution was filtered. After concentrating the reaction mixture, the residue was purified on silica gel column (hexane/ethyl acetate=4/1) to obtain the titled compound (138.8 mg).

### Reference Example 20

### methyl 2-7(4-aminophenyl)-2-methylpropanoate

The compound (116.4 mg) obtained in Reference Example 19 was dissolved in methanol (5 mL) in the nitrogen atmosphere, and 10% Pd/C (38.6 mg) was added to the solution. The nitrogen atmosphere was replaced with hydrogen under a reduced pressure, and the solution was stirred at room temperature for 3 hours. The mixture obtained by filtering the reaction solution was concentrated to obtain the titled compound (64.7 mg).

### Reference Example 21

### 5-(4-chloro-5-ethyl-6-methylpyrimidin-2-yl)thiophene-3-ol

4-chloro-5-ethyl-2-(4-methoxythiophen-2-yl)-6-methyl pyrimidine (860 mg) was dissolved in chloroform (45 mL), and 1.0 M tribromoborone/dichloromethane solution (15 mL, manufactured by Aldrich Co.) was added dropwise at 0° C. in nitrogen atmosphere with stirring at 0° C. for 30 minutes. Stirring was further continued for 1 hour at room temperature. After diluting the reaction mixture with chloroform (45 mL), a 5% aqueous sodium hydrogen carbonate solution (100 mL) was added to the solution with stirring at room temperature for 30 minutes. The aqueous solution was extracted with chloroform, washed with saturated brine, and the organic layer was concentrated after drying over anhydrous magnesium sulfate to obtain the titled compound (800 mg).

### Reference Example 22

### 4-chloro-5-ethyl-2-(5-hydroxymethylthiophen-2-yl) -6-methylpyrimidine

The compound (90 mg) obtained in Reference Example 23 was dissolved in tetrahydrofuran (10 mL), and sodium borohydride (53 mg, manufactured by Wako Pure Chemical Industries, Inc.) was added at room temperature followed by stirring for 4 hours. Water (30 mL) was added to the reaction mixture, and the aqueous solution was extracted with ethyl acetate. The organic layer was washed with saturated brine, dried over anhydrous magnesium sulfate, and concentrated to obtain the titled compound (83 mg).

### Reference Example 23

### 4-chloro-5-ethyl-2-(5-formylthiophen-2-yl)-6-methylpyrimidine

2-(5-(diethoxymethyl)thiophene-2-yl)-5-ethyl-6-pyrimidin-4(3H)-one (201 mg) was dissolved in phosphorous oxychloride (10 mL), and the solution was stirred at 100° C. for 2.5 hours in nitrogen atmosphere. Saturated aqueous sodium hydrogen carbonate (15 mL) and water (25 mL) were added to the residue obtained by concentrating the reaction mixture, and the aqueous layer was extracted with ethyl acetate. The ethyl acetate solution was washed with saturated brine, dried over anhydrous magnesium sulfate, and concentrated to obtain the titled compound (165 mg).

### Reference Example 24

### 2-(5-dimethoxymethyl)thiophene-2-yl)-5-ethyl-6-methylpyrimidin-4(3H)-one

2-(5-diethoxymethyl)thiophene-2-yl)-5-ethyl-6-methylpyrimidin-4(3H)-one (150 mg) was dissolved in methanol (8 mL) and tetrahydrofuran (4 mL), and 5 M hydrochloric acid (1.5 mL) was added to the solution at room temperature followed by stirring for 1.5 hours. After neutralizing the solution by adding 5 M aqueous sodium hydroxide solution (1.5 mL), water (25 mL) was added. The aqueous solution was extracted with ethyl acetate, the organic layer was washed with saturated burin, and was concentrated after drying over anhydrous magnesium sulfate to obtain the titled compound (122 mg).

### Reference Example 25

### 5-ethyl-2-(5-ethoxymethylthiophen-2-yl)-6-methylpyrimidin-4(3H)-one

2-(5-(diethoxymethyl)thiophen-2-yl)-5-ethyl-6-methylpyrimidin-4(3H)-one (52 mg) was dissolved in dichloromethane (5 mL), and triethylsilane (124 μL, manufactured by Tokyo Chemical Industry Co.) and trifluoroborone diethylether complex (98 μL, manufactured by Tokyo Chemical Industry Co.) were sequentially added at room temperature followed by stirring for 1.5 hours. Saturated aqueous sodium hydrogen carbonate solution (2 mL), and then water (25 mL) were added to the reaction mixture, and the aqueous solution was extracted with chloroform. The chloroform layer was washed with saturated brine, dried over anhydrous magnesium sulfate and concentrated. The residue obtained was purified by preparative thin layer chromatography (chloroform/methanol=20/1) to obtain the titled compound (23 mg).

### Reference Example 28

### 5-(4-(4-carboxyphenylamino)-5-ethyl-6-methylpyrimidin-2-yl)thiophene-2-carboxykic acid

Methyl 5-(4-(4-carboxyphenylamino)-5-ethyl-6-methylpyrimidin-2-yl)thiophene-2-carboxyate (10 mg) was dissolved in methanol (4 mL), and 2 M aqueous sodium hydroxide solution (389 mL) was added to the solution with stirring at 60° C. for 11 hours. The reaction mixture was cooled in an ice bath, and 2 M hydrochloric acid (389 mL) and water (25 mL) were added to the solution. The aqueous solution was extracted with ethyl acetate, and the organic layer was concentrated after washing with saturated brine and drying over anhydrous magnesium sulfate to obtain the titled compound (10 mg). LC-MS:HPLC retention time 4.20 min, m/z=384 (M+H).

### Example 29

### ethyl 2-(4-(2-(5-bromothiophen-2-yl)-5-ethyl-6-methylpyrimidin-4-ylamino)phenyl)acetate

2-(5-bromothiophen-2-yl)-4-chloro-5-ethyl-6-methylpyrimidine (2 g) was dissolved in ethanol (20 mL), and the solution was stirred at 55° C. for 23 hours after adding ethyl 4-aminophenylacetate (2.27 g, manufactured by Tokyo Chemical Industry Co.) and 35 to 37% aqueous hydrochloric acid solution (0.9 mL, manufactured by Wako Pure Chemical Industries, Inc.). The reaction mixture was concentrated and, after stirring for 15 minutes by adding ethyl acetate, water and saturated aqueous sodium hydrogen carbonate, the aqueous layer was extracted with ethyl acetate. The organic layer was concentrated after drying over anhydrous magnesium sulfate, and the residue obtained was purified by column chromatography (hexane/ethyl acetate=5/1 to 2/1) to obtain the titled compound (2.29 g). LC-MS:HPLC retention time 5.88 min(LC condition 1), m/z=460 (M+H).

### Example 30

### methyl 2-(4-(5-ethyl-2-(5-(4-ethylphenyl)thiophene-2-yl)-6-methylpyridin-4-ylamino)phenyl)acetate

The compound (60.3 mg) obtained in Example 29 was dissolved in toluene (0.388 mL) and methanol (0.135 mL), and 2M aqueous sodium hydrogen carbonate (122 μL), tetrakis triphenylphosphine palladium (15.6 mg, manufactured by Aldrich Co.) and 4-ethylphenylboric acid (30.5 mg, manufactured by Aldrich Co.) were added with stirring at 85° C. for 13 hours. The reaction mixture was filtered with celite after allowing to cool to room temperature, and the solution was concentrated after washing with methanol several times. Water was added to the residue obtained, and the reaction product was extracted with ethyl acetate. The organic layer was concentrated after drying over anhydrous magnesium sulfate, and the residue obtained was purified by column chromatography (hexane/ethyl acetate=2/1) to obtain the titled compound (15.4 mg). LC-MS:HPLC, retention time 6.3 min (LC condition 1), m/z 472 (M+H).

### Example 31

### 2-(4-(5-(4-ethylphenyl)thiophene-2-yl)-6-methylpyrimidin-4-ylamino)phenyl)acetic acid

The compound (15.4 mg) obtained in example 30 was dissolved in methanol (0.4 mL), and 1 N aqueous sodium hydroxide solution (0.1 mL, manufactured by Wako Pure Chemical Industries, Inc.) was added with stirring at room temperature for 16 hours. The solvent was removed by distillation after adding 1 N aqueous hydrochloride solution (0.1 mL, manufactured by Wako Pure Chemical Industries, Inc.), and the aqueous solution was extracted with chloroform after adding water. The organic layer was concentrated after drying over anhydrous magnesium sulfate. The residue obtained was purified by column chromatography (chloroform/methanol=15/1) to obtain the titled compound (10.9 mg). LC-MS:HPLC, retention time 5.18 min (LC condition 1), m/z 458 (M+H).

### Example 32

### 4-(2-(5-ethyl-6-methyl-2-(thiophene-2-yl)pyrimidin-4-ylamino)ethyl)benzoic acid

2-(thiophene-2-yl)-4-chloro-5-ethyl-6-methylpyrimidine (20.5 mg) was dissolved in 1-butanol (1.5 mL), and triethylamine (116.9 μL) and 4-(2-aminoethyl)benzoic acid (85.0 mg, manufactured by Aldrich-Co.) was added with stirring at 120° C. for 17 hours. After allowing the reaction mixture to cool to room temperature, 1,8-diazabicyclo[5.4.0]undec-7-ene (500 μL, manufactured by Aldrich Co.) was added with stirring at 120° C. for 3 hours. After allowing the reaction mixture to cool to room temperature, it was neutralized with 2 M hydrochloric acid. The aqueous layer was extracted with ethyl acetate, and the organic layer was concentrated. The residue obtained was purified on a silica gel column (chloroform/methanol=95/5) to obtain the titled compound (7.8 mg). LC-MS:HPLC, retention time 3.42 min (LC condition 1), m/z 368 (M+H).

### Example 33

### 2-(4-(5-ethyl-6-methyl-2-(thiophene-2-yl) pyrimidin-4-ylamino)phenyl)acetamide

2-(4-(5-ethyl-6-methyl-2-(thiophene-2-yl) pyrimidin-4-ylamino)phenyl)acetic acid (10.7 mg) was dissolved in tetrahydrofuran (1 mL). Diisopropylethylamine (6.35 μL) and O-(7-azabenzotriazo-1-yl)N,N,N′,N′-tetramethyluronium hexafluorophosphate (13.7 mg, manufactured by Applied Biosystems Co.) were added to the solution and, after adding 28% aqueous ammonia solution (100 μL), stirring was further continued at room temperature for 23.5 hours. The reaction mixture was purified over a silica gel column (Chloroform/methanol=95/5) to obtain the titled compound (2.2 mg). LC-MS:HPLC, retention time 3.34 min (LC condition 1), m/z 353 (M+H).

### Example 34

### ethyl 2-(4-(5-ethyl-6-methyl-2-(5-propylthiophen-2-yl)pyrimidin-4-ylamino)phenyl)acetate

Ethyl (Z)-2-(4-(5-ethyl-6-methyl-2-(5-(propen-1-yl)thiophene-2-yl)pyrimidin-4-ylamino)phenyl)acetate (22 mg) was dissolved in tetrahydrofuran (0.5 mL), methanol (0.5 mL) and dichloromethane (0.1 mL), and the solution was stirred for 2 hours in hydrogen atmosphere after adding 10% Pd—C (5 mg, manufactured by Merck Co.). The reaction mixture was filtered with celite, which was sequentially washed with ethyl acetate, chloroform and methanol, and was concentrated to obtain the titled compound (20.5 mg). LC-MS:HPLC, retention time 1.96 min (LC condition 4), m/z 424 (M+H).

### Example 35

### ethyl 2-(4-(2-(5-aminothiophen-2-yl)5-ethyl-6-methylpyrimidin-4-ylamino)phenyl)acetate

Ethyl 2-(4-(2-(5-bromothiophen-2-yl)-5-ethyl-6-methylpyrimidin-4-ylamino)phenyl)acetate (30 mg), benzophenonimine (22 μL, manufactured by Tokyo Chemical Industry Co.), 9,9′-dimehtyl-4,5-bis(diphenylphosphino)-xanthene (15.1 mg, manufactured by STREM Co.), cesium carbonate (42.6 mg, manufactured by Wako Pure Chemical Industries, Inc.) and palladium acetate (2.9 mg, manufactured by Aldrich Co.) were dissolved in 1,4-dioxane (654 μL), and the solution was stirred at 90° C. for 16 hours in nitrogen atmosphere. The reaction mixture was allowed to cool to room temperature, and was concentrated after filtration with celite. The residue obtained was dissolved in methanol (654 μL), and sodium acetate (21.5 mg, manufactured by Wako Pure Chemical Industries, Inc.) and hydroxyamine hydrochloride (13.6 mg, manufactured by Wako Pure Chemical Industries, Inc.) were added with stirring at room temperature for 3 hours. The reaction mixture was concentrated after filtration with celite, and the residue obtained was purified by column chromatography (chloroform/methanol=90/1) to obtain the titled compound (21.2 mg). LC-MS:HPLC, retention time 3.55 min (LC condition 1), m/z 397 (M+H).

### Example 36

### ethyl 2-(4-(2-(5-acetamidethiophen-2-yl)-5-etyl-6-methylpyrimidin-4-ylamino)phenyl)acetate

The compound (10 mg) obtained in Example 35 was dissolved in dichloromethane (500 μL), and acetic anhydride (2.87 μL, manufactured by Wako Pure Chemical Industries, Inc.) was added dropwise at 0° C. after dripping triethylamine (4. 23 μL, manufactured by TCI Co.) with stirring at room temperature for 4 hours. Saturated aqueous sodium hydrogen carbonate solution was added to the reaction mixture, which was extracted with dichloromethane. The organic layer was concentrated after drying over anhydrous magnesium sulfonate, and the residue obtained was purified by column chromatography (chloroform/methanol=90/1) to obtain the titled compound (10.0 mg). LC-MS:HPLC, retention time 3.58 min (LC condition 1), m/z 439 (M+H).

### Example 37

### ethyl 2-(4-(5-ethyl-6-methyl-2-(thiophene-2-yl)pyrimidin-4-ylamino)phenyl)-2-oxoacetate

Ethyl 2-(4-(5-ethyl-6-methyl-2-(thiophene-2-yl)pyrimidin-4-ylamino)phenyl)-2-hydroxyacetate (20.0 mg) was dissolved in a mixed solvent (2 ml) of carbon tetrachloride/dichloromethane (1/1), and was stirred at room temperature for 18 hours by adding chromic acid (polymer-supported, 203.8 mg, manufactured by Aldrich Co., 35,982-3, 20-50 mesh, about 2.5 mmol/g). The reaction solution was concentrated after filtration, and the residue obtained was purified on a silica gel column (hexane/ethyl acetate=2/1) to obtain the titled compound (11.3 mg). LC-MS:HPLC, retention time 5.58 min (LC condition 1), m/z 396 (M+H).

### Example 38

### ethyl (E)-3-(5-(4-(4-(2-ethoxy-2-oxoethyl)phenylamino)-5-ethyl-6-methylpyrimidin-2-yl)thiophene-2-yl)acrylate

The compound (23 mg) obtained in Example 29 was dissolved in dimethylformamide (0.5 mL), and methyl acrylate (6.7 μL, manufactured by Tokyo Chemical Industry Co.), palladium acetate (1 mg, manufactured by Wako Pure Chemical Industries, Inc.), sodium hydrogen carbonate (12.6 mg, manufactured by Kokusan Chemical Co.) and tetrabutylammonium chloride (13.9 mg, manufactured by Tokyo Chemical Industry Co.) were added with stirring at 100° C. for 18 hours in nitrogen atmosphere. The reaction mixture was filtered with celite, which was washed with ethyl acetate. Water was added to the filtrate, and the aqueous layer was extracted with ethyl acetate. The organic layer was washed with water, and was concentrated after drying over anhydrous magnesium sulfate. The residue obtained was purified by column chromatography (hexane/ethyl acetate=2/1) to obtain the titled compound (14.5 mg). LC-MS:HPLC, retention time 3.55 min (LC condition 1), m/z 397 (M+H).

### Example 39

### ethyl (E)-2-(4-(5-ethyl-2-(5-(4-hydroxybuten-1-yl)thiophen-2-yl)-6-methylpyrimidin-4-ylamino)phenyl)acetate

Ethyl (E)-2-(4-(2-(5-(4-(t-butyldimethylsiloxy)buten-1-yl)thiophene-2-yl)-5-ethyl-6-methylpyrimidin-4-ylamino)phenyl)acetate (42.5 mg) was dissolved in tetrahydrofuran (0.5 mL), and the solution was stirred at room temperature for 2 hours by adding tetrabutylammonium fluoride (19.6 mg, manufactured by Tokyo Chemical Industry Co.). Water and saturated burin were added to the reaction mixture, and the aqueous layer was extracted with ethyl acetate. The organic layer was washed with saturated burin, and was concentrated after drying over anhydrous magnesium sulfate. The residue obtained was purified by column chromatography (hexane/ethyl acetate=2/1 to 1/1) to obtain the titled compound (14.5 mg). LC-MS:HPLC, retention time 3.69 min (LC condition 1), m/z 453 (M+H).

### Example 40

### Ethyl 2-(4-(2-(5-cyclopropylthiophen-2-yl)-5-ethyl-6-methylpyrimidin-4-ylamino)phenyl)acetate

The compound (45.9 mg) obtained in-Example 29 was dissolved in toluene (0.5 mL), and cyclopropyl boric acid (11.2 mg, manufactured by Aldrich Co.), potassium phosphate (74.3 mg, manufactured by Aldrich Co.), tricyclohexyl phosphine (2.8 mg, manufactured by Aldrich Co.), palladium acetate (0.5 mg, manufactured by Wako Pure Chemical Industries, Inc.) and water (25 μL) were added followed by stirring the solution at 100° C. for 19 hours in nitrogen atmosphere. The reaction mixture was filtered with celite after allowing the solution to cool to room temperature, then washed with ethyl acetate, and water was added to the filtrate. The aqueous layer was extracted with ethyl acetate. The organic layer was washed with saturated aqueous ammonium chloride, and the extracted solution was concentrated after drying over anhydrous magnesium sulfate. The residue obtained was purified by preparative thin layer chromatography (hexane/ethyl acetate=4/1) to obtain the titled compound (11.8 mg). LC-MS:HPLC, retention time 1.82 min (LC condition 4), m/z 422 (M+H).

### Example 41

### ethyl 2-(4-(5-ethyl-2-(5-(1-hydroxyethyl)thiophene-2-yl)-6-methylpyrimidin-4-ylamino)phenyl)acetate

Ethyl 2-(4-(2-(5-acetylthiophen-2-yl)-5-ethyl-6-mehtylpyrimidin-4-ylamino)phenyl)acetate (8.1 mg) was dissolved in methanol (1 mL), and sodium borohydride (5 mg, manufactured by Wako Pure Chemical Industries, Inc.) was added to the solution with stirring at room temperature for 15 minutes. The solvent was removed by evaporation after adding a small amount of water, and the residue was neutralized by adding 1 N aqueous hydrochloric acid solution after adding ethyl acetate. The reaction product was extracted with ethyl acetate. The organic layer was washed with saturated burin, and concentrated after drying over magnesium sulfate to obtain the titled compound (10.6 mg). LC-MS:HPLC, retention time 0.52 min (LC condition 4), m/z 426 (M+H).

### Example 42

### ethyl 2-(4-(2-(5-dimethylamino)thiophene-2-yl)-5-ethyl-6-methylpyrimidin-4-ylamino)phenyl)acetate

Ethyl 2-(4-(2-(5-bromothiopnen-2-yl)-5-ethyl-6-methylpyrimidin-4-ylamino)phenyl)acetate (30 mg), (±)-2,2′-bis(diphenylphosphino)-1,1′-binaphthalene (20.8 mg, manufactured by Aldrich Co.), tripotassium phosphate (12.2 mg, manufactured by Wako Pure Chemical Industries, Inc.), dimethylamine (261 μL, 2.0 M tetrahydrofuran solution) and tris(dibenzylideneacetone)dipalladium (6.0 mg, manufactured by Aldrich Co.) were dissolved in toluene (654 μL), and the solution was stirred at 90° C. for 16 hours in nitrogen atmosphere. The reaction mixture was allowed to cool to room temperature, and was concentrated after filtering with celite. The residue obtained was purified by column chromatography (chloroform/methanol=90/1) to obtain the titled compound (10.0 mg). LC-MS:HPLC, retention time 3.79 min (LC condition 1), m/z 425 (M+H).

### Example 43

### ethyl 2-(4-(2-(5-ethyl-2-(4-(2-methoxyethylamino)thiophene-2-yl)-6-methylpyrimidin-4-ylamino)phenyl)acetate

The compound (41.5 mg) obtained in Example 35, 1-bromo-2-methoxyethane (19.8 μL, Wako Pure Chemical Industries, Inc.) and 2,6-litidine (36.6 μL, manufactured by TCI Co.) were dissolved in N,N-dimethylformamide (1.0 mL), and the solution was stirred at 80° C. for 48 hours. After allowing the reaction mixture to cool to room temperature, water (5 mL) was added, and the reaction product was extracted with ethyl acetate. The organic layer was washed with saturated burin, and was concentrated after drying over anhydrous magnesium sulfate. The residue obtained was purified by column chromatography (chloroform/methanol=90/1) to obtain the titled compound (16.0 mg). LC-MS:HPLC, retention time 3.55 min (LC condition 1), m/z 455 (M+H).

### Example 44

### ethyl 2-(4-(5-ethyl-6-methyl-2-(5-vinylthiophen-2-yl)pyrimidin-4-ylamino)phenyl)acetate

The compound (91.8 mg) obtained in Example 29 was dissolved in 1-propanol (2 mL), and potassium vinyltrifluoroboran (32.2 mg, manufactured by Lancaster Co.), (1,1′-bis(diphenylphosphino)ferrocene)dichloropalladium-dichloromethane complex (3.26 mg, manufactured by Aldrich Co.) and triethylamine (27.9 μL, manufactured by Wako Pure Chemical Industries, Inc.) were added with stirring at 105° C. for 4.5 hours. Water was added to the reaction mixture, and the reaction product was extracted with ethyl acetate. The organic layer was washed with saturated burin, and was concentrated after drying over anhydrous magnesium sulfate. The residue obtained was purified by column chromatography (hexane/ethyl acetate=5/1) to obtain the titled compound (52.9 mg). LC-MS:HPLC, retention time 2.43 min (LC condition 4), m/z 408 (M+H).

### Example 45

### ethyl 2-(4-(5-ethyl-2-(5-(2-hydroxyethyl)thiophene-2-yl)-6-methylpyrimidin-4-ylamio)phenyl)acetate

The compound (23.4 mg) obtained in Example 44 was dissolved in tetrahydrofuran (0.4 mL), and a tetrahydrofuran solution of borane/tetrahydrofuran complex (66.2 μL, manufactured by Kanto Chemical Co.) was cooled in an ice bath and was added to the solution with stirring for 5 minutes, followed by stirring at room temperature for 22 hours. The reaction mixture was cooled to −15° C. , and 30% hydrogen peroxide (39 μL, manufactured by Junsei Chemical Co.) was added to the solution with stirring for 30 minutes. Then, 2N aqueous sodium hydroxide solution (92 μL, manufactured by Wako Pure Chemical Industries, Inc.) was added with stirring for 1 hour at −15° C., followed by stirring at room temperature for additional 2 hours. Water was added to the reaction temperature, and the reaction product was extracted with ethyl acetate. The organic layer was washed with saturated burine, and was concentrated after drying over anhydrous magnesium sulfate. The residue obtained was purified by preparative thin layer chromatography (hexane/acetic acid=2/1) to obtain the titled compound (5.7 mg). LC-MS:HPLC, retention time 3.79 min (LC condition 1), m/z 426 (M+H).

### Example 46

### ethyl 2-(4-(5-ethyl-2-(5-iodothiophen-2-yl)-6-methylpyrimidin-4-ylamino)phenyl)acetate

The compound (45.9 mg) obtained in Example 29 was dissolved in 1,4-dioxane (0.5 mL), and copper iodide (1 mg, manufactured by Wako Pure Chemical Industries, Inc.), sodium iodide (30 mg, manufactured by Wako Pure Chemical Industries, Inc.) and (1S,2S)-(+)-N,N′-dimethylcyclohexane-1,2-diamine (1.42 μL, manufactured by Tokyo Chemical Industry Co.) were added to the-solution with stirring at 110° C. for 23 hours. After allowing the reaction mixture to cool to room temperature, 25% aqueous ammonia (0.5 mL, manufactured by Wako Pure Chemical Industries, Inc.) was added with stirring. Water was further added, and the reaction product was extracted with dichloromethane. The organic layer was washed with saturated burin, and was concentrated after drying over anhydrous magnesium sulfate to obtain the titled compound (39.5 mg). LC-MS:HPLC, retention time 5.76 min (LC condition 1), m/z 508 (M+H).

### Example 47

### ethyl 2-(4-(5-ethyl-2-(5-(3-methoxy-1-propinyl)thiophene-2-yl)-6-methylpyrimidin-4-ylamino)phenyl)acetate

The compound (22.9 mg) obtained in Example 46 was dissolved in benzene (0.3 mL), and dichlorobis(triphenylphosphine)palladium (3.2 mg, manufactured by Aldrich Co.), methyl propargylether (7.6 μL, manufactured by Aldrich Co.) and triethylamine (0.1 mL, manufactured by Wako Pure Chemical Industries, Inc.) were added to the solution with stirring at room temperature for 21 hours. The reaction mixture was concentrated, and the residue obtained was purified by preparative thin layer chromatography (hexane/ethyl acetate=5/1) to obtain the titled compound (7.9 mg). LC-MS:HPLC, retention time 5.56 min (LC condition 1), m/z 450 (M+H).

### Example 48

### ethyl 2-(4-(5-ethyl-2-(5-(3-methoxypropylthiophen)-2-yl)-6-methylpyrimidin-4-ylamino)phenyl) acetate

The compound (7.9 mg) obtained in Example 47 was dissolved in methanol (0.5 mL), and the solution was stirred in a hydrogen atmosphere for 13 hours after adding palladium hydroxide (2 mg, manufactured by N. E. Chemcat Co., the type Pd). The reaction mixture was filtered with celite, which was washed with ethyl acetate and chloroform. The filtrate was concentrated to obtain the titled compound (8.1 mg). LC-MS:HPLC, retention time 4.47 min (LC condition 1), m/z 454 (M+H).

### Example 49

### Ethyl 2-(4-(5-ethyl-6-methyl-2-(5-(3-(methylamino)propyl)thiophene-2-yl)pyrimidin-4-ylamino)phenyl)acetate

Ethyl 2-(4-(5-ethyl-6-methyl-2-(5-(3-oxopropyl)thiophene-2-yl)pyrimidin-4-ylamino)phenyl)acetate (15 mg) was dissolved in methanol (1.5 mL), and palladium hydroxide (5 mg, manufactured by N. E. Chemcat Co., the type Pd) and benzylmethyl amine (8.8 μL, manufactured by Tokyo Chemical Industry Co.) were added to the solution. The reaction mixture was stirred at room temperature for 24 hours in a hydrogen atmosphere at a pressure in the range from 3.4 to 4 atm. The reaction mixture was filtered with celite, which was washed with methanol. The filtrate was concentrated to obtain the titled compound (20 mg). LC-MS:HPLC, retention time 3.73 min (LC condition 1), m/z 453 (M+H).

### Production of Intermediate Compounds used in Examples 28 to 49

The production methods of intermediate compounds in Examples 28 to 49 are shown in Table 2. Notations in Table 2 mean as follows: Exp; No. of examples, Ref; production methods of corresponding intermediate compounds. Notations in the column of Ref denote the production methods of intermediate compounds as follows:
A; production method shown in Reference Example 1,
B; production method shown in Reference Example 2,
C; production method shown in Reference Example 3,
D; production method shown in Reference Example 4,
E; production method shown in Reference Example 5,
F; production method shown in Reference Example 6,
G; production method shown in Reference Example 7,
H; production method shown in Reference Example 8,
I; production method shown in Reference Example 9,
J; production method shown in Reference Example 10,
K; production method shown in Reference Example 11,
L; production method shown in Reference Example 12,
M; production method shown in Reference Example 13,
N; production method shown in Reference Example 14,
O; production method shown in Reference Example 15,
P; production method shown in Reference Example 16,
Q; production method shown in Reference Example 17,
R; production method shown in Reference Example 18,
S; production method shown in Reference Example 19,
T; production method shown in Reference Example 20,
U; production method shown in Reference Example 21,
V; production method shown in Reference Example 22,
W; production method shown in Reference Example 23,
X; production method shown in Reference Example 24,
Y; production method shown in Reference Example 25,
a; production method shown in Example 1,
b; production method shown in Example 28,
c; production method shown in Example 29,
d; production method shown in Example 30,
e; production method shown in Example 31,
f; production method shown in Example 32,
g; production method shown in Example 33,
h; production method shown in Example 34,
i; production method shown in Example 35,
j; production method shown in Example 36,
k; production method shown in Example 37,
l; production method shown in Example 38,
m; production method shown in Example 39,
n; production method shown in Example 40,
o; production method shown in Example 41,
p; production method shown in Example 42,
q; production method shown in Example 43,
r; production method shown in Example 44,
s; production method shown in Example 45,
t; production method shown in Example 46,
u; production method shown in Example 47,
v; production method shown in Example 48, and

w; production method shown in Example 49.

TABLE 2

| Exp. | Ref. |
|---|---|
| 28 | G, H, F, B, C, a |
| 29 | K, B, C |
| 30 | K, B, C, c |
| 31 | K, B, C, c, d |
| 32 | B, C |
| 33 | B, C, a |
| 34 | K, B, C, c, d |
| 35 | K, B, C, c |
| 36 | K, B, C, c, i |
| 37 | B, C, Q, c |
| 38 | K, B, C, c |
| 39 | K, B, C, c, d |
| 40 | K, B, C, o |
| 41 | K, B, C, o, l |
| 42 | K, B, C, c |
| 43 | H, K, B, C, c, i |
| 44 | K, B, C, c |
| 45 | K, B, C, c, r |
| 46 | K, B, C, c |
| 47 | K, B, C, c, t |
| 48 | K, B, C, c, t, u |
| 49 | K, B, C, |
| | c, l |

### Examples 50 to 667

Production of the compounds in Examples 50 to 267 will be shown below. Details of Examples 50 to 267 are shown in Table 3. Notations in Table 3 mean as follows: Exp; No of example, Str; compound in example, R.T; retention time (minute) in liquid chromatography in LCMS, LC; solvent condition of liquid chromatography in LCMS, MS; mass spectrum data in LCMS, Ref; production method of corresponding intermediate compound, Ex; production method of corresponding compound in example, Notations in the columns in Ref and Ex denote the production methods as follows;
A; production method shown in Reference Example 1,
B; production method shown in Reference Example 2,
C; production method shown in Reference Example 3,
D; production method shown in Reference Example 4,
E; production method shown in Reference Example 5,
F; production method shown in Reference Example 6,
G; production method shown in Reference Example 7,
H; production method shown in Reference Example 8,
I; production method shown in Reference Example 9,
J; production method shown in Reference Example 10,
K; production method shown in Reference Example 11,
L; production method shown in Reference Example 12,
M; production method shown in Reference Example 13,
N; production method shown in Reference Example 14;
O; production method shown in Reference Example 15,
P; production method shown in Reference Example 16,
Q; production method shown in Reference Example 17,
R; production method shown in Reference Example 18,
S; production method shown in Reference Example 19,
T; production method shown in Reference Example 20,
U; production method shown in Reference Example 21,
V; production method shown in Reference Example 22,
W; production method shown in Reference Example 23,
X; production method shown in Reference Example 24,
Y; production method shown in Reference Example 25,
a; production method shown in Example 1,
b; production method shown in Example 28,
c; production method shown in Example 29,
d; production method shown in Example 30,
e; production method shown in Example 31,
f; production method shown in Example 32,
g; production method shown in Example 33,
h; production method shown in Example 34,
i; production method shown in Example 35,
j; production method shown in Example 36,
k; production method shown in Example 37,
l; production method shown in Example 38,
m; production method shown in Example 39,
n; production method shown in Example 40,
o; production method shown in Example 41,
p; production method shown in Example 42,
q; production method shown in Example 43,
r; production method shown in Example 44,
s; production method shown in Example 45,
t; production method shown in Example 46,
u; production method shown in Example 47,
v; production method shown in Example 48, and

w; production method shown in Example 49.

TABLE 3

| Exp. | Str. | R.T. | LC | MS | Ref. | Ex. |
|---|---|---|---|---|---|---|
| 50 | 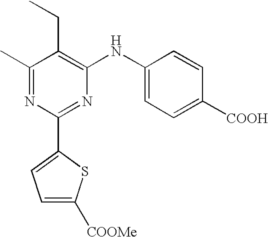 | 5.21 | 1 | 398 (M + H) | G, H, F, B, C | a |
| 51 | 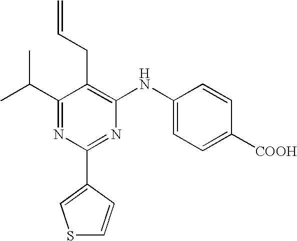 | 5.55 | 1 | 380 (M + H) | I, B, C | a |
| 52 | 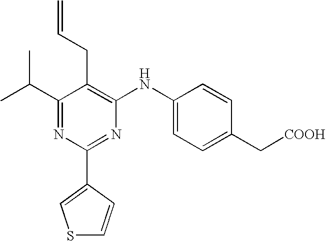 | 4.97 | 1 | 394 (M + H) | I, B, C | a |
| 53 | 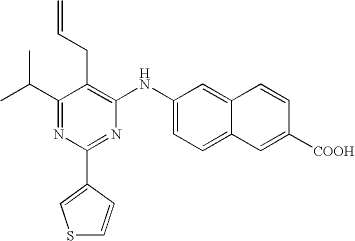 | 6.02 | 1 | 430 (M + H) | I, B, C | a |
| 54 | 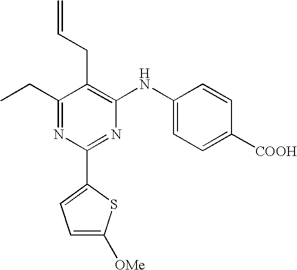 | 4.66 | 1 | 395 (M + H) | D, E, F, I, B, C | a |
| 55 | 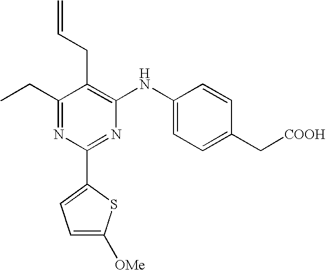 | 4.01 | 1 | 410 (M + H) | D, E, F, I, B, C | a |
| 56 | 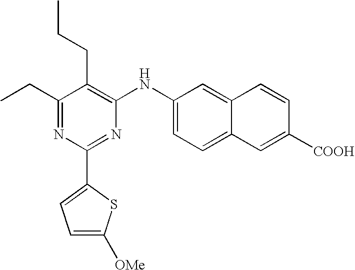 | 5.21 | 1 | 446 (M + H) | D, E, F, I, B, C | a |
| 57 | 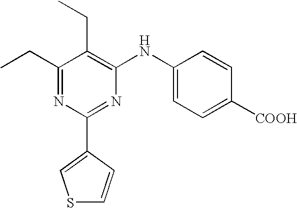 | 3.99 | 1 | 354 (M + H) | B, C | a |
| 58 | 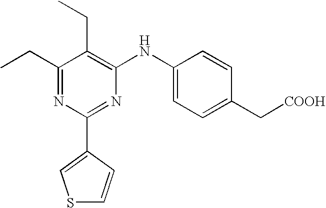 | 3.54 | 1 | 368 (M + H) | B, C | a |
| 59 | 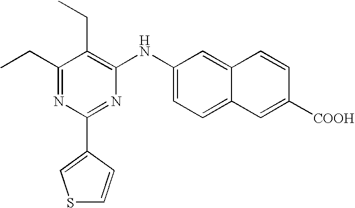 | 4.33 | 1 | 404 (M + H) | B, C | a |
| 60 | 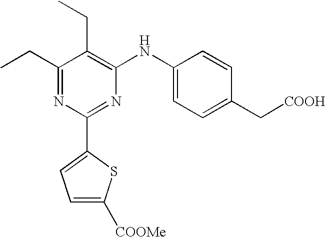 | 4.31 | 1 | 412 (M + H) | G, H, F, B, C | a |
| 61 | 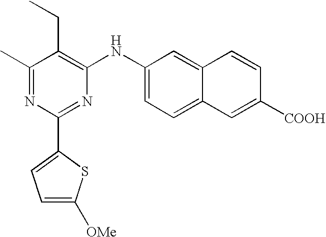 | 5.69 | 1 | 448 (M + H) | G, H, F, B, C | a |
| 62 | 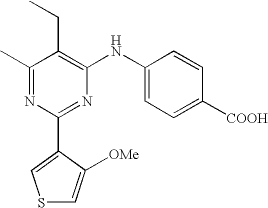 | 2.62 | 1 | 372 (M + H) | J, B, C | a |
| 63 | 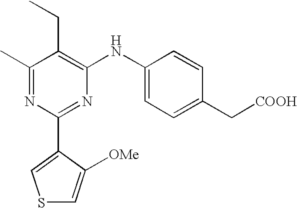 | 2.66 | 1 | 388 (M + H) | J, B, C | a |
| 64 | 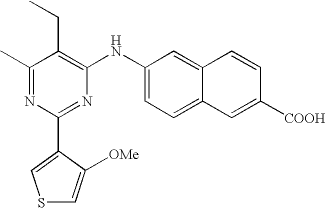 | 2.85 | 1 | 422 (M + H) | J, B, C | a |
| 65 | 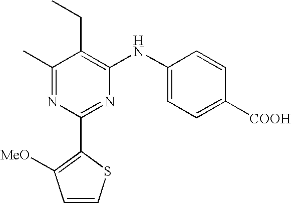 | 3.38 | 1 | 372 (M + H) | D, H, K, B, C | a |
| 66 | 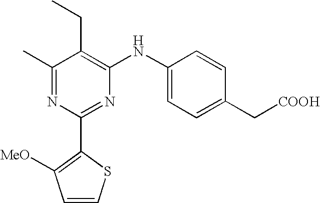 | 3.36 | 1 | 388 (M + H) | D, H, K, B, C | a |
| 67 | 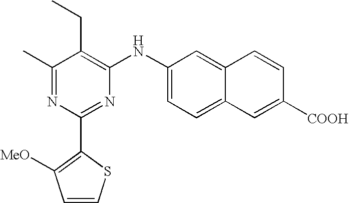 | 3.61 | 1 | 422 (M + H) | D, H, K, B, C | a |
| 68 | 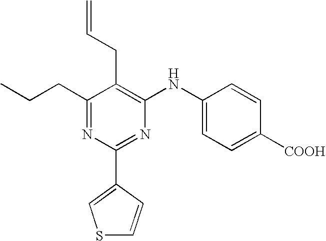 | 4.31 | 1 | 380 (M + H) | I, B, C | a |
| 69 | 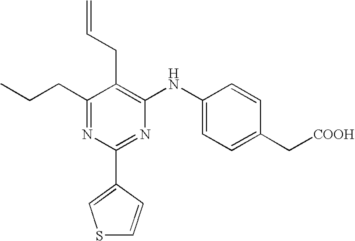 | 3.50 | 1 | 394 (M + H) | I, B, C | a |
| 70 | 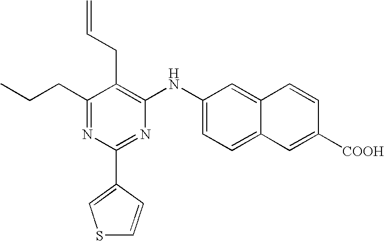 | 4.65 | 1 | 430 (M + H) | I, B, C | a |
| 71 | 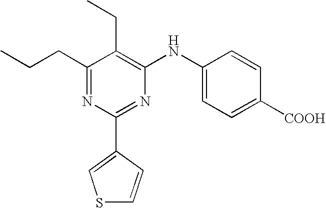 | 3.83 | 1 | 368 (M + H) | L, B, C | a |
| 72 | 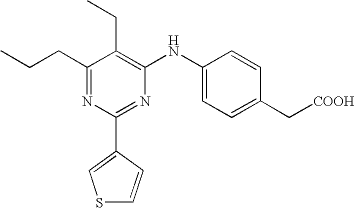 | 3.07 | 1 | 382 (M + H) | L, B, C | a |
| 73 | 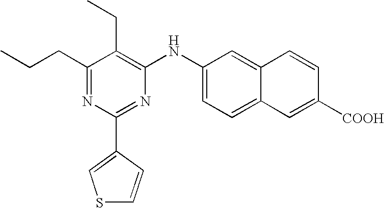 | 4.24 | 1 | 418 (M + H) | L, B, C | a |
| 74 | 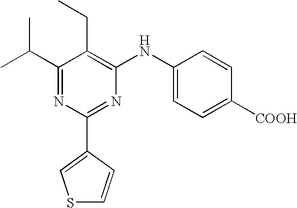 | 4.88 | 1 | 368 (M + H) | L, B, C | a |
| 75 | 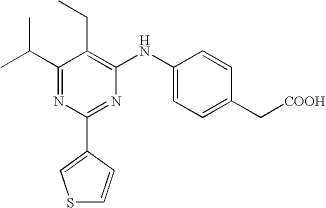 | 4.29 | 1 | 382 (M + H) | L, B, C | a |
| 76 | 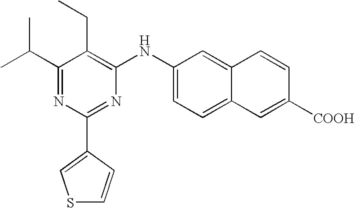 | 5.77 | 1 | 418 (M + H) | L, B, C | a |
| 77 | 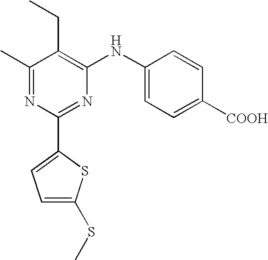 | 4.34 | 1 | 386 (M + H) | J, B, C | a |
| 78 | 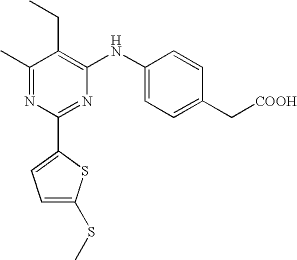 | 3.6 | 1 | 400 (M + H) | J, B, C | a |
| 79 | 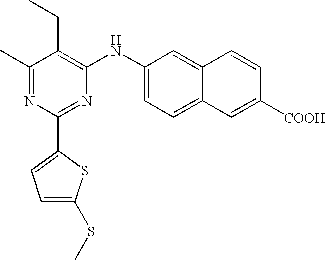 | 5.09 | 1 | 436 (M + H) | J, B, C | a |
| 80 | 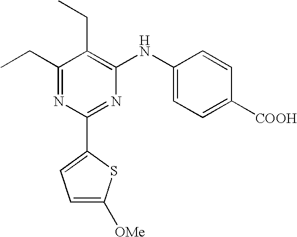 | 3.77 | 1 | 384 (M + H) | D, E, K, B, C | a |
| 81 | 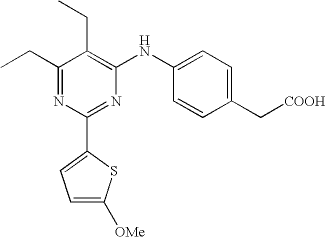 | 3.22 | 1 | 398 (M + H) | D, E, K, B, C | a |
| 82 | 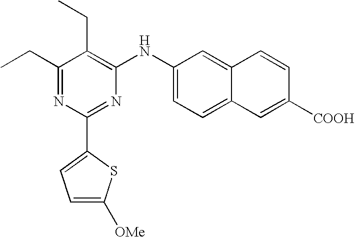 | 4.55 | 1 | 434 (M + H) | D, E, K, B, C | a |
| 83 | 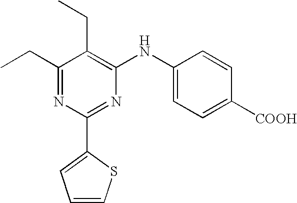 | 4.18 | 1 | 354 (M + H) | B, C | a |
| 84 | 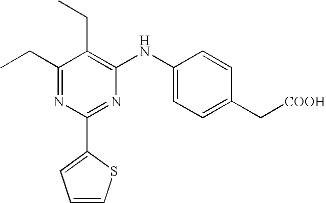 | 3.41 | 1 | 368 (M + H) | B, C | a |
| 85 | 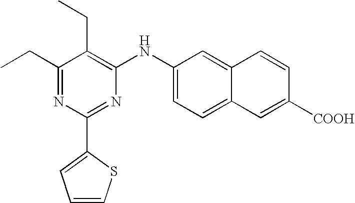 | 4.89 | 1 | 404 (M + H) | B, C | a |
| 86 | 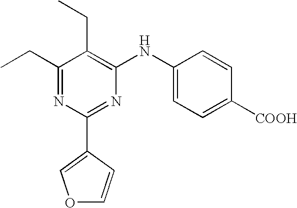 | 3.22 | 1 | 338 (M + H) | B, C | a |
| 87 | 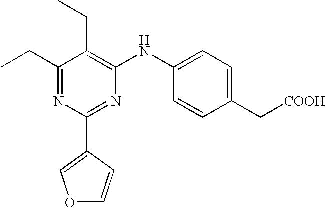 | 2.69 | 1 | 352 (M + H) | B, C | a |
| 88 | 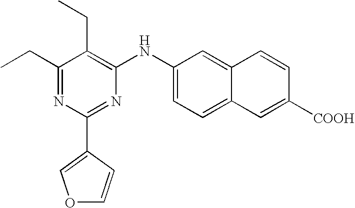 | 3.59 | 1 | 368 (M + H) | B, C | a |
| 89 | 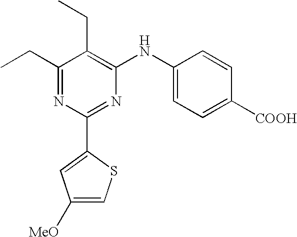 | 4.93 | 1 | 384 (M + H) | M, N, O, E, K, B, C | a |
| 90 | 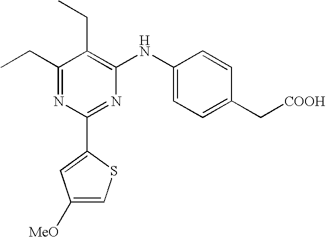 | 4.27 | 1 | 398 (M + H) | M, N, O, E, K, B, C | a |
| 91 | 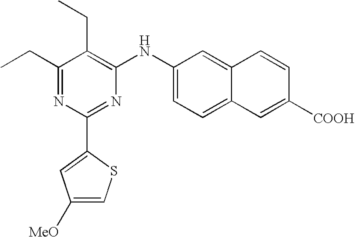 | 5.5 | 1 | 435 (M + H) | M, N, O, E, K, B, C | a |
| 92 | 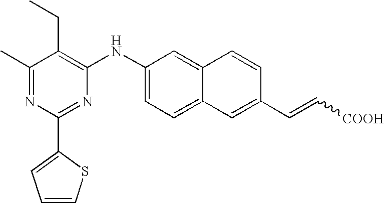 | 4.58 4.75 | 1 | — | B, C | a |
| 93 | 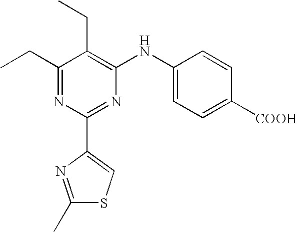 | 2.70 | 1 | 369 (M + H) | B, C | a |
| 94 | 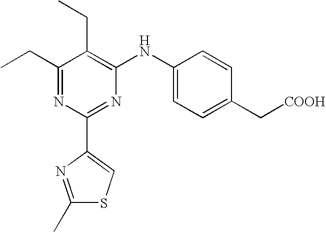 | 2.55 | 1 | 383 (M + H) | B, C | a |
| 95 | 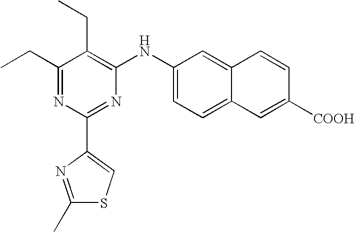 | 3.07 | 1 | 419 (M + H) | B, C | a |
| 96 | 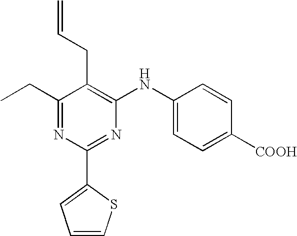 | 4.64 | 1 | 368 (M + H) | I, B, C | a |
| 97 | 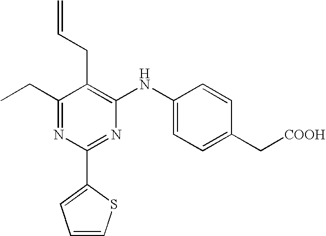 | 3.83 | 1 | 380 (M + H) | I, B, C | a |
| 98 | 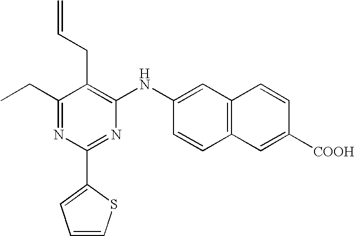 | 5.34 | 1 | 416 (M + H) | I, B, C | a |
| 99 | 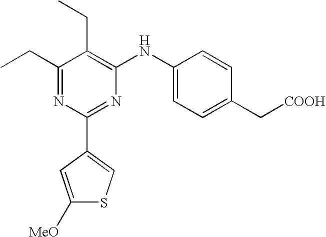 | 3.04 | 1 | 398 (M + H) | P, M, N, O, E, K | a |
| 100 | 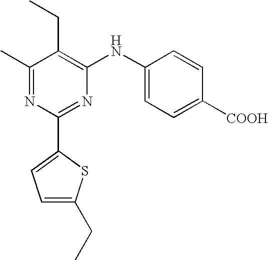 | 3.93 | 1 | 368 (M + H) | H, F, B, C | a |
| 101 | 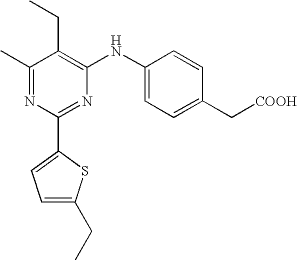 | 3.32 | 1 | 382 (M + H) | H, F, B, C | a |
| 102 | 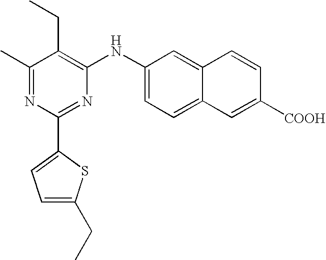 | 4.58 | 1 | 418 (M + H) | H, F, B, C | a |
| 103 | 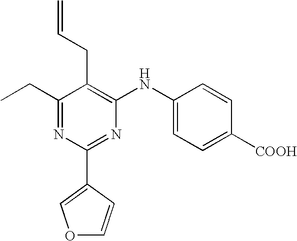 | 3.60 | 1 | 350 (M + H) | I, B, C | a |
| 104 | 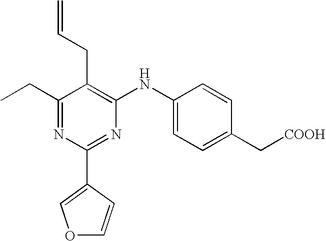 | 2.99 | 1 | 364 (M + H) | I, B, C | a |
| 105 | 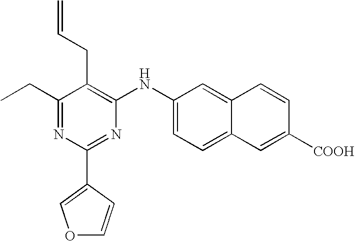 | 4.14 | 1 | 400 (M + H) | I, B, C | a |
| 106 | 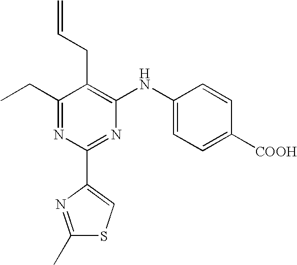 | 2.95 | 1 | 381 (M + H) | I, B, C | a |
| 107 | 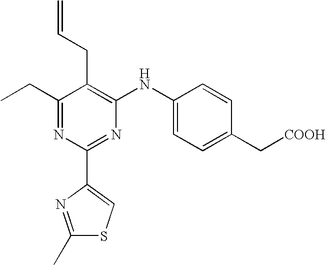 | 2.68 | 1 | 395 (M + H) | I, B, C | a |
| 108 | 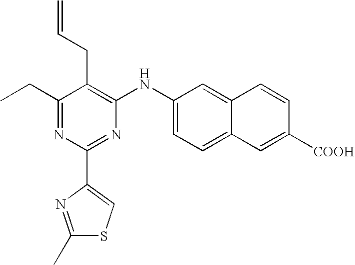 | 3.38 | 1 | 431 (M + H) | I, B, C | a |
| 109 | 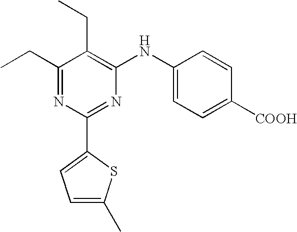 | 4.13 | 1 | 368 (M + H) | H, F, B, C | a |
| 110 | 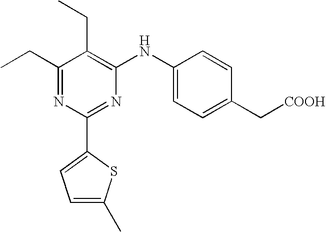 | 3.35 | 1 | 382 (M + H) | H, F, B, C | a |
| 111 | 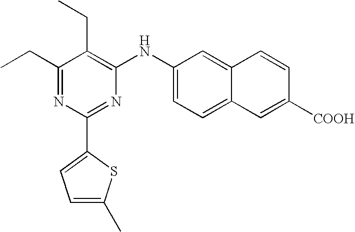 | 4.81 | 1 | 418 (M + H) | H, F, B, C | a |
| 112 | 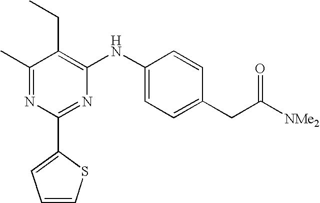 | 3.58 | 1 | 381 (M + H) | B, C, a | g |
| 113 | 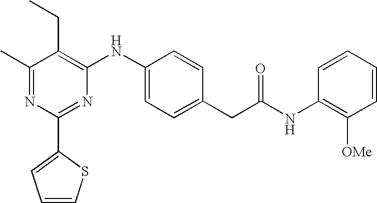 | 4.35 | 1 | 459 (M + H) | B, C, a | g |
| 114 | 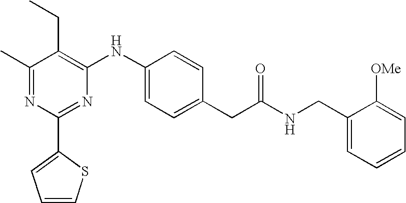 | 4.11 | 1 | 473 (M + H) | B, C, a | g |
| 115 | 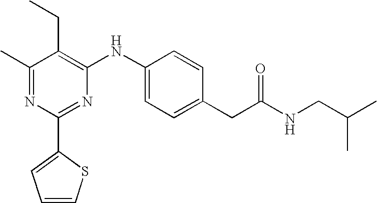 | 3.85 | 1 | 409 M + H | B, C, a | g |
| 116 | 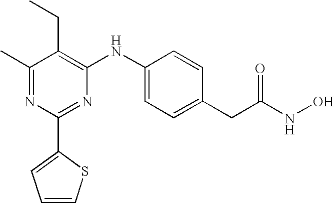 | 3.26 | 1 | 369 (M + H) | B, C, a | g |
| 117 | 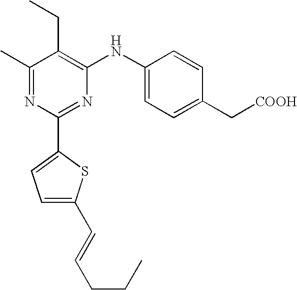 | 3.69 | 1 | 394 (M + H) | K, B, C, c, d | e |
| 118 | 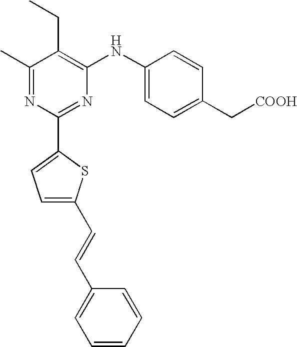 | 4.44 | 1 | 422 (M + H) | K, B, C, c, d | e |
| 119 | 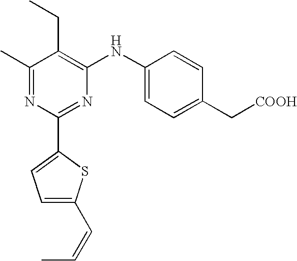 | 5.14 | 1 | 456 (M + H) | K, B, C, c, d | e |
| 120 | 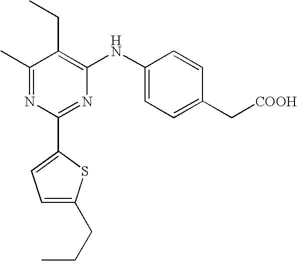 | 3.64 | 1 | 396 (M + H) | K, B, C, c, d, h | e |
| 121 | 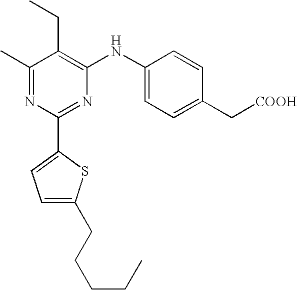 | 4.11 | 1 | 424 (M + H) | K, B, C, c, d, h | e |
| 122 | 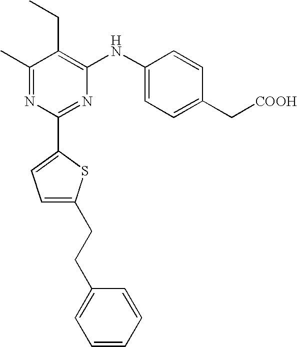 | 3.97 | 1 | 458 (M + H) | K, B, C, c, d, h | e |
| 123 | 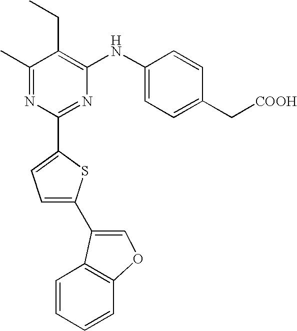 | 5.93 | 1 | 470 (M + H) | K, B, C, c, d | e |
| 124 | 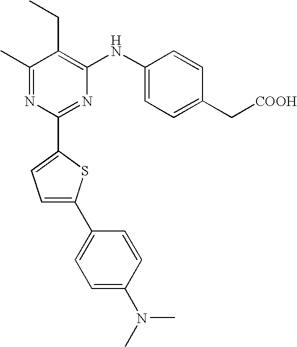 | 4.3 | 1 | 473 (M + H) | K, B, C, c, d | e |
| 125 | 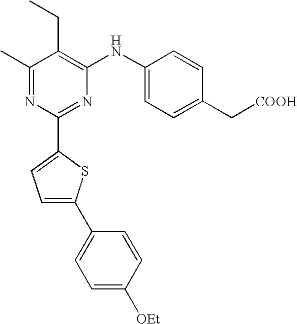 | 4.81 | 1 | 474 (M + H) | K, B, C, c, d | e |
| 126 | 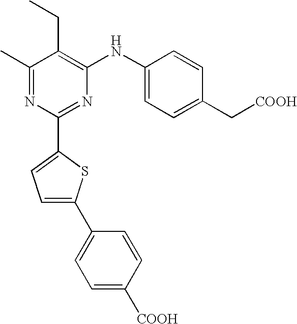 | 4.06 | 1 | 474 (M + H) | K, B, C, c, d | e |
| 127 | 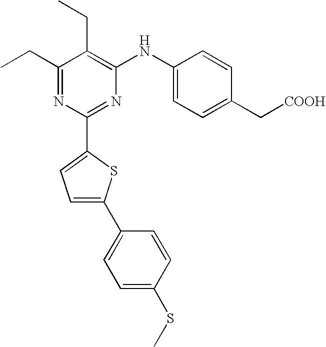 | 5.04 | 1 | 400 (M + H) | F, B, C | a |
| 128 | 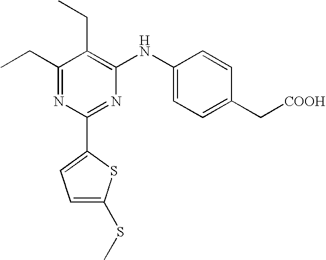 | 4.15 | 1 | 414 (M + H) | F, B, C | a |
| 129 | 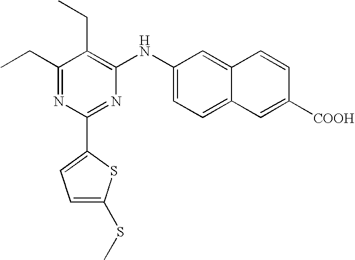 | 5.79 | 1 | 450 (M + H) | F, B, C | a |
| 130 | 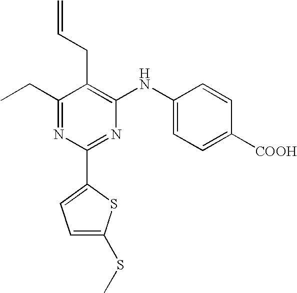 | 5.48 | 1 | 412 (M + H) | F, I, B, C | a |
| 131 | 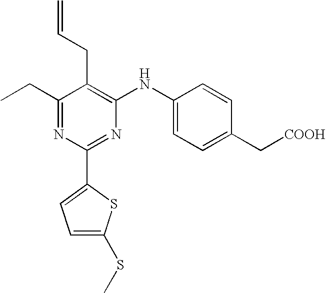 | 4.92 | 1 | 426 (M + H) | F, I, B, C | a |
| 132 | 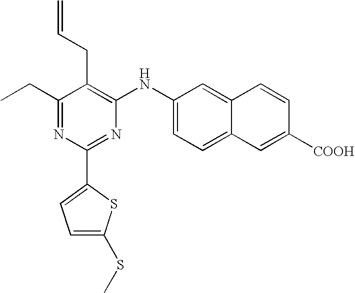 | 6.11 | 1 | 462 (M + H) | F, I, B, C | a |
| 133 | 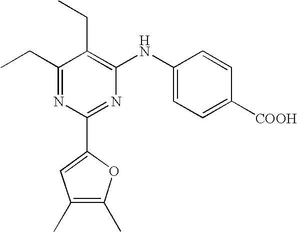 | 3.25 | 1 | 355 (M + H) | H, F, B, C | a |
| 134 | 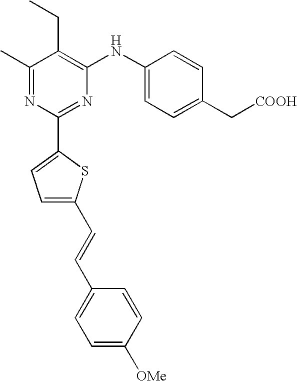 | 5.09 | 1 | 486 (M + H) | K, B, C, c, d | e |
| 135 | 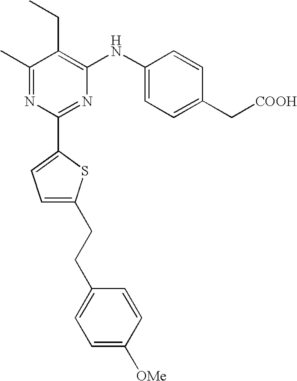 | 3.97 | 1 | 488 (M + H) | K, B, C, c, d, h | e |
| 136 | 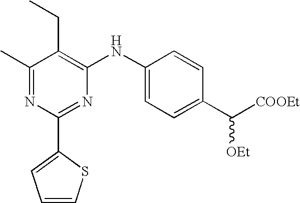 | 4.58 | 1 | 426 (M + H) | B, C, Q | c |
| 137 | 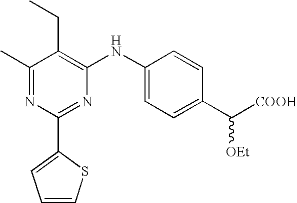 | 3.79 | 1 | 398 (M + H) | B, C, Q, c | e |
| 138 | 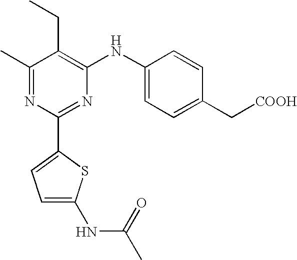 | 3.34 | 1 | 411 (M + H) | K, B, C, c, i, j | e |
| 139 | 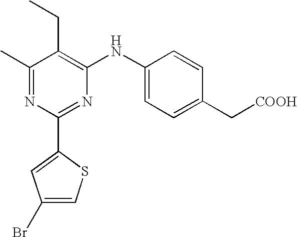 | 5.92 | 1 | 460 (M + H) | H, K, B, C | c |
| 140 | 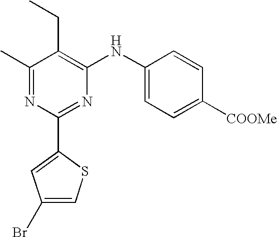 | 6.22 | 1 | 432 (M + H) | H, K, B, C | c |
| 141 | 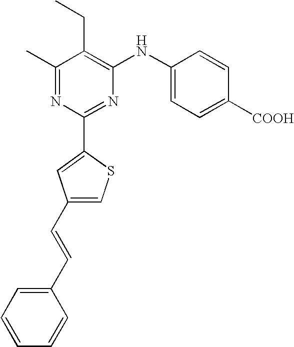 | 4.48 | 4 | 442 (M + H) | H, K, B, C, c, d | e |
| 142 | 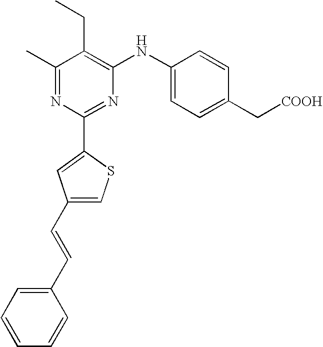 | 3.04 | 4 | 456 (M + H) | H, K, B, C, c, d | e |
| 143 | 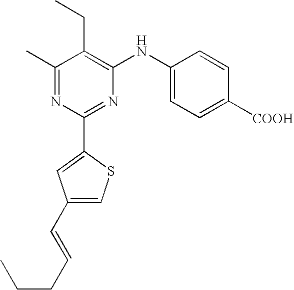 | 3.69 | 4 | 408 (M + H) | H, K, B, C, c, d | e |
| 144 | 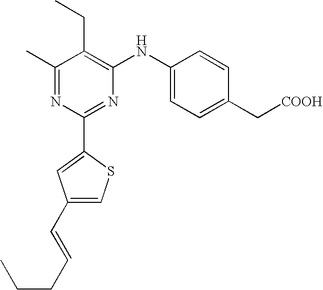 | 2.43 | 4 | 422 (M + H) | H, K, B, C, c, d | e |
| 145 | 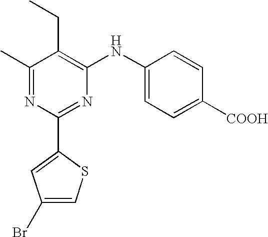 | 5.14 | 1 | 418 (M + H) | H, K, B, C, c | e |
| 146 | 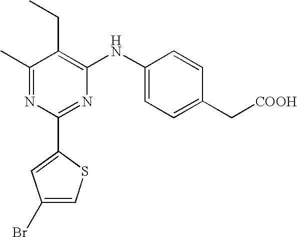 | 4.93 | 1 | 432 (M + H) | H, K, B, C, c | e |
| 147 | 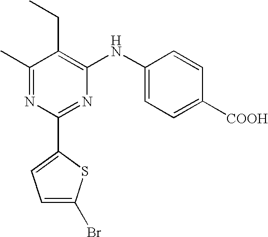 | 2.94 | 1 | 418 (M + H) | K, B, C, c | e |
| 148 | 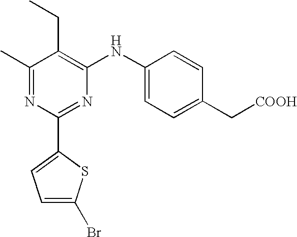 | 4.78 | 1 | 432 (M + H) | K, B, C, c | e |
| 149 | 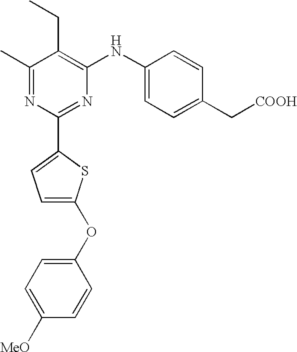 | 3.78 | 1 | 476 (M + H) | R, K, B, C, c | e |
| 150 | 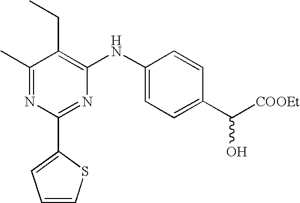 | 3.84 | 1 | 398 (M + H) | B, C, Q | c |
| 151 | 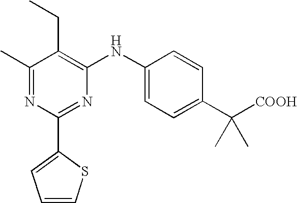 | 4.09 | 1 | 382 (M + H) | S, T, B, C, c | e |
| 152 | 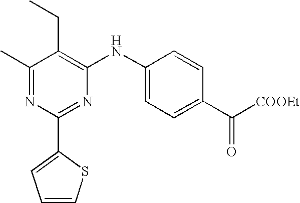 | 3.60 | 1 | 368 (M + H) | B, C, Q, c, k | e |
| 153 | 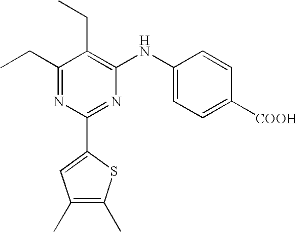 | 4.40 | 1 | 382 (M + H) | H, F, B, C | a |
| 154 | 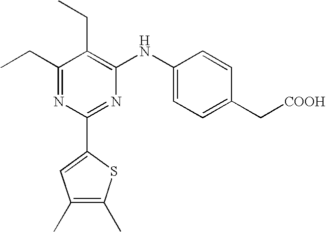 | 3.42 | 1 | 396 (M + H) | H, F, B, C | a |
| 155 | 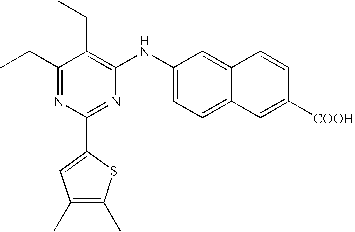 | 5.03 | 1 | 432 (M + H) | H, F, B, C | a |
| 156 | 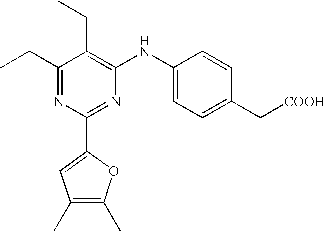 | 2.88 | 1 | 380 (M + H) | H, F, B, C | a |
| 157 | 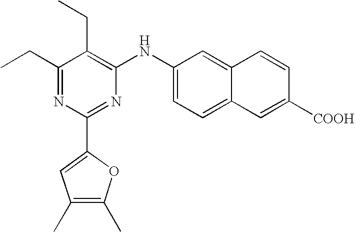 | 3.77 | 1 | 416 (M + H) | H, F, B, C | a |
| 158 | 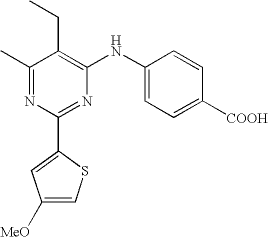 | 4.03 | 1 | 370 (M + H) | M, N, O, E K, B, C | a |
| 159 | 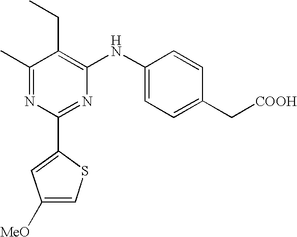 | 3.28 | 1 | 384 (M + H) | M, N, O, E K, B, C | a |
| 160 | 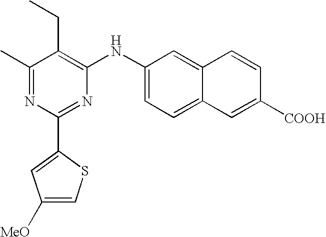 | 4.72 | 1 | 420 (M + H) | M, N, O, E K, B, C | a |
| 161 | 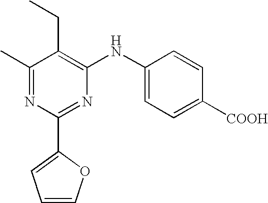 | 2.79 | 1 | 324 (M + H) | B, C | a |
| 162 | 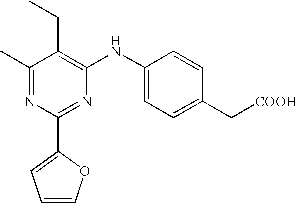 | 2.55 | 1 | 338 (M + H) | B, C | a |
| 163 | 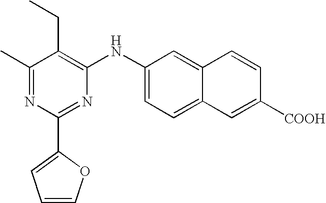 | 3.31 | 1 | 374 (M + H) | B, C | a |
| 164 | 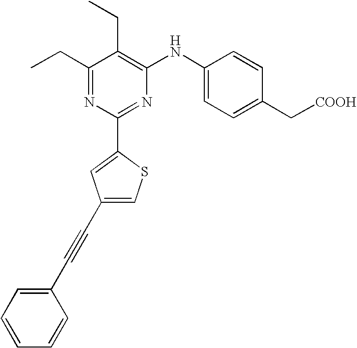 | 6.19 | 2 | 468 (M + H) | H, F, B, C | a |
| 165 | 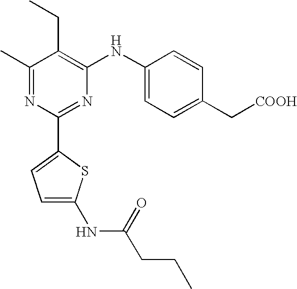 | 2.8 | 1 | 439 (M + H) | K, B, C, c, i, j | e |
| 166 | 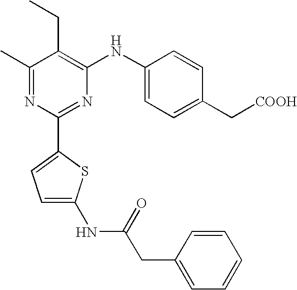 | 2.99 | 1 | 487 (M + H) | K, B, C, c, i, j | e |
| 167 | 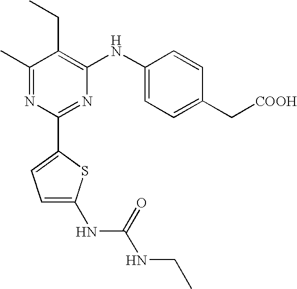 | 2.62 | 1 | 440 (M + H) | K, B, C, c, i, j | e |
| 168 | 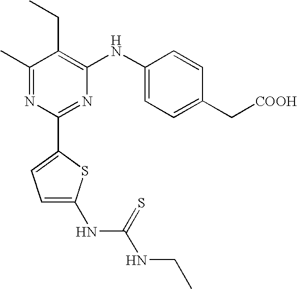 | 2.8 | 1 | 456 (M + H) | K, B, C, c, i, j | e |
| 169 | 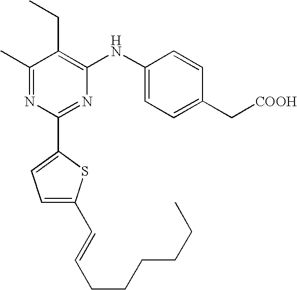 | 5.93 | 1 | 464 (M + H) | K, B, C, c, d | e |
| 170 | 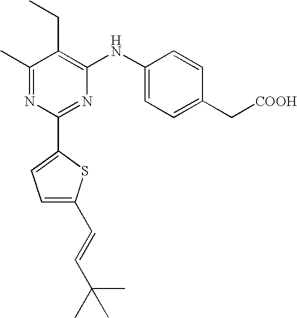 | 4.76 | 1 | 437 (M + H) | K, B, C, c, d | e |
| 171 | 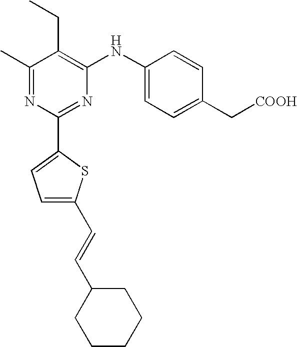 | 5.65 | 1 | 463 (M + H) | K, B, C, c, d | e |
| 172 | 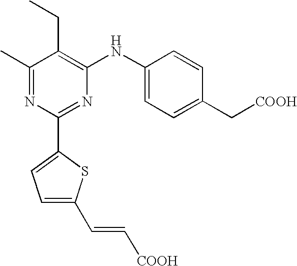 | 3.46 | 1 | 424 (M + H) | K, B, C, c, l | e |
| 173 | 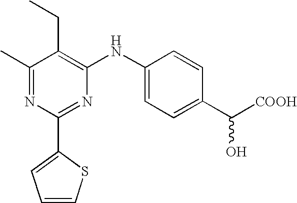 | 3.26 | 1 | 370 (M + H) | B, C, Q, c | e |
| 174 | 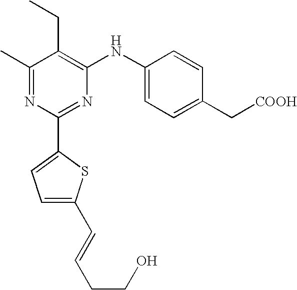 | 3.67 | 1 | 424 (M + H) | K, B, C, c, d, m | e |
| 175 | 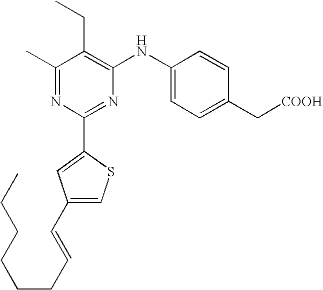 | 6.26 | 1 | 465 (M + H) | H, K, B, C, c, d | e |
| 176 | 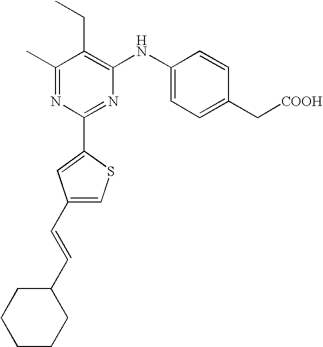 | 5.74 | 1 | 463 (M + H) | H, K, B, C, c, d | e |
| 177 | 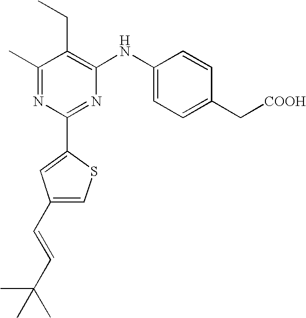 | 4.9 | 1 | 437 (M + H) | H, K, B, C, c, d | e |
| 178 | 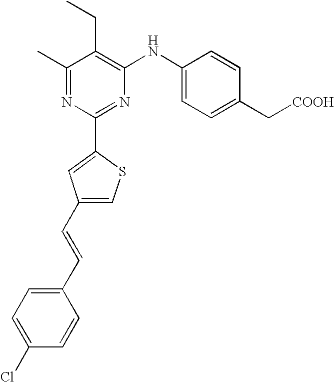 | 6.4 | 1 | 491 (M + H) | H, K, B, C, c, d | e |
| 179 | 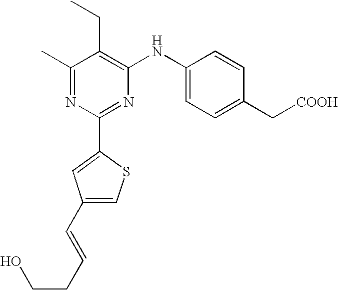 | 0.47 | 4 | 424 (M + H) | H, K, B, C, c, d, m | e |
| 180 | 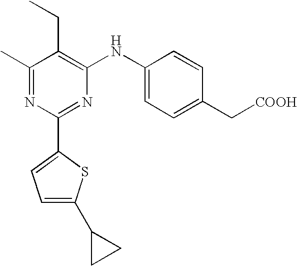 | 0.7 | 4 | 395 (M + H) | K, B, C, c, n | e |
| 181 | 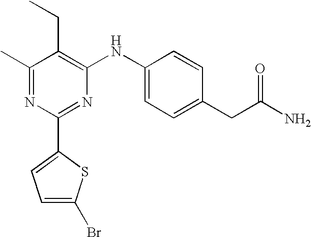 | 4.17 | 1 | 430 (M) | K, B, C, c, e | g |
| 182 | 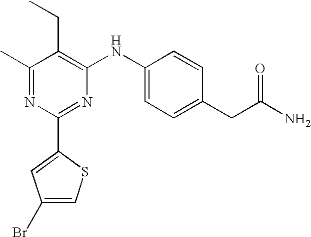 | 4.31 | 1 | 431 (M + H) | H, K, B, C, c, e | g |
| 183 | 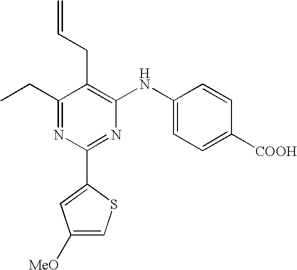 | 5.03 | 1 | 396 (M + H) | M, N, O, E, K, I, B, C | a |
| 184 | 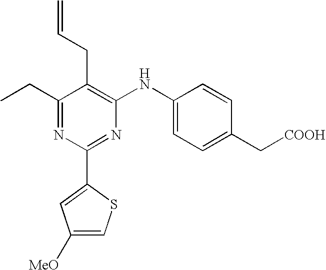 | 4.56 | 1 | 410 (M + H) | M, N, O, E, K, I, B, C | a |
| 185 | 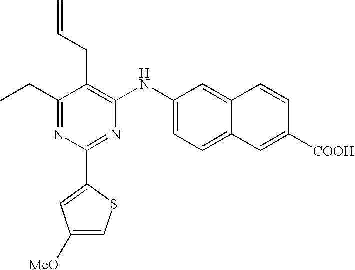 | 5.62 | 1 | 446 (M + H) | M, N, O, E, K, I, B, C | a |
| 186 | 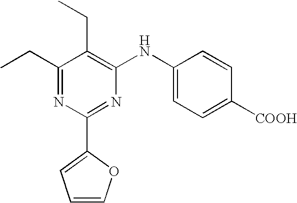 | 3.19 | 1 | 338 (M + H) | B, C | a |
| 187 | 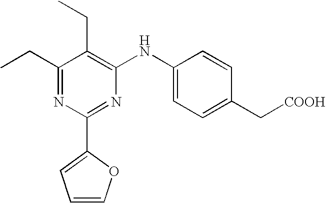 | 2.67 | 1 | 352 (M + H) | B, C | a |
| 188 | 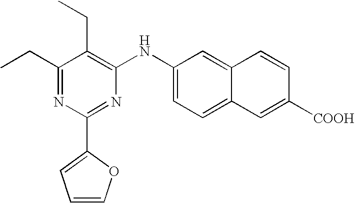 | 3.70 | 1 | 388 (M + H) | B, C | a |
| 189 | 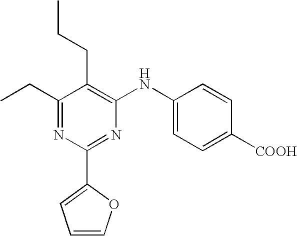 | 3.64 | 1 | 350 (M + H) | I, B, C | a |
| 190 | 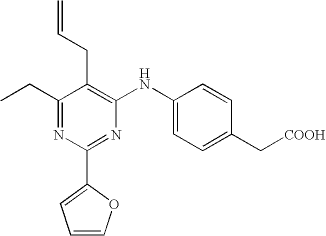 | 3.01 | 1 | 364 (M + H) | I, B, C | a |
| 191 | 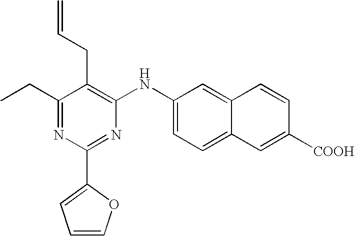 | 4.20 | 1 | 400 (M + H) | I, B, C | a |
| 192 | 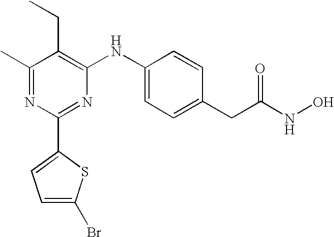 | 4.01 | 1 | 447 (M + H) | K, B, C, c, e | g |
| 193 | 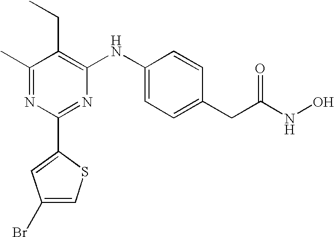 | 4.17 | 1 | 447 (M + H) | H, K, B, C, c, e | g |
| 194 | 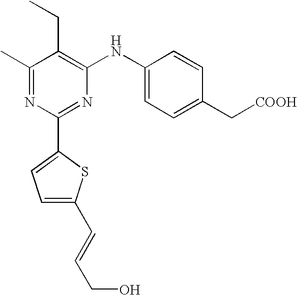 | 2.94 | 1 | 411 (M + H) | K, B, C, c, m | e |
| 195 | 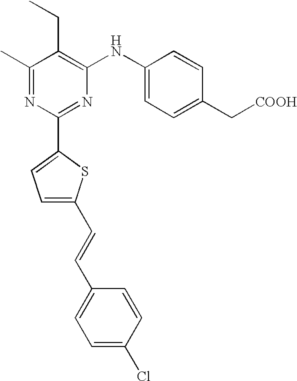 | 6.3 | 1 | 491 (M + H) | K, B, C, c, d | e |
| 196 | 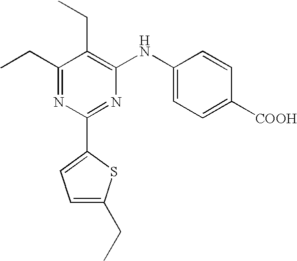 | 4.55 | 1 | 382 (M + H) | H, F, B, C | a |
| 197 | 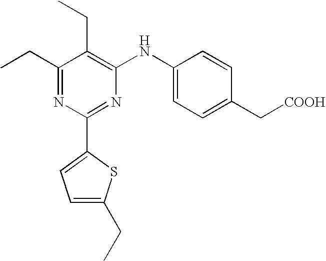 | 3.59 | 1 | 396 (M + H) | H, F, B, C | a |
| 198 | 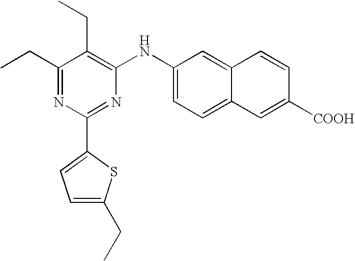 | 5.23 | 1 | 432 (M + H) | H, F, B, C | a |
| 199 | 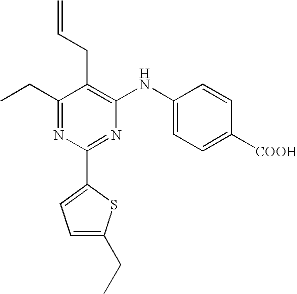 | 5.09 | 1 | 394 (M + H) | H, F, I, B, C | a |
| 200 | 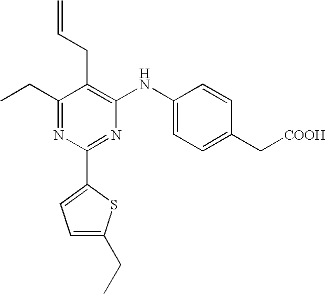 | 4.07 | 1 | 408 (M + H) | H, F, I, B, C | a |
| 201 | 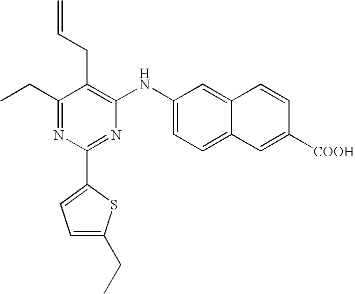 | 5.84 | 1 | 444 (M + H) | H, F, I, B, C | a |
| 202 | 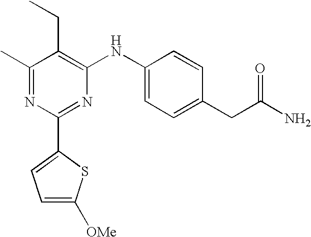 | 3.32 | 1 | 383 (M + H) | D, E, F, B, C, a | g |
| 203 | 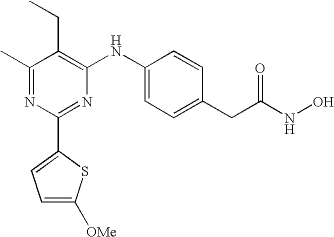 | 3.25 | 1 | 399 (M + H) | D, E, F, B, C, a | g |
| 204 | 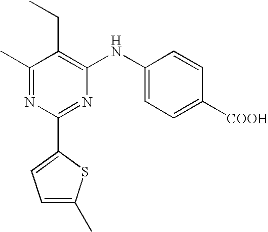 | 4.11 | 1 | 354 (M + H) | H, F, B, C | a |
| 205 | 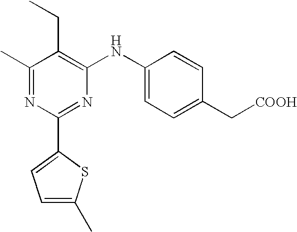 | 3.72 | 1 | 368 (M + H) | H, F, B, C | a |
| 206 | 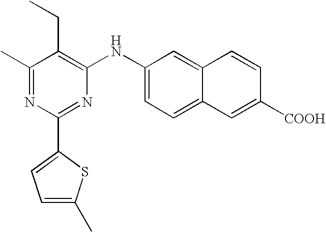 | 4.55 | 1 | 404 (M + H) | H, F, B, C | a |
| 207 | 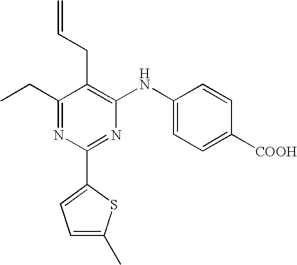 | 4.94 | 1 | 380 (M + H) | H, F, I, B, C | a |
| 208 | 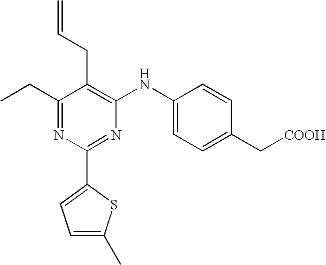 | 4.27 | 1 | 394 (M + H) | H, F, I, B, C | a |
| 209 | 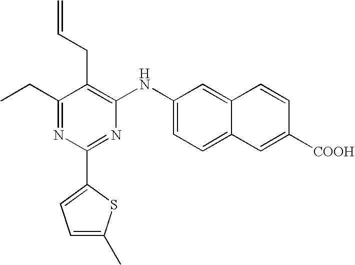 | 5.49 | 1 | 430 (M + H) | H, F, I, B, C | a |
| 210 | 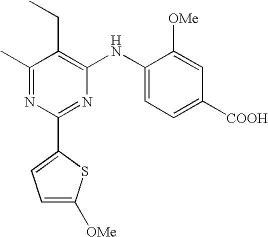 | 4.50 | 1 | 400 (M + H) | D, E, F, B, C | a |
| 211 | 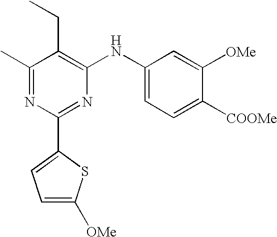 | 4.54 | 1 | 414 (M + H) | D, E, F, B, C | c |
| 212 | 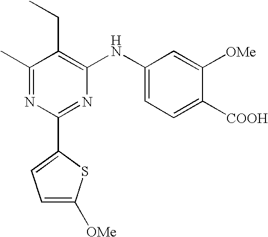 | 4.15 | 1 | 400 (M + H) | D, E, F, B, C, c | c |
| 213 | 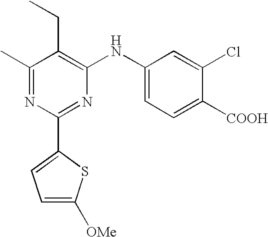 | 4.29 | 1 | 404 (M + H) | D, E, F, B, C | a |
| 214 | 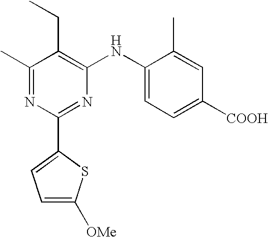 | 3.70 | 1 | 384 (M + H) | D, E, F, B, C | a |
| 215 | 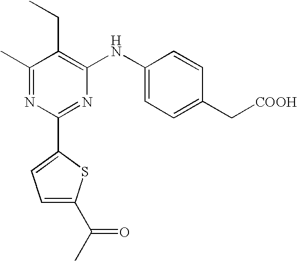 | 4.07 | 1 | 396 (M + H) | K, B, C, c, l | e |
| 216 | 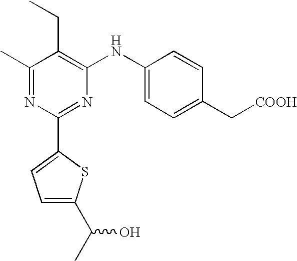 | 2.66 | 1 | 398 (M + H) | K, B, C, c, l, o | e |
| 217 | 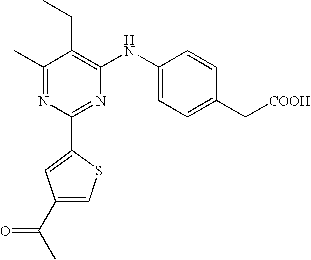 | 3.55 | 1 | 396 (M + H) | H, K, B, C, c, l | c |
| 218 | 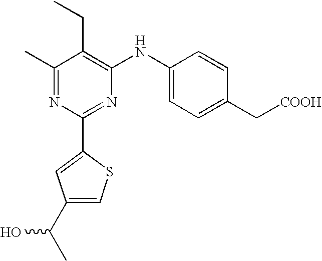 | 2.71 | 1 | 398 (M + H) | H, K, B, C, c, l, o | e |
| 219 | 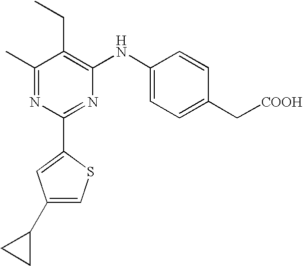 | 3.55 | 1 | 394 (M + H) | H, K, B, C, c, n | e |
| 220 | 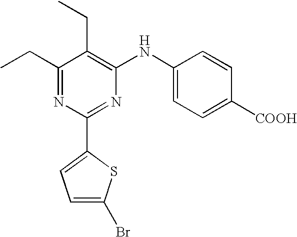 | 5.68 | 2 | 432 (M + H) | K, B, C, c | e |
| 221 | 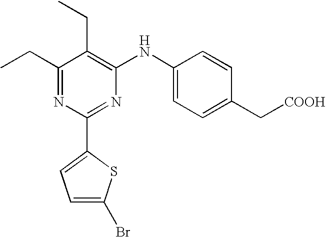 | 5.12 | 2 | 446 (M + H) | K, B, C, c | e |
| 222 | 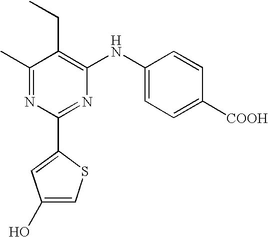 | 3.70 | 1 | 356 (M + H) | M, N, O, E, K, B, C, U, c | e |
| 223 | 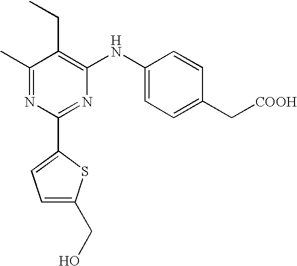 | 2.53 | 1 | 384 (M + H) | M, F, B, W, V, c | e |
| 224 | 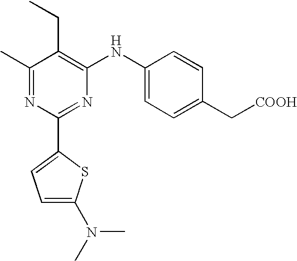 | 3.67 | 1 | 397 (M + H) | K, B, C, c, p | e |
| 225 | 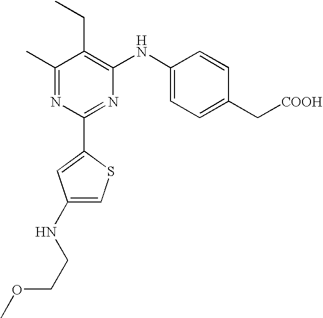 | 2.9 | 1 | 427 (M + H) | H, K, B, C, c, i, q | e |
| 226 | 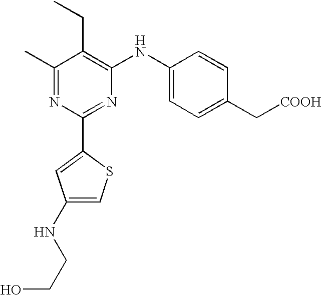 | 3.05 | 1 | 413 (M + H) | H, K, B, C, c, i, q, m | e |
| 227 | 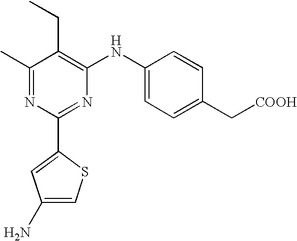 | 3.34 | 1 | 369 (M + H) | H, K, B, C, c, i | e |
| 228 | 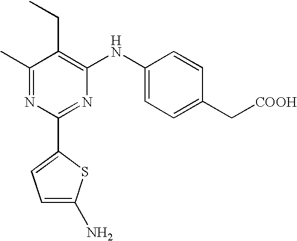 | 3.4 | 1 | 369 (M + H) | K, B, C, c, i | e |
| 229 | 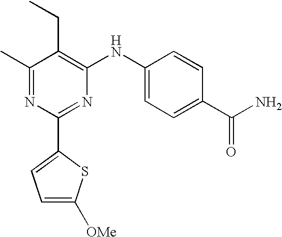 | 3.52 | 1 | 369 (M + H) | D, E, K, B, C, a | g |
| 230 | 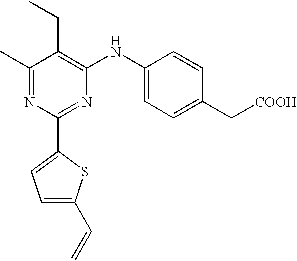 | 3.69 | 1 | 380 (M + H) | K, B, C, c, r | e |
| 231 | 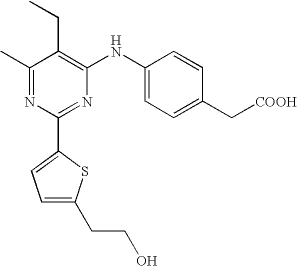 | 3.37 | 1 | 398 (M + H) | K, B, C, c, r, s | e |
| 232 | 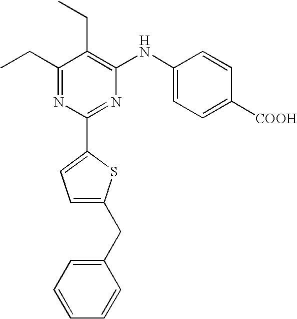 | 5.28 | 1 | 444 (M + H) | H, F, B, C | a |
| 233 | 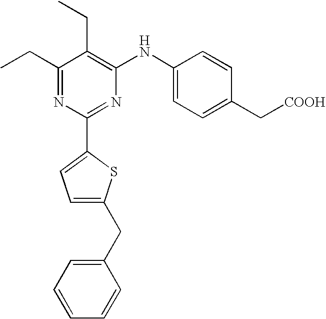 | 4.50 | 1 | 458 (M + H) | H, F, B, C | a |
| 234 | 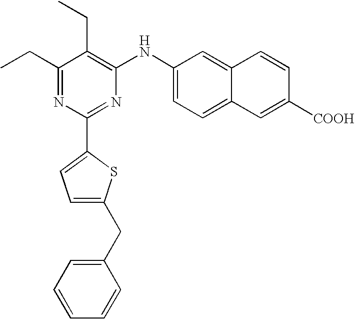 | 5.86 | 2 | 494 (M + H) | H, F, B, C | a |
| 235 | 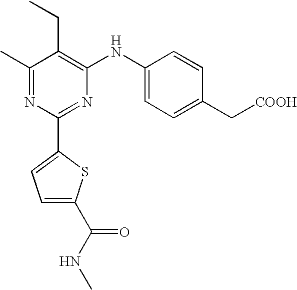 | 3.73 | 1 | 411 (M + H) | G, H, F, B, b, g, C, c | e |
| 236 | 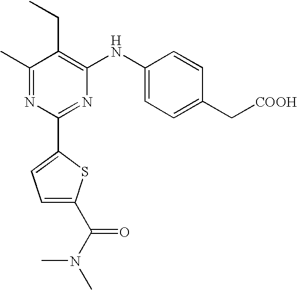 | 3.79 | 1 | 425 (M + H) | G, H, F, B, b, g, C, c | e |
| 237 | 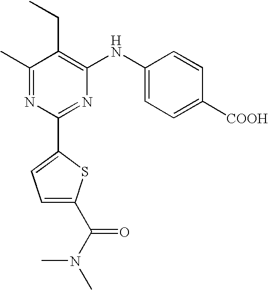 | 4.09 | 1 | 411 (M + H) | G, H, F, B, b, g, C, c | e |
| 238 | 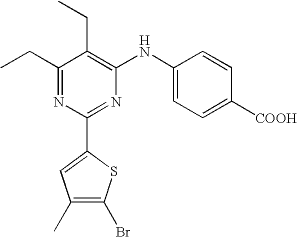 | 5.52 | 3 | 446 (M + H) | H, K, B, C, c | e |
| 239 | 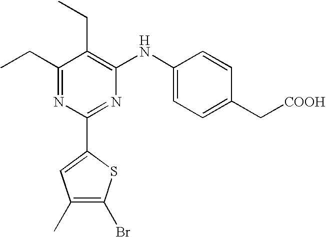 | 4.86 | 3 | 460 (M + H) | H, K, B, C, c | e |
| 240 | 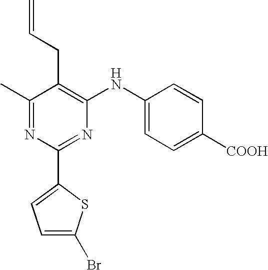 | 4.81 | 3 | 430 (M + H) | K, B, C, c | e |
| 241 | 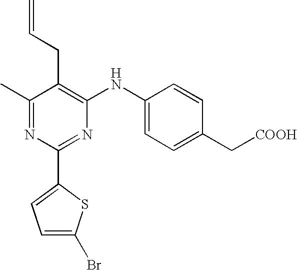 | 4.34 | 3 | 444 (M + H) | K, B, C, c | e |
| 242 | 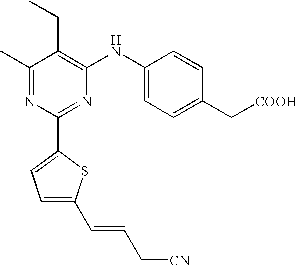 | 5.02 | 1 | 419 (M + H) | K, B, C, c, l | e |
| 243 | 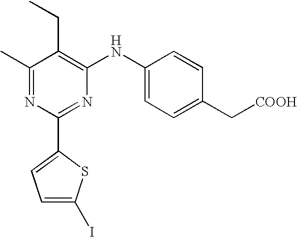 | 4.75 | 1 | 480 (M + H) | K, B, C, c, t | e |
| 244 | 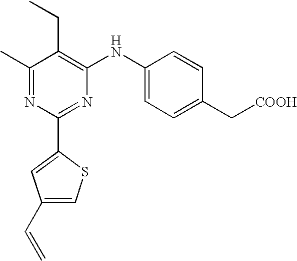 | 4.18 | 1 | 380 (M + H) | H, K, B, C, c, r | e |
| 245 | 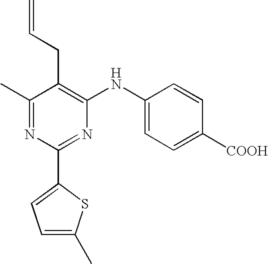 | 4.45 | 1 | 366 (M + H) | H, F, B, C | a |
| 246 | 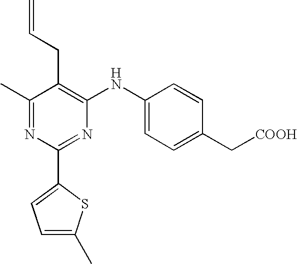 | 3.69 | 1 | 380 (M + H) | H, F, B, C | a |
| 247 | 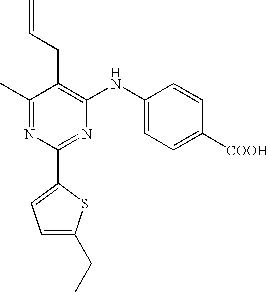 | 4.88 | 1 | 380 (M + H) | H, F, B, C | a |
| 248 | 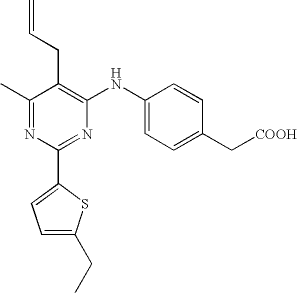 | 4.08 | 1 | 394 (M + H) | H, F, B, C | a |
| 249 | 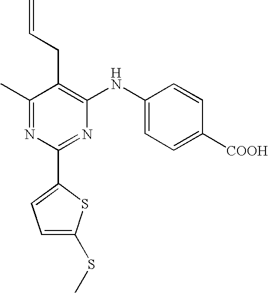 | 5.00 | 1 | 398 (M + H) | F, B, C | a |
| 250 | 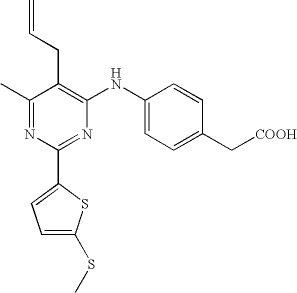 | 4.42 | 1 | 412 (M + H) | F, B, C | a |
| 251 | 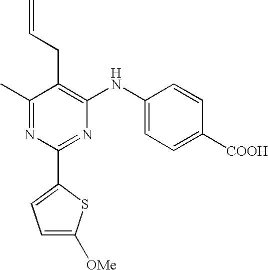 | 4.20 | 1 | 382 (M + H) | D, E, F, B, C | a |
| 252 | 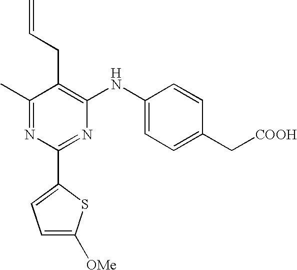 | 3.74 | 1 | 396 (M + H) | D, E, F, B, C | a |
| 253 | 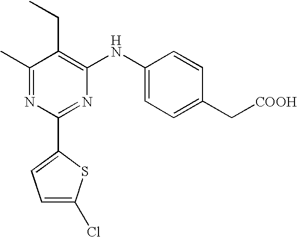 | 4.70 | 1 | 388 (M + H) | H, K, B, C, c | e |
| 254 | 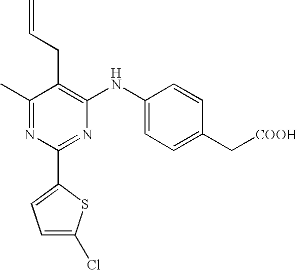 | 5.01 | 1 | 400 (M + H) | H, K, B, C, c | e |
| 255 | 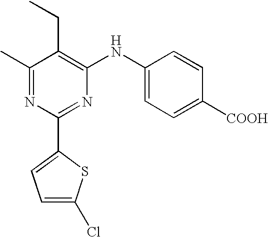 | 5.16 | 1 | 374 (M + H) | H, K, B, C, c | e |
| 256 | 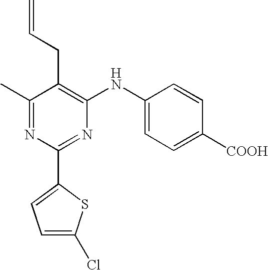 | 5.33 | 1 | 386 (M + H) | H, K, B, C, c | e |
| 257 | 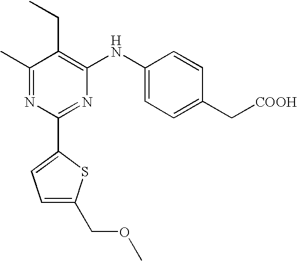 | 3.68 | 1 | 398 (M + H) | M, F, B, X, Y, C, c | e |
| 258 | 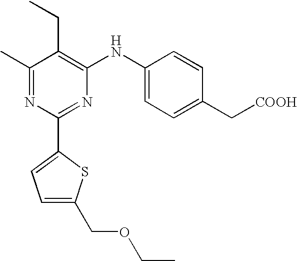 | 3.83 | 1 | 412 (M + H) | M, F, B, Y, C, c | e |
| 259 | 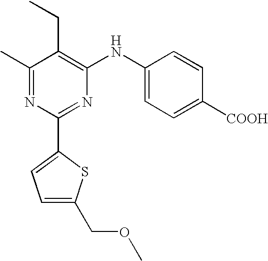 | 4.15 | 1 | 384 (M + H) | M, F, B, X, Y, C, c | e |
| 260 | 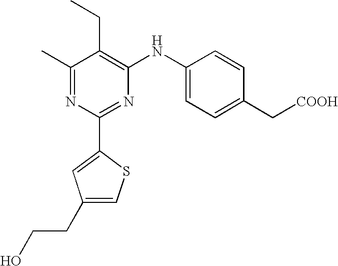 | 3.4 | 1 | 398 (M + H) | H, K, B, C, c, r, s | e |
| 261 | 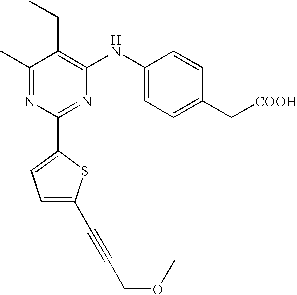 | 4.66 | 1 | 422 (M + H) | K, B, C, c, t, u | e |
| 262 | 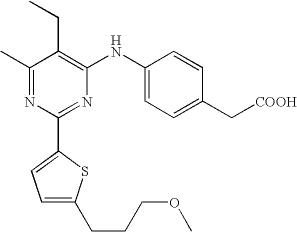 | 3.85 | 1 | 426 (M + H) | K, B, C, c, t, u, v | e |
| 263 | 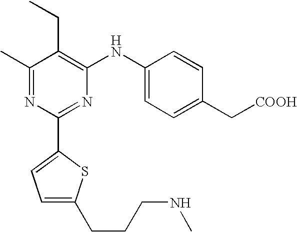 | 3.37 | 1 | 425 (M + H) | K, B, C, c, l, w | e |
| 264 | 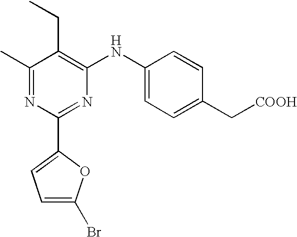 | 3.99 | 1 | 416 (M + H) | H, K, B, C, c | e |
| 265 | 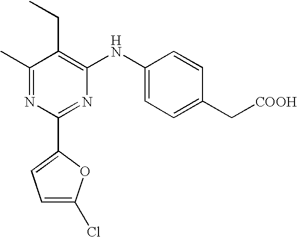 | 3.93 | 1 | 372 (M + H) | H, K, B, C, c | e |
| 266 | 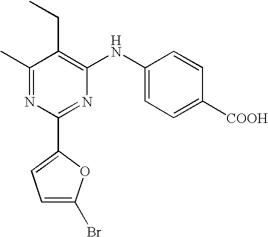 | 4.47 | 1 | 402 (M + H) | H, K, B, C, c | e |
| 267 | 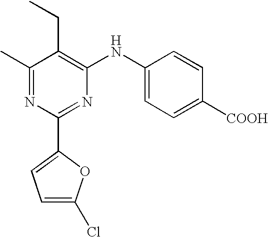 | 4.41 | 1 | 358 (M + H) | H, K, B, C, c | e |

### Reference Example 26

### 4-chloro-2-(4-ethoxythiophen-2-yl)-5-ethyl-6-methyl pyrimidine

5-(4-chloro-5-ethyl-6-methylpyrimidin-2-yl)thiophen-3-ol (49.1 mg) was dissolved in tetrahydrofuran (2 mL), and ethanol (35 μL) and triphenylphosphine (159 mg) were added to the solution. Diisopropylazodicarboxylate (120 μL) was added dropwise in nitrogen atmosphere at 0° C. with stirring for 5 minutes, and the reaction mixture was further stirred at room temperature overnight. Distilled water (1 mL) was added to the reaction mixture, and the reaction product was extracted with ethyl acetate. The organic layer was washed with saturated burin, and was concentrated after drying over anhydrous magnesium sulfate to obtain the titled compound (43.5 mg).

### Reference Example 27

### 4-chloro-5-ethyl-2-(5-chloromethylthiophen-2-yl)-6-methyl pyrimidine

The compound (61 mg) obtained in Reference Example 22 was dissolved in phosphorous oxychloride (4 mL), and the solution was stirred at 100° C. for 2.5 hours in nitrogen atmosphere. Saturated aqueous sodium hydrogen carbonate solution (5 mL) and water (25 mL) were added to the residue obtained by concentrating the reaction mixture, and the reaction product was extracted with ethyl acetate. The organic layer was washed with saturated burin, and concentrated after drying over anhydrous magnesium sulfate to obtain the titled compound (62 mg).

### Reference Example 28

### 4-chloro-5-ethyl-2-(5-dimethylaminomethylthiophen-2-yl)-6-methyl pyrimidine

The compound (32 mg) in Reference Example 27 was dissolved in tetrahydrofuran/N,N-dimethylformamide (1/1, 6 mL), and triethylamine (76 μg, manufactured by Wako Pure Chemical Industries, Inc.), potassium carbonate (75 mg, manufactured by Wako Pure Chemical Industries, Inc.) and a tetrahydrofuran solution of dimethylamine (2.0 M, 2.19 mL, manufactured by Aldrich Co.) were added to the solution with stirring at 50° C. for 61 hours. Water (25 mL) was added to the reaction mixture, and the reaction product was extracted with ethyl acetate. The organic layer was washed with saturated burin, and concentrated after drying over anhydrous magnesium sulfate. The residue obtained was purified by preparative thin layer chromatography (chloroform/methanol=20/1) to obtain the titled compound (9 mg).

### Reference Example 29

### methyl 2-(3-fluorophenyl)acetate

2-(3-fluorophenyl)acetic acid (5.53 g, manufactured by Tokyo Chemical Industry Co.) was dissolved in methanol (11 mL), and the solution was stirred at 80° C. for 1 hour by adding conc. hydrochloric acid (1.1 mL). The reaction mixture was concentrated, and was purified with a silica gel column (hexane/ethyl acetate=9/1 to 4/1) to obtain the titled compound (5.61 g).

### Reference Example 30

### methyl 2-(3-fluoro-4-nitrophenyl)acetate

Methyl 2-(3-fluorophenyl)acetate (10.76 g) was dissolved in conc. sulfuric acid (16.5 mL), and was stirred for 4 hours by adding conc. nitric acid (2.99 mL) cooled at 0° C. The reaction mixture was poured onto ice-water, and extracted with ethyl acetate. The organic layer obtained was washed with saturated burin, and was concentrated after drying over anhydrous sodium sulfate. The residue obtained was purified with a silica gel column (hexane/ethyl acetate=93/7 to 79/21) to obtain the titled compound (1.18 g).

### Reference Example 31

### Methyl 2-(4-amino-3-fluorophenyl)acetate

Methyl 2-(3-fluoro-4-nitrophenyl)acetate (532.1 mg) and nickel chloride (37.0 mg) were dissolved in methanol (26.6 mL), and the solution was cooled to 0° C. Sodium borohydride (354.2 mg) was added in three portions with stirring for 1 hour. After stopping the reaction by adding saturated aqueous sodium hydrogen carbonate solution to the reaction mixture, the reaction product was extracted with ethyl acetate. The residue obtained was purified by preparative thin layer chromatography (chloroform/methanol=99/1×2) to obtain the titled compound (312.0 mg).

### Reference Example 32

### 4-(2-(5-bromothiophen-2-yl)-5-ethyl-6-methylpyrimidin-4-ylamino)-N-(2-methoxypropan-2-yloxy)benzamide

(1-methoxy-1-methylethyl)oxyamine obtained by a method described in the published report (Tetrahedron, 1988, 44, 6013-6020) was dissolved in N,N-dimethylformamide (2 mL), and 4-(2-(5-bromothiophen-2-yl)-5-ethyl-6-methylpyrimidin-4-ylamino)benzoic acid (50 mg), triethylamine (22 μg, manufactured by Wako Pure Chemical Industries, Inc.), 1-ethyl-3-(3-dimethylaminopropyl)carbodiimide hydrochloride (30 mg, manufactured by Dojin Chemical Co.) and 1-hydroxybenzotriazole (21 mg, manufactured by Nacalai Tesque, Inc.) were sequentially added with stirring for 24 hours at. room temperature. Water (50 mL) was added to the reaction mixture, and the reaction product was extracted with ethyl acetate. The organic layer was washed with saturated burin, and was concentrated after drying over anhydrous magnesium sulfate. The residue obtained was purified by column chromatography (chloroform/methanol=19/1) to obtain the titled compound (40 mg).

### Reference Example 33

### 2-(3-methyl-4-nitrophenyl)acetic acid

2-(3-methyl-4-nitrophenyl)acetonitrile (232.4 mg) synthesized according to the method described in the published report (J. Org. Chem. 1995, 60, 6389-6396) was dissolved in conc. sulfuric acid (5 mL), and was stirred at 115° C. for 1.5 hours by adding water (5 ml). The reaction mixture was allowed to cool to room temperature, and was poured onto ice-water. The reaction reaction product was extracted with chloroform three times. The organic layer was concentrated after drying over anhydrous sodium sulfate to obtain the titled compound (226.6 mg).

### Reference Example 34

### 4-bromo-5-methylthiophene-2-carboxaldehyde.

5-methylthiophene-2-carboxaldehyde (1.01 g, manufactured by Tokyo Chemical Industry Co.) was dissolved in acetic acid (6 ml), and a solution prepared-by dissolving bromine (512 μL) in acetic acid (6 mL) was slowly added dropwise while shielding the light. The reaction mixture was stirred at room temperature for 18 hours, and slowly poured into an aqueous saturated sodium carbonate solution (100 mL). The reaction product was extracted with diethylether (50 mL), and the ether layer was sequentially washed with saturated aqueous sodium hydrogen carbonate solution and water followed by drying over anhydrous magnesium sulfate. The organic layer was concentrated, and the residue obtained was purified by column chromatography (hexane/ethyl acetate=20/1) to obtain the titled compound (775 mg).

### Reference Example 35

### ethyl 2-methoxyacetoacetate

Borontrifluoride diethylether complex (1.27 mL, manufactured by Tokyo Chemical Industry Co.) and ethyl acetoacetate (1.28 mL, manufactured by Tokyo Chemical Industry Co.) were sequentially added to a suspension of iodobenzene (2.20 g, manufactured by Tokyo Chemical Industry Co.) in methanol (40 mL) with stirring at room temperature for 6.5 hours in nitrogen atmosphere. The reaction mixture was concentrated, and saturated aqueous sodium hydrogen carbonate solution (20 mL) was added to the residue. The reaction product was extracted with dichloromethane, and the organic layer was concentrated after drying over anhydrous magnesium sulfate. The residue was purified by column chromatography (hexane/ethyl acetate=5/1) to obtain the titled compound. (1.27 g).

### Reference Example 36

### methyl 2-(4-(tert-butoxycarbonyl)-2-nitrophenyl)acetate

Methyl 2-(4-bromo-2-nitrophenyl)acetate (1.1662 g) synthesized according to a known methyl esterification method was dissolved in 1,4-dioxane (40 mL), and cesium carbonate (1.9398 g), tris(benzylideneacetone)palladium (0) (393.7 mg), Xanthophos (991.4 mg, commercially available) and tertiary-butyl carbamate (506.7 mg) were added to the solution. The mixture was stirred at 100° C. for 17.5 hours in nitrogen atmosphere. The reaction solution was filtered after adding a large excess of methylene chloride, and the filtrate&was concentrated. The residue obtained was purified with a silica gel column (hexane/ethyl acetate=4/1 to 2/1) to obtain the titled compound (1.0659 g).

### Reference Example 37

### methyl 2-(4-amino-2-nitrophenyl)acetate

Methyl 2-(4-(tert-butoxycarbonyl)-2-nitrophenyl)acetate (202.6 mg) was dissolved in 4 N hydrochloric acid/ethyl acetate solution (4.72 mL) with stirring at room temperature for 3 hours: Diethylether was added to the reaction mixture, and the precipitated solid was filtered and washed with diethylether. The residue obtained was dried in vacuum to obtain the titled compound (118.9 mg).

### Reference Example 38

### 2-(2-methoxy-4-nitrophenyl)acetonitrile

Tris(benzylideneacetone)dipalladium (0)(207.3 mg) and xanthophos (259.0 mg, commercially available) were added to 1-bromo-2-methoxy-4-nitrobenzene (3.99 g, manufactured by Wako Pure Chemical Industries, Inc.), and a solution of trimethylsilyl acetonitrile (2.9523 g, manufactured by Aldrich Co.) dissolved in DMF (25 ml) was added in nitrogen atmosphere. After stirring the solution at room temperature for a while, zinc fluoride (1.3231 g, manufactured by Aldrich Co.) was added, and the mixture was stirred at 90° C. for 8 hours in nitrogen atmosphere. The mixture was concentrated, and the residue was purified with a silica gel column (hexane/ethyl acetate=4/1 to 2/1) to obtain the titled compound (1.0346 g).

### Reference Example 39

### Ethyl 2-(methylthio)thiazole-5-carboimidate

2-bromothiazole-5-carbonitrile (300 mg) was dissolved in ethanol (10 mL), and sodium thiomethoxide (334 mg, manufactured by Aldrich Co.) was added to the solution with stirring for 4 hours at room temperature. The reaction mixture was concentrated, and water (50 mL) was added to the residue, which was extracted with ethyl acetate. The organic layer was washed with saturated burin, and was concentrated after drying over anhydrous magnesium sulfate. The residue obtained was purified by column chromatography (hexane/ethyl acetate=1/1) to obtain the titled compound (231 mg).

### Reference Example 40

### 5-(bromomethyl)-2-(5-chlorothiophen-2-yl)-6-methylpyrimidin-4(3H)-one

N-bromosuccimide (444.4mg) and 2,2′-azobis(isobtylonitrile) (34.0 mg) were added to 2-(5-chlorothiophen-2-yl)-5,6-dimethylpyrimidin-4(3H)-one (499.5 mg), the mixture was dissolved in carbon tetrachloride (10 mL)and heated at 100° C. with stirring for 2 hours. The reaction mixture was filtered while it is still hot, and the filter was washed with chloroform. The residue obtained was dried in vacuum to obtain the titled compound (468.4 mg).

### Example 268

### 4-(2-(5-bromothiophen-2-yl)-5-ethyl-6-methylpyrimidin-4-ylamino)-N-hydroxybenzamide

The compound (40 mg) obtained in Reference Example 32 was dissolved in methanol (2 mL), and the solution was stirred at room temperature for 1 hour after adding 2 N hydrochloric acid (70 μL, manufactured by Wako Pure Chemical Industries, Inc.). The residue obtained by concentrating the reaction mixture was washed with a mixed solvent of diethylether—methanol to obtain the titled compound (22 mg). LC-MS:HPLC retention time 4.00 minutes (LC condition 1), m/z 433 (M +H).

### Example 269

### ethyl 2-(4-(2-(5-chloro-4-methylthiophen-2-yl)-5,6-diethylpyrimidin-4-ylamino)phenyl)acetate

Ethyl 2-(4-(2-(5-bromo-4-methylthiophen-2-yl)-5,6-diethylpyrimidin-4-ylamino)phenyl)acetate (51 mg) was dissolved in N,N-dimethylformamide (2 mL), and copper (I) chloride (21 mg, manufactured by Wako Pure Chemical Industries, Inc.) was added with stirring at 110° C. for 71 hours in nitrogen atmosphere. Water (25 mL) and 28% aqueous ammonia (1 mL) were added to the reaction mixture, and the solution was extracted with ethyl acetate. The organic layer was washed with saturated burin, and concentrated after drying over magnesium sulfate. The residue obtained was purified by column chromatography (hexane/ethyl acetate=3/1) to obtain the titled compound (32.mg). LC-MS:HPLC retention time 6.94 minutes (LC condition 2), m/z 444 (M+H).

### Example 270

### methyl 2-(4-(2-(5-chlorothiophen-2-yl)-5-ethyl-6-methylpyrimidin-4-ylamino)-2-hydroxyphenyl)acetate

Pyridinium hydrochloride (191.5.mg) was added to methyl 2-(4-(2-(5-chlorothiophen-2-yl)-5-ethyl-6-methylpyrimidin-4-ylamino)-2-methoxyphenyl)acetate (9.0 mg) with stirring at 200° C. for 5 minutes. After allowing the reaction mixture to cool to room temperature, water was added to the reaction mixture. The reaction product was extracted with ethyl acetate, and the organic layer was concentrated. The residue obtained was purified by preparative thin layer chromatography (chloroform/methanol=90/10) to obtain the titled compound (4.0 mg). LC-MS:HPLC retention time 5.01 minutes (LC condition 1), m/z 418 (M+H).

### Example 271

### ethyl 2-(4-(5-ethyl-6-methyl-2-(5-(methylsulfonyl)thiphen-2-yl)pyrimidin-4-ylamino)-phenyl)acetate

Ethyl 2-(4-(5-ethyl-6-methyl-2-(5-(methylthio)-thiophen-2-yl)pyrimidin-4-ylamino)-phenyl)acetate (75 mg) was dissolved in dichloromethane (5 mL), and 3-chloroperbenzoic acid (66 mg, manufactured by Tokyo Chemical Industry Co.) was added with stirring at room temperature for 40 minutes. Aqueous solution (5%, 5 mL) of sodium sulfite and water (10-mL) were added to the reaction mixture, and the reaction product was extracted with dichloromethane. The organic layer was washed with saturated aqueous sodium hydrogen carbonate solution, and was concentrated after drying over anhydrous magnesium sulfate. The residue obtained was purified by preparative thin layer chromatography (hexane/ethyl acetate=1/2) to obtain the titled compound (49 mg). LC-MS:HPLC retention time 5.01 minutes (LC condition 1), m/z 460 (M+H).

### Example 272

### methyl 2-(4-(2-(5-(dimethylamino)-4-methylthiophen-2-yl)-5-ethyl-6-methylpyrimidin-4-ylamino)phenyl)acetate

Methyl 2-(4-(2-(5-amino-4-methylthiophen-2-yl)-5-ethyl-6-methylpyrimidin-4-ylamino)phenyl)acetate (16 mg) was dissolved in methanol (2 mL), and 37% formaldehyde (48 μL, manufactured by Wako Pure Chemical Industries, Inc.) and sodium cyanoborohydide (19 mg, manufactured by Tokyo Chemical Industry Co.) were added at 60° C. followed by stirring for 12 hours. Water (15 mL) and saturated aqueous sodium hydrogen carbonate solution (5 mL) were added to the reaction mixture, and the reaction product was extracted with chloroform. The organic layer was washed with saturated brine, and was concentrated after drying over anhydrous magnesium sulfate to obtain the titled compound (10 mg). LC-MS:HPLC retention time 3.81 minutes (LC condition 1), m/z 425 (M+H).

### Production Method of Intermediate Compounds Used in Examples 268 to 272

The production methods used in Examples 268 to 272 are shown in Table 4. Notations in Table 4 mean as follows: Exp; No of example, Ref; production method of corresponding intermediate compound. Notations in the column of Ref denote production methods of following intermediate compounds:
A; production method shown in Reference Example 1,
B; production method shown in Reference Example 2,
C; production method shown in Reference Example 3,
D; production method shown in Reference Example 4,
E; production method shown in Reference Example 5,
F; production method shown in Reference Example 6,
G; production method shown in Reference Example 7,
H; production method shown in Reference Example 8,
I; production method shown in Reference Example 9,
J; production method shown in Reference Example 10,
K; production method shown in Reference Example 11,
L; production method shown in Reference Example 12,
M; production method shown in Reference Example 13,
N; production method shown in Reference Example 14,
O; production method shown in Reference Example 15,
P; production method shown in Reference Example 16,
Q; production method shown in Reference Example 17,
R; production method shown in Reference Example 18,
S; production method shown in Reference Example 19,
T; production method shown in Reference Example 20,
U; production method shown in Reference Example 21,
V; production method shown in Reference Example 22,
W; production method shown in Reference Example 23,
X; production method shown in Reference Example 24,
Y; production method-shown in Reference Example 25,
Z; production method shown in Reference Example 26,
AA; production method shown in Reference Example 27,
AB; production method shown in Reference Example 28,
AC; production method shown in Reference Example 29,
AD; production method shown in Reference Example 30,
AE; production method shown in Reference Example 31,
AF; production method shown in Reference Example 32,
AG; production method shown in Reference Example 33,
AH; production method shown in Reference Example 34,
AI; production method shown in Reference Example 35,
AJ; production method shown in Reference Example 36,
AK; production method shown in Reference Example 37,
AL; production method shown in Reference Example 38,
AM; production method shown in Reference Example 39,
AN; production method shown in Reference Example 40,
a; production method show I Example 1,
b; production method show I Example 28,
c; production method show I Example 29,
d; production method show I Example 30,
e; production method show I Example 31,
f; production method show I Example 32,
g; production method show I Example 33,
h; production-method show I Example 34,
i; production method show I Example 35,
j; production method show I Example 36,
k; production method show I Example 37,
l; production method show I Example 38,
m; production method show I Example 39,
n; production method show I Example 40,
o; production method show I Example 41,
p; production method show I Example 42,
q; production method show I Example 43,
r; production method show I Example 44,
s; production method show I Example 45,
t; production method show I Example 46,
u; production method show I Example 47,
v; production method show I Example 48,
w; production method show I Example 49,
x; production method show I Example 268,
y; production method show I Example 269,
z; production method show I Example 270,
aa; production method show I Example 271, and

ab; production method show I Example 272.

TABLE 4

| Exp. | Ref. |
|---|---|
| 268 | K, B, C, c, e, AF |
| 269 | H, K, B, C, c |
| 270 | AL, AG, AE, AC, H, K, B, C, c |
| 271 | J, B, C, c |
| 272 | H, K, B, C, c, i |

### Examples 273 to 389

The production methods used in Examples 273 to 389 are shown in Table 5. Notations in Table 5 mean as follows: Exp; No of example, Str.; compounds in examples, RT; retention time (minutes) of liquid chromatography in LCMS, LC; solvent conditions of liquid chromatography in LCMS, MS; mass spectrum data in LCMS, Ref; production method of corresponding intermediate compound, Ex; production method of compound in corresponding example.

Notations in the column of Ref and EX denote the following production methods:
A; production method shown in Example 1,
B; production method shown in Example 2,
C; production method shown in Example 3,
D; production method shown in Example 4,
E; production method shown in Example 5,
F; production method shown in Example 6,
G; production method shown in Example 7,
H; production method shown in Example 8,
I; production method shown in Example 9,
J; production method shown in Example 10,
K; production method shown in Example 11,
L; production method shown in Example 12,
M; production method shown in Example 13,
N; production method shown in Example 14,
p; production method shown in Example 15,
P; production method shown in Example 16,
Q; production method shown in Example 17,
R; production method shown in Example 18,
S; production method shown in Example 19,
T; production method shown in Example 20,
U; production method shown in Example 21,
V; production method shown in Example 22,
W; production method shown in Example 23,
X; production method shown in Example 24,
Y; production method shown in Example 25,
Z; production method shown in Example 26,
AA; production method shown in Example 27,
AB; production method shown in Example 28,
AC; production method shown in Example 29,
AD; production method shown in Example 30,
AE; production method shown in Example 31,
AF; production method shown in Example 32,
AG; production method shown in Example 33,
AH; production method shown in Example 34,
AI; production method shown in Example 35,
AJ; production method shown in Example 36,
AK; production method shown in Example 37,
AL; production method shown in Example 38,
AM; production method shown in Example 39,
AN; production method shown in Example 40,
a; production method show in Example 1,
b; production method show in Example 28,
c; production method show in Example 29,
d; production method show in Example 30,
e; production method show in Example 31,
f; production method show in Example 32,
g; production method show in Example 33,
h; production method show in Example 34,
i; production method show in Example 35,
j; production method show in Example 36,
k; production method show in Example 37,
l; production method show in Example 38,
m; production method show in Example 39,
n; production method show in Example 40,
o; production method show in Example 41,
p; production method show in Example 42,
q; production method show in Example 43,
r; production method show in Example 44,
s; production method show in Example 45,
t; production method show in Example 46,
u; production method show in Example 47,
v; production method show in Example 48,
w; production method show in Example 49,
x; production method show in Example 268,
y; production-method show in Example 269,
z; production method show in Example 270,
aa; production method show in Example 271, and

ab; production method show in Example 272.

TABLE 5

| Exp. | Str. | R.T. | LC | MS | Ref. | Ex. |
|---|---|---|---|---|---|---|
| 273 | 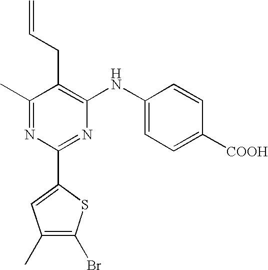 | 5.17 | 3 | 444 (M + H) | H, K, B, C, c | e |
| 274 | 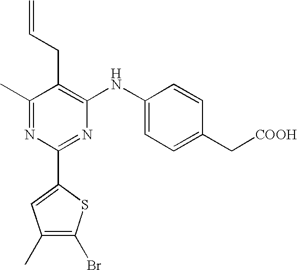 | 4.70 | 3 | 458 (M + H) | H, K, B, C, c | e |
| 275 | 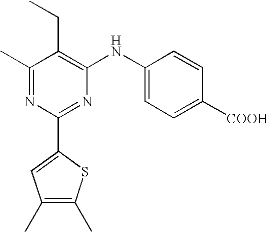 | 3.25 | 3 | 368 (M + H) | H, F, B, C | a |
| 276 | 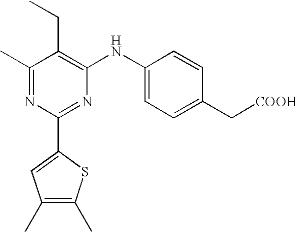 | 2.52 | 3 | 382 (M + H) | H, F, B, C | a |
| 277 | 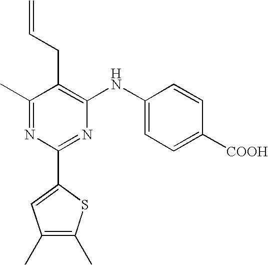 | 3.80 | 3 | 380 (M + H) | H, F, B, C | a |
| 278 | 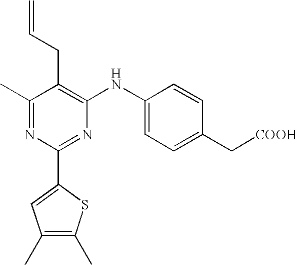 | 2.97 | 3 | 394 (M + H) | H, F, B, C | a |
| 279 | 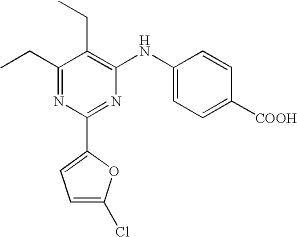 | 4.70 | 2 | 372 (M + H) | H, K, B, C, c | e |
| 280 | 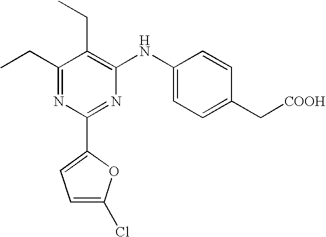 | 3.92 | 2 | 386 (M + H) | H, K, B, C, c | e |
| 281 | 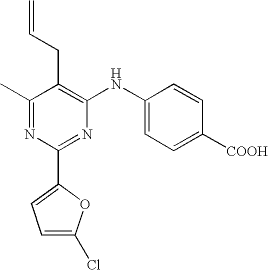 | 4.77 | 2 | 370 (M + H) | H, K, B, C, c | e |
| 282 | 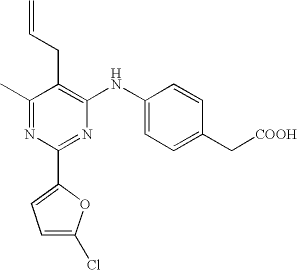 | 4.22 | 2 | 384 (M + H) | H, K, B, C, c | e |
| 283 | 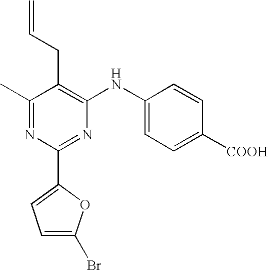 | 3.88 | 2 | 414 (M + H) | H, K, B, C, c | e |
| 284 | 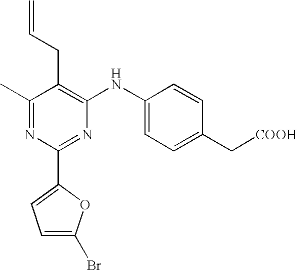 | 3.22 | 2 | 428 (M + H) | H, K, B, C, c | e |
| 285 | 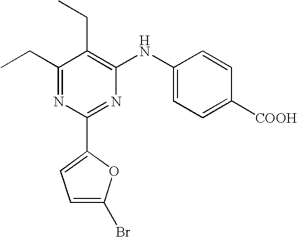 | 4.71 | 2 | 416 (M + H) | H, K, B, C, c | e |
| 286 | 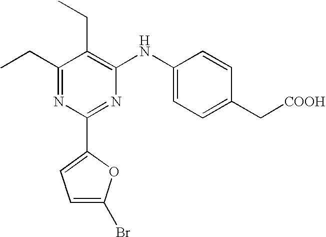 | 3.97 | 2 | 430 (M + H) | H, K, B, C, c | e |
| 287 | 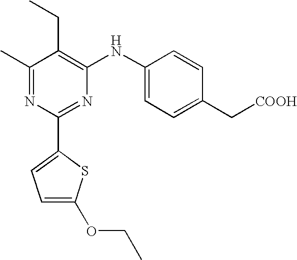 | 3.74 | 1 | 398 (M + H) | D, E, F, B, C, U, Z, c | e |
| 288 | 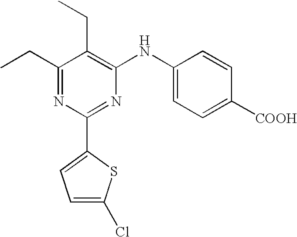 | 5.56 | 2 | 368 (M + H) | H, K, B, C, c | e |
| 289 | 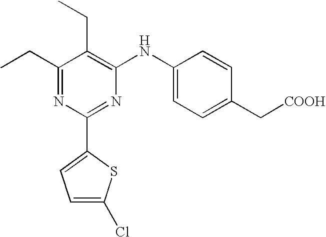 | 5.04 | 2 | 402 (M + H) | H, K, B, C, c | e |
| 290 | 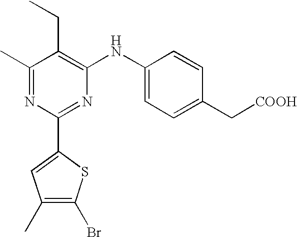 | 4.70 | 2 | 446 (M + H) | H, K, B, C, c | e |
| 291 | 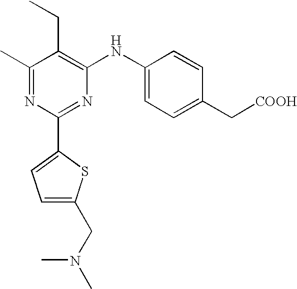 | 2.72 | 1 | 411 (M + H) | M, F, B, W, V, AA, AB, c | e |
| 292 | 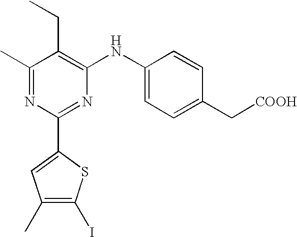 | 4.65 | 2 | 494 (M + H) | H, K, B, C, c, t | e |
| 293 | 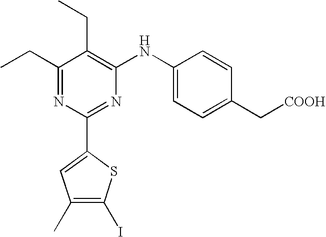 | 5.32 | 2 | 508 (M + H) | H, K, B, C, c, t | e |
| 294 | 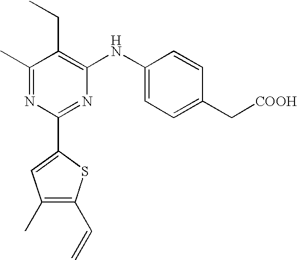 | 3.84 | 2 | 394 (M + H) | H, K, B, C, c, r | e |
| 295 | 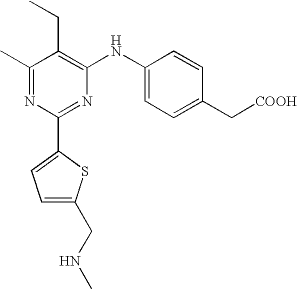 | 3.16 | 1 | 397 (M + H) | M, F, B, W, V, AA, AB, c | e |
| 296 | 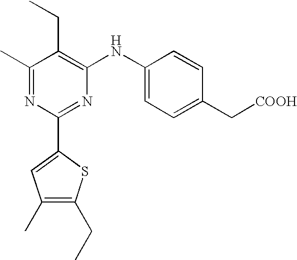 | 3.35 | 2 | 396 (M + H) | H, K, B, C, c, r, h | e |
| 297 | 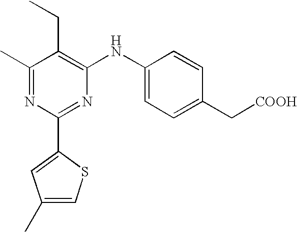 | 3.19 | 2 | 368 (M + H) | H, K, B, C, c, h | e |
| 298 | 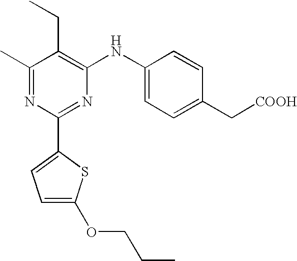 | 3.94 | 1 | 412 (M + H) | D, E, F, B, C, U, Z, c | e |
| 299 | 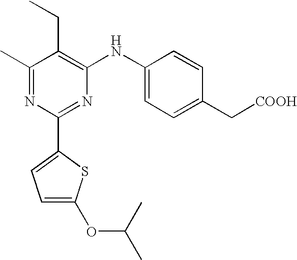 | 3.83 | 1 | 412 (M + H) | D, E, F, B, C, U, Z, c | e |
| 300 | 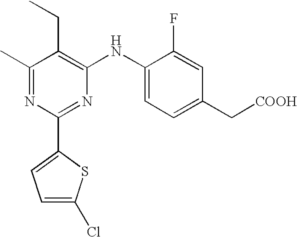 | 4.99 | 1 | 406 (M + H) | H, K, B, C, AC, AD, AE, c | e |
| 301 | 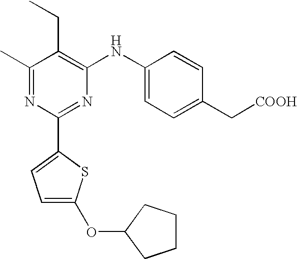 | 4.02 | 1 | 438 (M + H) | D, E, F, B, C, U, Z, c | e |
| 302 | 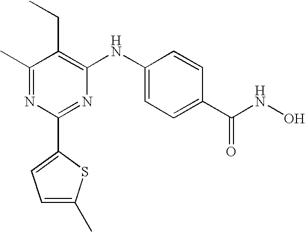 | 3.11 | 1 | 383 (M + H) | H, F, B, C, a, AF | x |
| 303 | 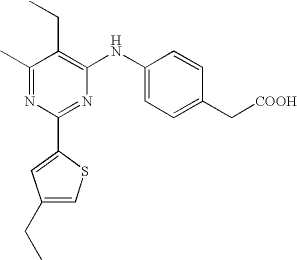 | 3.42 | 2 | 382 (M + H) | H, K, B, C, c, r, h | e |
| 304 | 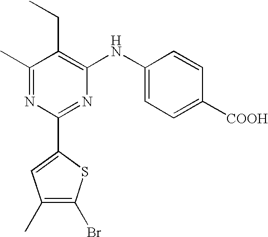 | 5.50 | 2 | 432 (M + H) | H, K, B, C, c | e |
| 305 | 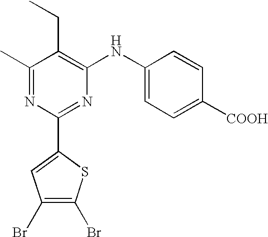 | 6.17 | 2 | 498 (M + H) | H, K, B, C, c | e |
| 306 | 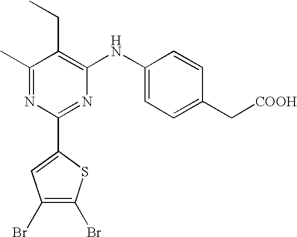 | 5.87 | 2 | 510 (M + H) | H, K, B, C, c | e |
| 307 | 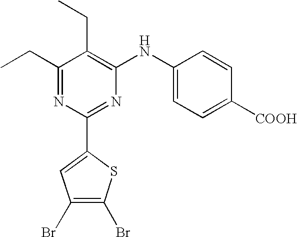 | 6.86 | 2 | 510 (M + H) | H, K, B, C, c | e |
| 308 | 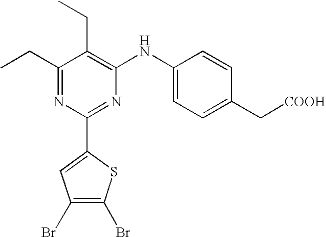 | 6.47 | 2 | 524 (M + H) | H, K, B, C, c | e |
| 309 | 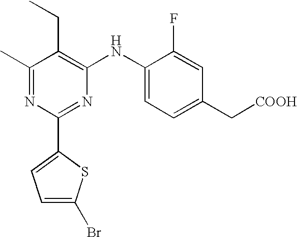 | 4.81 | 2 | 450 (M + H) | K, B, C, AC, AD, AE, c | e |
| 310 | 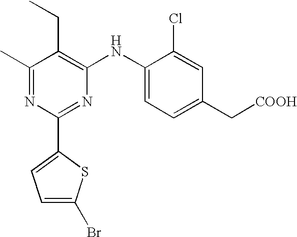 | 5.63 | 2 | 466 (M + H) | K, B, C, AC, AD, AE, c | e |
| 311 | 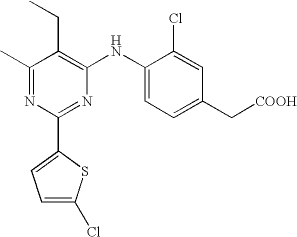 | 5.55 | 2 | 422 (M + H) | H, K, B, C, AC, AD, AE, c | e |
| 312 | 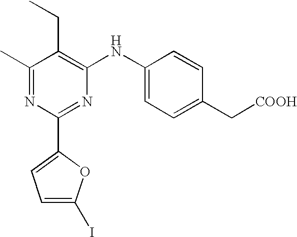 | 3.65 | 1 | 484 (M + 1) | H, K, B, C, c | e |
| 313 | 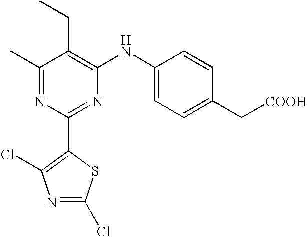 | 5.01 | 1 | 422 (M + 1) | H, K, B, C, c | e |
| 314 | 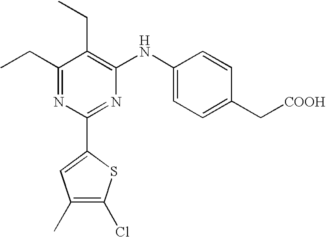 | 5.43 | 2 | 416 (M + H) | H, K, B, C, c, y | e |
| 315 | 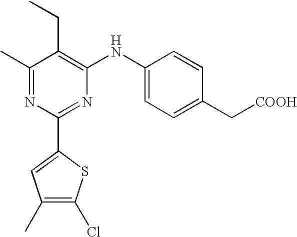 | 4.67 | 2 | 402 (M + H) | H, K, B, C, c, y | e |
| 316 | 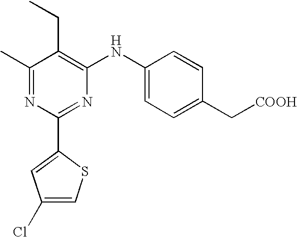 | 4.52 | 2 | 388 (M + H) | H, K, B, C, c, y | e |
| 317 | 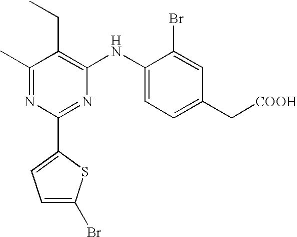 | 5.73 | 2 | 510 (M + H) | K, B, C, AC, AD, AE, c | e |
| 318 | 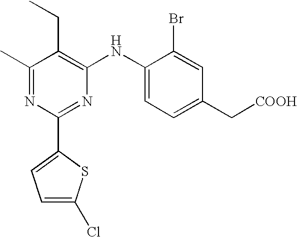 | 5.61 | 2 | 466 (M + H) | H, K, B, C, AC, AD, AE, c | e |
| 319 | 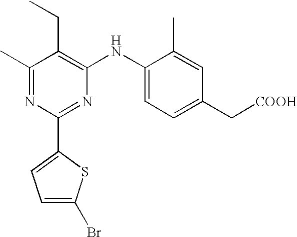 | 4.09 | 2 | 446 (M + H) | K, B, C, AD, AG, AC, Q, c | e |
| 320 | 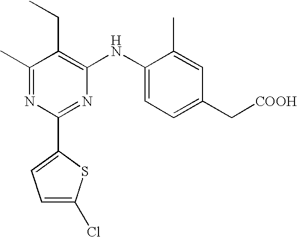 | 3.92 | 2 | 402 (M + H) | H, K, B, C, AD, AG, AC, Q, c | e |
| 321 | 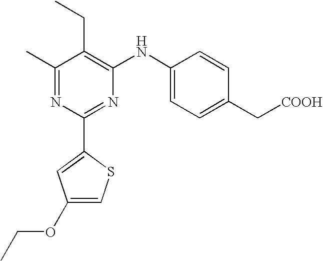 | 4.15 | 1 | 398 (M + H) | M, N, O, E, K, B, U, Z, c | e |
| 322 | 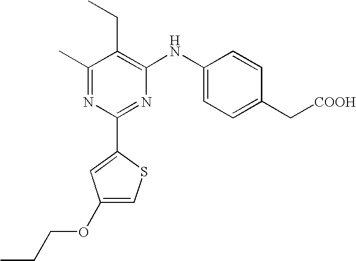 | 4.43 | 1 | 412 (M + H) | M, N, O, E, K, B, U, Z, c | e |
| 323 | 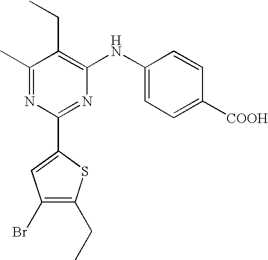 | 5.83 | 2 | 446 (M + H) | H, K, B, C, c | e |
| 324 | 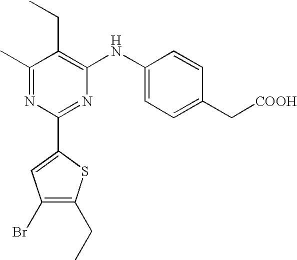 | 5.25 | 1 | 460 (M + H) | H, K, B, C, c | e |
| 325 | 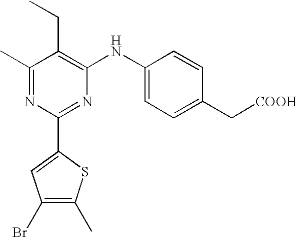 | 4.95 | 1 | 446 (M + 1) | AH, H, K, B, C, c | e |
| 326 | 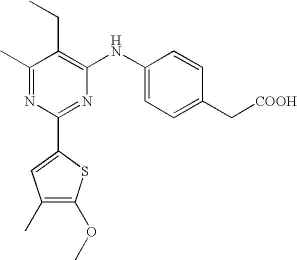 | 3.47 | 1 | 398 (M + 1) | M, N, O, H, K, B, C, c | e |
| 327 | 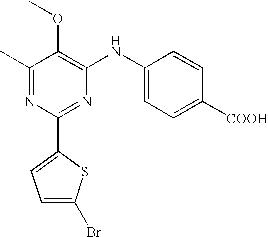 | 5.10 | 1 | 420 (M + H) | AI, K, B, C, c | e |
| 328 | 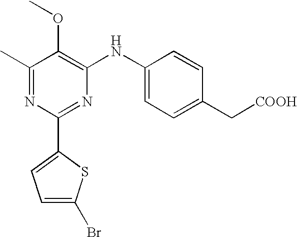 | 4.92 | 1 | 434 (M + H) | AI, K, B, C, c | e |
| 329 | 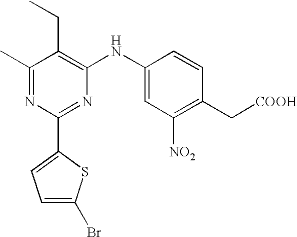 | 5.69 | 1 | 477 (M + H) | AJ, AK, K, B, C, c | e |
| 330 | 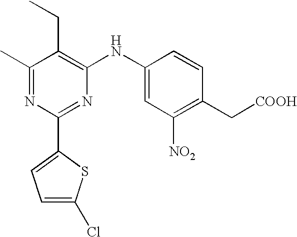 | 5.57 | 1 | 433 (M + H) | AJ, AK, H, K, B, C, c | e |
| 331 | 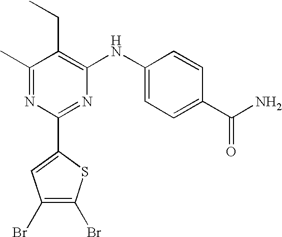 | 5.58 | 1 | 495 (M + H) | H, K, B, C, c, e | g |
| 332 | 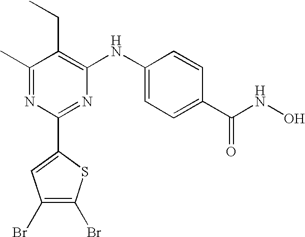 | 5.16 | 1 | 511 (M + H) | H, K, B, C, c, e, AF | x |
| 333 | 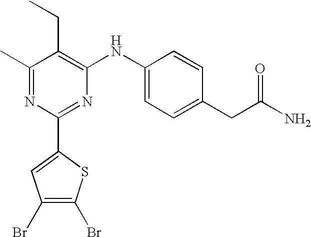 | 5.31 | 1 | 509 (M + H) | H, K, B, C, c, e | g |
| 334 | 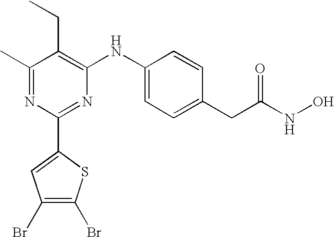 | 4.98 | 1 | 525 (M + H) | H K, B, C, c, e, AF | x |
| 335 | 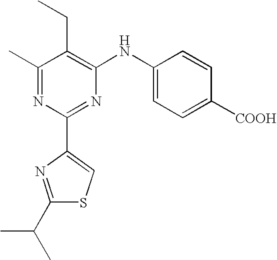 | 3.10 | 1 | 383 (M + H) | H, K, B, C, c | e |
| 336 | 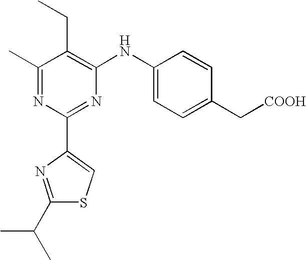 | 3.04 | 1 | 397 (M + H) | H, K, B, C, c | e |
| 337 | 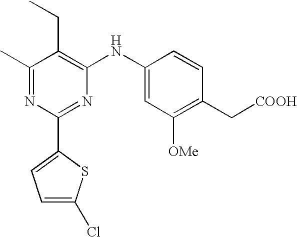 | 4.79 | 1 | 418 (M + H) | AL, AG, AC, AE, H, K, B, C, c | e |
| 338 | 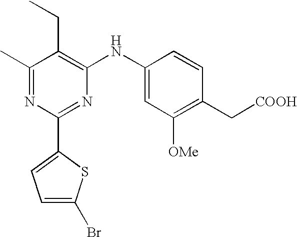 | 4.88 | 1 | 462 (M + H) | AL, AG, AC, AE, K, B, C, c | e |
| 339 | 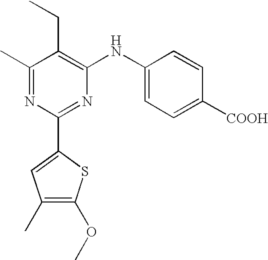 | 3.65 | 1 | 384 (M + 1) | M, N, O, H, K, B, C, c | e |
| 340 | 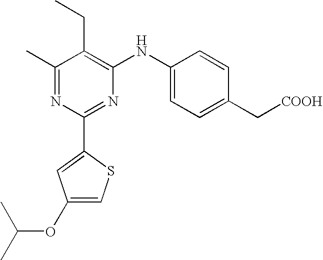 | 4.23 | 1 | 412 (M + H) | M, N, O, E, K, B, U, Z, c | e |
| 341 | 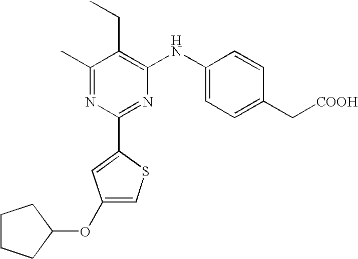 | 4.68 | 1 | 438 (M + H) | M, N, O, E, K, B, U, Z, c | e |
| 342 | 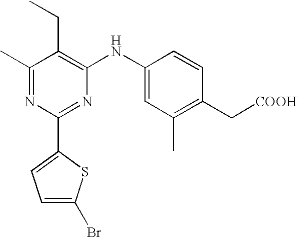 | 4.81 | 2 | 446 (M + H) | AL, AG, AC, AE, H, K, B, C, c | e |
| 343 | 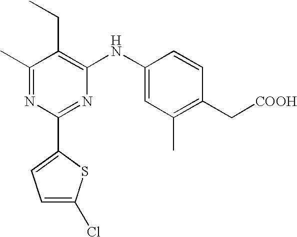 | 4.69 | 2 | 402 (M + H) | AL, AG, AC, AE, K, B, C, c | e |
| 344 | 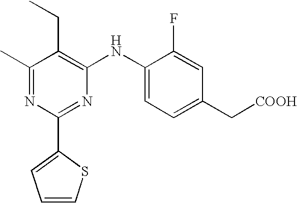 | 3.38 | 1 | 372 (M + H) | K, B, C, AC, AD, AE, c, h | e |
| 345 | 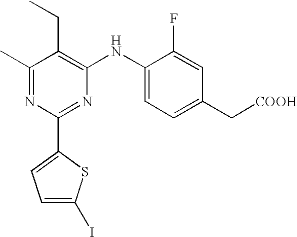 | 4.93 | 1 | 498 (M + H) | K, B, C, AC, AD, AE, c, t | e |
| 346 | 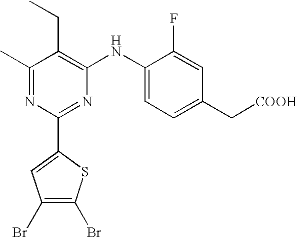 | 6.09 | 2 | 528 (M + H) | H, K, B, C, AC, AD, AE, c | e |
| 347 | 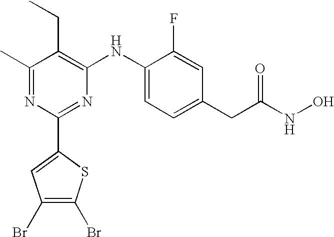 | 5.16 | 2 | 543 (M + H) | K, B, C, AC, AD, AE, c, e, AF | x |
| 348 | 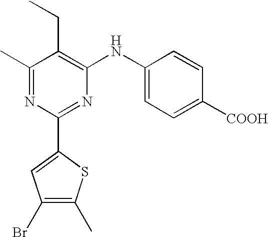 | 5.65 | 1 | 432 (M + 1) | AH, H, K, B, C, c | e |
| 349 | 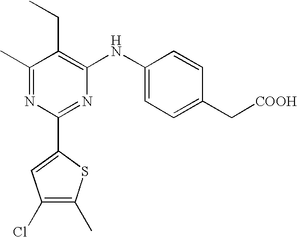 | 4.91 | 1 | 402 (M + 1) | AH, H, K, B, C, c, y | e |
| 350 | 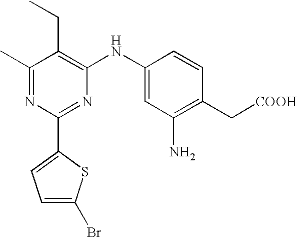 | 4.32 | 1 | 446 (M + H) | AJ, AK, K, B, C, c, AE | e |
| 351 | 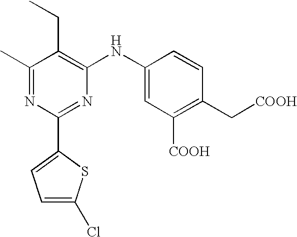 | 4.51 | 1 | 432 (M + H) | H, K, B, C, AD, h | a |
| 352 | 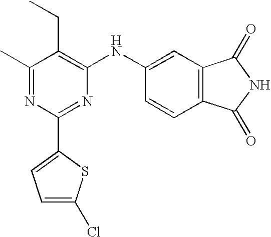 | 5.77 | 1 | 399 (M + H) | H, K, B, C | a |
| 353 | 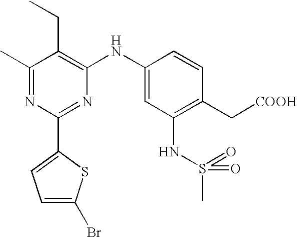 | 4.60 | 1 | 525 (M + H) | AJ, AK, K, B, C, c, AE, j | e |
| 354 | 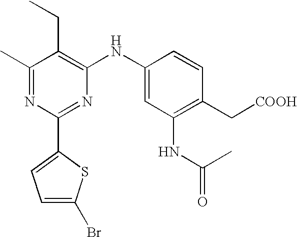 | 4.14 | 1 | 489 (M + H) | AJ, AK, K, B, C, c, AE, j | e |
| 355 | 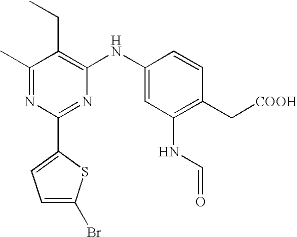 | 4.32 | 1 | 474 (M + H) | AJ, AK, K. B, C, c, AE, j | e |
| 356 | 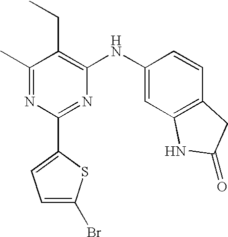 | 4.67 | 1 | 429 (M + H) | AJ, AK, K, B, C, c, AE, j | e |
| 357 | 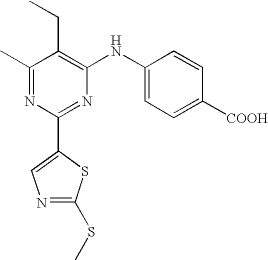 | 4.70 | 1 | 387 (M + H) | H, AM, K, B, C, c | e |
| 358 | 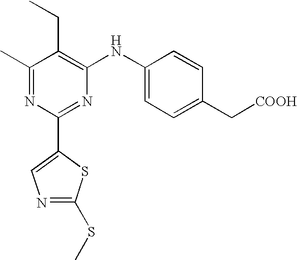 | 4.20 | 1 | 401 (M + H) | H, AM, K, B, C, c | e |
| 359 | 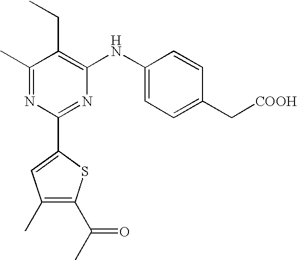 | 4.75 | 1 | 410 (M + H) | H, K, B, C, c, l | e |
| 360 | 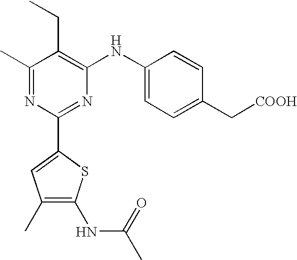 | 2.99 | 1 | 425 (M + H) | H, K, B, C, c, i, j | e |
| 361 | 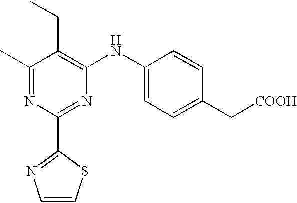 | — | — | — | H, K, B, C, c | e |
| 362 | 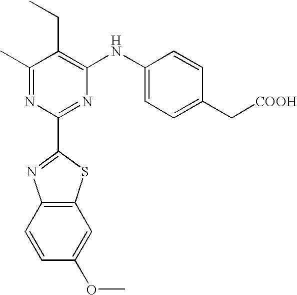 | — | — | — | H, K, B, C, c | e |
| 363 | 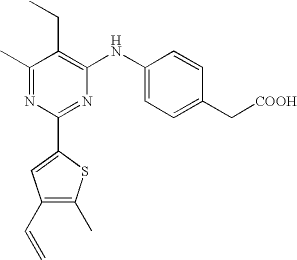 | 4.05 | 1 | 394 (M + 1) | AH, H, K, B, C, c, r | e |
| 364 | 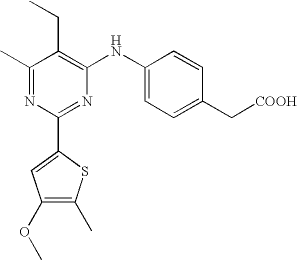 | 3.48 | 1 | 398 (M + 1) | AH, M, N, O, H, K, B, C, c | e |
| 365 | 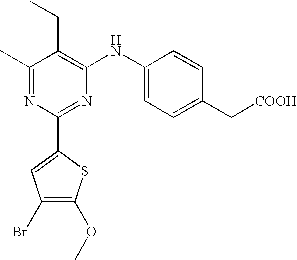 | 4.33 | 1 | 462 (M + 1) | AH, K, B, C, c | e |
| 366 | 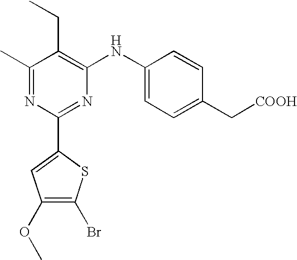 | 4.86 | 1 | 462 (M + 1) | M, N, O, AN, H, K, B, C, c | e |
| 367 | 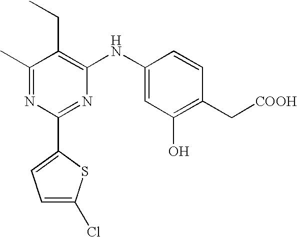 | 4.34 | 1 | 404 (M + H) | AL, AG, AE, AC, H, K, B, C, c, z | e |
| 368 | 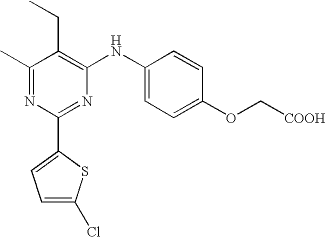 | 4.01 | 1 | 404 (M + H) | Q, H, K, B, C | a |
| 369 | 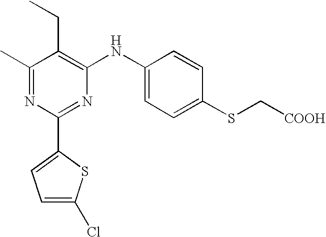 | 5.17 | 1 | 420 (M + H) | H, K, B, C, c | a |
| 370 | 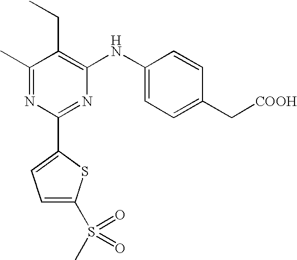 | 4.13 | 1 | 432 (M + H) | J, B, C, c, aa | e |
| 371 | 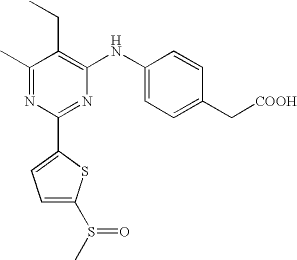 | 3.32 | 1 | 416 (M + H) | J, B, C, c, aa | e |
| 372 | 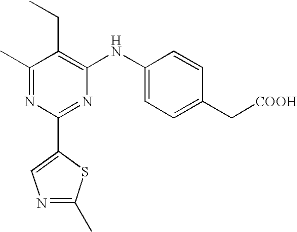 | 3.45 | 1 | 369 (M + 1) | H, K, B, C, c | e |
| 373 | 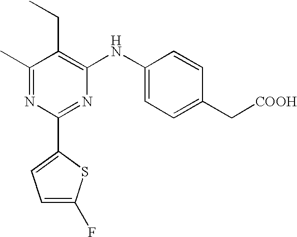 | 4.1 | 1 | 372 (M + 1) | K, B, C, c | e |
| 374 | 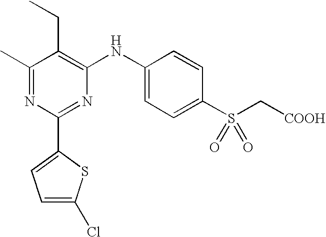 | 5.61 | 1 | 452 (M + H) | H, K, B, C, c, a | aa |
| 375 | 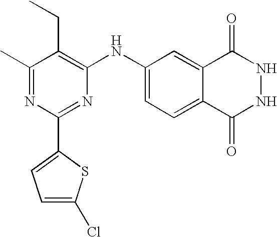 | 4.93 | 1 | 414 (M + 1) | H, K, B, C | c |
| 376 | 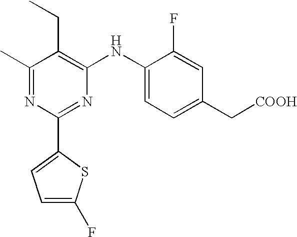 | 4.26 | 1 | 390 (M + H) | K, B, C, AC, AD, AE, c | e |
| 377 | 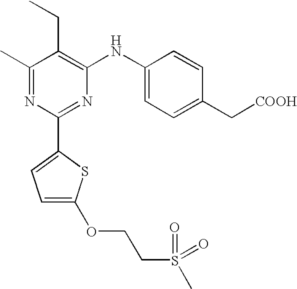 | 2.91 | 1 | 476 (M + H) | D, E, F, B, C, U, Z | a |
| 378 | 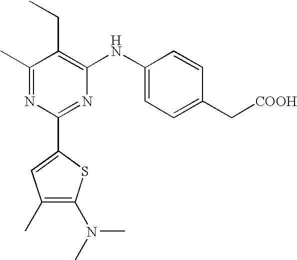 | 3.32 | 1 | 411 (M + H) | H, K, B, C, c, i, ab | e |
| 379 | 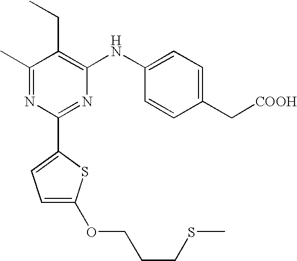 | 3.79 | 2 | 458 (M + H) | D, E, F, B, C, U, c, Z | e |
| 380 | 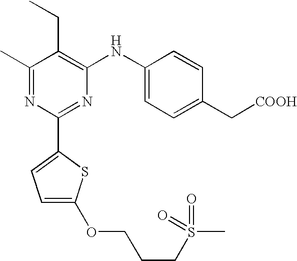 | 3.10 | 1 | 490 (M + H) | D, E, F, B, C, U, c, Z, e | aa |
| 381 | 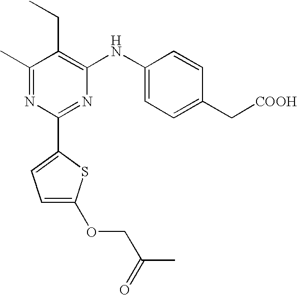 | 3.05 | 1 | 426 (M + H) | D, E, F, B, C, U, c, Z | e |
| 382 | 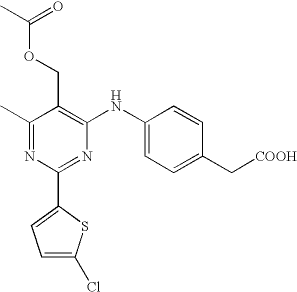 | 5.13 | 1 | 432 (M + H) | H, K, B, AN, C | a |
| 383 | 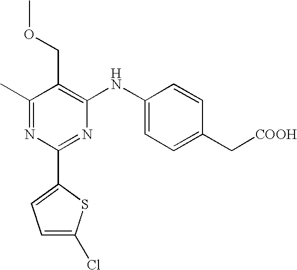 | 5.10 | 1 | 404 (M + H) | H, K, B, AN, C, c | e |
| 384 | 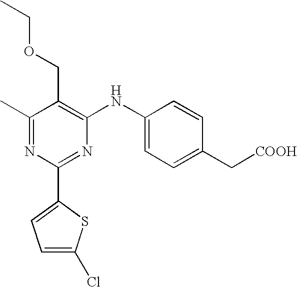 | 5.38 | 1 | 418 (M + H) | H, K, B, AN, C, c | e |
| 385 | 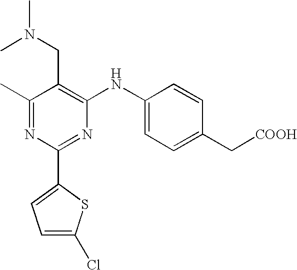 | 3.59 | 1 | 417 (M + H) | H, K, B, AN, C, AB | a |
| 386 | 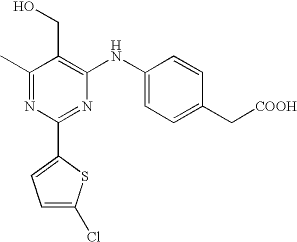 | 4.36 | 1 | 390 (M + H) | H, K, B, AN, C, a | e |
| 387 | 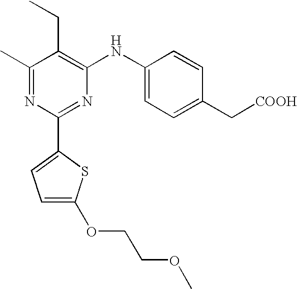 | 3.12 | 1 | 428 (M + H) | D, E, F, B, C, U, c, Z | e |
| 388 | 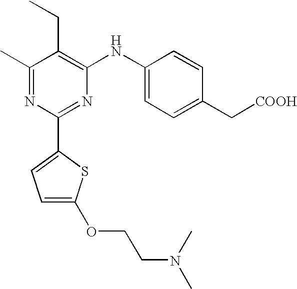 | 2.41 | 1 | 441 (M + H) | D, E, F, B, C, U, c, Z | e |
| 389 | 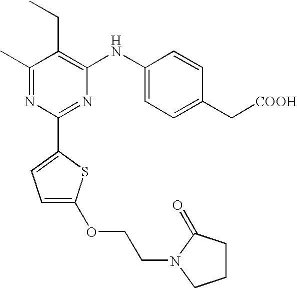 | 2.93 | 1 | 481 (M + H) | D, E, F, B, C, U, c, Z | e |

### Example 390

### ethyl 2-(4-(2-(5-(difluoromethoxy)thiophene-2-yl)-5-ethyl-6-methylpyrimidin-4-ylamino)phenyl)acetate

Ethyl 2-(4-(2-(5-hydroxythiophene-2-yl)-5-ethyl-6-methylpyrimidin-4-ylamino)phenyl)acetate (61 mg) was dissolved in N,N-dimethylformamide (4 mL), and lithium carbonate (34 mg, manufactured by Kanto Chemical Co.) and t-butyl chlorodifluoroacetate (86 mg, manufactured by Apollo Co.) were sequentially added to the solution. The mixed solution was stirred at 90° C. for 1 hour in nitrogen atmosphere. Water (15 mL) was added to the reaction mixture, which was extracted with saturated brine. The organic layer was concentrated after drying over anhydrous magnesium sulfate. The residue obtained was purified by preparative thin layer chromatography (hexane/ethyl acetate=2/1) to obtain the titled compound (22 mg). Retention time of LC-MS:HPLC; 5.08 minute (LC condition 1), m/z 448 (M+H).

The starting material ethyl 2-(4-(2-(5-hydroxythiophene-2-yl)-5-ethyl-6-methylpyrimidin-4-ylamino)phenyl)acetate used in this example was manufactured according to the production methods in the following example and reference examples: Reference Examples 4, 5, 6, 2, 3 and 21, and Example 29.

### Examples 391 to 393

Production of the compounds in Examples 391 to 393 is shown below. Details of Examples 391 to 393 are shown in Table 6. Notations in Table 6 mean as follows: ac; production method shown in Example 390. Other notations have the same meaning as those in Table 5.

TABLE 6

| Exp. | Str. | R.T. | LC | MS | Ref. | Ex. |
|---|---|---|---|---|---|---|
| 391 | 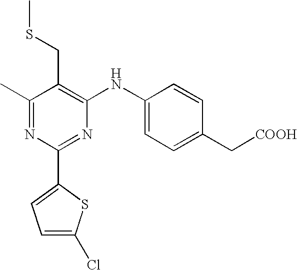 | 5.26 | 1 | 420 (M + H) | H, K, B, AN, C, AM | a |
| 392 | 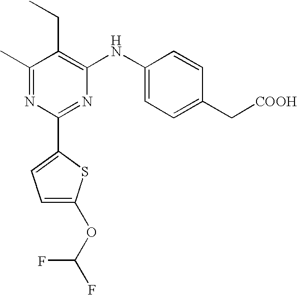 | 3.59 | 1 | 420 (M + H) | D, E, F, B, C, U, c, ac | e |
| 393 | 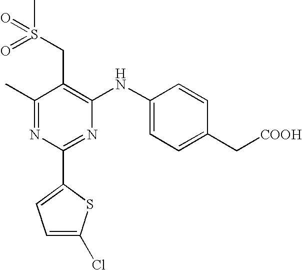 | 4.57 | 1 | 452 (M + H) | H, K, B, AN, C, AM, c, aa | e |

### Examples 394 to 402

Production of the compounds in Examples 394 to 402 is shown below. Details of Examples 394 to 403 are shown in Table 7. Notations in Table 7 have the same meaning as those in Table 6.

TABLE 7

| Exp. | Str. | R.T. | LC | MS | Ref. | Ex. |
|---|---|---|---|---|---|---|
| 394 | 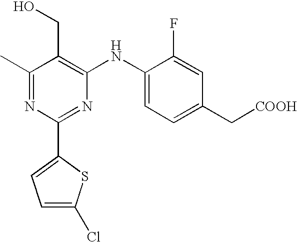 | 4.64 | 1 | 408 (M + H) | H, K, B, AN, C, AC, AD, AE, a | e |
| 395 | 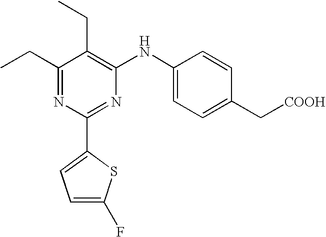 | 4.87 | 1 | 386 (M + H) | K, B, C, c | e |
| 396 | 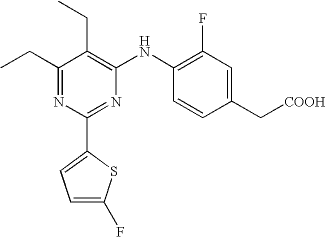 | 5.05 | 1 | 404 (M + H) | K, B, C, AC, AD, AE, c | e |
| 397 | 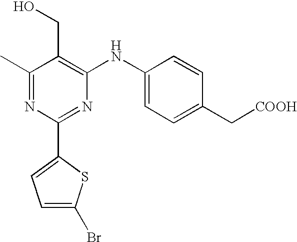 | 4.10 | 2 | 435 (M + H) | K, B, AN, C, a | e |
| 398 | 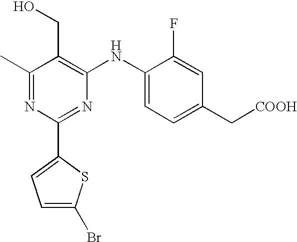 | 4.58 | 2 | 452 (M + H) | K, B, AN, C, AC, AD, AE, a | e |
| 399 | 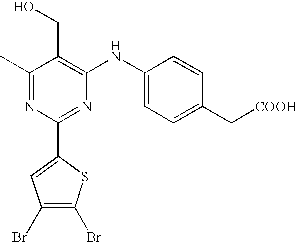 | 5.28 | 2 | 512 (M + H) | H, K, B, AN, C, a | e |
| 400 | 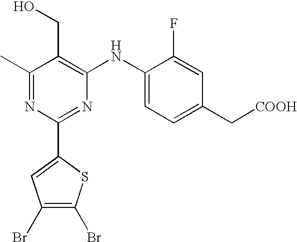 | 5.65 | 2 | 530 (M + H) | H, K, B, AN, C, AC, AD, AE, a | e |
| 401 | 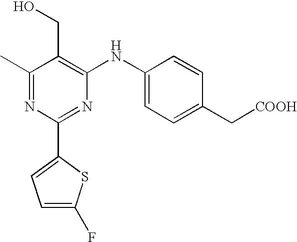 | 3.67 | 2 | 374 (M + H) | K, B, A, C, a | e |
| 402 | 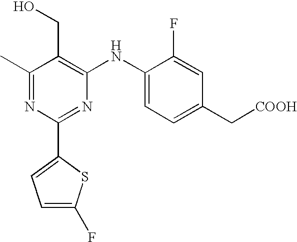 | 4.16 | 2 | 392 (M + H) | K, B, AN, C, AC, AD, AE, a | e |

### Test Example 1

### Inhibitory Action of PDE4 Enzyme Activity

(1) Purification of PDE4 from U937 Cells

The PDE4 enzyme was purified from U937 cells that are the cells derived from lymphoma. U937 cells were suspended in buffer solution A (20 mM Tris-HCl(pH 6.5), 1 mM MgCl₂, 0.1 mM EGTA, 3 mM 2-mercaptoethanol) containing protease. inhibitor cocktail (manufactured by Sigma Co.), and a soluble fraction was obtained by centrifugation after ultrasonic disintegration (100,000 G, 30 minutes, 4° C.). The soluble fraction obtained was charged on a Q-sepharose column (1.6×25 cm) equilibrated with buffer solution A. The charged soluble fraction was eluted with buffer solution A having a linear gradient of sodium acetate from 0 to 1 M. The elution rate was 1.33 ml/minute, and the eluate was collected in a volume of 11 mL in every fractions. Each fraction was tested for the cAMP metabolized PDE activity to identify the fractions containing PDE4 enzyme. It was confirmed that the PDE4 fraction does not decompose cGMP.

(2) Measurement of Inhibitory Activity of PDE4 Enzyme Activity

A desired concentration of the test compound was allowed to react in a reaction mixture (100 μL; 40 mM Tris-HCl (pH 7.4), 5 MM MgCl₂, 4 mM 2-mercaptoethanol, 3 TM cAMP, 0.83 μCi [³H]-cAMP and PDE4 fraction) at 30° C. for 10 minutes. The enzyme reaction was stopped by adding 25 μL of trichloroacetic acid to the reaction mixture. The reaction solution was charged on a neutral alumina column equilibrated with 0.1 M of 2-[[tris(hydroxylmethyl)methyl]-amino]-1-ethanesulfonic acid (TES) buffer (pH 8.0) and, after washing the column with a sufficient amount of 0.1 M TES buffer solution, the column was eluted with 2N NaOH. A 500 μL aliquot of the eluate containing [³H]-5′AMP product was filled in a scintillation vial containing the scintillation cocktail (5 ml, manufactured by Perkin Elmer Co.) to measure radioactivity. All the measurements were performed with a concentration range exhibiting a linear response. As shown in the results in Table 8, the compound of the invention, salts thereof or prodrugs thereof showed excellent inhibitory actions of PDE4 enzyme activity. The inhibitory action of PDE4 enzyme activity was expressed by IC50 (nM). Ex. No. in Table 8 denotes the example No.

TABLE 8

| Ex. No | IC50 (nM) |
|---|---|
| 3 | 356 |
| 5 | 1389 |
| 6 | 2937 |
| 7 | 409 |
| 11 | 1166 |
| 13 | 186 |
| 15 | 490 |
| 17 | 9 |
| 18 | 1679 |
| 20 | 191 |
| 23 | 633 |
| 24 | 3248 |
| 26 | 312 |
| 56 | 61 |
| 59 | 41 |
| 61 | 84 |
| 79 | 117 |
| 82 | 90 |
| 85 | 76 |
| 105 | 116 |
| 155 | 845 |
| 185 | 156 |
| 188 | 59 |
| 191 | 90 |
| 192 | 89 |
| 193 | 70 |
| 198 | 105 |
| 201 | 133 |
| 209 | 192 |
| 288 | 69 |

Another compounds showing 50% or more of inhibitory activity of PDE4.enzyme at a concentration of 20 μM are as follows:

Example Nos. 50,. 57, 58, 59, 60, 78, 81, 83, 84, 86, 96, 101, 103, 110, 111, 116,.120, 128, 133, 146, 147, 148, 154, 159, 169, 175, 180, 215, 220, 236, 241, 243, 244, 250, 253, 254, 257, 264 and 265.

### Test Example 2

### Inhibitory Action of TNF-α Production Using Mouse Mononuclear Cells (In Vitro)

Mouse mononuclear cells were isolated from the peripheral blood of C3H/He mouse (manufactured by Japan SLC Co., male), and were suspended in a culture medium (trade name RPMI 1640 manufactured by Gibco Co.) containing 10% bovine serum. The cells were seeded in a 96 well plate as 50,000 cells/well, the test compound was added at a desired concentration, and the plate was allowed to stand in an incubator containing 5% CO₂ at .37° C. for 15 minutes. LPS was added thereafter at a final concentration of 5 μg/mL, and the plate was allowed to stand in the incubator for 21 hours.

The culture medium containing produced TNF-α was retrieved, and the amount of TNF-α was measured according to the protocol attached to mouse TNF-α ELISA kit (manufactured by R & D Systems Co.). As shown in the results in Table 9, the compound of the invention, salts thereof or prodrugs thereof showed excellent suppression action of TNF-α production. The suppression action of TNF-α was expressed-by IC5 (nM). Ex. No. in Table 9 denotes the example No.

TABLE 9

| ex. No. | IC50 (nM) |
|---|---|
| 3 | 1900 |
| 5 | 265 |
| 6 | 4929 |
| 7 | 267 |
| 11 | 790 |
| 13 | 1600 |
| 15 | 55 |
| 17 | 24 |
| 18 | 202 |
| 20 | 289 |
| 23 | 2110 |
| 24 | 865 |
| 26 | 219 |
| 50 | 249 |
| 54 | 239 |
| 56 | 171 |
| 57 | 44 |
| 58 | 695 |
| 59 | 69 |
| 61 | 133 |
| 68 | 247 |
| 73 | 211 |
| 78 | 171 |
| 80 | 73 |
| 82 | 57 |
| 83 | 46 |
| 84 | 533 |
| 85 | 29 |
| 86 | 67 |
| 88 | 63 |
| 91 | 65 |
| 96 | 69 |
| 111 | 58 |
| 113 | 34 |
| 116 | 10 |
| 127 | 72 |
| 129 | 85 |
| 148 | 676 |
| 157 | 80 |
| 181 | 57 |
| 186 | 80 |
| 188 | 46 |
| 191 | 44 |
| 193 | 39 |
| 203 | 86 |
| 220 | 59 |
| 221 | 53 |
| 234 | 48 |
| 253 | 504 |

### Test Example 3

### Action on Gastric Acid Secretion Ability(In Vitro)

Gastric acid secretion ability (as an index of the side effect) was tested according to the methods by BergLindh and Obrink (Acta Physiol. Scand. 97:401-414, 1976) and Sack and Speney (Am. J. Physiol. 243:G313-G319, 1982).

Gastric gland cells were prepared by collagenase treatment using male rabbit (Japanese white strain, manufactured by Oriental Yeast Co.).

The test compound, ¹⁴C-aminopyrine (1.0 nmol/mL; 0.1 μCi/nmol) and histamine (0.3 to 1.0 μM) were incubated for a given time period, and gastric gland cell pellets obtained after centrifugation and washing were lysed with 100 μL of 1N-KOH. The lysate was filled in a scintillation vial containing a scintillation cocktail (manufactured by Perkin Elmer Co.) to measure the radioactivity. The gastric acid secretion action by the test compound was calculated as EC50, and the ratio of the activity relative to IC50 of suppression of TNF-α production described in Test Example 2 (degree of dissociation A) was determined from the following equation. The larger degree of dissociation A is more preferable. The results in Table 10 represent that secretion of the gastric acid ability(EC50) was lower activity in the compound of the invention, salts thereof or prodrugs thereof as compared with the suppression effect of TNF-α production(IC50). Ex. No. in Table 10 denotes the example No.

Degree of dissociation A=EC50 of gastric acid secretion ability/IC50 of suppression of TNF-α production

TABLE 10

| | EC50 (nM) of gastric | Degree of |
|---|---|---|
| Ex No. | acid secretion ability | dissociation A |
| 17 | 1692 | 71 |
| 57 | 460 | 11 |
| 58 | 1309 | 2 |
| 59 | 227 | 3 |
| 78 | 38413 | 225 |
| 83 | 169 | 4 |
| 84 | 1665 | 3 |
| 148 | 10070 | 15 |

### Test Example 4

### Inhibitory Action of TNF-α Production (In Vivo) Induced by LPS Using Mouse

C3H/He mouse (manufactured by Japan SLC Co., male) was used as a test animal. A desired concentration of the test sample was suspended in 0.5% methyl cellulose solution. The weight of the mouse was measured, and the test compound solution was orally administered at a dosage of 10 mL/kg while only 0.5% methyl cellulose was administered to the reference group. One group contained 5 to 7 mice. LPS was dissolved in physiological saline, and 10 μg was intraperitoneally administered to each mouse. The test compound was administered first, then LPS was administered 60 minutes thereafter. After 90 minutes, the blood was sampled from the large abdominal vein with the heparin, and a plasma was obtained by centrifugation. The content of TNF-α in the plasma was measured according to the protocol attached to TNF-α ELISA kit (manufactured by R & D System Co.). The representative results in Table 11 show that the compound of the invention, salts thereof or prodrugs thereof exhibited an excellent suppression action of TNF-α production. The suppression action of TNF-α production was represented by ED50 (mg/kg). Ex. No. in Table 11 denotes the example No.

TABLE 11

| Ex. No. | ED50 (mg/kg) |
|---|---|
| 11 | 26 |
| 15 | 13 |
| 17 | 3 |
| 18 | 37 |
| 57 | 24 |
| 59 | 6 |
| 83 | 22 |
| 86 | 8 |
| 58 | 8 |
| 84 | 17 |
| 103 | 37 |
| 110 | 31 |
| 111 | 15 |
| 116 | 12 |
| 128 | 24 |
| 133 | 27 |
| 148 | 12 |
| 205 | 21 |
| 220 | 41 |
| 221 | 24 |
| 238 | 20 |
| 239 | 22 |
| 241 | 39 |
| 253 | 14 |
| 254 | 38 |
| 293 | 18 |
| 274 | 25 |
| 289 | 18 |
| 292 | 25 |
| 344 | 27 |
| 345 | 37 |
| 373 | 19 |
| 376 | 12 |
| 386 | 50 |

### Test Example 5

### Gastric Emptying Test of Mouse (In Vivo)

C3H/He mouse (manufactured by Japan SLC Co., male) fasted for 21 to 24 hours but allowed free access to water, was used as an experimental animal. A desired concentration of the test sample was suspended in 0.5% methyl cellulose solution. The weight of the mouse was measured, and the test compound solution was orally administered at a dosage of 10 mL/kg while only 0.5% methyl cellulose solution was administered to the reference group. One group contained 5 mice. A 1.5% methyl cellulose solution containing 0.05% phenol, red was used as a test meal solution. The test compound was administered, and 0.2 ml of the test meal was orally administered at 60 minutes thereafter. The stomach was extracted 15 minutes after the administration of the test meal so that the test meal does not leak, and the stomach was homogenized in 0.1 N NaOH (10 mL) to remove the tissue by centrifugation. A 10% trichloroacetic acid solution (0.5 mL) was added to 5 mL of the supernatant, which was further centrifuged to remove proteins. A solution prepared by adding 125 μL of 2 N NaOH to the supernatant (1 mL) obtained by centrifugation was measured with a spectrophotometer (550 nm), and inhibition of transfer from the stomach to the small intestine was expressed by IC50 (mg/kg). The result indicated that the compound of the invention, salts thereof or prodrugs thereof has fewer side effects. For example, gastric emptying test IC50 of the compound in Example 17 is 14 mg/kg, which is 4.7 times higher compared with suppression of TNF-α production(ED50:3mg/kg) shown in Table 11. IC50 of the compound in Example 148 is 85 mg/kg, which is 7.1 times higher compared with suppression of TNF-α production(ED50:12mg/kg) shown in Table 11. The degree of dissociation of compounds in Examples 253, 373 and 376 are the same level as that of the compound in Example 17.

### Test Example 6

### Vomiting Action Using Ferret

Marshall ferret (male, weight 1 to 1.5 kg, manufactured by Charles River Laboratories Japan, Inc.) was used. A desired concentration of the test compound was suspended in 0.5% methyl cellulose solution. The weight of a ferret was measured, and the solution of the test compound was orally administered at a dosage of 1 to 8 ml/kg. Only 0.5% methyl cellulose solution was administered to the reference group. One group contained 4 animals. Vomiting behavior was observed using an acrylate observation box, in which behavior of the animal was observed for more than 6 hours with respect to number of vomiting and retching. The results showed that the compound of the invention, salts thereof or prodrugs thereof showed an apparent dissociation between drug anti-inflammatory effects and vomiting as a side effect, and that the compound of the invention exhibits fewer side effect(emetogenicity). For example, while ED50 of suppression of mouse TNF-α production of the compound in Example 17 shown in Table 11 was 3 mg/kg, no incidence of vomiting by the ferret was observed even at a dosage of 50 mg/kg. Likewise, while ED50 of suppression of mouse TNF-α production of the compound in Example 148 shown in Table 11 was 12 mg/kg, no incidence of vomiting by the ferret was observed even at a dosage of 100 mg/kg.

1. A compound represented by the following general formula (1), possible stereoisomers thereof or racemates thereof, pharmacologically acceptable salts thereof, hydrates thereof, solvated compounds thereof, or prodrugs thereof, 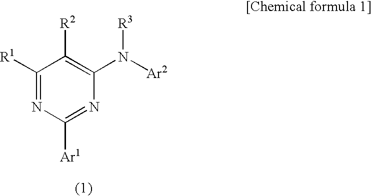
where, in general formula (1), Ar¹ represents a furyl group, thienyl group, triazolyl group, thiazolyl group, oxazolyl group or benzothiazolyl group; Ar¹ may be substituted; Ar² represents -E-Ar²-G-Q (Ar²¹ represents a benzene ring or naphthalene ring; E represents a single bond or an alkylene group; G represents a single bond, an alkylene group or an alkenylene group; Q represents a carboxy group, —CON(R⁴¹)(R⁴²) (R⁴¹ and R⁴² may be the same or different, each independently represents a hydrogen atom, a hydroxy group, an alkyl group that may be substituted or an aryl group that may be substituted, or R⁴¹ and R⁴² are combined to form a 3 to 7 membered ring that represents a cyclic amine as N(R⁴¹)(R⁴²), where R⁴² is a group other than a hydroxy group when R⁴¹ is the hydroxy group), or —COOR⁴³ (R⁴³ represents an alkyl group that may be substituted or an aryl group that may be substituted)), -E-Ar²¹-G²-G-Q (E, Ar²¹, G and Q are the same as described above, and G² represents —O—, —S—, —SO—, —SO²— or —NR^{G21}— (R^{F21} represents a hydrogen atom, an alkyl group that may be substituted, an acyl group that may be substituted, or a sulfonyl group that may be substituted)), or a monocyclic aromatic heterocyclic ring except the pyrazolyl group; Ar² may be substituted; R¹ and R² may be-the same or different, and each independently represents a hydrogen atom, an alkyl group that may be substituted, an alkenyl group that may be substituted, an alkynyl group that may be substituted, an alkoxy group that may be substituted, an alkylthio group that may be substituted, an alkylsulfinyl group that may be substituted, or an alkylsulfonyl group that may be substituted, and R³ represents a hydrogen atom or an alkyl group that may be substituted.

2. The compound according to claim 1, possible stereoisomers thereof or racemates thereof, pharmacologically acceptable salts thereof, hydrates thereof, solvated compounds thereof, or prodrugs thereof, wherein Ar¹ represents a furyl group or thienyl group that may be substituted.

3. The compound according to claim 1, possible stereoisomers thereof or racemates thereof, pharmacologically acceptable salts thereof, hydrates thereof, solvated compounds thereof, or prodrugs thereof, wherein Ar¹ may be substituted with one or a plurality of groups each independently selected from the group consisting of a halogen atom, an alkyl group that may be substituted, an alkoxy group that may be substituted, an alkylthio group that may be substituted, an amino group that may be substituted, or an acyl group that may be substituted.

4. The compound according to claim 1, possible stereoisomers thereof or racemates thereof, pharmacologically acceptable salts thereof, hydrates thereof, solvated compounds thereof, or prodrugs thereof, wherein R¹ represents a lower alkyl group.

5. The compound according to claim 1, possible stereoisomers thereof or racemates thereof, pharmacologically acceptable salts thereof, hydrates thereof, solvated compounds thereof, or prodrugs thereof, wherein R² represents a lower alkyl group or a lower alkenyl group.

6. The compound according to claim 1, possible stereoisomers thereof or racemates thereof, pharmacologically acceptable salts thereof, hydrates thereof, solvated compounds thereof, or prodrugs thereof, wherein R³ represents a hydrogen atom.

7. The compound according to claim 1, possible stereoisomers thereof or racemates thereof, pharmacologically acceptable salts thereof, hydrates thereof, solvated compounds thereof, or prodrugs thereof, wherein Ar² represents -E-Ar²¹-G-Q (E, Ar²¹, G and Q are the same as described above).

8. The compound according to claim 1, possible stereoisomers thereof or racemates thereof, pharmacologically acceptable salts thereof, hydrates thereof, solvated compounds thereof, or prodrugs thereof, wherein Ar²¹ represents a benzene ring.

9. The compound according to claim 1, possible stereoisomers thereof or racemates thereof, pharmacologically acceptable salts thereof, hydrates thereof, solvated compounds thereof, or prodrugs thereof, wherein E represents a single bond, and G represents a single bond or a lower alkylene group.

10. The compound according to claim 1, possible stereoisomers thereof or racemates thereof, pharmacologically acceptable salts thereof, hydrates thereof, solvated compounds thereof, or prodrugs thereof, wherein E represents a single bond and G represents a lower alkylene group.

11. The compound according to claim 1, possible stereoisomers thereof or racemates thereof, pharmacologically acceptable salts thereof, hydrates thereof, solvated compounds thereof, or prodrugs thereof, wherein Q represents a carboxy group.

12. The compound according to claim 1, possible stereoisomers thereof or racemates thereof, pharmacologically acceptable salts thereof, hydrates thereof, solvated compounds thereof, or prodrugs thereof, wherein Ar² may be substituted with one or a plurality of groups each independently selected from the group consisting of a hydroxy group, a halogen atom, an alkyl group that may be substituted and an alkoxy group that may be substituted.

13. The compound according to claim 1, possible stereoisomers thereof or racemates thereof, pharmacologically acceptable salts thereof, hydrates thereof, solvated compounds thereof, or prodrugs thereof, wherein Ar¹ represents a thienyl group, Ar¹ may be substituted with one or a plurality of groups each independently selected from the group consisting of a halogen atom, an alkyl group that may be substituted, an alkoxy group that may be substituted, an alkylthio group that may be substituted, an amino group that may be substituted and an acyl group that may be substituted; Ar² represents -E-Ar²¹-G-Q, Ar²¹ represents a benzene ring or a benzene ring substituted with one or a plurality of halogen atoms, E represents a single bond, G represents a methylene group, Q represents a carboxy group, Ar² has no additional substituents thereon, R¹ represents a methyl group or an ethyl group, R² represents an allyl group, an ethyl group or a hydroxymethyl group, and R³ represents a hydrogen atom.

14. The compound according to claim 1, possible stereoisomers thereof or racemates thereof, pharmacologically acceptable salts thereof, hydrates thereof, solvated compounds thereof, or prodrugs thereof, wherein Ar¹ may be substituted with one or a plurality of groups independently selected from the group consisting of a halogen atom, a lower alkyl group, a trifluoromethyl group, a hydroxymethyl group, a hydroxyethyl group, a lower alkoxy group,.a trifluoromethoxy group, a 2-methoxyethoxy group, a —NH₂ group, a lower alkylamino group, a lower dialkylamino group, an acylamino group and a lower alkylsulfonylamino group.

15. The compound according to claim 1, possible stereoisomers thereof or racemates thereof, pharmacologically acceptable salts thereof, hydrates thereof, solvated compounds thereof, or prodrugs thereof, wherein general formula (1) is represented by any one of the following structural formulae:

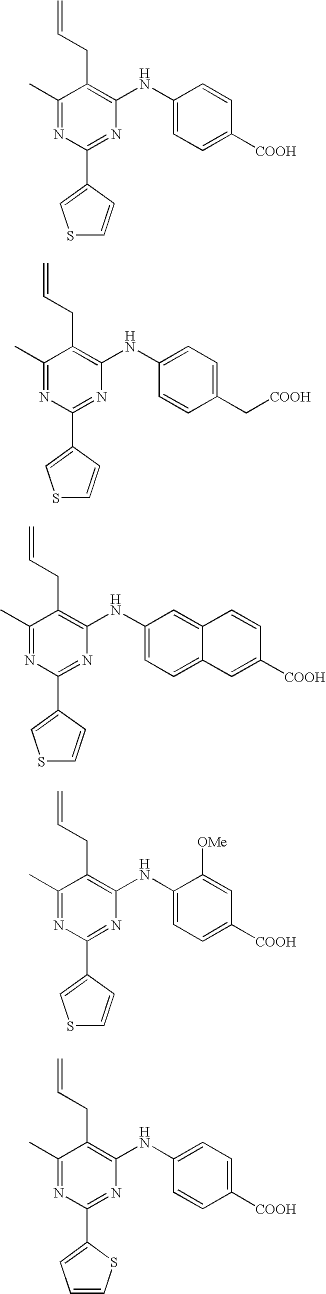

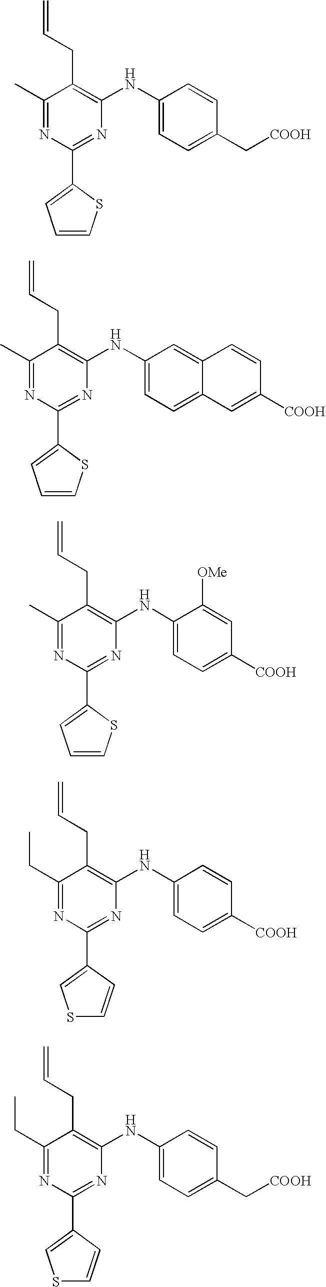

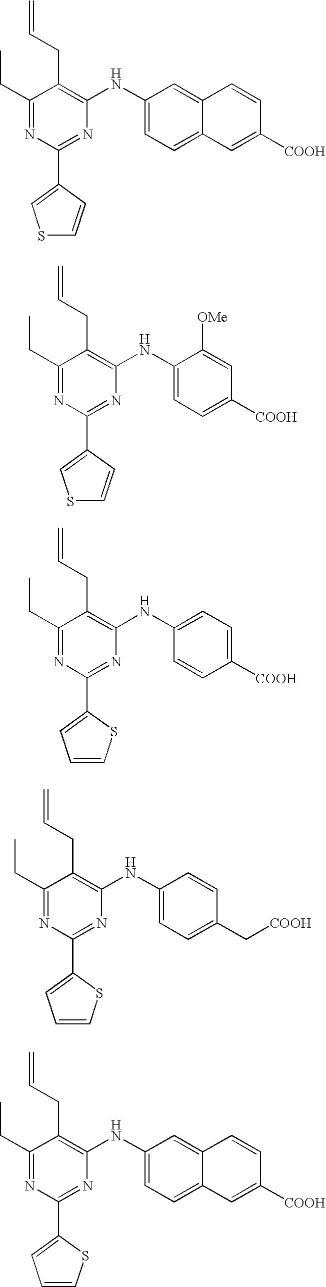

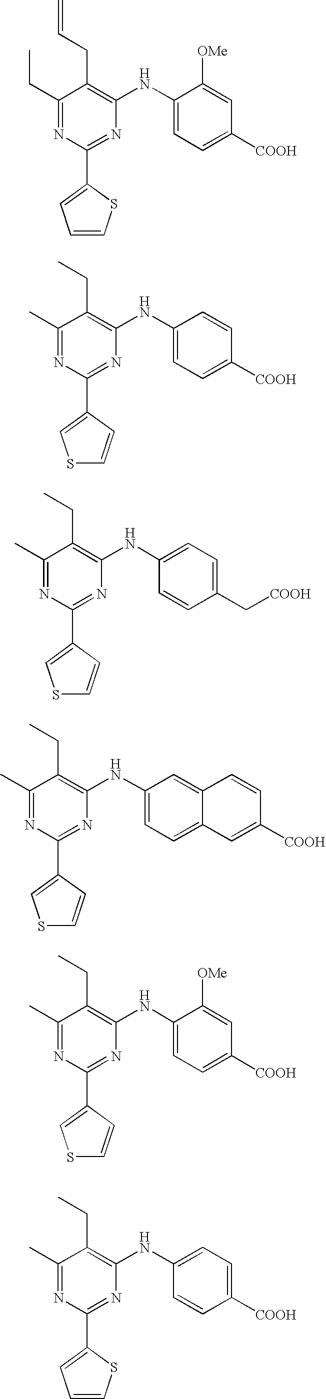

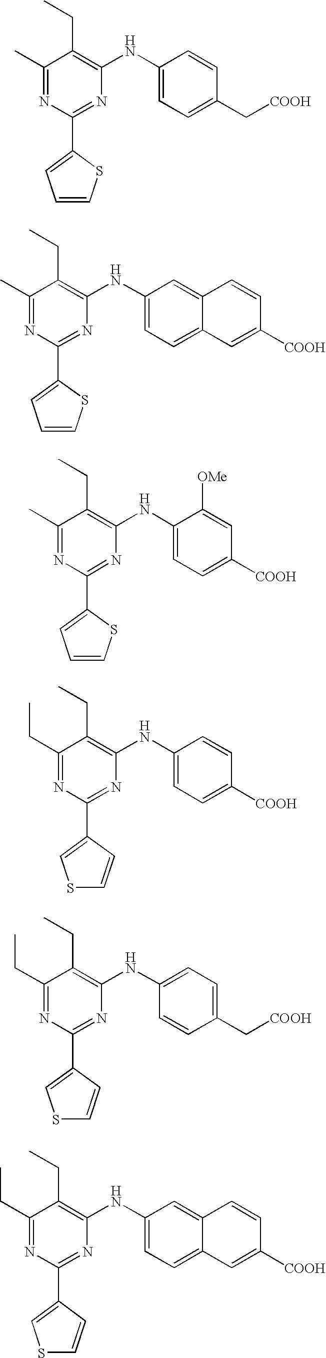

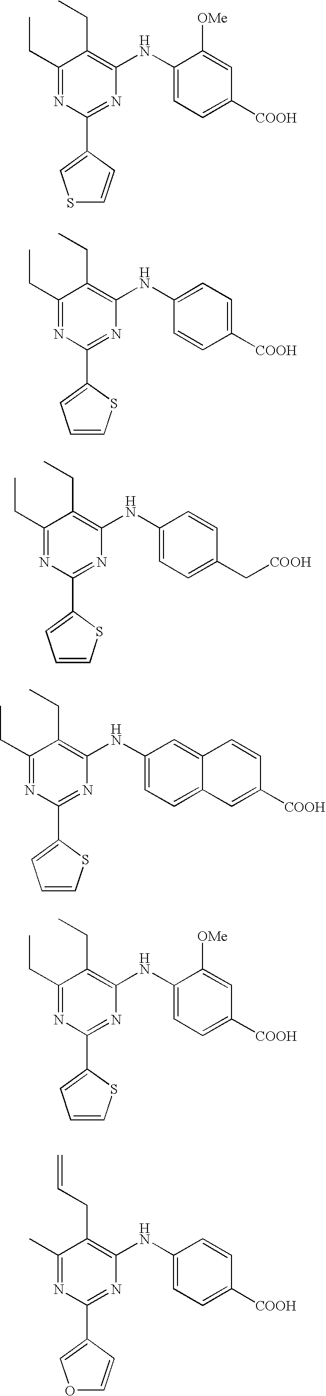

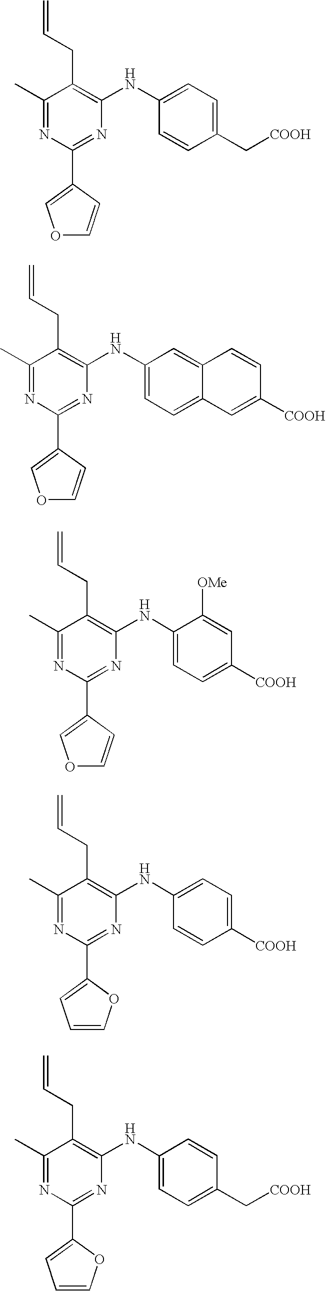

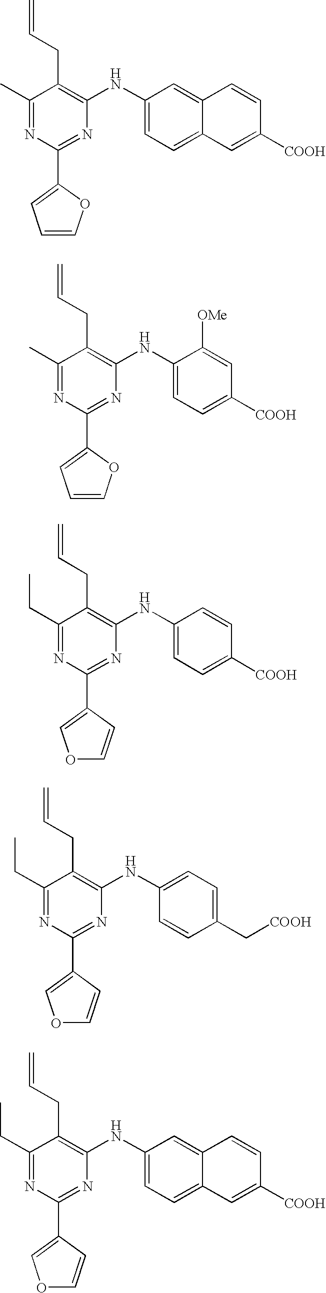

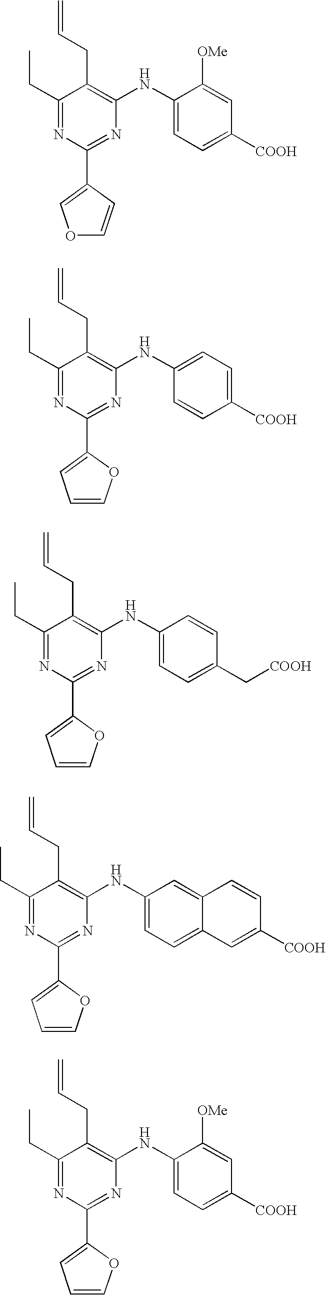

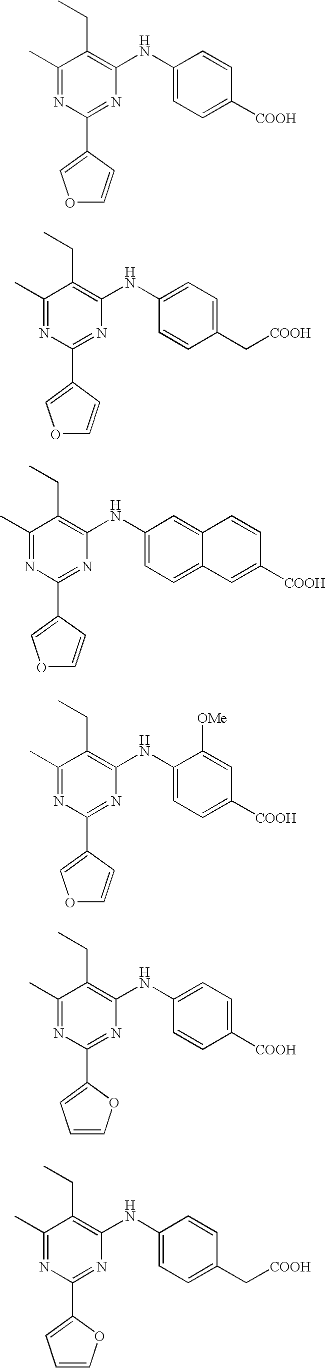

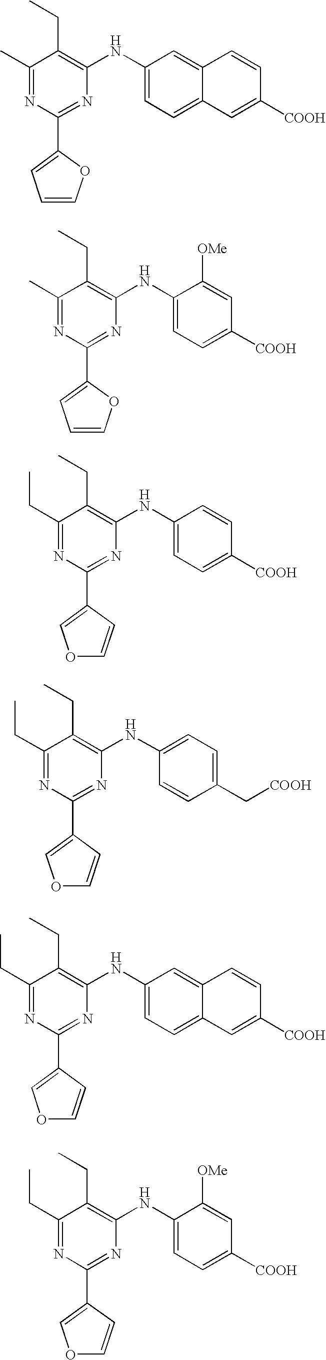

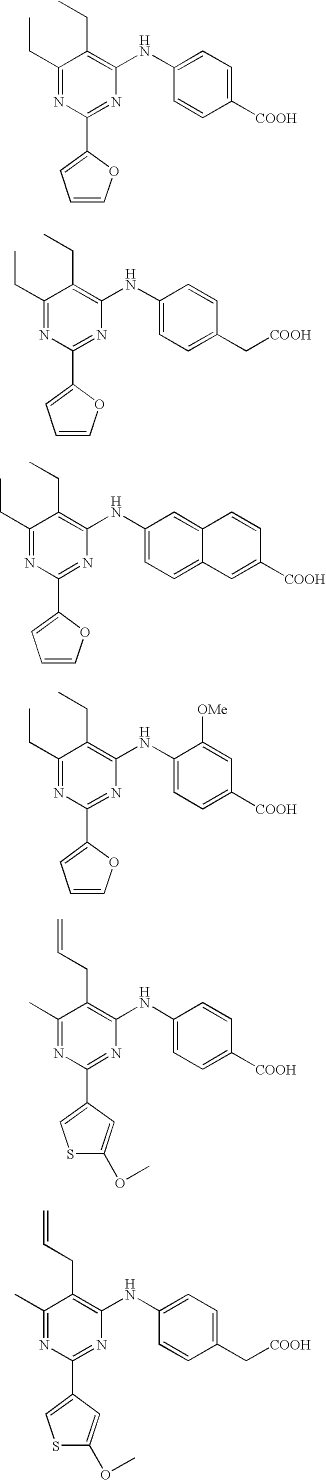

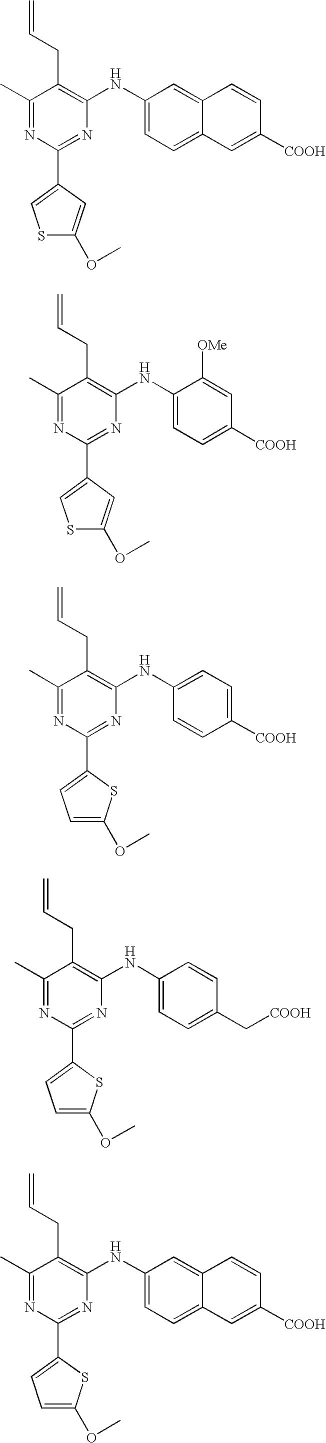

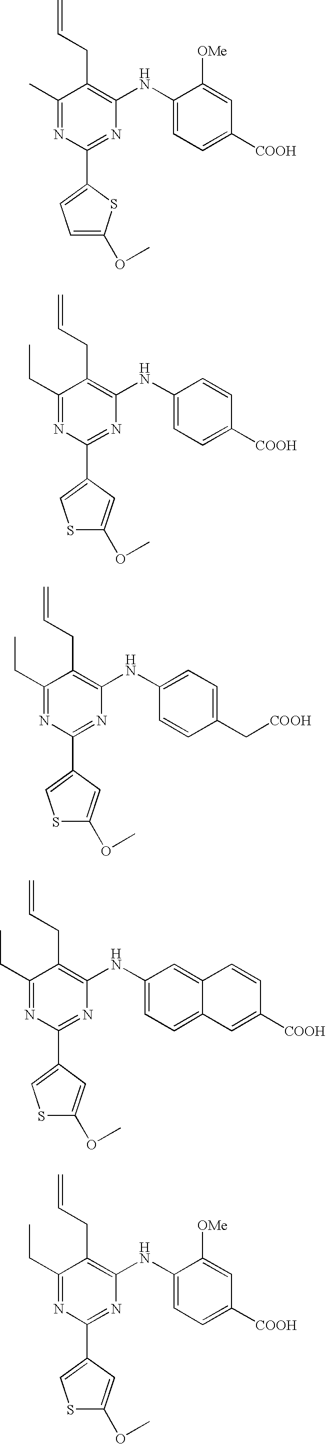

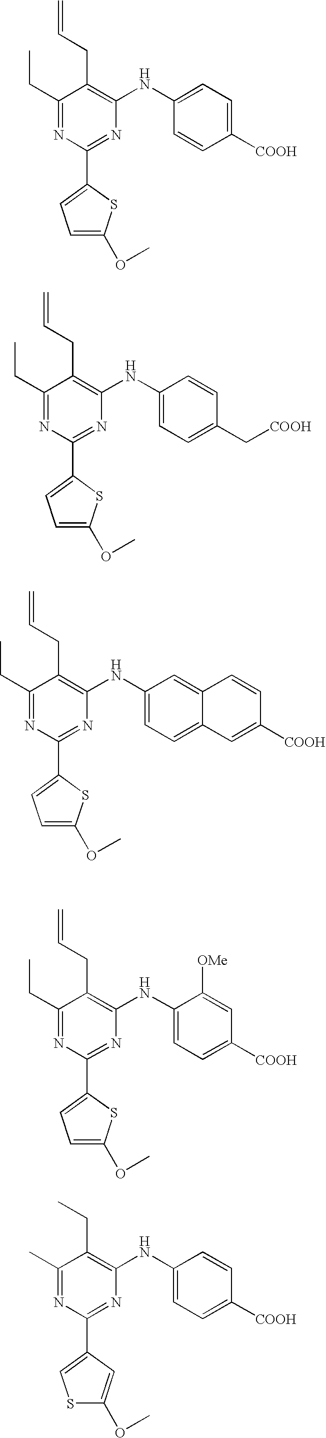

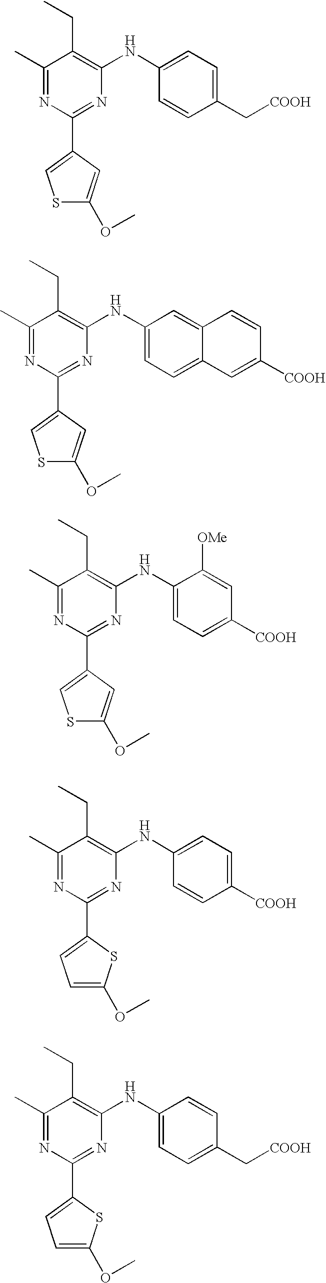

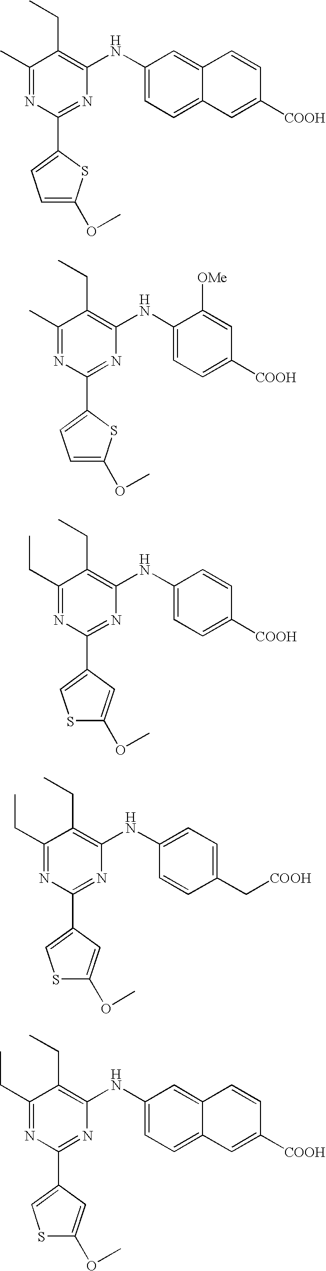

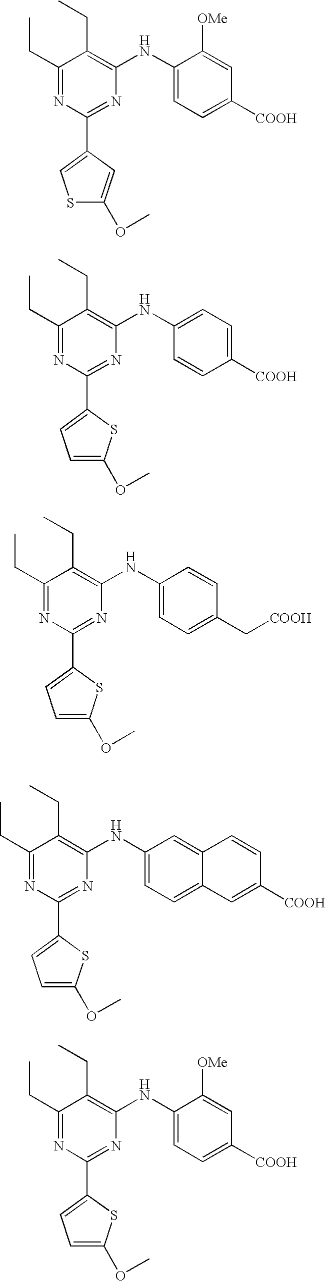

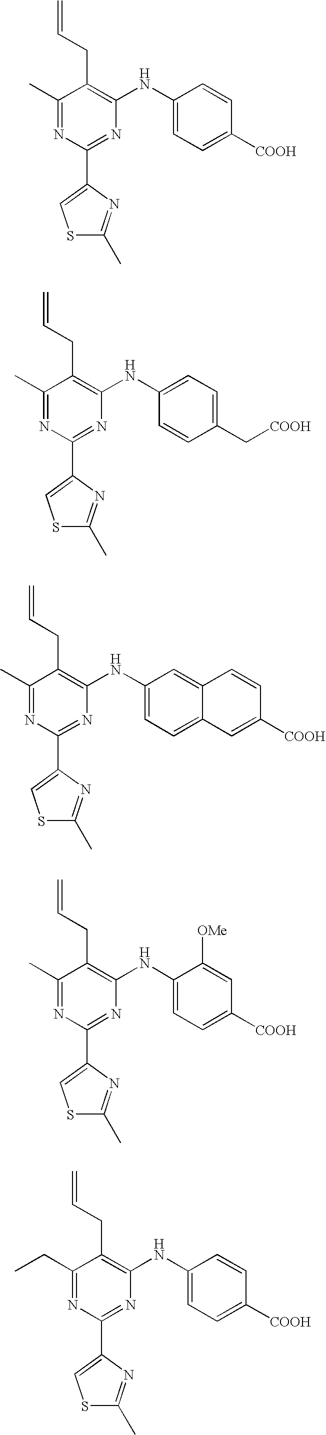

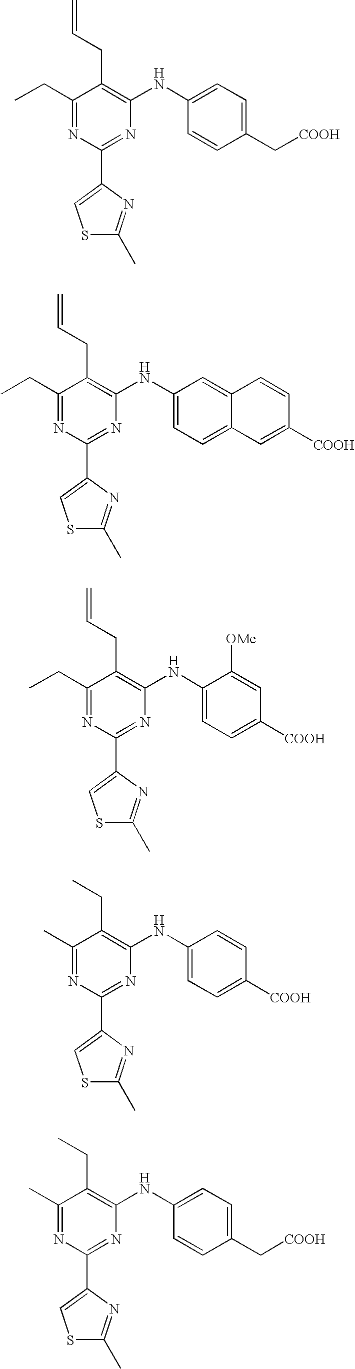

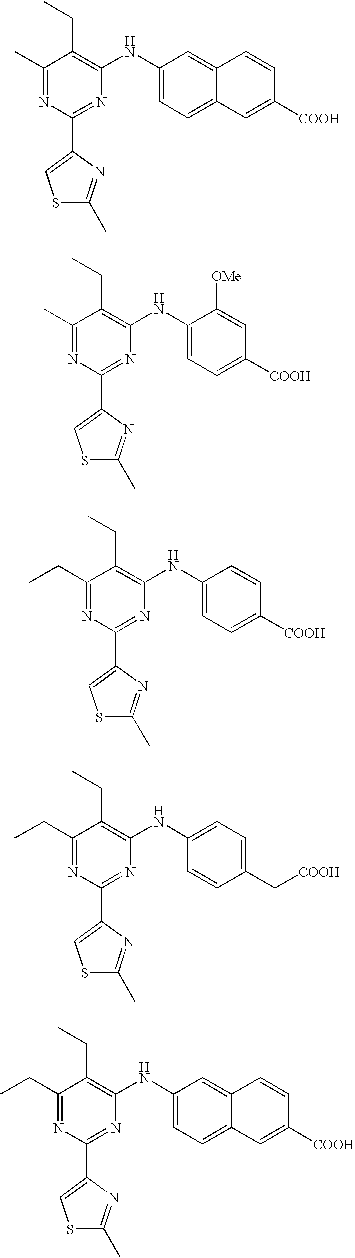

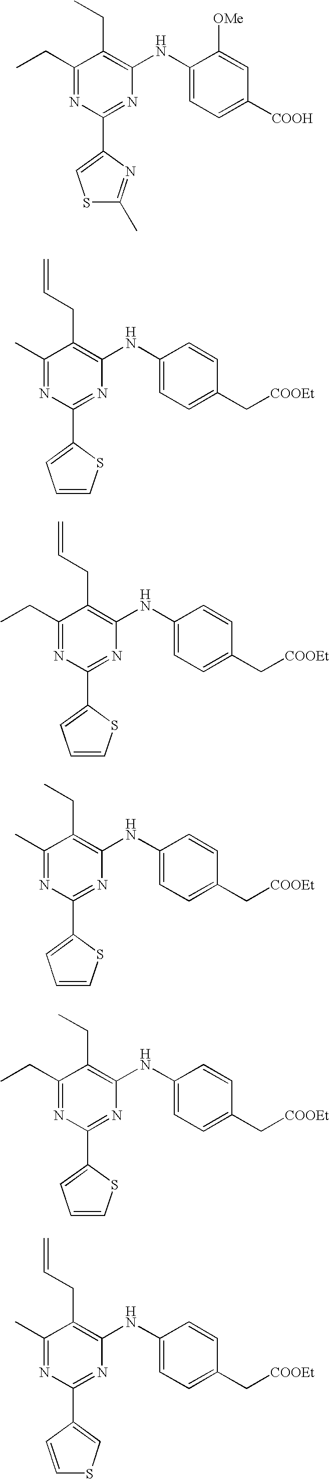

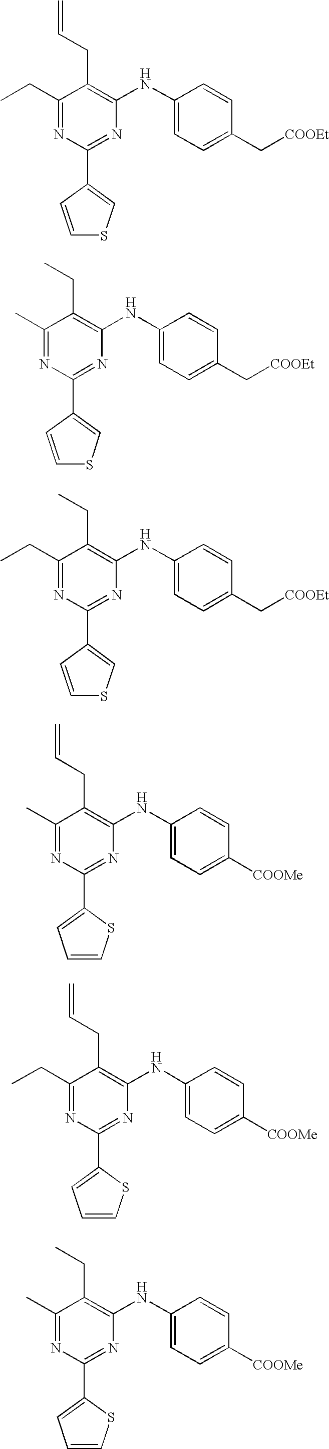

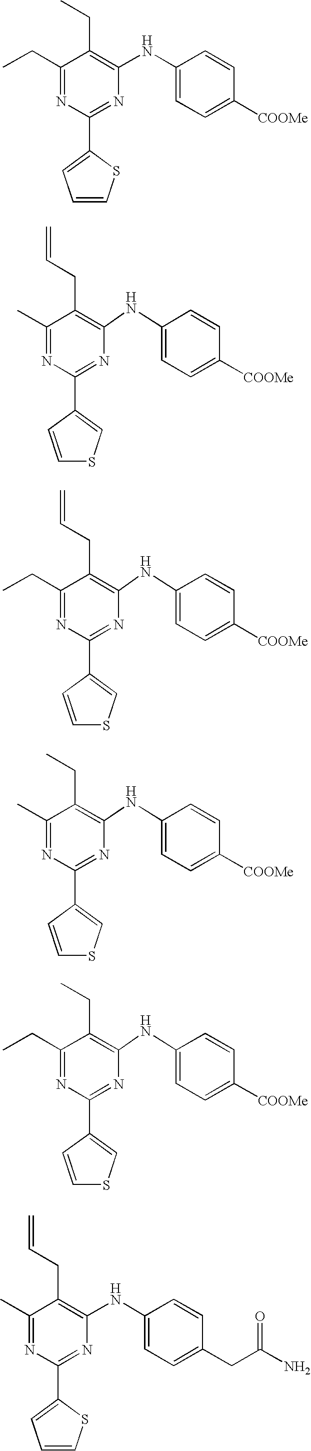

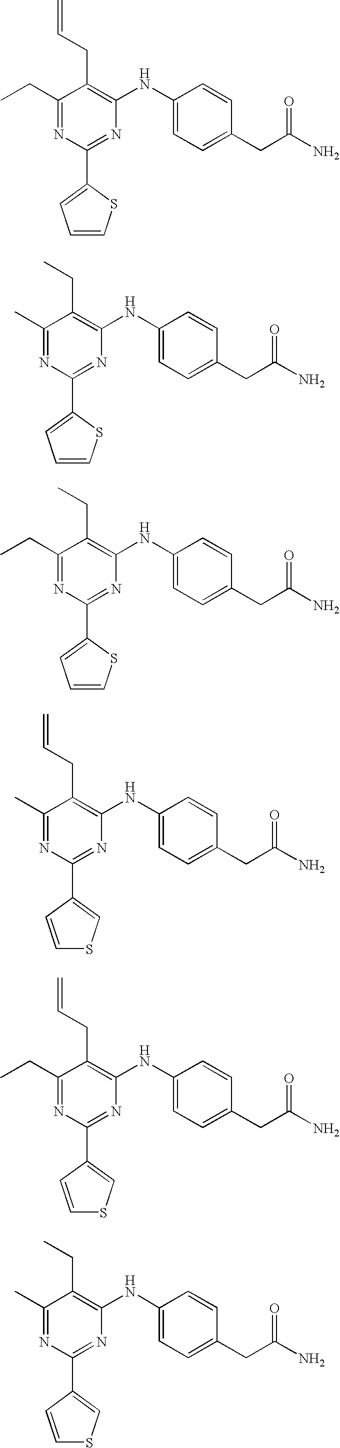

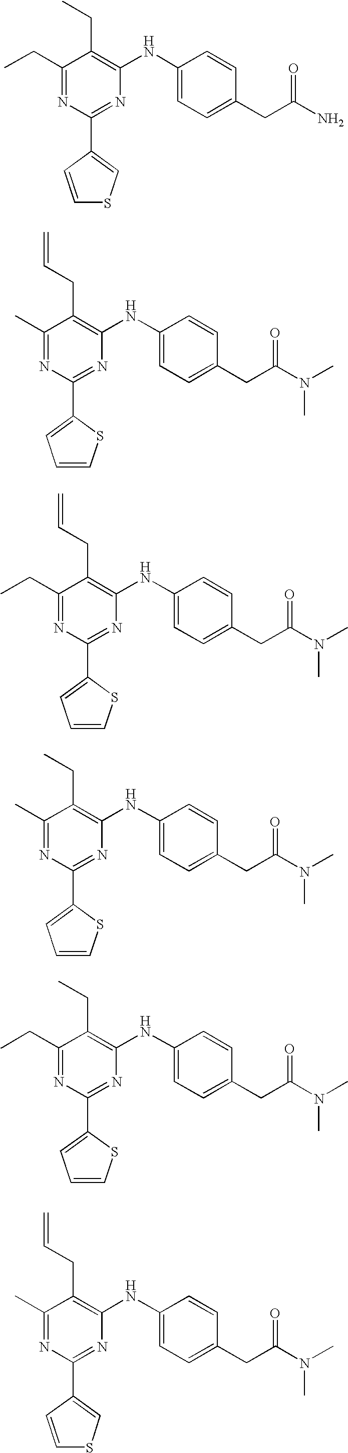

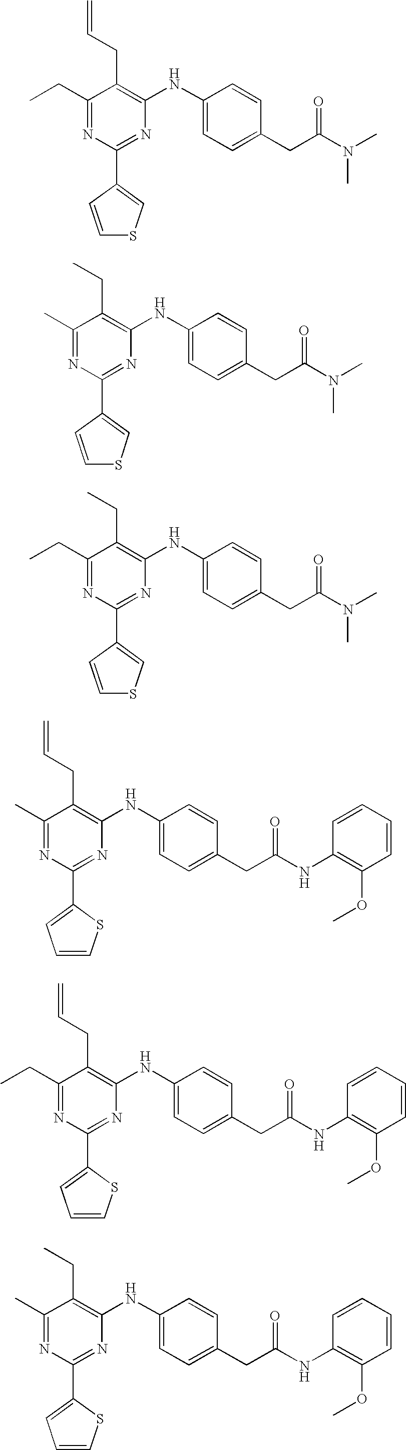

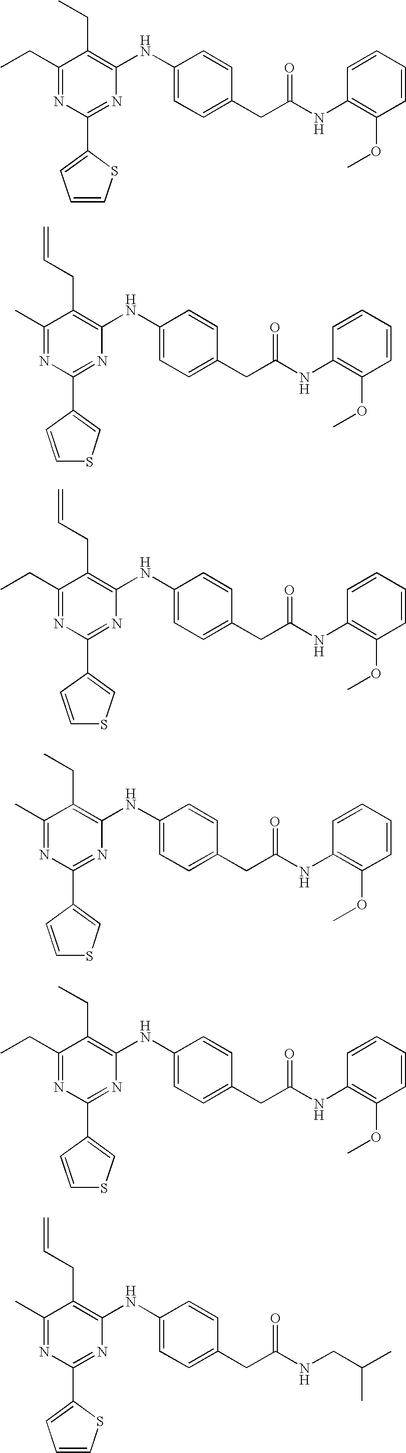

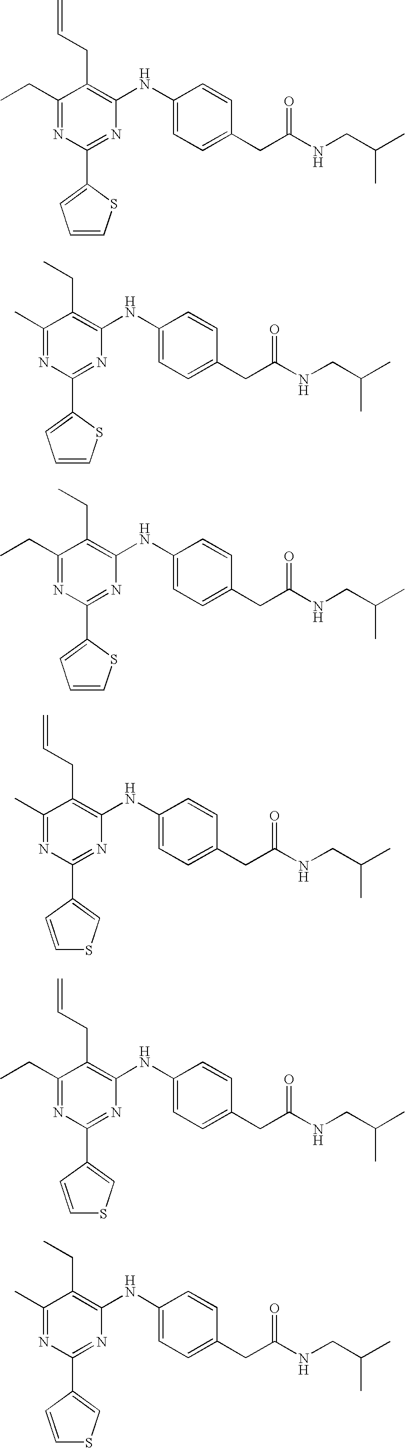

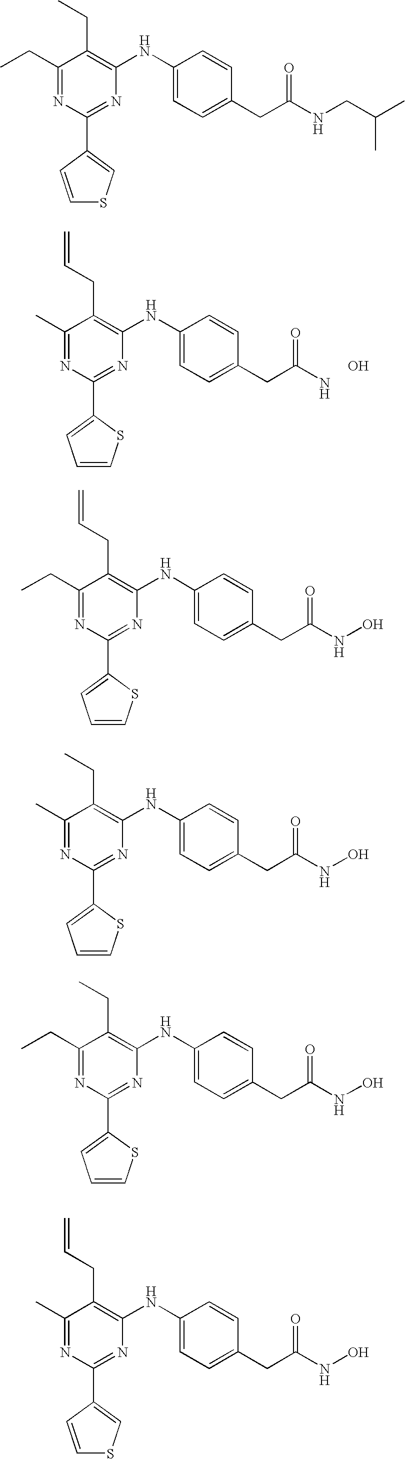

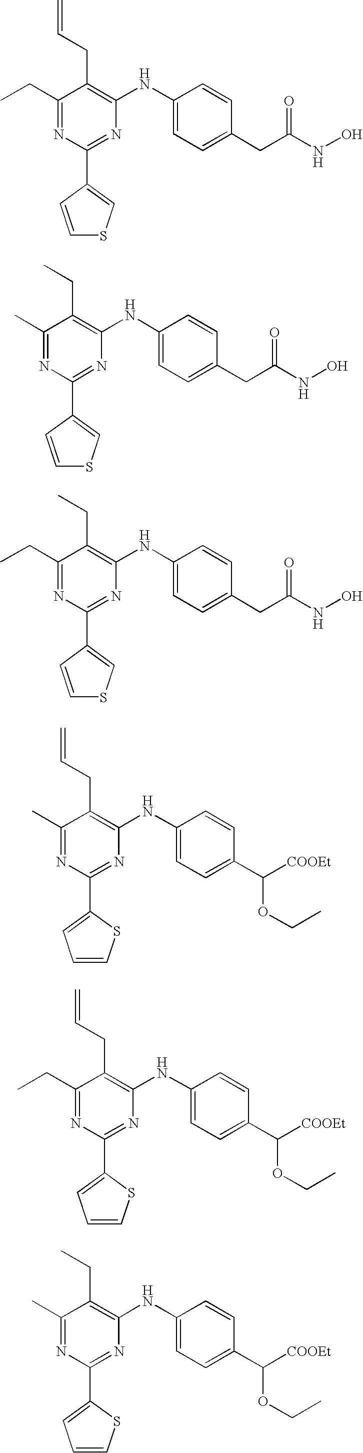

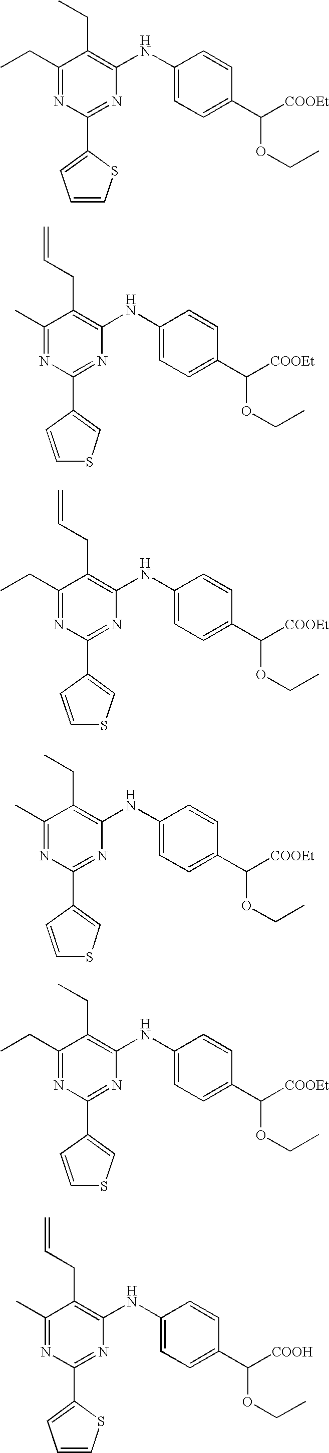

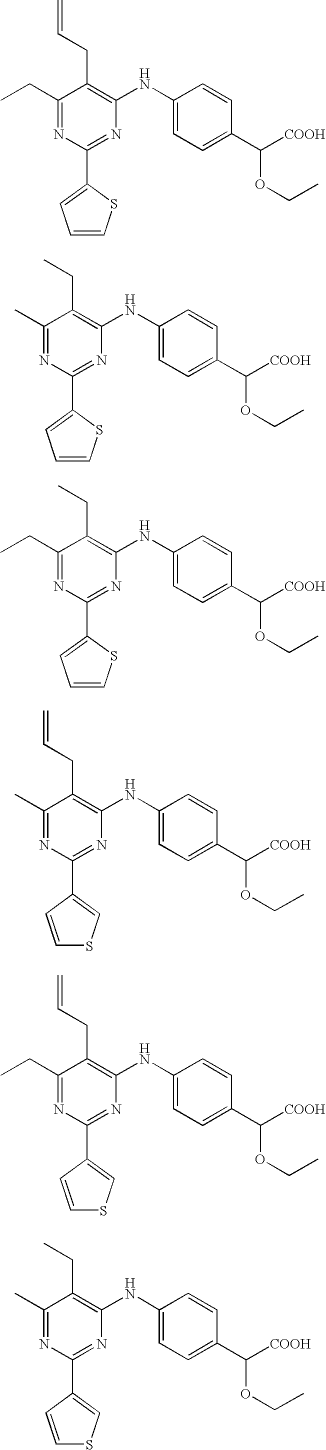

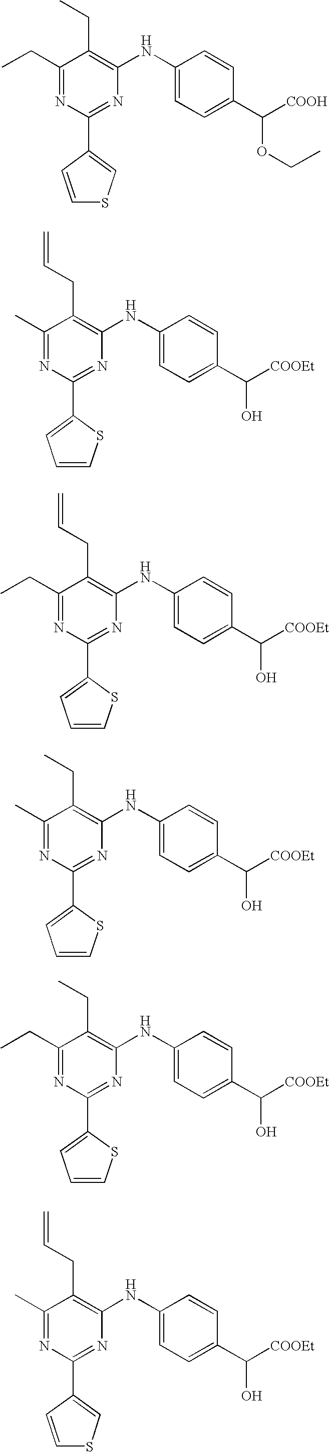

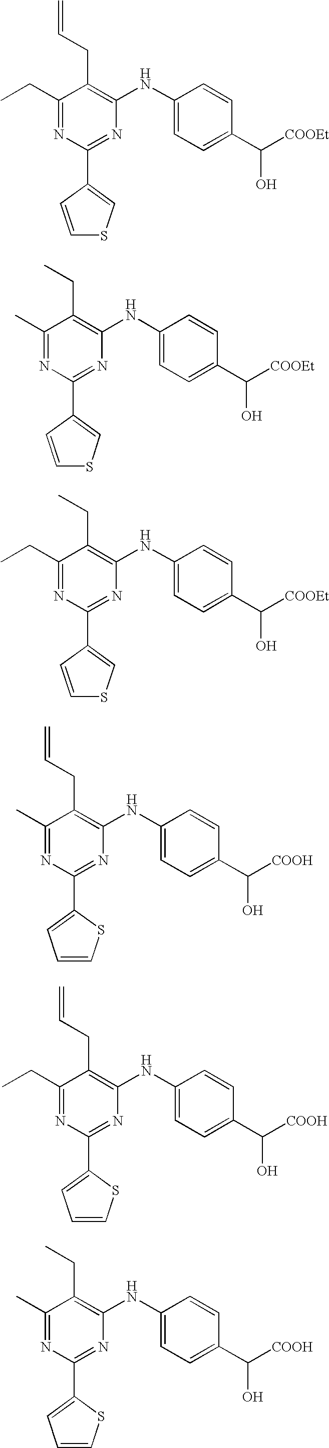

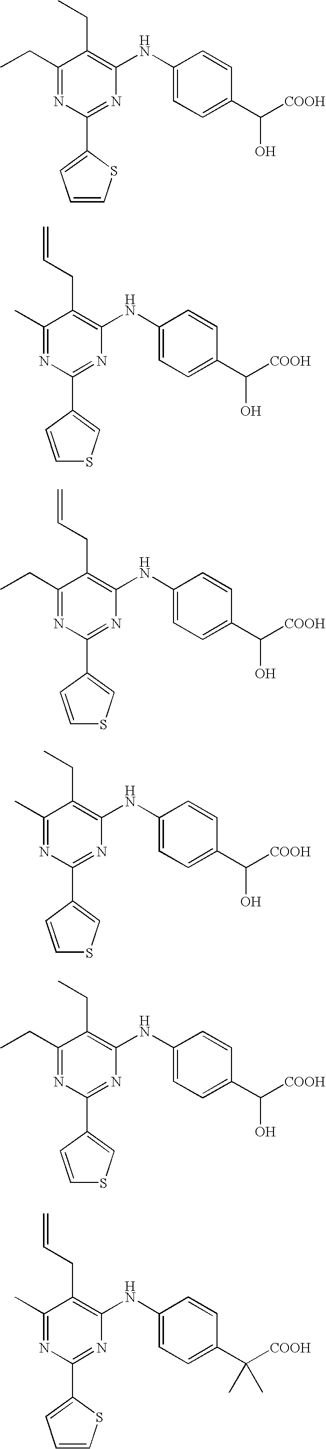

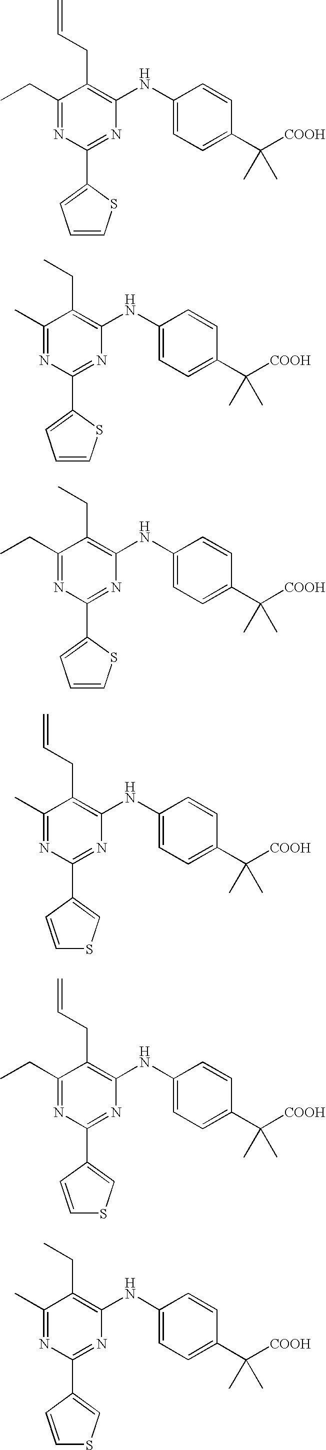

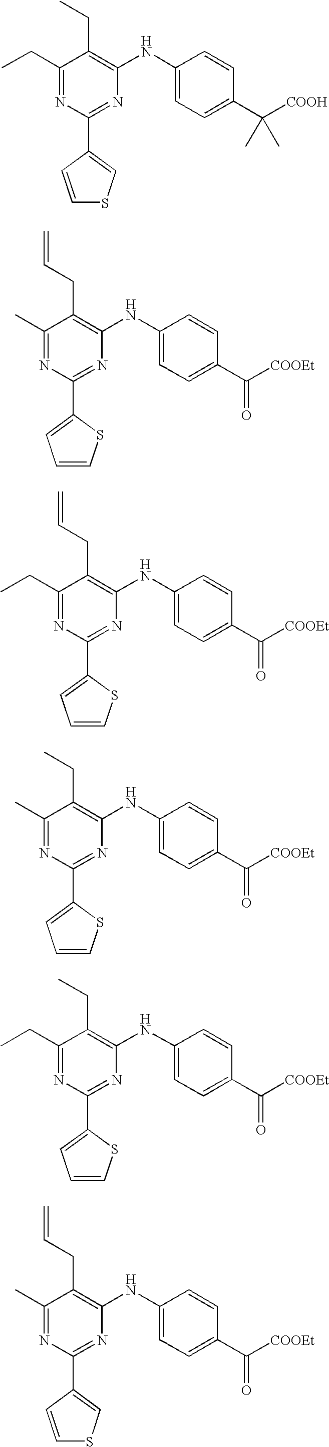

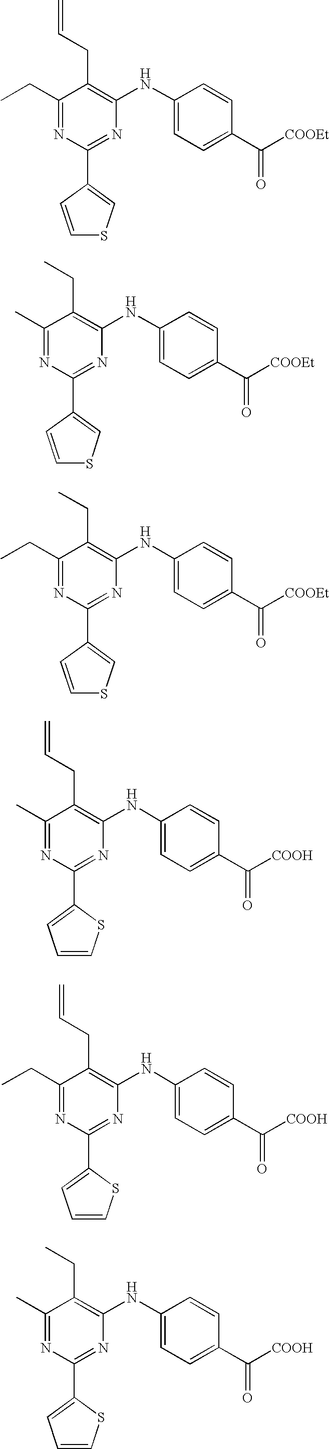

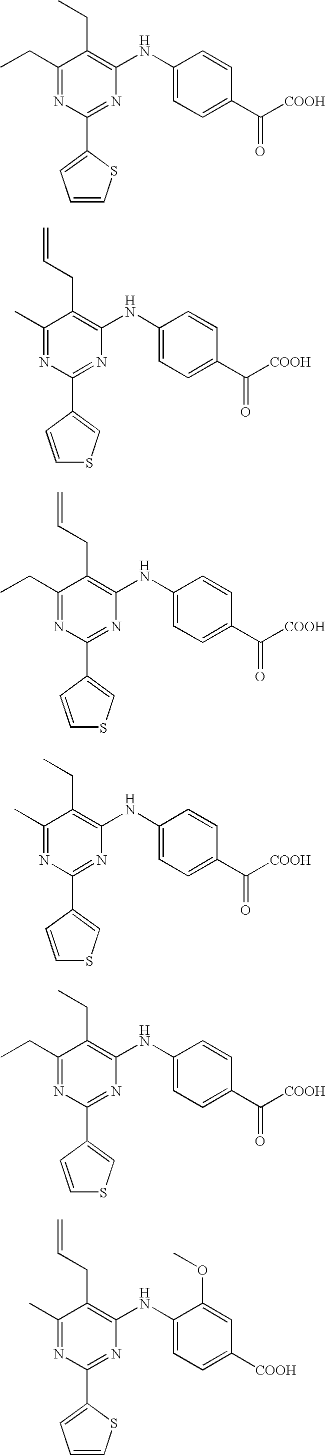

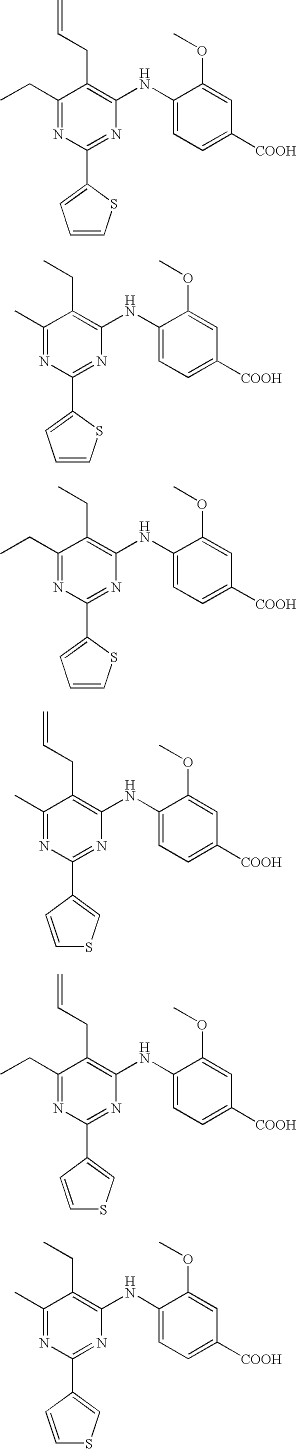

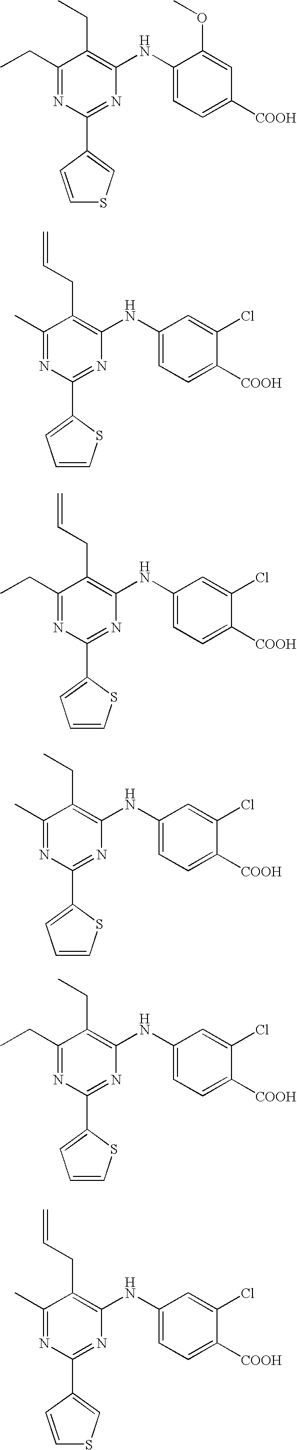

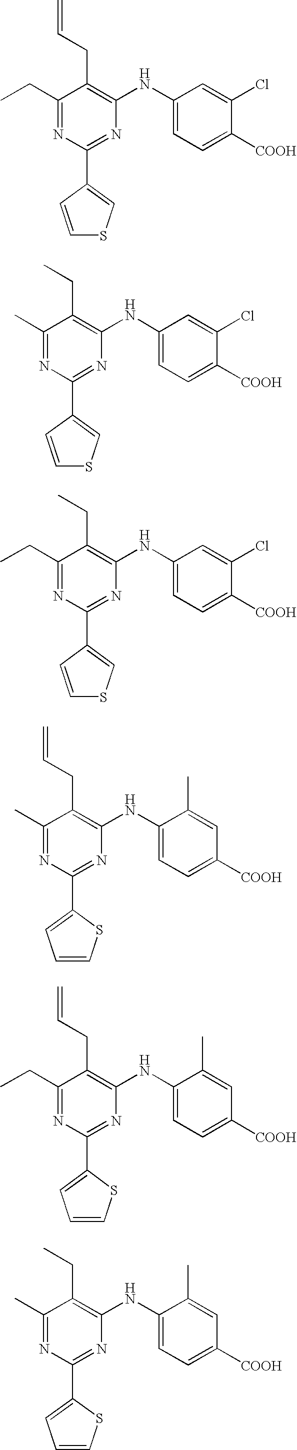

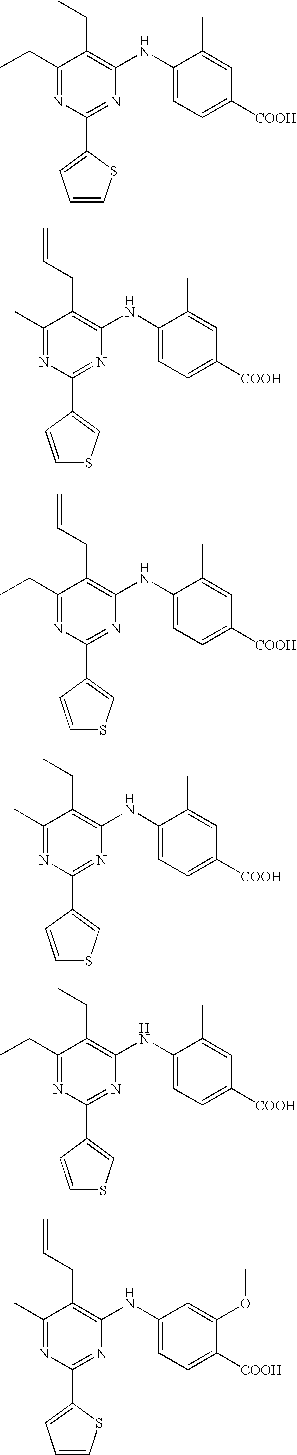

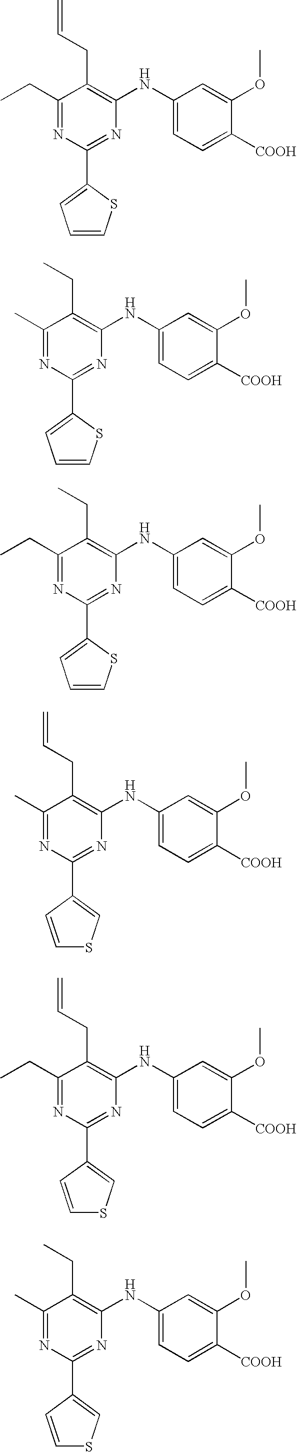

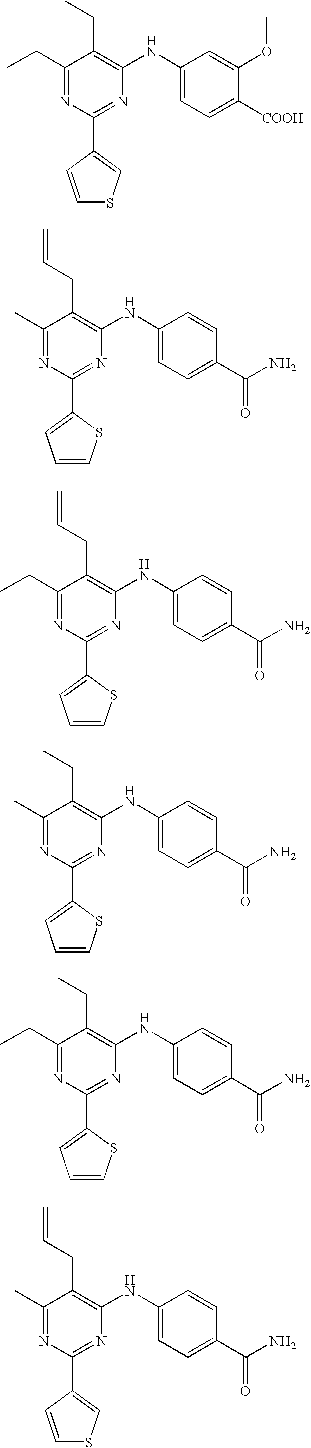

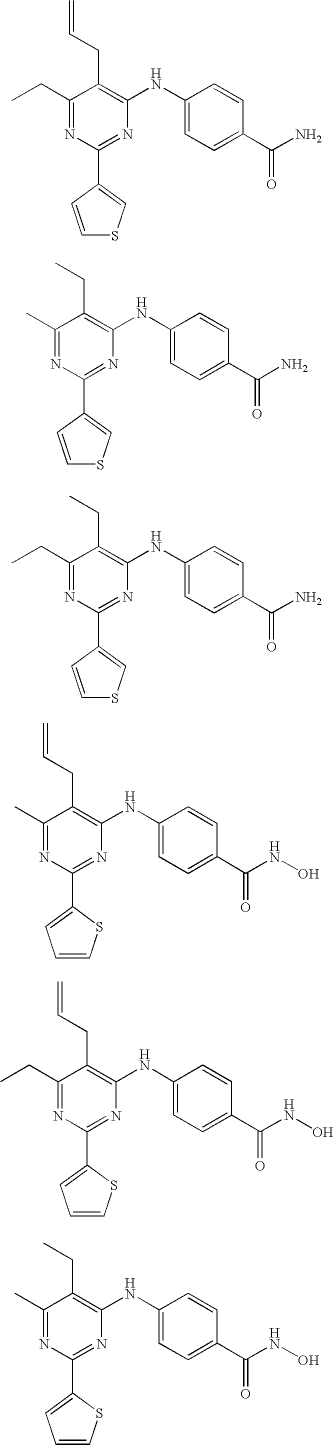

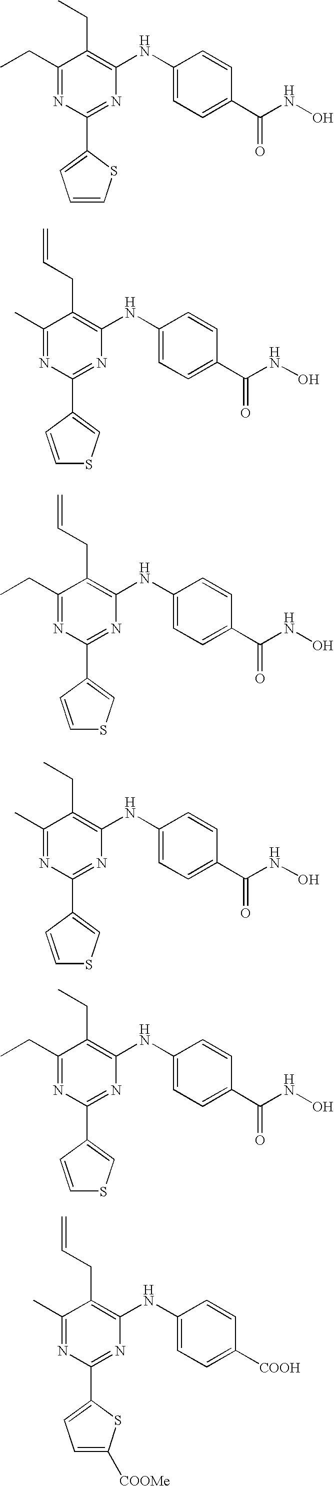

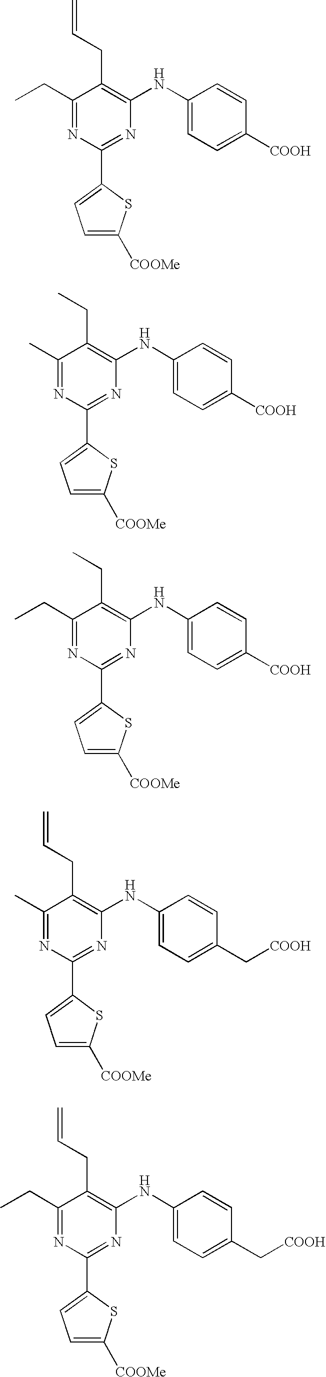

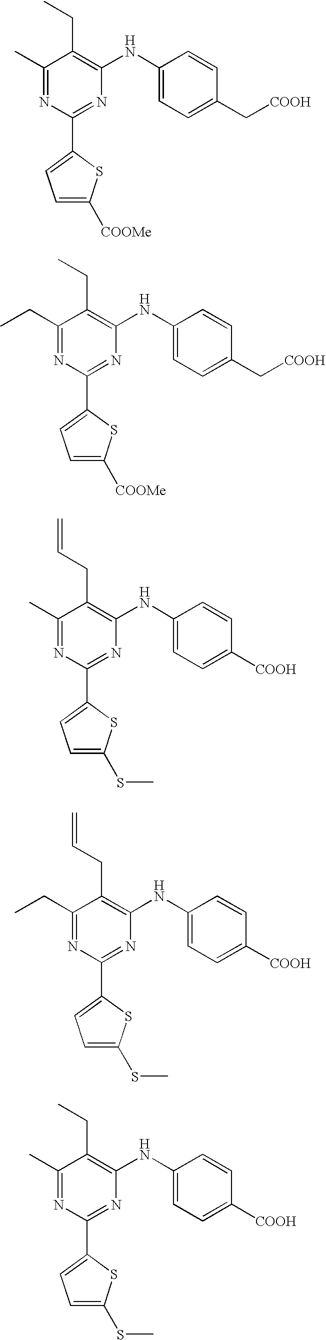

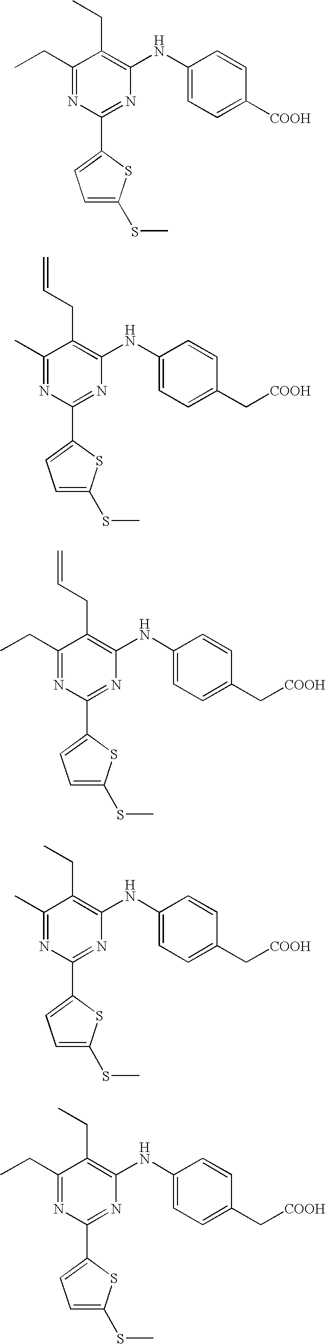

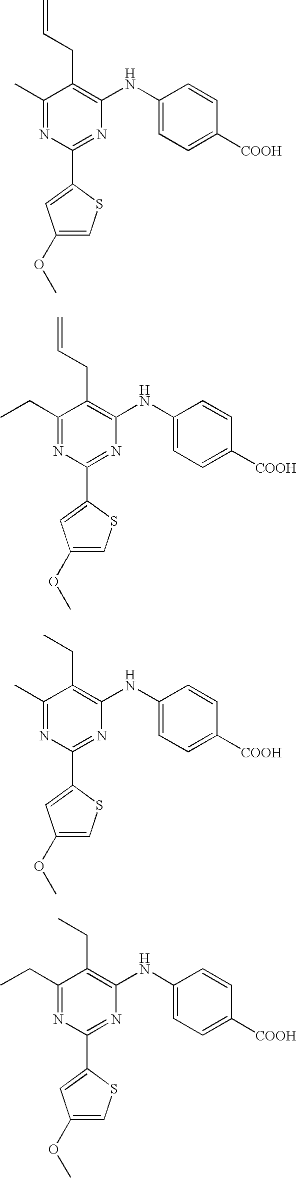

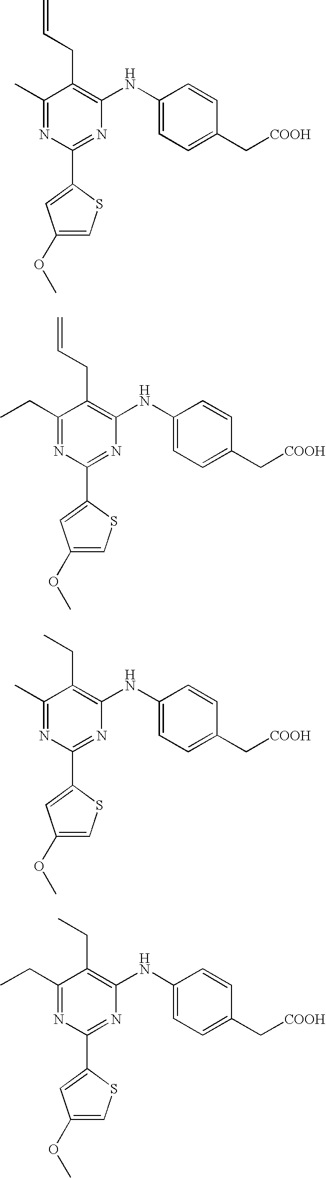

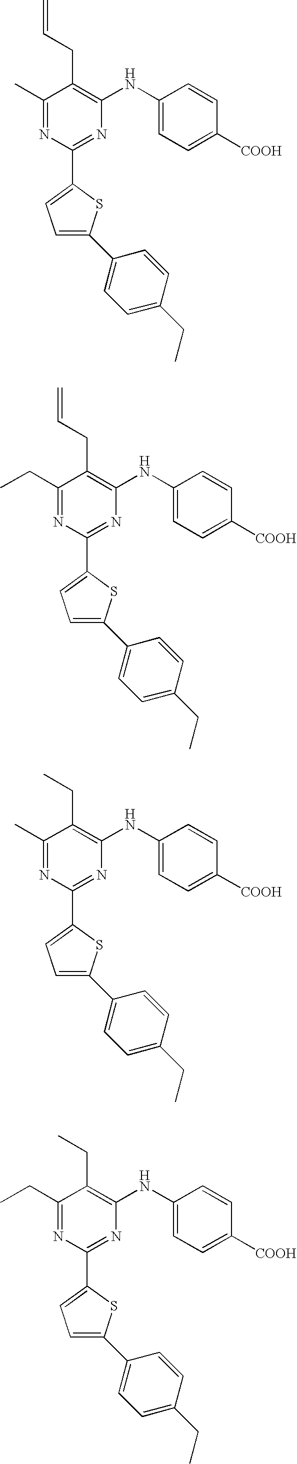

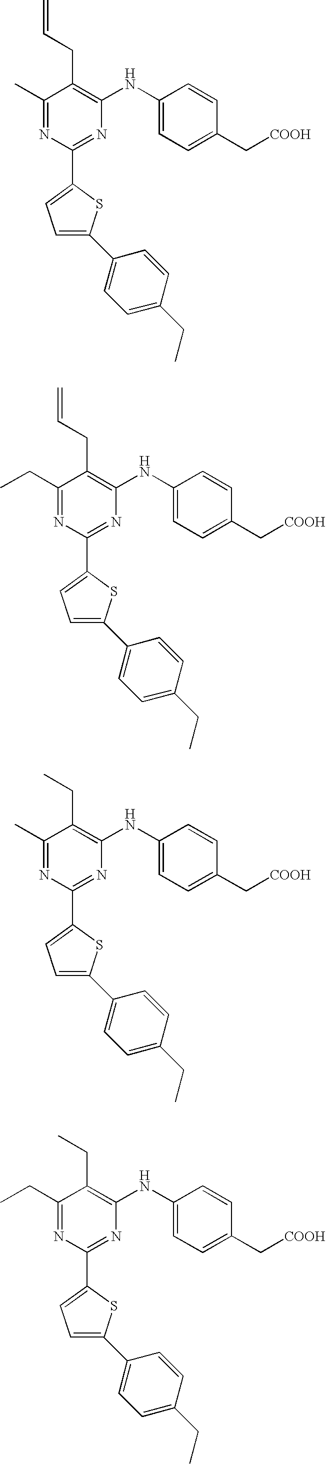

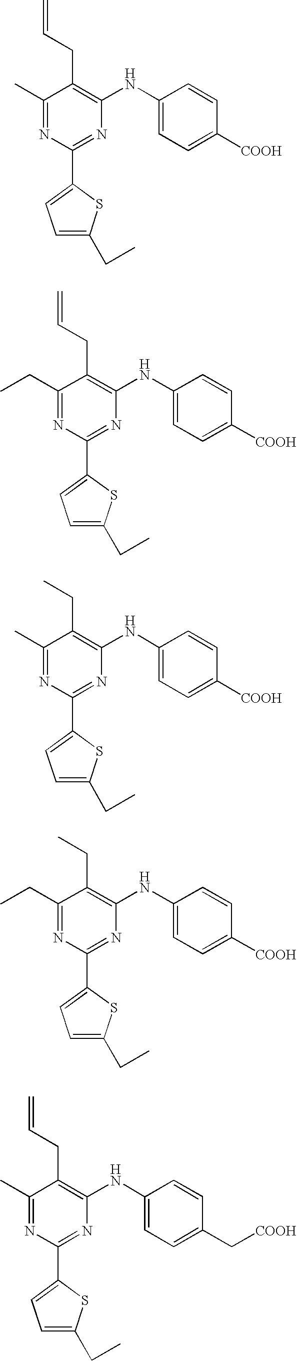

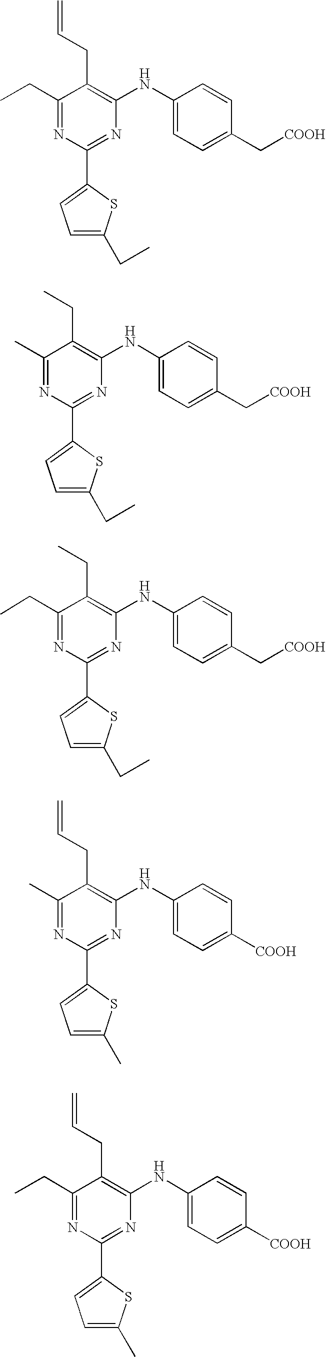

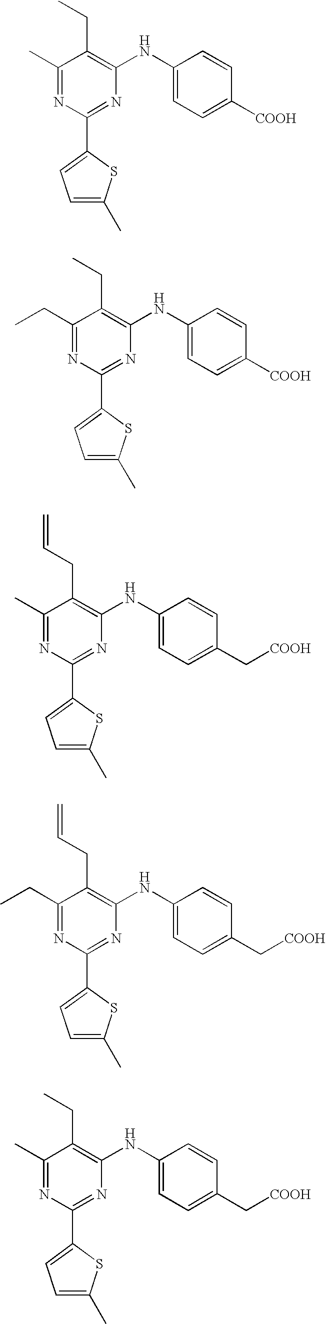

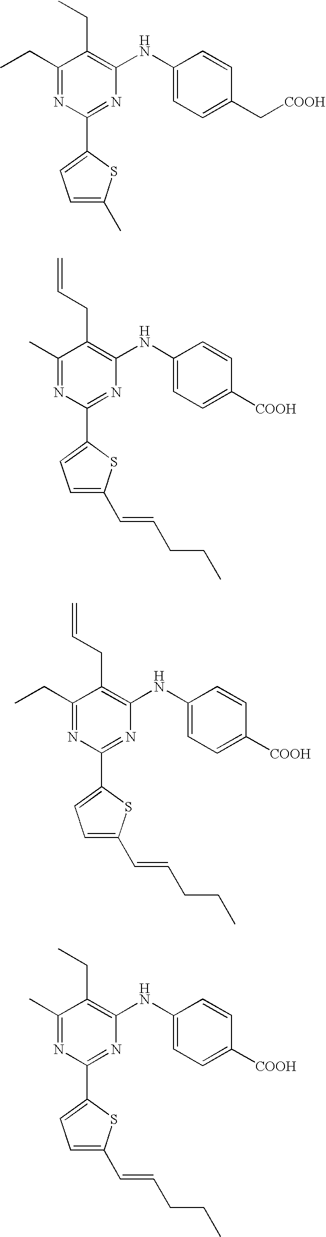

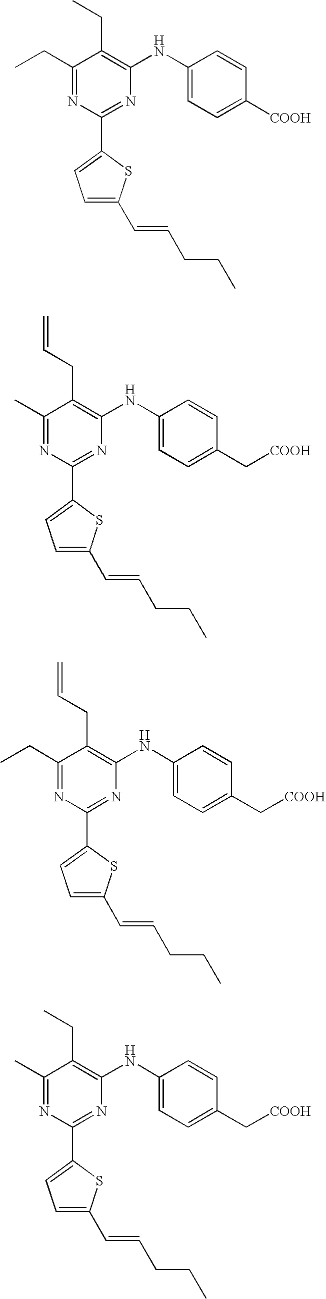

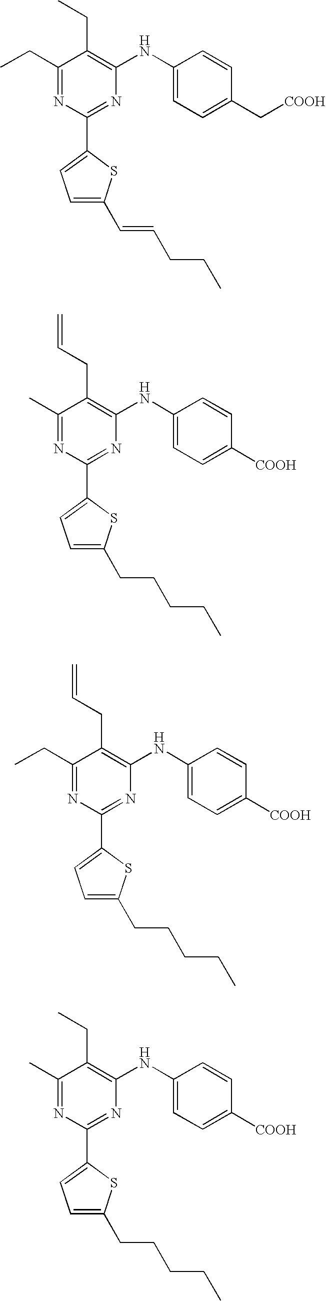

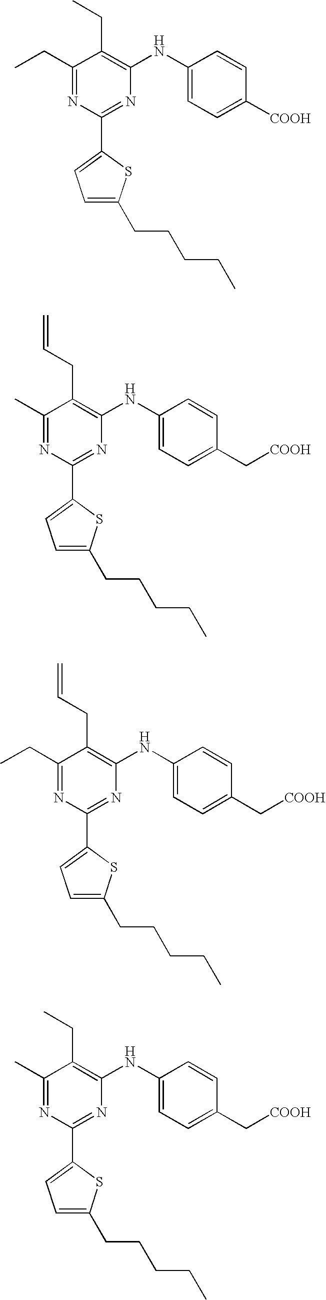

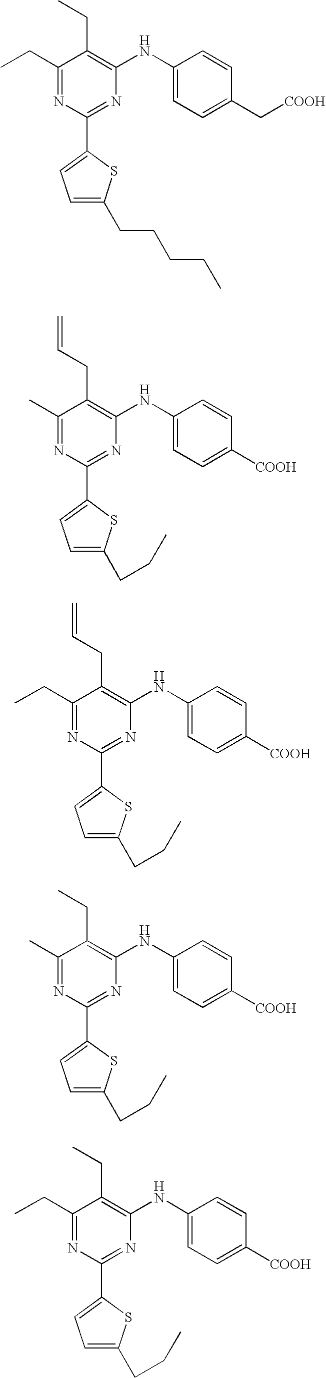

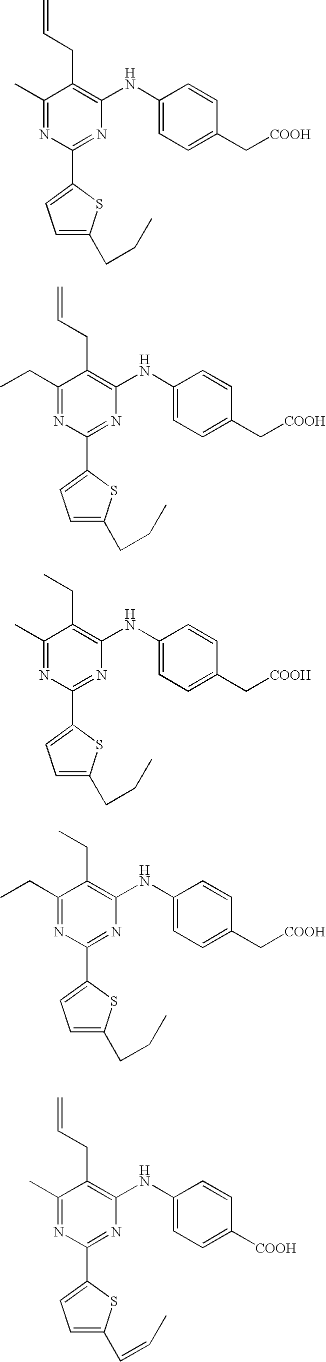

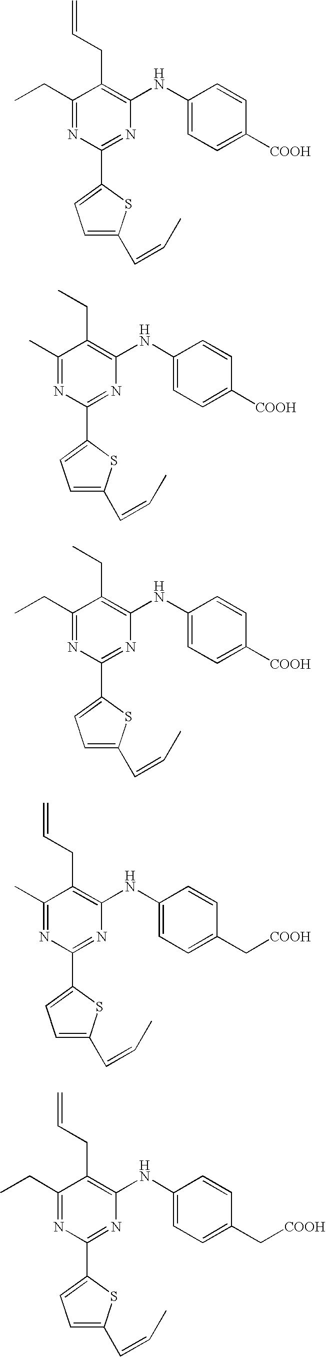

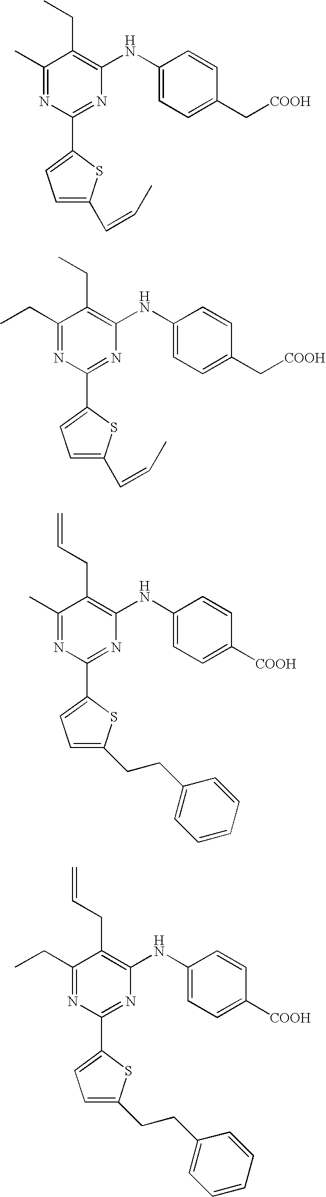

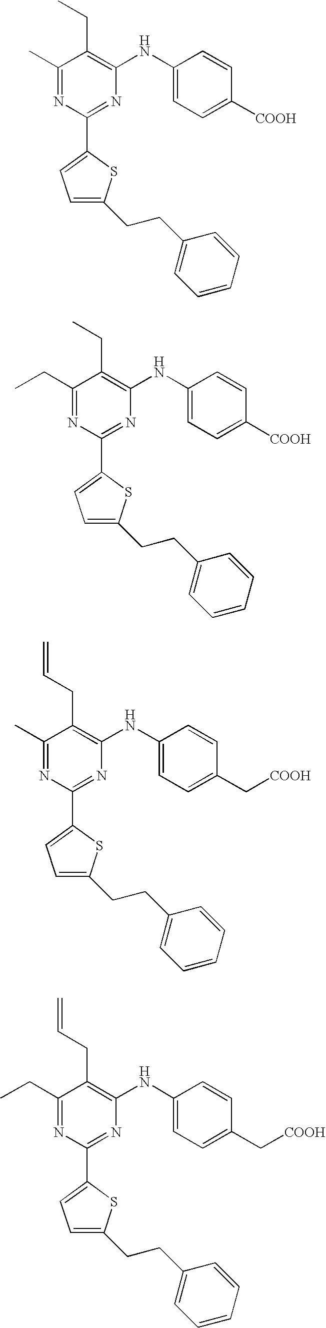

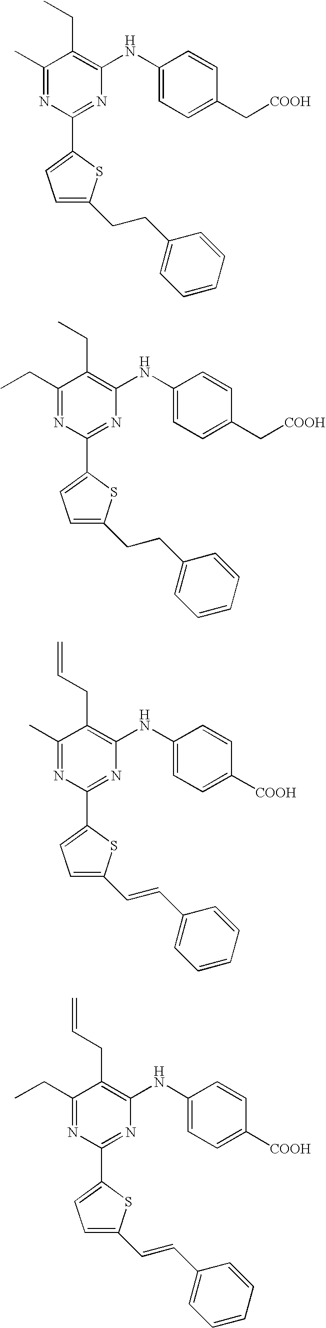

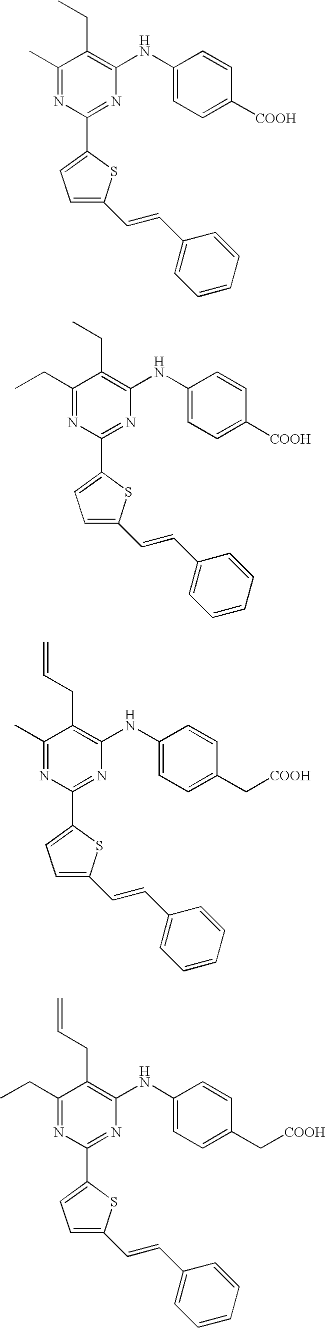

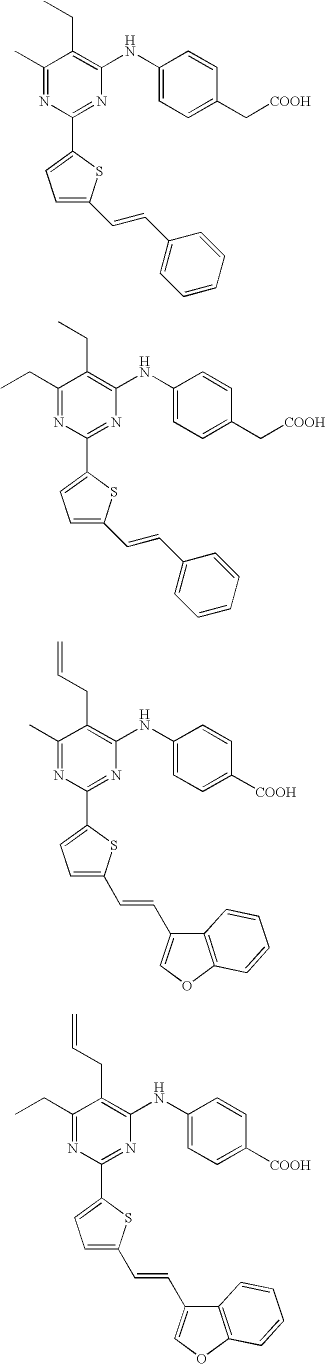

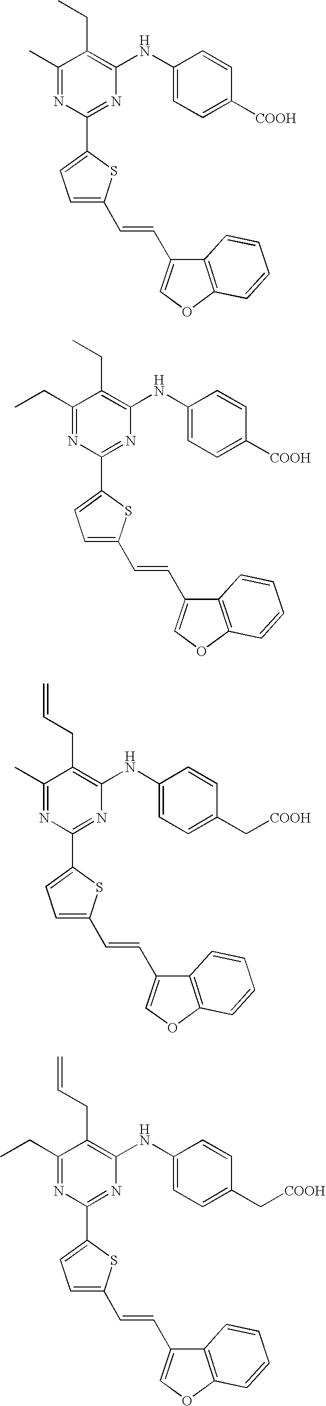

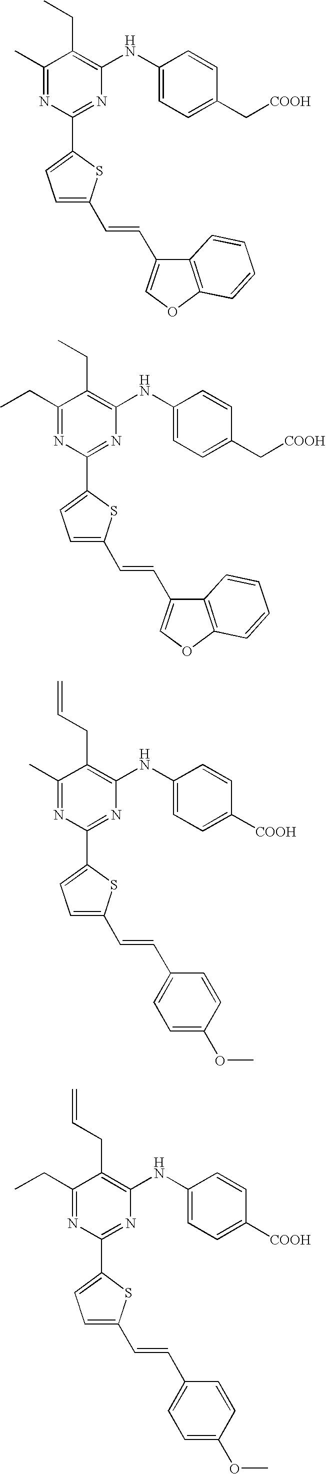

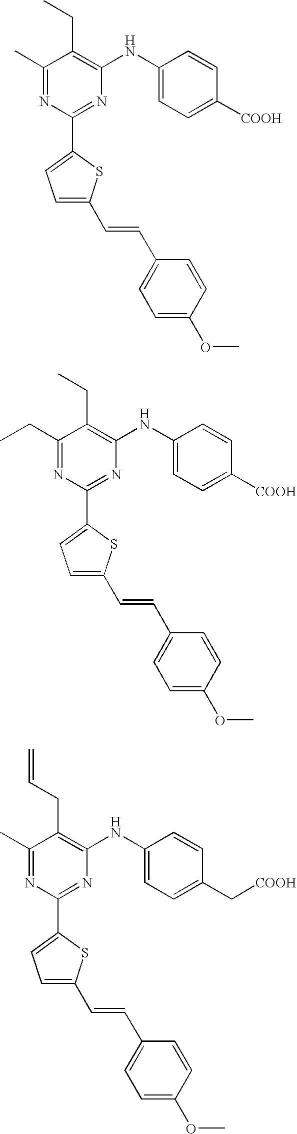

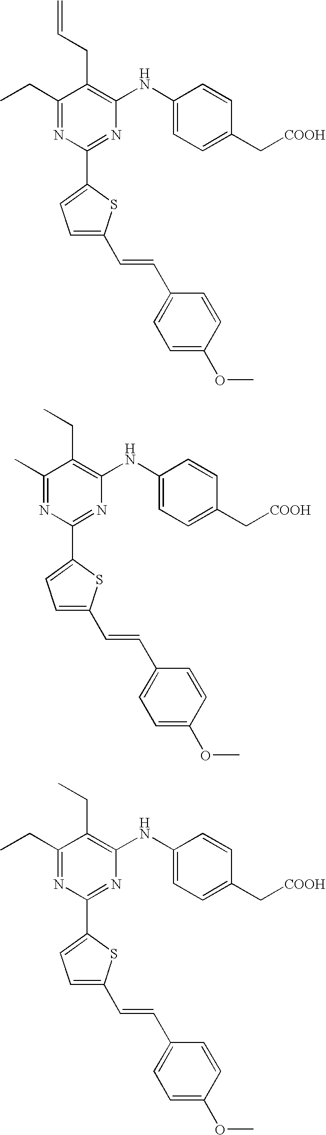

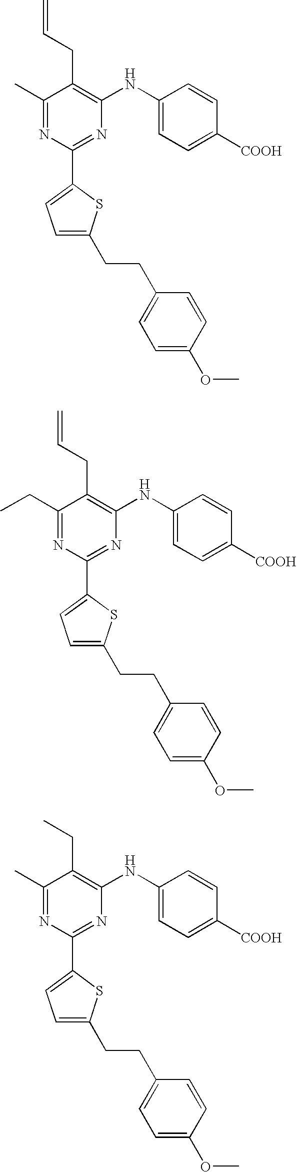

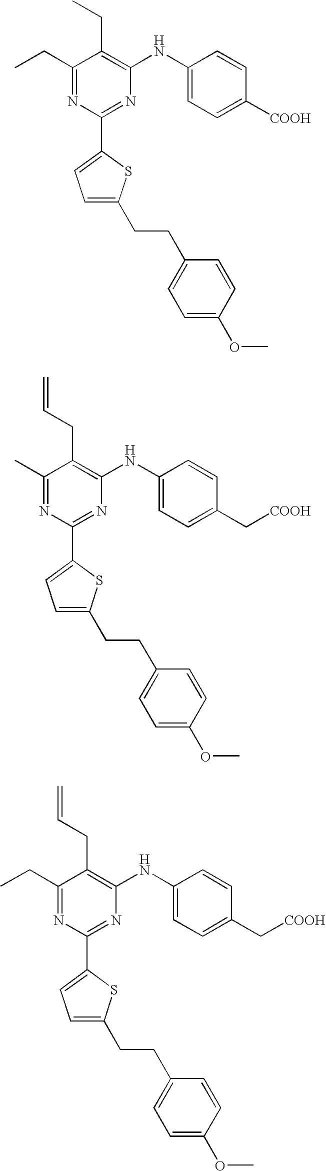

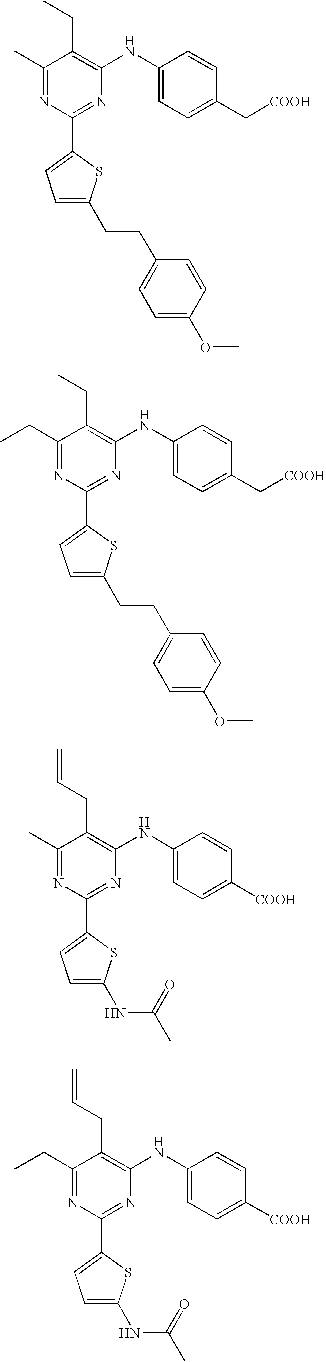

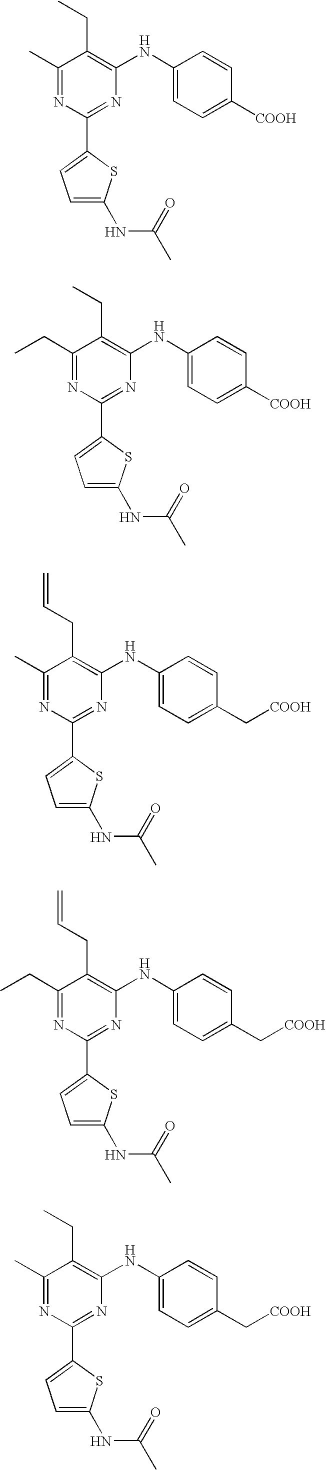

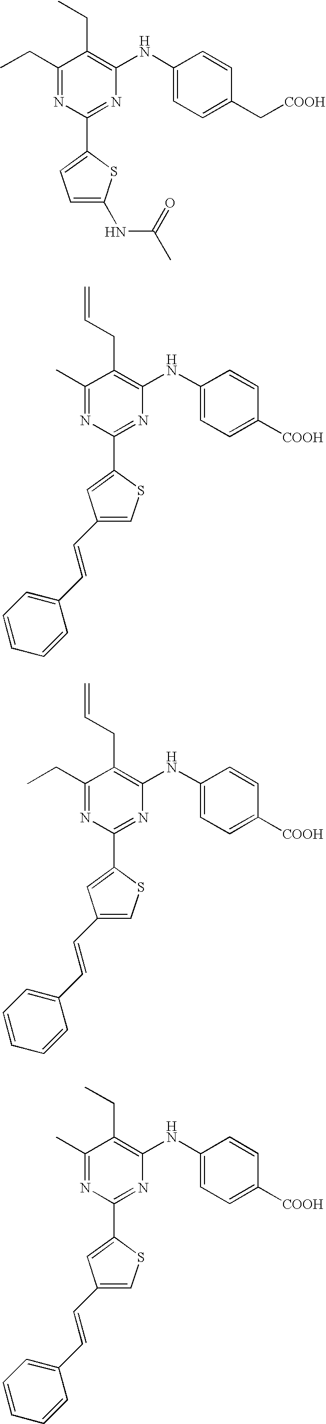

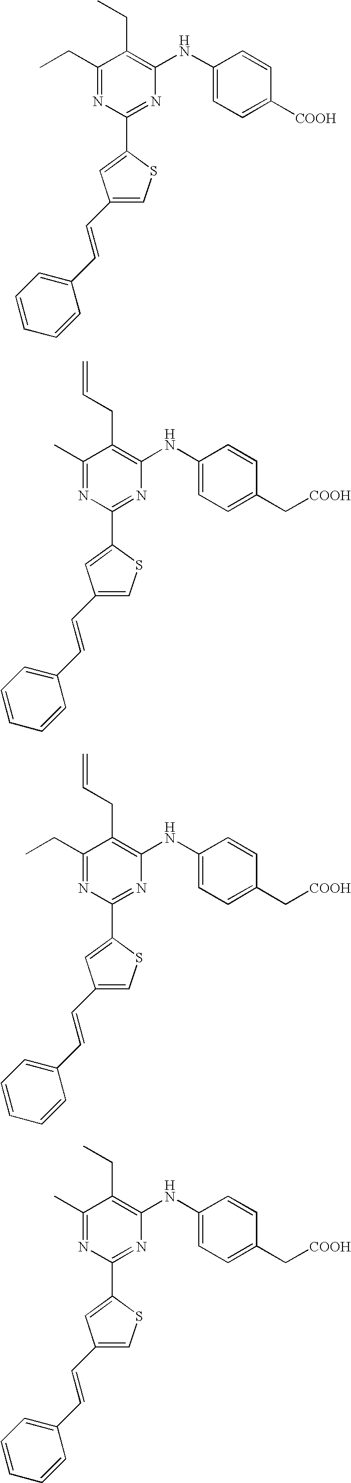

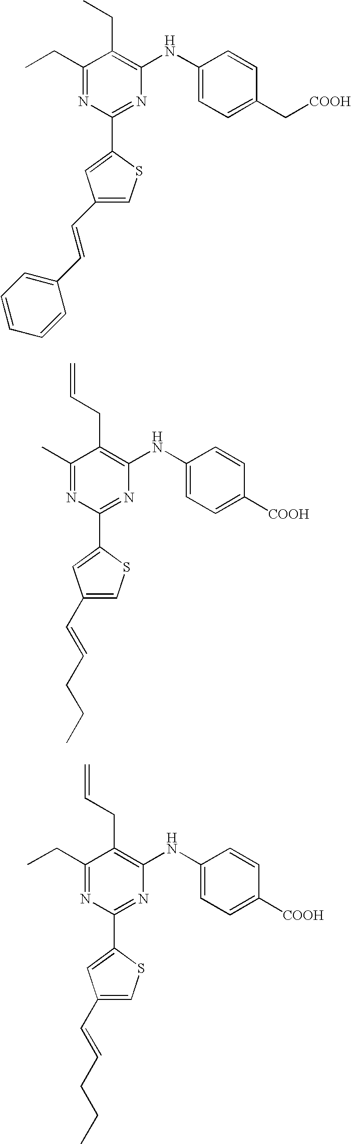

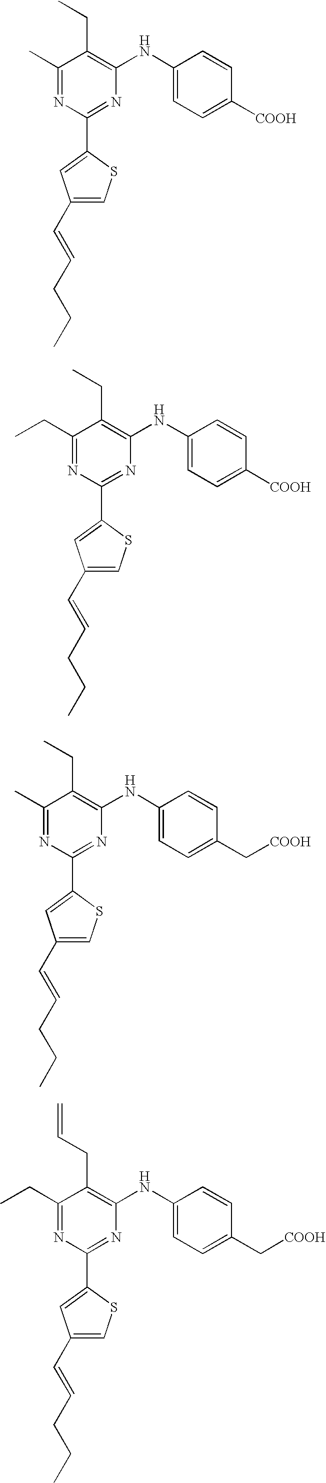

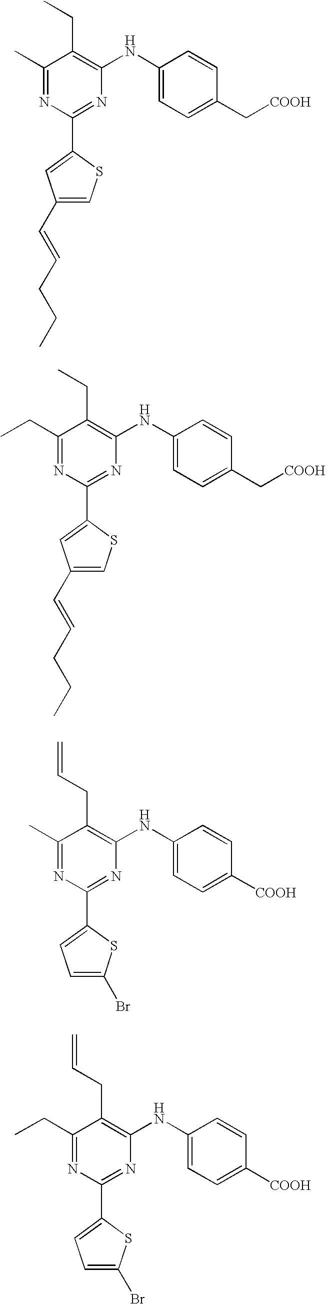

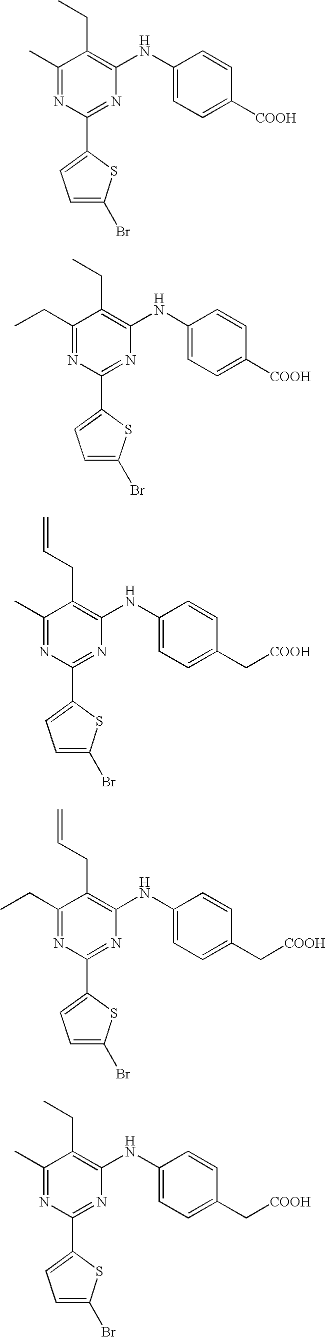

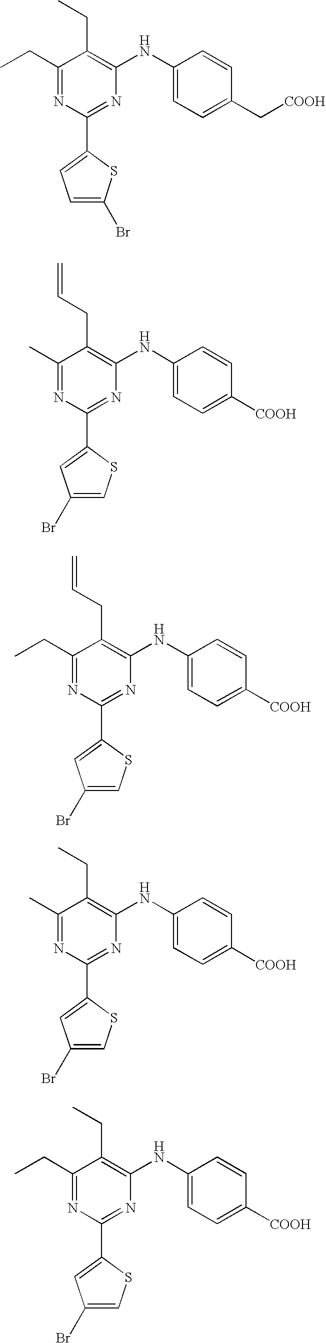

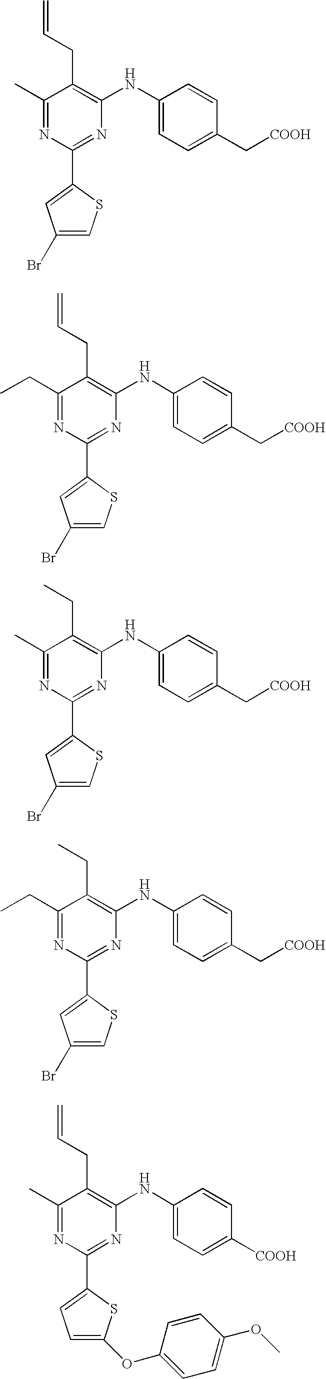

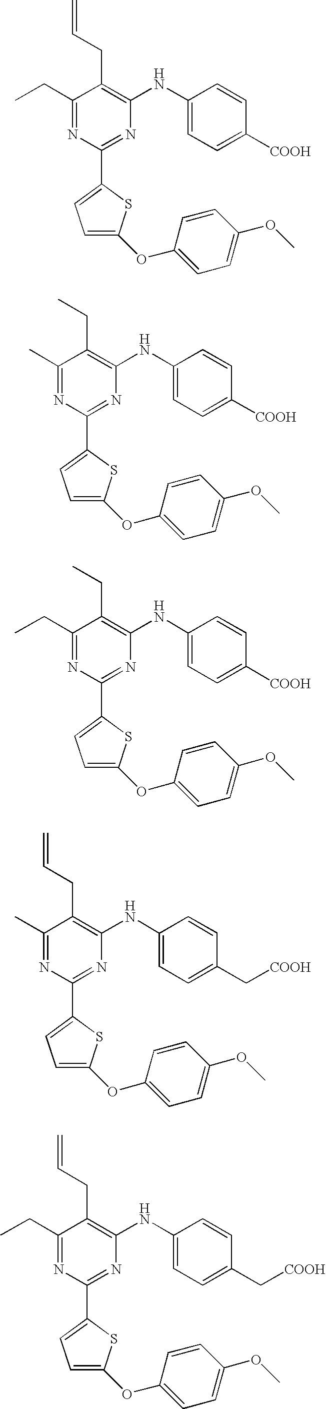

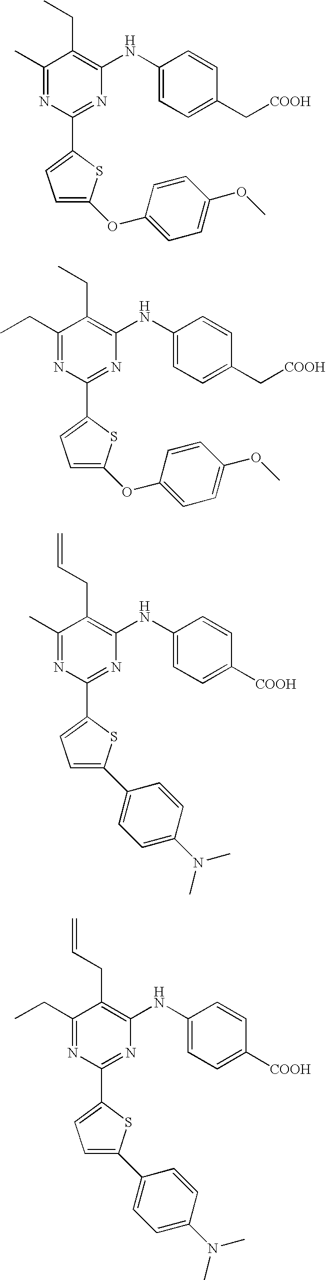

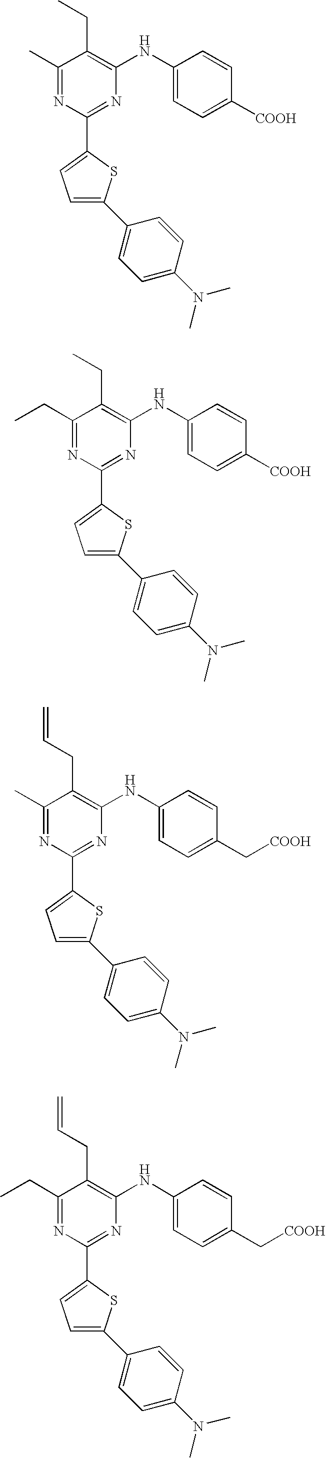

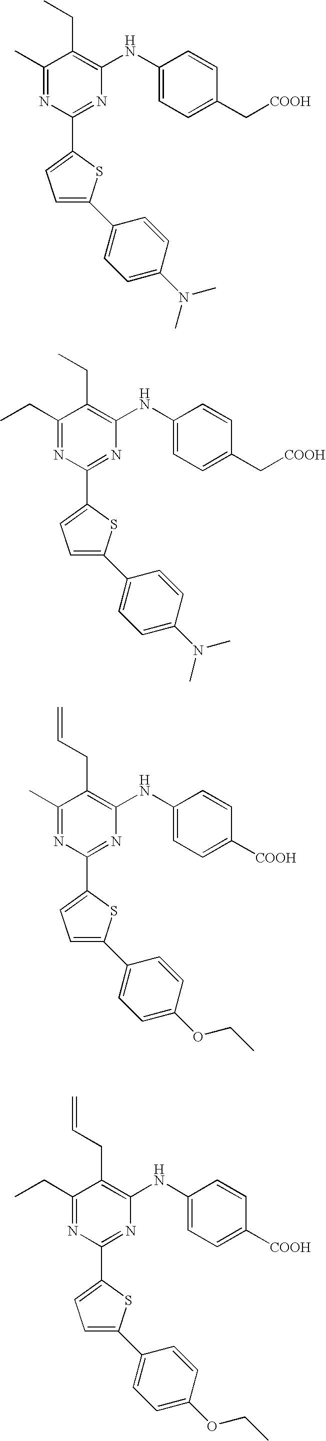

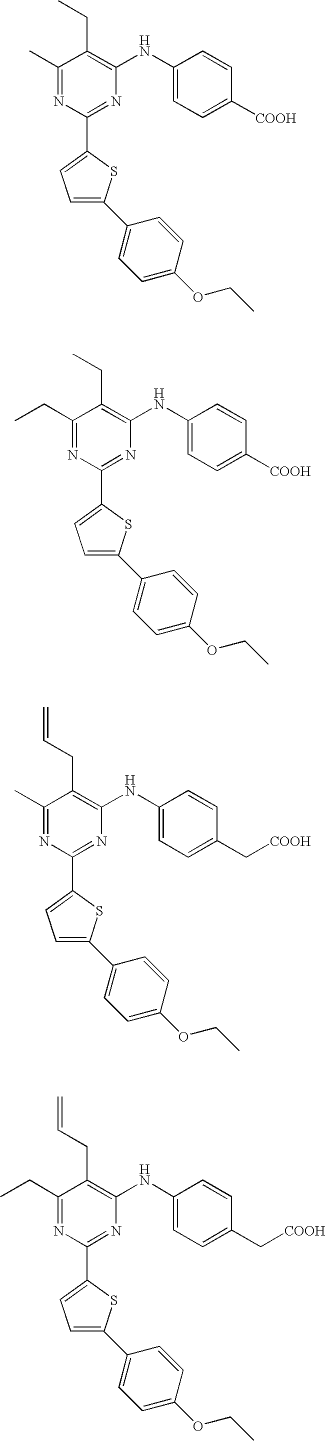

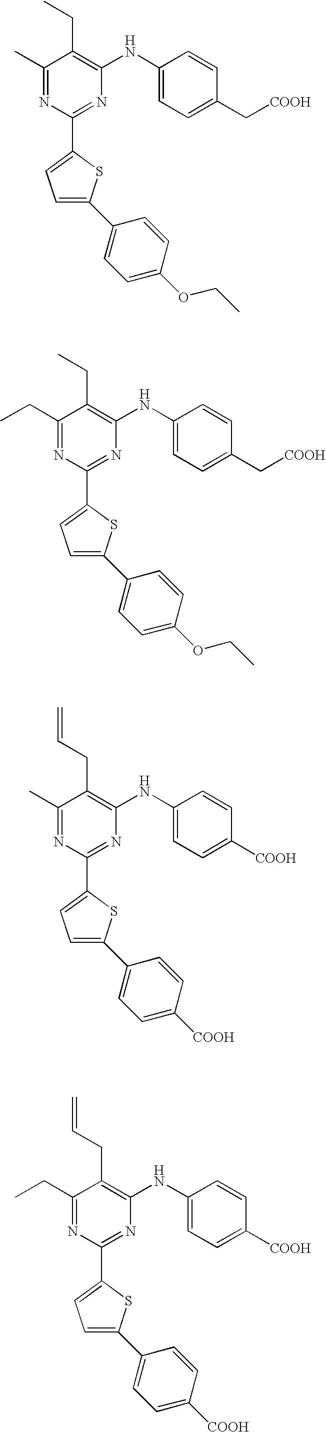

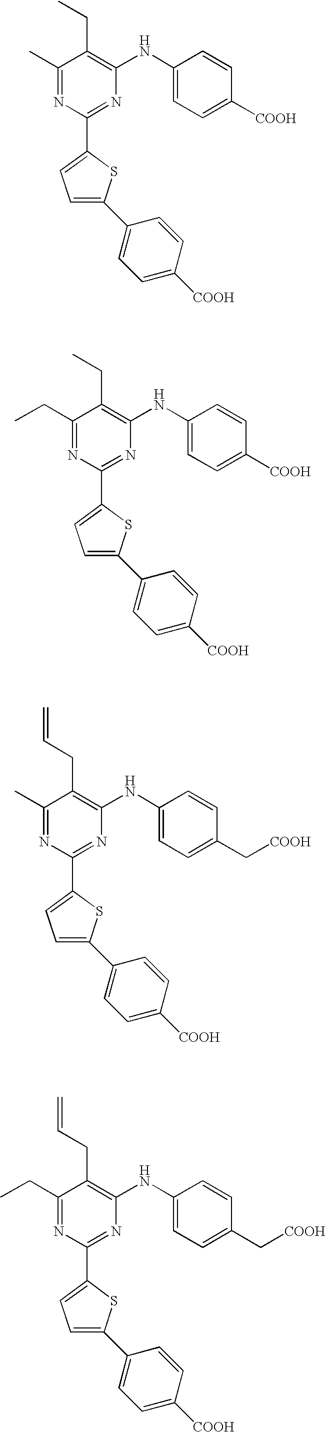

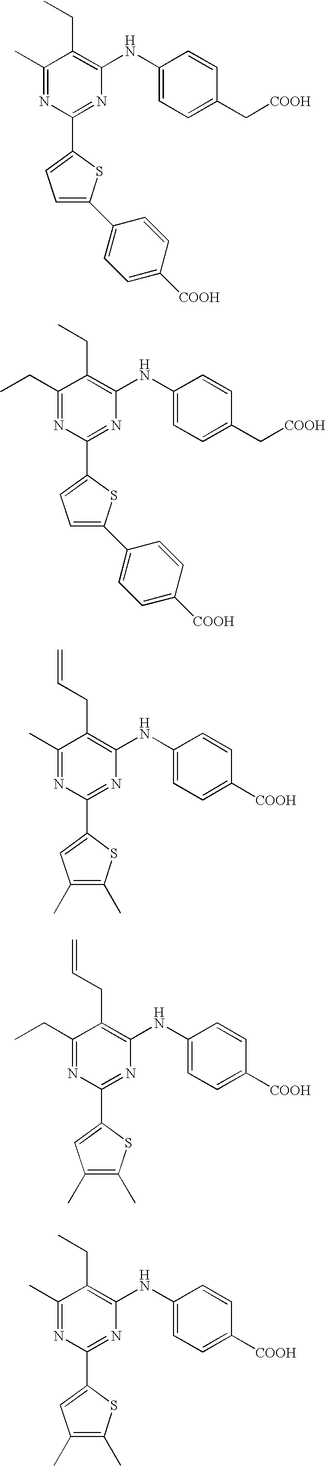

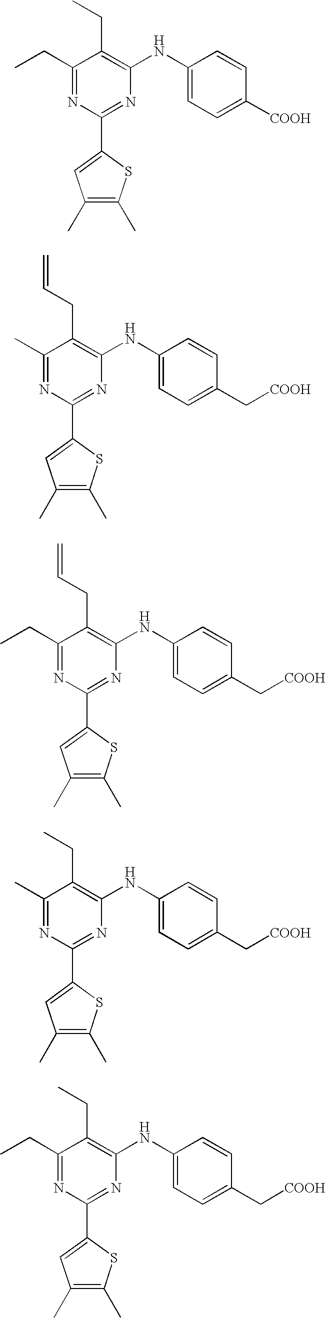

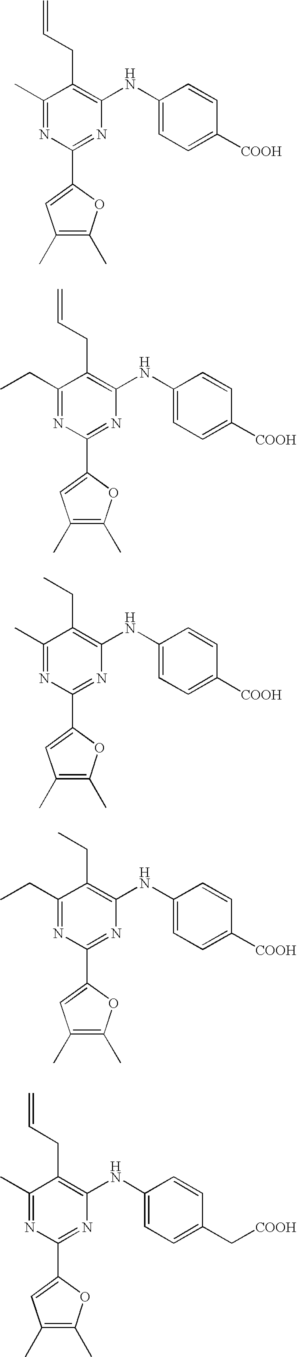

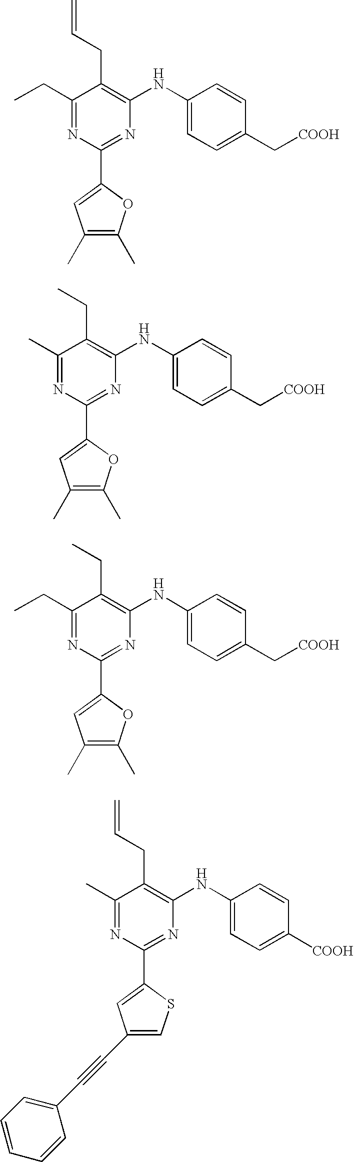

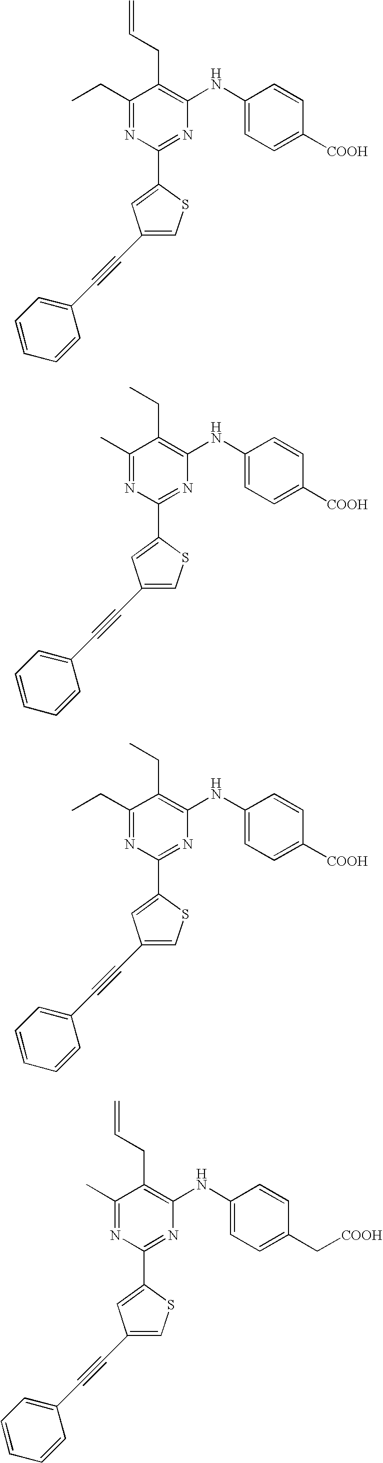

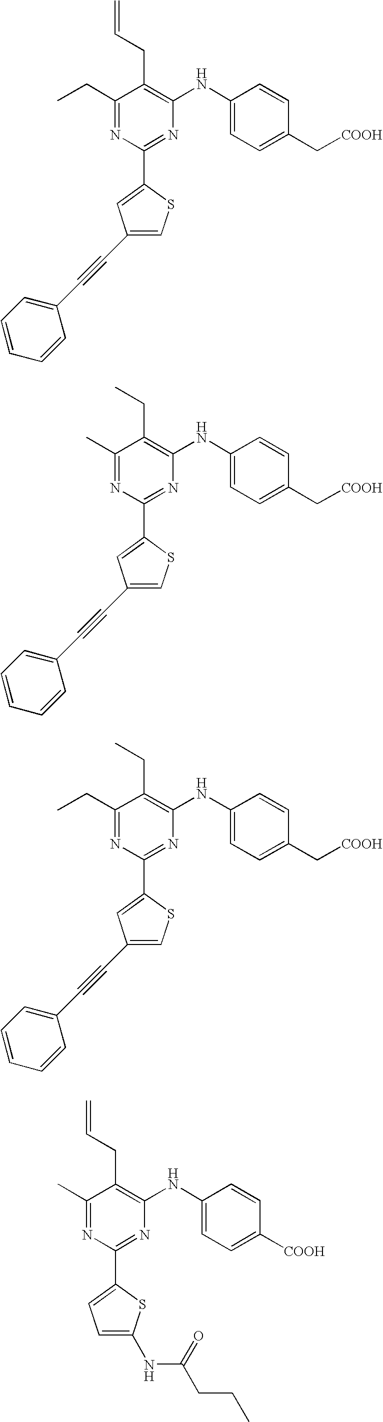

16. A medicine containing the compound according to claim 1, possible stereoisomers thereof or racemates thereof, pharmacologically acceptable salts thereof, hydrates thereof, solvated compounds thereof, or prodrugs thereof as effective ingredients.

17. The medicine according to claim 16 as a preventive and/or therapeutic agent of inflammatory diseases.

18. The medicine according to claim 16 as a preventive and/or therapeutic agent of chronic obstructive lung diseases.

19. A medical composition comprising the compound according to claim 1, possible stereoisomers thereof or racemates thereof, pharmacologically acceptable salts thereof, hydrates thereof, solvated compounds thereof, or prodrugs thereof in an amount effective for prevention and/or therapy, and a pharmacologically acceptable carrier.

20. An inhibitor of PDE4 activity comprising the compound according to claim 1, possible stereoisomers thereof or racemates thereof, pharmacologically acceptable salts thereof, hydrates thereof, solvated compounds thereof, or prodrugs thereof as effective ingredients.

21. A preventive and/or therapeutic method of inflammatory diseases comprising the step of administering, to mammals including a human, the compound according to claim 1, possible stereoisomers thereof or racemates thereof, pharmacologically acceptable salts thereof, hydrates thereof, solvated compounds thereof, or prodrugs thereof in an amount effective for prevention and/or therapy.

22. A method for inhibiting PDE4 activity in the body of mammals including a human comprising the step of administering, to mammals including a human, the compound according to claim 1, possible stereoisomers thereof or racemates thereof, pharmacologically acceptable salts thereof, hydrates thereof, solvated compounds thereof, or prodrugs thereof in an amount effective for prevention and/or therapy.
